(19) United States
(12) Patent Application Publication
(10) Pub. No.: US 2026/0083672 A1
(43) Pub. Date: Mar. 26, 2026

(54) FORMULATIONS AND DOSAGE AMOUNTS FOR ADMINISTERING AN ANTI-DPP3 ANTIBODY OR FRAGMENT THEREOF TO HUMANS

(71) Applicant: 4TEEN4 Pharmaceuticals GmbH, Hennigsdorf (DE)

(72) Inventors: Andreas Bergmann, Berlin (DE); Karine Bourgeois, Berlin (DE)

(73) Assignee: 4TEEN4 Pharmaceuticals GmbH, Hennigsdorf (DE)

(21) Appl. No.: 19/310,528

(22) Filed: Aug. 26, 2025

Related U.S. Application Data
(60) Provisional application No. 63/687,260, filed on Aug. 26, 2024.
(60) Provisional application No. 63/687,255, filed on Aug. 26, 2024.
(60) Provisional application No. 63/687,250, filed on Aug. 26, 2024.
(60) Provisional application No. 63/767,995, filed on Mar. 6, 2025.
(60) Provisional application No. 63/767,967, filed on Mar. 6, 2025.

(51) Int. Cl. A61K 9/08; A61K 9/00; A61K 39/00; A61K 47/20; A61K 47/22; A61K 47/26; C07K 16/40

(52) U.S. Cl. CPC A61K 9/08; A61K 9/0019; A61K 47/20; A61K 47/22; A61K 47/26; C07K 16/40; A61K 2039/505; A61K 2039/545; C07K 2317/24; C07K 2317/76; C07K 2317/92

(57) ABSTRACT

The present inventions are directed to formulations and dosage amounts for the administration of a humanized anti-DPP3 antibody or fragment thereof to humans, including for the treatment of life-threatening circulatory failure conditions, such as shock.

### PRIORITY CLAIM

This application claims priority to U.S. Provisional Patent Application No. 63/767,995 filed on Mar. 6, 2025 and titled “FORMULATIONS AND DOSAGE AMOUNTS FOR ADMINISTERING AN ANTI-DPP3 ANTIBODY OR FRAGMENT THEREOF TO HUMANS,” U.S. Provisional Application No. 63/687,260 filed on Aug. 26, 2024 and titled “FORMULATIONS AND DOSAGE AMOUNTS FOR ADMINISTERING AN ANTI DPP3 ANTIBODY OR FRAGMENT THEREOF TO HUMAN SUBJECTS,” U.S. Provisional Application No. 63/687,255 filed on Aug. 26, 2024 and titled “DPP3 INHIBITOR FOR IMPROVEMENT OF PULMONARY FUNCTION, MYOCARDIAL PROTECTION AND PREVENTION OF MYOCARDIAL INJURY IN CRITICALLY ILL PATIENTS,” U.S. Provisional Patent Application No. 63/767,967 filed on Mar. 6, 2025 and titled “ANTI-DPP3 ANTIBODY DIRECTED TO AND BINDING TO DPP3 FOR THE TREATMENT OF SHOCK IN EXTREME-CRITICALLY ILL PATIENTS,” and U.S. Provisional Application No. 63/687,250 filed on Aug. 26, 2024 and titled “ANTI-DPP3 ANTIBODY DIRECTED TO AND BINDING TO DPP3 FOR THE TREATMENT OF SHOCK IN EXTREME-CRITICALLY ILL PATIENTS, the entire contents of all of which are hereby incorporated by reference herein.

### SEQUENCE LISTING

The instant application contains a Sequence Listing which has been submitted electronically in XML format and is hereby incorporated by reference in its entirety. Said XML copy, created on Aug. 18, 2025, is named T75420US.xml and is 70,993 bytes in size.

### FIELD OF THE INVENTION

The humanized anti-DPP3 antibody according to the present invention is a monoclonal immunoglobulin G1 (IgG1) antibody directed against Dipeptidyl Peptidase 3 (DPP3), also called circulating DPP3 (cDPP3). The humanized anti-DPP3 antibody is in the clinical stage of development and is intended for the treatment of life-threatening circulatory failure conditions, including shock. Among those, cardiogenic shock (CS) is of particular interest. The present inventions are directed to formulations and dosage amounts for the administration of a humanized anti-DPP3 antibody or fragment thereof to humans, including for the treatment of life-threatening circulatory failure conditions, such as shock.

### DESCRIPTION

Shock is characterized by decreased oxygen delivery and/or increased oxygen consumption or inadequate oxygen utilization leading to cellular and tissue hypoxia. It is a life-threatening condition of circulatory failure and most commonly manifested as hypotension (systolic blood pressure less than 90 mm Hg or mean arterial pressure (MAP) less than 65 mmHg). Shock is divided into four main types based on the underlying cause: hypovolemic, obstructive, distributive, and cardiogenic shock (Vincent & De Backer, 2014, N. Engl. J. Med. 370(6): 583).

Hypovolemic shock is characterized by decreased intravascular volume and can be divided into two broad subtypes: hemorrhagic and nonhemorrhagic. Common causes of hemorrhagic hypovolemic shock include gastrointestinal bleed, trauma, vascular etiologies (e.g. ruptured abdominal aortic aneurysm, tumor eroding into a major blood vessel), and spontaneous bleeding in the setting of anticoagulant use. Common causes of nonhemorrhagic hypovolemic shock include vomiting, diarrhea, renal loss, skin losses/insensible losses (e.g. burns, heat stroke), or third-space loss in the setting of pancreatitis, cirrhosis, intestinal obstruction, trauma. For review see Koya and Paul, 2018, Shock, StatPearls [Internet], Treasure Island (FL): StatPearls Publishing; 2019-2018 Oct. 27 (Koya 2018).

Obstructive shock is due to a physical obstruction of the great vessels or the heart itself. Several conditions can result in this form of shock (e.g. cardiac tamponade, tension pneumothorax, pulmonary embolism, aortic stenosis). For review see Koya 2018.

According to the underlying cause, there are four types of distributive shock: neurogenic shock (decreased sympathetic stimulation leading to decreased vasal tone), anaphylactic shock, septic shock, and shock due to adrenal crisis. In addition to sepsis, distributive shock can be caused by systemic inflammatory response syndrome (SIRS) due to conditions other than infection, such as pancreatitis, burns, or trauma. Other causes include, toxic shock syndrome (TSS), anaphylaxis (a sudden, severe allergic reaction), adrenal insufficiency (acute worsening of chronic adrenal insufficiency, destruction or removal of the adrenal glands, suppression of adrenal gland function due to exogenous steroids, hypopituitarism, and metabolic failure of hormone production), reactions to drugs or toxins, heavy metal poisoning, hepatic (liver) insufficiency, and damage to the central nervous system. For review see Koya 2018.

Septic shock, a form of distributive shock, is a potentially fatal medical condition that occurs when sepsis, which is organ injury or damage in response to infection, leads to dangerously low blood pressure and abnormalities in cellular metabolism. The Third International Consensus Definitions for Sepsis and Septic Shock (Sepsis-3) defines septic shock as a subset of sepsis in which particularly profound circulatory, cellular, and metabolic abnormalities are associated with a greater risk of mortality than with sepsis alone. Patients with septic shock can be clinically identified by a vasopressor requirement to maintain a mean arterial pressure of 65 mm Hg or greater and serum lactate level greater than 2 mmol/L (>18 mg/dL) in the absence of hypovolemia. This combination is associated with hospital mortality rates greater than 40% (Singer, M, et al., 2016, JAMA 315 (8): 801-810 (Singer 2016)). The primary infection is most commonly caused by bacteria, but also may be by fungi, viruses, or parasites. It may be located in any part of the body, but most commonly in the lungs, brain, urinary tract, skin, or abdominal organs. It can cause multiple organ dysfunction syndrome (formerly known as multiple organ failure) and death. Frequently, people with septic shock are cared for in intensive care units. It most commonly affects children, immunocompromised individuals, and the elderly, as their immune systems cannot deal with infection as effectively as those of healthy adults. The mortality rate from septic shock is approximately 25-50%.

Cardiogenic shock (CS) is defined as a state of critical end-organ hypoperfusion due to reduced cardiac output. Among the classical shock types, CS is the second most common cause of shock among patients in the ICU (De Backer; D., et al, 2010, N. Engl. J. Med. 362, 779-789). CS is a syndrome of life-threatening peripheral hypoperfusion and organ dysfunction due to primary cardiac dysfunction (Vincent, J. L. & De Backern D., 2013, D. Circulatory shock, N. Engl. J. Med. 369, 1726-1734; Soussi, S. et al., 2023, Intensive Care Med. Exp. 11; Arrigo, M, et al., 2021, Lancet Respir Med. 9, 1192-1202 (Arrigo 2021)). Several underlying cardiac conditions may induce CS, with acute myocardial infarction accounting for approximately 30% of CS while other acute and chronic heart disease accounts for the remaining 70%. (Mebazaa, A., et al., 2018 Intensive Care Med. 44, 760-773 (Mebazaa 2018); Van Diepen, S. et al., 2017, Circulation vol. 136 (Van Diepen 2017); Berg, D. D. et al., 2019, Circ. Cardiovasc. Qual. Outcomes 12, 1-10). CS is accompanied by relevant morbidity and mortality rates of up to 50%. (Aissaoui, N., et al., 2020, European Journal of Heart Failure 22 (4): 664-72. (Aissaoui 2020); Thiele, H. et al., 2023, N. Engl. J Med. 1286-1297 (Thiele 2023)).

Notably, CS forms a spectrum that ranges from mild hypoperfusion to profound shock. Criteria for the diagnosis of CS are: (i) systolic blood pressure, ≤90 mmHg for >30 min or vasopressors required to achieve a blood pressure ≥90 mmHg; (ii) pulmonary congestion or elevated left-ventricular filling pressures; (iii) signs of impaired organ perfusion with at least one of the following criteria: (a) altered mental status; (b) cold, clammy skin; (c) oliguria (<0.5 mL/kg/h or <30 mL/h); (d) increased serum-lactate. (Reynolds, H. R. & Hochman, J. S., 2008, Circulation 117, 686-697 (Reynolds & Hochman 2008)). Acute myocardial infarction (AMI) with subsequent ventricular dysfunction is the most frequent cause of CS accounting for approximately 80% of cases. Mechanical complications such as ventricular septal (4%) or free wall rupture (2%), and acute severe mitral regurgitation (7%) are less frequent causes of CS after AMI. (Hochman et al., 2000, J Am Coll Cardiol 36: 1063-1070 (Hochman 2000)). Non-AMI-related CS may be caused by decompensated valvular heart disease, acute myocarditis, arrhythmias, etc. with heterogeneous treatment options.

CS represents a huge unmet medical need as it is accompanied by relevant morbidity and high mortality rates. Furthermore, limited CS management strategies are available, and even fewer lead to mortality reduction. Around 150,000 patients in the US, and 250,000 in Europe suffer from CS per year. The prognosis of CS, despite best standard of care (SOC), remains very poor, with up to 50% 30-day mortality (Muzafarova, T., et al., 2023, Deutsches Arzteblatt International 120 (31-32): 538), and no effective, causal treatment options available (Arrigo, M, et al., 2023, European Journal of Heart Failure 25 (5): 609-15. Arrigo 2021). The current SOC for CS is largely supportive, focused on providing hemodynamic support (Thiele, H., et al., 2012, New England Journal of Medicine 367 (14): 1287-96; Van Diepen 2017; Aissaoui 2020). Current CS management encompasses a mix of pharmacological and mechanical interventions including implantation of mechanical circulatory support devices (MCS) and organ replacement therapies for secondary organ failure (such as dialysis and mechanical ventilation) (Chioncel, O., et al., 2020, Epidemiology, European Journal of Heart Failure 22 (8): 1315-41 (Chioncel 2020)). However, even with optimal treatment following best practice guidelines, many CS patients still do not survive or suffer significant complications and functional limitations (Van Diepen 2017), as none of the currently available interventions, neither pharmaceutical nor mechanical, address the underlying pathophysiological pathways leading to CS.

CS management is largely based on experience rather than evidence-based recommendations as few adequately designed randomized clinical trials to guide treatment exist. (Chioncel 2020; Arrigo, M & Mebazaa, A., 2015, Intensive Care Med. 41, 912-915). Best management of CS is usually accomplished in tertiary cardiogenic shock centers and encompasses revascularization strategies, treatment with inotropes and vasopressors, implantation of mechanical circulatory support, and intensive care support for other organs (mostly lungs and kidneys). So far, revascularization is the only evidence-based therapy with proven survival benefit. (Thiele, H., et al., 2015, Eur Heart J. 36, 1223-1230; Thiele, H. et al., 2017, N. Engl. J. Med. 377, 2419-2432; Hochman, J. S., et al., 2006, JAMA—J. Am. Med. Assoc. 295, 2511-2515; Hochman, J. S., et al., 1999, N. Engl. J. Med. 341, 625-634). Active mechanical circulatory support use, in particular veno-arterial extracorporeal membrane oxygenation, coined extracorporeal life support (ECLS), has increased by a factor of 10 during the past decade despite the lack of evidence for its benefit. (Becher P M, et al., 2018, Circulation 138, 2298-2300). Recent results from a large randomized clinical trial, ECLS-shock, have shown that ECLS does not reduce mortality in acute myocardial infarction complicated by CS. (Thiele 2023). These results suggest that the benefits associated with the hemodynamic support offered by ECLS may be outweighed by device-associated complications (bleeding, stroke, ischemia and hemolysis) and deleterious inflammatory response. (Mebazaa 2018; Thiele 2023).

The role of inflammation in shock has been studied extensively in CS. (Diakos, N. A. et al., 2021, J. Cardiovasc. Transl. Res. 14, 476-483; Montero, S. & Bayes-Genis, A., 2022, Eur J. Prev. Cardiol. 29, 2052-2054; Cuinet, J., et al., 2020, Sci. Rep. 10, 1-9; Jentzer; J. C., et al., 2020, Circ. Cardiovasc. Qual. Outcomes 13, E006956). It is known that the vicious circle of myocardial ischemia, left ventricular dysfunction, and systemic hypoperfusion leads to systemic inflammation and, ultimately, to derangements in the entire circulatory system. (Thiele, H., et al., 2010, Eur Heart J. 31, 1828-1835). This systemic inflammatory response, initiated by the release of damage-associated molecular patterns (DAMPs), leads to the release of cytokines and mediators exerting direct or indirect toxic effects on the vascular endothelium and the myocardium. As an example of these toxic mediators, nitric oxide has been shown to directly inhibit myocardial contractility, disturb mitochondrial respiration, induce vasodilation, and reduce catecholamine response. (Hochman, J. S., 2003, Circulation 107, 2998-3002).

Finally, the renin-angiotensin-aldosterone system (RAAS) (also referred to as the renin-angiotensin system (RAS)) is also disordered in patients with shock and mostly studied in septic shock. (Chawla, L. S., et al., 2014, Crit. Care 18, 1-9; Zhang, W, et al., 2014, Exp. Ther Med. 7, 1342-1348; Khanna, A., et al., 2017, N. Engl. J. Med. 377, 419-430). Angiotensin II (Ang II) is the main effector of the RAS. The RAS is activated in cardiovascular diseases (Dostal, D. E., et al., 1997, J of Molecular and Cellular Cardiology 29 (11): 2893-2902; Roks, A, et al., 1997, Heart and Vessels, 119-24), sepsis, and septic shock (Correa, T D., et al., 2015, Critical Care 19 (1): 1-6. (Correa 2015)). Ang II, in particular, has been shown to modulate many cardiovascular functions including the control of blood pressure and cardiac remodeling. In CS, it is hypothesized that reduced cardiac function triggers neurohumoral activation (of both adrenergic and RAAS systems) aimed at restoring hemodynamic stability. (Geevarghese, M, et al., 2023, Front. Physiol. 14, 1-9). This partially adaptive response in supporting vital organs is eventually impaired during the downward spiral of CS. Disturbances in the RAAS have been presumed to be due to inadequate activation of the Ang II type-I receptor (ATR1) in some shock patients. Such inadequate activation could be due to a blockade of the receptor of the ATR1, decreased Ang II formation or increased Ang II degradation. (Kono, T., et al., 1979, Endocrinol. Jpn. 26, 411-418; Goldberg, M R., et al., 1993, Hypertension 21, 704-713; Reynolds & Hochman 2008). RAAS imbalance during CS could lead to further deterioration of the cardiovascular system and kidneys. Further studies are needed to decipher the interplay between RAAS and hemodynamic instability in CS.

Taking into consideration the high mortality rates and the neutral trials in CS (IABP-SHOCK II (Thiele, H., et al., 2012, Am. Heart J. 163, 938-945), percutaneous left ventricular assist devices (Thiele, H., et al., 2017, Eur Heart J. 38, 3523-3531), veno-arterial extracorporeal membrane oxygenation in ECMO-CS³⁹ and the most recent ECLS-SHOCK trial (Thiele 2023), it is clear the CS as a syndrome represents a huge unmet medical need. Randomized trials and observational studies have shown that mechanical circulatory support alone is not sufficient to improve prognosis in CS. Thorough understanding of CS pathobiology should spearhead the identification of new therapeutic targets and finally enable a more personalized and pathophysiologically-tailored approach to CS treatment.

All forms of shock can become refractory, as evidenced by unresponsiveness to high-dose vasopressors. (Udupa & Shetty, 2018, Indian J Respir Care 7: 67-72 (Udupa & Shetty 2018)). In general, refractory shock has been defined as requirement of noradrenaline infusion of >0.5 μg/kg/min despite adequate volume resuscitation. Mortality in these patients may be as high as 94% and the assessment and management of these patients requires a much more aggressive approach for survival. The term “refractory shock” is used when the tissue perfusion cannot be restored with the initial corrective measures employed (e.g., vasopressors) and may therefore be referred to as “high vasopressor-dependent” or “vasopressor-resistant” shock. (Udupa & Shetty 2018). Patients with refractory shock have features of inadequate perfusion such as hypotension (mean arterial blood pressure <65 mmHg), tachycardia, cold peripheries, prolonged capillary refill time, and tachypnea consequent to hypoxia and acidosis. Fever may be seen in septic shock. Patients with refractory shock may have other signs of hypoperfusion such as altered sensorium, hyperlactatemia, and oliguria. Different types of shock may coexist in a patient.

Dipeptidyl peptidase 3—also known as Dipeptidyl aminopeptidase III, Dipeptidyl arylamidase III, Dipeptidyl peptidase III, Enkephalinase B or red cell angiotensinase; short name: DPP3, DPPIII—is a metallopeptidase that removes dipeptides from physiologically active peptides, such as enkephalins and angiotensins. DPP3 was identified and its activity measured in extracts of purified bovine anterior pituitary. (Ellis, S. & Nuenke, J M, 1967, J of Biological Chemistry 242 (20): 4623-29 (Ellis & Nuenke 1967)). The enzyme, which is listed as EC 3.4.14.4, has a molecular mass of about 83 kDa and is highly conserved in procaryotes and eucaryotes. (Prajapati, S. C. & Chauhan, S. S., 2011, FEBS Journal 278 (18): 3256-76. (Prajapati & Chauhan 2011)). The amino acid sequence of the human variant is depicted in SEQ ID NO: 1. DPP3 is a mainly cytosolic peptidase which is ubiquitously expressed. Despite lacking a signal sequence, a few studies reported membranous activity. (Lee, C. M & Snyder; S. H., 1982, J. Biol. Chem. 257, 12043-12050 (Lee & Snyder 1982)).

DPP3 is a zinc-depending exo-peptidase belonging to the peptidase family M49. It has a broad substrate specificity for oligopeptides from three/four to ten amino acids of various compositions and is also capable of cleaving after proline. DPP3 is known to hydrolyze dipeptides from the N-terminus of its substrates, including angiotensin II, III, and IV; Leu- and Met-enkephalin; endomorphin 1 and 2. The metallopeptidase DPP3 has its activity optimum at pH 8.0-9.0 and can be activated by addition of divalent metal ions, such as Co²⁺ and Mg²⁺.

Structural analysis of DPP3 revealed the catalytic motifs HELLGH (human DPP3 [hDPP3]450-455; SEQ ID No. 14) and EECRAE (hDPP3 507-512; SEQ ID No. 15), as well as following amino acids, that are important for substrate binding and hydrolysis: Glu316, Tyr, 318, Asp366, Asn391, Asn394, His568, Arg572, Arg577, Lys666, and Arg669 (Prajapati & Chauhan 2011; Kumar; P, et al., 2016, Scientific Reports 6 (December 2015): 1-10.); numbering refers to the sequence of human DPP3, see SEQ ID NO: 1). Considering all known amino acids or sequence regions that are involved in substrate binding and hydrolysis, the active site of human DPP3 can be defined as the area between amino acids 316 and 669.

DPP3 is mainly a soluble, cytosolic peptidase which is ubiquitously expressed. Despite lacking a signal sequence, a few studies reported membranous activity (Lee & Snyder 1982). Extracellularly, DPP3 (i.e., circulating DPP3 (cDPP3)) is found in various extracellular compartments including cerebrospinal fluid, seminal plasma, and retroplacental plasma. (Cruz-Diaz, N., et al., 2016, Peptides 83, 29-37; Shimamori, Y, et al., 1986, Chem. Pharm. Bull. (Tokyo), 34, 3333-3340; Vanha-Perttula, T., 1988, Clin. Chim. Acta 177, 179-195; Sato, Hiroshi, et al., 2003, Masui 52, 257-63). It has been detected in plasma of healthy adults at a median concentration of 14 ng/mL and upper normal range of 40 ng/mL, as defined by the 97.5th percentile. (Rehfeld, L., et al., 2019, J. Appl. Lab. Med. 3, 943-953 (Rehfeld 2019)).

### Level of DPP3

Recently, two assays were generated, characterized, and validated to specifically detect DPP3 in human bodily fluids (e.g., blood, plasma, serum): a luminescence immunoassay (LIA) to detect DPP3 protein concentration and an enzyme capture activity assay (ECA) to detect specific DPP3 activity. (Rehfeld 2019). A washing step removes all interfering substances before the actual detection of DPP3 activity is performed. Both methods are highly specific and allow the reproducible detection of DPP3 in blood samples.

The level of DPP3 as amount of DPP3 protein and/or DPP3 activity in a sample of bodily fluid of said subject may be determined for example by one of the following methods:

Luminescence immunoassay for the quantification of DPP3 protein concentrations (LIA) (Rehfeld 2019). The LIA is a one-step chemiluminescence sandwich immunoassay that uses white high-binding polystyrene microtiter plates as solid phase. These plates are coated with monoclonal anti-DPP3 antibody AK2555 (capture antibody). (See, e.g., U.S. Pat. No. 11,726,094, Example 4). The tracer anti-DPP3 antibody AK2553 is labeled with MA70-acridinium-NHS-ester and used at a concentration of 20 ng per well. (See, e.g., U.S. Pat. No. 11,726,094, Example 4). Twenty microliters of samples (e.g., serum, heparin-plasma, citrate-plasma or EDTA-plasma derived from patients' blood) and calibrators are pipetted into coated white microtiter plates. After adding the tracer antibody AK2553, the microtiter plates are incubated for 3 h at room temperature and 600 rpm. Unbound tracer is then removed by 4 washing steps (350 μL per well). Remaining chemiluminescence is measured for is per well by using a microtiter plate luminometer. The concentration of DPP3 is determined with a 6-point calibration curve. Calibrators and samples are preferably run in duplicate.

Enzyme capture activity assay for the quantification of DPP3 activity (ECA) (Rehfeld 2019). The ECA is a DPP3-specific activity assay that uses black high-binding polystyrene microtiter plates as solid phase. These plates are coated with monoclonal anti-DPP3 antibody AK2555 (capture antibody). Twenty microliters of samples (e.g., serum, heparin-plasma, citrate-plasma, EDTA-plasma, cerebrospinal fluid and urine) and calibrators are pipetted into coated black microtiter plates. After adding assay buffer (200 μL), the microtiter plates are incubated for 2 h at 22° C. and 600 rpm. DPP3 present in the samples is immobilized by binding to the capture antibody. Unbound sample components are removed by 4 washing steps (350 μL per well). The specific activity of immobilized DPP3 is measured by the addition of the fluorogenic substrate, Arg-Arg-β-Naphthylamide (Arg2-DNA), in reaction buffer followed by incubation at 37° C. for 1 h. DPP3 specifically cleaves Arg2-DNA into Arg-Arg dipeptide and fluorescent β-naphthylamine. Fluorescence is measured with a fluorometer using an excitation wavelength of 340 nm and emission is detected at 410 nm. The activity of DPP3 is determined with a 6-point calibration curve. Calibrators and samples are preferably run in duplicates.

Liquid-phase assay for the quantification of DPP3 activity using the Liquid Activity Assay (LAA) (see Jones, TH. D. & Kapralou, A., 1982, Analytical Biochemistry 119 (2): 418-23 (Jones & Kapralou 1982)). The LAA is a liquid phase assay that uses black non-binding polystyrene microtiter plates to measure DPP3 activity. 20 μl of samples (e.g., serum, heparin-plasma, citrate-plasma) and calibrators are pipetted into non-binding black microtiter plates. After addition of fluorogenic substrate, Arg2-DNA, in assay buffer (200 μL), the initial DNA fluorescence (T=0) is measured in a fluorimeter using an excitation wavelength of 340 nm and emission is detected at 410 nm. The plate is then incubated at 37° C. for 1 hour. The final fluorescence of (T=60) is measured. The difference between final and initial fluorescence is calculated. The activity of DPP3 is determined with a 6-point calibration curve. Calibrators and samples are preferably run in duplicates.

The DPP3 levels have been determined with the described DPP3-assays as outlined in the examples using the LAA. The mentioned threshold values above might be different in other assays, if these have been calibrated differently from the assay systems used in the present invention. Therefore, the mentioned cut-off values above shall apply for such differently calibrated assays accordingly, taking into account the differences in calibration. One possibility of quantifying the difference in calibration is a method comparison analysis (correlation) of the assay in question with the respective biomarker assay used in the present invention by measuring the respective biomarker (e.g., DPP3) in samples using both methods. Another possibility is to determine with the assay in question, given this test has sufficient analytical sensitivity, the median biomarker level of a representative normal population, compare results with the median biomarker levels as described in the literature and recalculate the calibration based on the difference obtained by this comparison. With the calibration used in the present invention, samples from 5,400 normal (healthy) subjects (swedish single-center prospective population-based Study (MPP-RES)) have been measured: median (interquartile range) plasma DPP3 was 14.5 ng/ml (11.3 ng/ml-19 ng/ml).

Threshold levels may be obtained, for instance, from a Kaplan-Meier analysis, where the occurrence of a disease is correlated with the quartiles of the biomarker in the population. According to this analysis, subjects with biomarker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th, or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives,” or whether it is considered more appropriate to identify mainly the subjects at high risk at the expense of missing several subjects at moderate risk.

DPP3 activity can be measured by detection of cleavage products of DPP3 specific substrates. Known peptide hormone substrates include Leu-enkephalin, Met-enkephalin, endomorphin 1 and 2, valorphin, β-casomorphin, dynorphin, proctolin, ACTH (Adrenocorticotropic hormone) and MSH (melanocyte-stimulating hormone. (Abramid, M, et al., 2000, Biological Chemistry 381 (12): 1233-43, (Abramid 2000); Barsun, M., et al., 2007, Biol. Chem. 388, 343-348 (Barsun 2007); Dhanda, S., 2008, Cell Biochemistry and Function: Cellular Biochemistry and Its Modulation by Active Agents or Disease 26 (3): 339-45 (Dhanda 2008)). The cleavage of mentioned peptide hormones as well as other untagged oligopeptides (e.g., Ala-Ala-Ala-Ala, (Dhanda 2008)) can be monitored by detection of the respective cleavage products. Detection methods include, but are not limited to, HPLC analysis (e.g., Lee & Snyder 1982), mass spectrometry (e.g., Abramić 2000), H1-NMR analysis (e.g., Vandenberg, J. I, et al., 1985, Archives of Biochemistry and Biophysics 242 (2): 515-22), capillary zone electrophoresis (CE) (e.g., Barsun 2007), thin layer chromatography (e.g., Dhanda 2008), or reversed-phase chromatography (e.g., Mazzocco, C., et al., 2006, FEBS Journal 273 (5): 1056-64).

Detection of fluorescence due to hydrolysis of fluorogenic substrates by DPP3 is a standard procedure to monitor DPP3 activity. Those substrates are specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) coupled to a fluorophore. Fluorophores include but are not limited to β-naphtylamide (2-naphtylamide, βNA, 2NA), 4-methoxy-β-naphtylamide (4-methoxy-2-naphtylamide) and 7-amido-4-methylcoumarin (AMC, MCA) (Abramić 2000; Ohkubo, I, et al., 1999).

Cleavage of these fluorogenic substrates leads to the release of fluorescent β-naphtylamine or 7-amino-4-methylcoumarin respectively. In a liquid phase assay or an ECA substrate and DPP3 are incubated in for example a 96 well plate format and fluorescence is measured using a fluorescence detector. (Ellis & Nuenke 1967).

Additionally, DPP3 carrying samples can be immobilized and divided on a gel by electrophoresis, gels stained with fluorogenic substrate (e.g., Arg-Arg-ONA) and Fast Garnet GBC and fluorescent protein bands detected by a fluorescence reader (Ohkubo et al. 1999). The same peptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) can be coupled to chromophores, such as p-nitroanilide diacetate. Detection of color change due to hydrolysis of chromogenic substrates can be used to monitor DPP3 activity.

Another option for the detection of DPP3 activity is a Protease-Glo™ Assay (commercially available at Promega). In this embodiment of said method DPP3 specific di- or tripeptides (Arg-Arg, Ala-Ala, Ala-Arg, Ala-Phe, Asp-Arg, Gly-Ala, Gly-Arg, Gly-Phe, Leu-Ala, Leu-Gly, Lys-Ala, Phe-Arg, Suc-Ala-Ala-Phe) are coupled to aminoluciferin. Upon cleavage by DPP3, aminoluciferin is released and serves as a substrate for a coupled luciferase reaction that emits detectable luminescence.

DPP3 activity may be measured by addition of the fluorogenic substrate Arg-Arg-ONA and monitoring fluorescence in real time.

The level of DPP3 may be determined by contacting said sample of bodily fluid with a capture binder that binds specifically to DPP3. The capture binder my bind specifically to full-length DPP3. The capture binder may be immobilized on a solid phase. The test sample is passed over the immobile binder, and DPP3, if present in the sample, binds to the binder and is itself immobilized for detection. A substrate may then be added, and the reaction product may be detected to indicate the presence or amount of DPP3 in the test sample. Alternatively, the DPP3 bound to said capture molecule on a solid phase is detected with a second capture molecule specifically binding to DPP3.

The term “solid phase” may be used to include any material or vessel in which or on which the assay may be performed and includes, but is not limited to porous materials, nonporous materials, test tubes, wells, slides, agarose resins (e.g., Sepharose from GE Healthcare Life Sciences), magnetic particles (e.g., Dynabeads™ or Pierce™ magnetic beads from Thermo Fisher Scientific), etc.

The method for determining DPP3 activity in a bodily fluid sample of said subject comprises the steps of:
contacting said sample with a capture-binder that binds specifically to full-length DPP3,
separating DPP3 bound to said capture binder,
adding substrate of DPP3 to said separated DPP3, and
quantifying of said DPP3 activity by measuring and quantifying the conversion of a substrate of DPP3.

The capture binder for determining the level of DPP3 may be selected from the group of antibody, antibody fragment or non-IgG scaffold.

The separation step is a washing step that removes ingredients of the sample that are not bound to said capture-binder from the captured DPP3.

The binder exhibits a binding affinity to DPP3 of at least 10⁷ M⁻¹, preferred 10⁸ M⁻¹, more preferred affinity is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention.

The DPP3 substrate conversion is detected by a method selected from the group comprising: fluorescence of fluorogenic substrates (e.g. Arg-Arg-ONA, Arg-Arg-AMC), color change of chromogenic substrates, luminescence of substrates coupled to aminoluciferin, mass spectrometry, HPLC/FPLC (reversed phase chromatography, size exclusion chromatography), thin layer chromatography, capillary zone electrophoresis, gel electrophoresis followed by activity staining (immobilized, active DPP3) or western blot (cleavage products).

The substrate may be selected from the group comprising: angiotensin II, III and IV, Leu-enkephalin, Met-enkephalin, endomorphin 1 and 2, valorphin, β-casomorphin, dynorphin, proctolin, ACTH and MSH, or di-peptides coupled to a fluorophore, a chromophore or aminoluciferin wherein the dipeptide is Arg-Arg.

To determine the affinity of the antibodies to DPP3 the kinetics of binding of DPP3 to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare), (Lorenz et al., 2011, Antimicrob Agents Chemother 55 (1):165-173).

The assay for determining the level of DPP3 may be a sandwich immunoassay using any kind of detection technology including but not restricted to enzyme label, chemiluminescence label, electrochemiluminescence label, preferably a fully automated assay. In one embodiment of the diagnostic method such an assay is an enzyme labelled sandwich assay. Examples of automated or fully automated assay comprise assays that may be used for one of the following systems: Roche Elecsys®, Abbott Architect®, Siemens Centauer®, Brahms Kryptor®, BiomerieuxVidas®, Alere Triage®.

A variety of immunoassays are known and may be used for the disclosed assays and methods, these include: mass spectrometry (MS), luminescence immunoassay (LIA), radioimmunoassays (“RIA”), homogeneous enzyme-multiplied immunoassays (“EMIT”), enzyme linked immunoadsorbent assays (“ELISA”), apoenzyme reactivation immunoassay (“ARIS”), luminescence-based bead arrays, magnetic beads based arrays, protein microarray assays, rapid test formats such as for instance dipstick immunoassays, immuno-chromatographic strip tests, rare cryptate assay and automated systems/analyzers.

A so-called POC (point-of-care)-test may be used that allows for performing the test within less than 1 hour near the patient without the requirement of a fully automated assay system. One example for this technology is the immunochromatographic test technology, e.g., a microfluidic device.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labelled, e.g. with a dye, with a radioisotope, or a reactive or catalytically active moiety. The amount of labelled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13:978-0080445267; Hultschig, C., et al., 2006, Curr Opin Chem Biol., February, 10(1):4-10).

The assay may comprise two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample. The labeling system may comprise rare earth cryptates or rare earth chelates in combination with fluorescence dye or chemiluminescence dye, in particular a dye of the cyanine type.

Fluorescence-based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

Chemiluminescence based assays comprise the use of dyes, based on the physical principles described for chemiluminescent materials in (Kirk-Othmer, Encyclopedia of chemical technology, 4th ed., executive editor, J. I. Kroschwitz; editor, M. Howe-Grant, John Wiley & Sons, 1993, vol. 15, p. 518-562, incorporated herein by reference, including citations on pages 551-562). Preferred chemiluminescent dyes are acridiniumesters.

An “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 108 M.

Circulating DPP3 (cDPP3) levels were shown to be increased in septic, cardiogenic, and vasodilatory shock patients. (Rehfeld 2019). Moreover, this increase in cDPP3 levels was associated with an increased risk of short-term mortality and severe organ dysfunction in patients with CS. (Deniau, B., et al., 2020, European J. of Heart Failure 22 (2): 290-99. (Deniau 2020 Circulating DPP3)). Moreover, patients with severe sepsis or septic shock showed a correlation between a higher initial cDPP3 and the greater the need for organ support and vasopressors upon admission and the longer the need for vasopressor(s), mechanical ventilation, or renal replacement therapy (RRT) and the higher the need for fluid load. (Blet, A., et al., 2021, Critical Care 25 (1): 1-10, (Blet 2021)).

WO2017/182561 describes methods for determining the total amount or active DPP3 in a sample of a patient for the diagnosis of a disease related to necrotic processes. It further describes a method of treatment of necrosis-related diseases by antibodies directed to DPP3.

WO2019/081595 describes DPP3 binder directed to and binding to specific DPP3 epitopes and its use in the prevention or treatment of diseases that are associated with oxidative stress.

WO2021/185786 describes methods for determining DPP3 in a sample of a patient for the diagnosis, risk prediction, prognosis and monitoring in a patient infected with a coronavirus. It further describes an inhibitor of the activity of DPP3 for use in therapy or intervention in a patient infected.

During shock, production of Ang II is enhanced in order to restore blood pressure. (Corréa 2015). However, this feedback mechanism is usually impaired by myocardial infarction medications and endothelial dysfunction with consequent angiotensin converting enzyme (ACE) dysfunction. (Reynolds & Hochman 2008; Bellomo, R. et al., 2020, Am. J Respir Crit. Care Med. 202, 1253-1261). Additionally, upon hypoperfusion and tissue death, Ang II levels may also be compromised by increasing levels of DPP3 in the blood. (Picod, A. et al., 2021, Am J Respir Crit Care Med. 203, 526-527 (Picod 2021); Deniau, B., et al., 2020 Eur J. Heart Fail. 1-10 (Note: this article was electronically published in 2019, so to avoid confusion with the other Deniau article, this article will be referred to as “Deniau 2019”)). Although there is limited data describing Ang II or ACE levels in CS, it is likely that patients with shock, who present with high levels of DPP3 in the blood, have a DPP3-dependent deficiency in Ang II which can significantly contribute to the worsening of circulatory function. (Picod 2021).

In support of a DPP3-dependent deficiency in Ang II in shock, a strong correlation between cDPP3 concentration and activity in the blood of critically ill patients confirms that DPP3 is released in its active form. (Rehfeld 2019; Deniau 2019). Consequently, DPP3 could contribute to circulatory failure by enhanced cleavage of vasoactive peptides such as Ang II. (Picod 2021). This hypothesis is reinforced by (1) a plethora of retrospective study analyses showing that high cDPP3 concentration in the bloodstream (>40 ng/ml) is associated with myocardial depression, multi-organ dysfunction, disease severity, and poor outcome, in patients with circulatory failure (Van Lier D. & Pickkers, P, 2021, Curr Opin. Crit. Care 27, 261-268 (Van Lier & Pickkers 2021); Deniau 2019; Wenzl, F A., et al., 2023, Eur Heart J. 44, 3859-3871 (Wenzl 2023); Pöss, J., et al., 2023, Eur Heart J. 44, 3872-3874; Ye, P, et al., 2022, Front. Cardiovasc. Med. 9; Ozden, O., et al., 2022, Clin. Appl. Thromb. 28, 1-4; Mendez H., et al., 2023, J. Pers. Med. 13; Takagi, K. et al., 2019, Eur J. Heart Fail. 1-8; Blet 2021; Van Lier D., et al., 2023, J. Crit. Care 78, 154383; Van Lier D., et al., 2022, ERJ Open Res 9; Iborra-Egea, O., et al., 2020, Curr Opin. Crit. Care 26, 392-397); or (2) by in vivo studies demonstrating that intravenous administration of DPP3 to healthy rodents leads to impairment of cardiac and renal function (Deniau 2019). These findings are aligned with the ascending role of cDPP3 as a marker of CS and mortality in patients with acute coronary syndrome (Wenzl 2023).

Binders that inhibit DPP3 have been described. (Abramid, M & Agid, D., 2022, Molecules 27 (9)). Except for the polypeptide aprotinin, all others are small molecules and include flavonoids, coumarin, and benzimidazole derivatives. The most potent inhibitors yet reported (propioxatin A and B, Tyr-Phe- and Phe-Phe-NHOH, and JMV-390) are active in low nanomolar range and contain a hydroxamic acid moiety. High inhibitory potential possesses oligopeptides from the hemorphin group, valorphin and tynorphin, which are poor substrates of DPP3.

FIG. 1 shows the structure of the humanized anti-DPP3 antibody as an IgG1 molecule including positions of the disulfide bridges. The heavy chain domains are depicted in dark grey and light chain domains in light grey. Inter- and intra-molecule disulfide bridges are marked with the respective position in the sequence.

The humanized anti-DPP3 antibody's mode of action is relevant in acute diseases that are associated with massive cell death and uncontrolled release of intracellular DPP3 into the bloodstream. A massive increase in cDPP3 due to tissue injury and release from dying cells, as observed during CS, is believed to promote uncontrolled depletion of Ang II and, subsequently, lead to hemodynamic instability and organ failure. Translocated DPP3 remains active in the circulation where it cleaves bioactive peptides in an uncontrolled manner.

The humanized anti-DPP3 antibody is able to inhibit cDPP3 thereby inhibiting bioactive peptide degradation in the bloodstream. This novel treatment approach is intended to offer a treatment option for a patient population that, to date, has only very limited pharmacological treatment therapies available. The humanized anti-DPP3 antibody is designed to block the enzymatic activity of cDPP3 in the bloodstream, thereby inhibiting DPP3-dependent Ang II degradation (FIG. 2). This inhibition of cDPP3 results in stabilization of cardiovascular and renal function and reduction of short-term mortality.

Preclinical studies of anti-DPP3 antibodies in animal models of cardiovascular failure showed impressive and instant efficacy. (Malovan, G., et al., 2023, FEBS Journal 290 (9): 2246-62 (Malovan 2023); Deniau, B., et al., 2020, PLoS ONE15 (8 August): 1-12 (Deniau 2020 Inhibition); Deniau 2020 Circulating DPP3; Garcia, B., et al., 2024, Intensive Care Medicine Experimental 12 (1): 53 (Garcia 2024)). The humanized anti-DPP3 antibody used in studies in a large animal model of cardiovascular dysfunction showed that said anti-DPP3 antibody-treated animals required lower doses of vasopressors and fluids to maintain adequate tissue perfusion and target mean arterial pressure (MAP) at 65 mmHg (Garcia 2024). These effects were associated with an increase in circulating Ang II concentrations, a preserved Ang I/Ang II ratio, prevention of ATIR downregulation and higher alpha-1, beta-1, and beta-2 adrenergic receptor expression (Garcia 2024). Reduction in catecholamine exposure by the humanized anti-DPP3 antibody lead to reduced inflammation and myocardial injury, exerting a cardioprotective effect (Garcia 2024). This aligns with the findings in rodents, where the humanized anti-DPP3 antibody administration reduces myocardial oxidative stress. (WO2019/081595; Menale, C. et al., 2019, J. Bone Miner Res. jbmr.3829; Deniau 2019; Deniau 2020 Inhibition). Finally, the humanized anti-DPP3 antibody treatment also resulted in improved PaO₂/FiO₂ ratio (respiratory function) (Garcia 2024). This positive effect on hypoxemia could be a better regional blood flow and distribution due to improved vascular tone caused by the normalized Ang II levels. In several preclinical cardiovascular failure models, the humanized murine anti-DPP3 antibody has shown to normalize ejection fraction and kidney function and to reduce mortality (Malovan 2023).

Included in the subject matter of the present invention is an inhibitor of the activity of DPP3 for use in therapy or intervention in a critically ill patient with a reduction in pulmonary function for the improvement of pulmonary function.

Included in the subject matter of the present invention is an inhibitor of the activity of DPP3 for use as therapy or intervention in a critically ill patient for the improvement of lung function, wherein said patient is having a level of DPP3 above a pre-determined threshold.

Included in the subject matter of the present invention is an inhibitor of the activity of DPP3 for use as therapy or intervention in a critically ill patient with a reduction in blood pressure for myocardial protection and/or prevention of myocardial injury, wherein said patient is having a level of DPP3 above a pre-determined threshold.

Examples provided below demonstrate that an injection of a humanized anti-DPP3 antibody in pigs with septic shock resulted in a marked improvement of pulmonary function (given by PaO2/FiO2 ratio). Furthermore, it has been demonstrated that DPP3 was significantly elevated in ICU all-corner patients under ventilation, especially mechanical ventilation. Therefore, the instant inventors determined that an inhibitor of DPP3, especially an anti-DPP3 antibody, is able to improve pulmonary function in patients with reduced lung function who are ventilated, irrespective of the indication.

It was the surprising finding that an inhibitor of DPP3 activity is suitable for use in therapy or intervention in a critically ill patient with a reduction in pulmonary function for the improvement of pulmonary function.

Additionally, the Examples provided below demonstrate that an injection of a humanized anti-DPP3 antibody in pigs with septic shock prevented myocardial injury (given by increases of myocardial IL-6 and troponin, respectively). Furthermore, it has been demonstrated that DPP3 was significantly elevated in patients with decline of blood pressure, especially in shock (e.g., septic shock, cardiogenic shock) and acute coronary syndrome (ACS). Therefore, the instant inventors determined that an inhibitor of DPP3, especially an anti-DPP3 antibody, is able to prevent myocardial injury in patients with decline of blood pressure, irrespective of the indication.

It is another surprising finding that an inhibitor of DPP3 activity is suitable for use in therapy or intervention in a critically ill patient with a decline in blood pressure for myocardial protection and/or prevention of myocardial injury.

It was the surprising finding that an anti-DPP3 antibody or fragment thereof binding to DPP3 could be used for the treatment of shock, including in an extreme-critically ill patient having shock.

An object of the present invention is the provision of an anti-DPP3 antibody or fragment thereof binding to DPP3 for the treatment of shock in a patient having shock, including wherein said patient is an extreme-critically ill patient. A specific finding of the present invention is that an anti-DPP3 antibody could be used for the treatment of shock in an extreme-critically ill patient having shock, wherein said patient has cardiogenic shock or combined cardiogenic and septic shock. A person of ordinary skill in the art would appreciate that the biosimilarity of an antibody to an anti-DPP3 antibody or fragment thereof claimed herein can be determined by, for example, following the U.S. FDA's Guidance “Questions and Answers on Biosimilar Development and the BPCI Act” dated September 2021, incorporated in full herein.

Another object of the present invention is a formulation and dosage amounts for the provision of an anti-DPP3 antibody or fragment thereof binding to DPP3, including the humanized anti-DPP3 antibody, to a human subject. It is a further object of the present invention to safely administer a pharmaceutical formulation and dosage amounts of an anti-DPP3 antibody or fragment thereof binding to DPP3, including the humanized anti-DPP3 antibody, to a human subject.

### SUMMARY

In one embodiment, the invention is a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer; and
(e) a solvent;
wherein the formulation provides a dose of the active ingredient in a range from about 1.0 mg/kg to about 12.0 mg/kg.

In a further embodiment, the invention comprises a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer; and
(e) a solvent;
wherein the formulation provides a dose of the active ingredient that is preferably about 3.0 mg/kg, or preferably about 6.0 mg/kg, or preferably about 12.0 mg/kg.

In a further embodiment, the invention comprises a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer; and
(e) a solvent;
wherein the formulation provides a dose of the active ingredient in a range from about 1.0 mg/kg to about 12.0 mg/kg, wherein the dose is administered with an infusion volume of about 1.0 to 1.2 mL/kg.

In a further embodiment, the formulations of the invention are contained in a glass vial.

In a further embodiment, the formulations of the invention are contained in a glass vial, wherein the glass vial contains about 400 mg of the active ingredient.

In a further embodiment, the formulations of the invention are contained in a glass vial, wherein the glass vial contains about 20 ml of the formulation.

In a further embodiment, the formulations of the invention are contained in a glass vial, wherein the glass vial contains the active ingredient at a concentration of about 20 mg/ml.

In a further embodiment, the invention comprises a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer; and
(e) a solvent;
wherein the formulation provides a dose of the active ingredient in a range from about 1.0 mg/kg to about 12.0 mg/kg, wherein the formulation is contained in a glass vial, wherein the glass vial contains about 20 ml of the formulation, and wherein the ingredients are in the following proportions:
(a) about 20 mg/mL of the active ingredient;
(b) about 90.7 mg/mL of the stabilizer;
(c) about 2.86 mg/mL of the antioxidant; and
(d) about 1.11 mg/mL of the buffer.

In one embodiment, the invention is a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer;
(e) a solvent; and
(f) a composition for pH adjustment; and
wherein the formulation provides a dose of the active ingredient in a range from about 1.0 mg/kg to about 12.0 mg/kg.

In a further embodiment, the formulations of the invention comprise a composition for pH adjustment that is L-Histidine HCl*H₂O.

In a further embodiment, the formulations of the invention comprise a composition for pH adjustment that is L-Histidine HCl*H₂O in a proportion of 0.51 mg/mL.

In one embodiment, the invention is a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer; and
(e) a solvent;
wherein the formulation provides a dose of the active ingredient in a range from about 1.0 mg/kg to about 12.0 mg/kg, and wherein the pH of the formulation is about 6.5.

In one embodiment, the invention is a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer;
(e) a solvent; and
(f) a composition for pH adjustment; and
wherein the formulation provides a dose of the active ingredient in a range from about 1.0 mg/kg to about 12.0 mg/kg, and wherein the pH of the formulation is about 6.5.

In one embodiment, the invention is a formulation for administering an anti-DPP3 antibody or fragment thereof to a human subject, wherein the formulation is a solution suitable for intravenous application and the formulation comprises:
(a) the anti-DPP3 antibody or fragment thereof as an active ingredient;
(b) a stabilizer;
(c) an antioxidant;
(d) a buffer; and
(e) a solvent;
wherein the formulation provides a dose of the active ingredient in a range from about 1.0 mg/kg to about 12.0 mg/kg;
wherein the formulations of the invention are contained in a glass vial, wherein the glass vial contains about 20 ml of the formulation; and
wherein the vial contains:
(a) about 400 mg of the anti-DPP3 antibody;
(b) about 1.8154 g of trehalose dihydrate;
(c) about 57.2 mg of L-methionine;
(d) about 22.2 mg of L-histidine;
(e) about 10.2 mg of L-Histidine HCl*H₂O; and
(f) about 20 ml of water for injection.

In a further embodiment, the formulations of the invention comprise the anti-DPP3 antibody or fragment thereof, wherein the anti-DPP3 antibody or fragment thereof has:
a heavy chain that comprises at least one complementarity determining regions (CDR) selected from the group comprising SEQ ID NO: 7, SEQ ID NO: 8, and SEQ ID NO: 9, or a sequence that is >95% identical to it; and
a light chain that comprises at least one CDR selected from the group comprising SEQ ID NO: 10, CDR2 comprising KVS, and SEQ ID NO: 11, or a sequence that is >95% identical to it.

In a further embodiment, the formulations of the invention comprise the anti-DPP3 antibody or fragment thereof, wherein anti-DPP3 antibody or fragment thereof comprises SEQ ID No. 12 and SEQ ID No. 13.

In a further embodiment, the anti-DPP3 antibody or fragment thereof is a humanized anti-DPP3 antibody or fragment thereof.

In one embodiment, an inhibitor of the activity of DPP3 is for use in therapy or intervention in a critically ill patient with a decline in blood pressure for myocardial protection and/or prevention of myocardial injury, wherein the inhibitor of the activity of DPP3 is selected from the group comprising small molecules, anti-DPP3 antibody, anti-DPP3 antibody fragment, or anti-DPP3 non-lg scaffold.

In a further embodiment, the inhibitor of the activity of DPP3 is for use in therapy or intervention in a critically ill patient with a decline in blood pressure for myocardial protection and/or prevention of myocardial injury, wherein the inhibitor of the activity of DPP3 is a small molecule selected from the group comprising spinorphin, tynorphin, propioxatin A and B, fluostatin A and B, enzimidazole, or derivatives or analogues thereof.

In a further embodiment, the inhibitor of the activity of DPP3 is for use in therapy or intervention in a critically ill patient with a decline in blood pressure for myocardial protection and/or prevention of myocardial injury, wherein said inhibitor is an anti-DPP3 antibody, anti-DPP3 antibody fragment, or anti-DPP3 non-lg scaffold that exhibits a minimum binding affinity to DPP3 of equal or less than 10⁻⁷ M.

In a further embodiment, the inhibitor of the activity of DPP3 is for use in therapy or intervention in a critically ill patient with a decline in blood pressure for myocardial protection and/or prevention of myocardial injury, wherein said antibody is a monoclonal antibody or monoclonal antibody fragment.

In a further embodiment, the inhibitor of the activity of DPP3 is for use in therapy or intervention of pulmonary function in a critically ill patient with a reduction in pulmonary function for the improvement of pulmonary function, wherein the inhibitor of the activity of DPP3 is selected from the group comprising small molecules, anti-DPP3 antibody, anti-DPP3 antibody fragment, or anti-DPP3 non-Ig scaffold.

In a further embodiment, the inhibitor of the activity of DPP3 is for use in therapy or intervention in a critically ill patient with a reduction in pulmonary function for the improvement of pulmonary function, wherein the inhibitor of the activity of DPP3 is a small molecule selected from the group comprising spinorphin, tynorphin, propioxatin A and B, fluostatin A and B, benzimidazol, or derivatives or analogues thereof.

In a further embodiment, the inhibitor of the activity of DPP3 is for use in therapy or intervention in a critically ill patient with a reduction in pulmonary function for the improvement of pulmonary function, wherein said inhibitor is an anti-DPP3 antibody, anti-DPP3 antibody fragment, or anti-DPP3 non-Ig scaffold that exhibits a minimum binding affinity to DPP3 of equal or less than 10 M.

In a further embodiment, the inhibitor of the activity of DPP3 is for use in therapy or intervention in a critically ill patient with a reduction in pulmonary function for the improvement of pulmonary function, wherein said antibody is a monoclonal antibody or monoclonal antibody fragment.

### BRIEF DESCRIPTION OF THE DRAWINGS

FIG. 1: Structure of the humanized anti-DPP3 antibody including location of disulfide bridges (S-S) depicted in cartoon form. The heavy chain sequence is depicted in blue and light chain sequence is depicted in orange. Inter- and intra-molecule disulfide bridges are marked with by their respective position in the amino acid sequence. V_{H}=Variable Heavy; C_{H}1=Constant Heavy 1; C_{H}2=Constant Heavy 2; C_{H}3=Constant Heavy 3; V_{L}=Variable Light, C_{L}=Constant Light. The V_{L} and the V_{H} make up the variable region. NH₂ and COOH denote the amino terminus and the carboxyl terminus, respectively.

FIG. 2: Schematic representation of the DPP3 and the anti-DPP3 antibody mode of action.

FIG. 3: Inhibition curve of native DPP3 from blood cells with inhibitory murine anti-DPP3 antibody. Inhibition of DPP3 by a specific antibody is concentration dependent, with an IC₅₀ at ˜15 ng/ml when analysed against 15 ng/ml DPP3.

FIG. 4: Association and dissociation curve of the murine anti-DPP3 antibody-DPP3 binding analysis using Octet. Murine Anti-DPP3 antibody loaded biosensors were dipped into a dilution series of recombinant GST-tagged human DPP3 (100, 33.3, 11.1, 3.7 nM) and association and dissociation monitored.

FIG. 5: Western Blot of dilutions of blood cell lysate and detection of dilutions of blood cell lysate and recombinant human DPP3 with a murine anti-DPP3 antibody as primary antibody.

FIG. 6: High concentrations of DPP3 levels 24 hours after admission of septic patients were associated with worst SOFA scores.

FIG. 7: High cDPP3 plasma levels correlate with organ dysfunction in septic patients. Bar plots of SOFA score in AdrenOSS-1 according to the evolution of DPP3 levels during ICU stay. HH: DPP3 above median on admission and at 24h; HL: above median on admission but below median at 24h; LL: below median on admission and at 24h; LH: below median on admission but above median at 24h.

FIGS. 8 A, 8 B, 8 C, 8 D, 8 E, and 8 F: High concentrations of cDPP3 levels 24 hours after admission of septic patients were associated with worst SOFA scores by organ. (FIG. 8 A) cardiac, (FIG. 8 B) renal, (FIG. 8 C) respiratory, (FIG. 8 D) liver, (FIG. 8 E) coagulation, and (FIG. 8 F) central nervous system SOFA scores values according to dynamics levels of cDPP3 between admission and 24h (HH: High/High, HL: High/Low, LH: Low/High, LL: Low/Low).

FIG. 9: Kaplan-Meyer-Plot showing the association of DPP3 levels measured at admission in patients in need of mechanical ventilation (n=390) with 28-day mortality with DPP3 levels above and below 40.0 ng/mL (HR 2.1, 95-% CI 1.5-2.9, p<0.0001).

FIGS. 10 A and 10 B: Kaplan-Meyer-Plot showing the association of DPP3 levels measured at admission in n=217 invasively ventilated patients (FIG. 10 A) and n=131 non-invasively ventilated patients (FIG. 10 B) with 28-day mortality with DPP3 levels above and below 40.0 ng/mL.

FIGS. 11 A, 11 B, and 11 C: Effect of DPP3 or anti-DPP3 antibody injection on systemic and renal hemodynamics in mice: mean arterial pressure (FIG. 11 A), renal blood flow (FIG. 11 B), and renovascular resistances variations (FIG. 11 C) after DPP3, PBS, or anti-DPP3 antibody administration. Data are presented as mean+/−standard error (SE). Comparison between groups was made by repeated-measures ANOVA followed by post-hoc Dunnett's multiple comparison test. DPP3=Dipeptidyl Peptidase 3; PBS=Phosphate buffered saline; AB=anti-DPP3 antibody; MAP=Mean arterial pressure; RBF=Renal blood flow; RVR=renovascular resistances.

FIGS. 12 A, 12 B, 12 C, 12 D, 12 E, and 12 F: Effect of DPP3 and of the anti-DPP3 antibody on systemic and renal vasoreactivity to Ang II in mice. Effect of DPP3 on the peak MAP (FIG. 12 A), RBF (FIG. 12 B), and RVR (FIG. 12 C) of increasing doses of Ang II. Effect of an anti-DPP3 antibody on the peak MAP (FIG. 12 D), RBF (FIG. 12 E), and RVR (FIG. 12 F) of increasing doses of Ang II. Comparison between groups was made with ANOVA. MAP=Mean arterial pressure; RBF=Renal blood flow; RVR=Renovascular resistance; Ang II=Angiotensin II; DPP3=Dipeptidyl Peptidase 3; AB=anti-DPP3 antibody.

FIGS. 13 A, 13 B, 13 C, 13 D, 13 E, and 13 F: Effect of DPP3 and anti-DPP3 antibody on systemic and renal vasoreactivity to norepinephrine in mice. Effect of DPP3 on the peak MAP (FIG. 13 A), RBF (FIG. 13 B), and RVR (FIG. 13 C) of 20 ng of norepinephrine. Effect of an anti-DPP3 antibody on the peak MAP (FIG. 13 D), RBF (FIG. 13 E), and RVR (FIG. 13 F) of 20 ng of norepinephrine. MAP=Mean arterial pressure; RBF=Renal blood flow; RVR=Renovascular resistance; Ang II=Angiotensin II; DPP3=Dipeptidyl Peptidase 3; AB=anti-DPP3 antibody; ns=not significant.

FIG. 14: DPP3 activity and circulating angiotensins 15 min after PBS, DPP3, or anti-DPP3 antibody administration. Comparison between groups was made by ANOVA or the Kruskal-Wallis test as appropriate. Ang=Angiotensin; DPP3=Dipeptidyl Peptidase 3; AB=anti-DPP3 antibody; ns=not significant.

FIG. 15 A, 15 B, 15 C, 15 D, 15 E, and 15 F: Effects of a pre-treatment by valsartan on the systemic and renal hemodynamic effects induced by DPP3: maximal effect (T+1 min) on mean arterial pressure (FIG. 15 A), renal blood flow (FIG. 15 B), and renovascular resistances (FIG. 15 C) variations after DPP3 in mice pretreated or not with the AT1R antagonist valsartan. Correlation between the effect induced by valsartan and the additional effect produced by DPP3 administration on mean arterial pressure, renal blood flow, and renovascular resistances. DPP3 effect on (FIG. 15 D) mean arterial pressure; (FIG. 15 E) renal blood flow; and (FIG. 15 F) renovascular resistance in mice pretreated or not with the AT1R antagonist valsartan. Data are presented as mean+/−SE. DPP3=Dipeptidyl Peptidase 3; MAP=Mean arterial pressure; RBF=Renal blood flow; RVR=renovascular resistances.

FIG. 16 A, 16 B, and 16 C: Effects of a pre-treatment by naloxone on the systemic and renal hemodynamic effects induced by DPP3: mean arterial pressure (FIG. 16 A), renal blood flow (FIG. 16 B), and renovascular resistances (FIG. 16 C) variations after DPP3 in mice pretreated or not with the pan-opioid receptors antagonist naloxone. Data are presented as mean+/−SE. DPP3=Dipeptidyl Peptidase 3; MAP=Mean arterial pressure; RBF=Renal blood flow; RVR=renovascular resistances.

FIG. 17 A, 17 B, and 17 C: Effects of a pre-treatment by labetalol on the systemic and renal hemodynamic effects induced by DPP3: mean arterial pressure (FIG. 17 A), renal blood flow (FIG. 17 B), and renovascular resistances (FIG. 17 C) variations after DPP3 in mice pretreated or not with the alpha- and beta-adrenergic receptors antagonist labetalol. Data are presented as mean+/−SE. Comparison between group was made by repeated-measures ANOVA followed by post-hoc Dunnett's multiple comparison test. DPP3=Dipeptidyl Peptidase 3; MAP=Mean arterial pressure; RBF=Renal blood flow; RVR=renovascular resistances.

FIG. 18 A, 18 B, 18 C, 18 D, 18 E, 18 F, and 18 G: Characterization of heart failure (HF) mice model. (FIG. 18 A) Schematic representation of the protocol used to provoke HF in mice. (FIG. 18 B) Left ventricular shortening fraction of sham and ISO mice at day 3, i.e., 12 h after the last isoproterenol injection. Comparison was made by Mann-Whitney rank-sum test. (FIG. 18 C) Renal resistivity index of sham and ISO mice at day 3. Comparison was made by Mann-Whitney rank-sum test. (FIG. 18 D) Plasma mouse circulating Dipeptidyl Peptidase 3 (DPP3) activity in sham and ISO mice. (FIG. 18 E) Cardiac output in sham and isoproterenol injected (ISO) mice 12 h after the last injection of isoproterenol. Comparison was made by Mann-Whitney test. (FIG. 18 F) Lung weight on tibia length (LW/TL) in sham and isoproterenol injected (ISO) mice 12 h after the last injection of isoproterenol. Comparison was made by Mann-Whitney test. (FIG. 18 G) E/A ratio (mitral Doppler wave) in sham and isoproterenol injected (ISO) mice 12h after the last injection of isoproterenol. Comparison was made by Mann-Whitney test.

FIGS. 19 A, 19 B, 19 C, and 19 D: Anti-DPP3 antibody rapidly and sustainably improves hemodynamics in isoproterenol-injected mice. (FIG. 19 A) Left ventricular shortening fraction in sham+PBS (n=5), isoproterenol-induced heart failure (ISO-HF)+PBS (n=5), and ISO-HF+anti-DPP3 antibody (n=5) mice until 24 h after treatment. Hemodynamics points were taken at baseline (B), at day 3 (D3; after isoproterenol administration), 1 hour, 6 hours, and 24 post injection of anti-DPP3 antibody, PBS, or non active IgG. Comparison was made by repeated measures ANO-VA. (FIG. 19 B) Difference between ISO-HF+PBS and ISO-HF+anti-DPP3 antibody groups in hemodynamics 6 h after anti-DPP3 antibody or PBS injection. (*P<0.05 between ISO+PBS and ISO+anti-DPP3 antibody groups). (FIG. 19 C) Evolution of shortening fraction after isoproterenol injection and PBS injection (vertical dotted line, n=5), anti-DPP3 antibody (n=5), and non-active Ig G (n=3), at day 3. Mice were monitored during the 14 days after the beginning of isoproterenol injection. Hemodynamics points were taken at baseline (B), at day 3 (D3; after isoproterenol administration), day 8 (D8), and day 14 (D14) post injection of anti-DPP3 antibody, PBS, or non active IgG. Animals were sacrificed at day 14. CO=cardiac output; E/A=mitral Doppler wave; HR=heart rate; LVDd=left ventricular diameter in diastole; LVDs=left ventricular diameter in systole; SV=stroke volume.

FIG. 20: Renal hemodynamics in mice after treatment with ISO+PBS or ISO+anti-DPP3 antibody compared to sham control (PBS). Renal resistance index was measured in mice continuously at baseline (B), and after induction of heart failure with ISO followed by a treatment with either PBS or anti-DPP3 antibody. HF=Heart Failure; ISO=Isoproterenol; AB=anti-DPP3 antibody; PBS=Phosphate buffered saline.

FIG. 21: Anti-DPP3 antibody reduces oxidative stress in ISO treated mice compared to ISO-HF+PBS mice. Heart tissue was collected from sham control mice or mice with induced heart failure (ISO) after treatment with anti-DPP3 antibody or PBS and stained with DHE to evaluate oxidative stress. DHE=dihydroethidium; Iso=Isoproterenol; PBS=Phosphate Buffered Saline; AB=anti-DPP3 antibody.

FIGS. 22 A and 22 B: Design of the polymicrobial sepsis induction by cecal ligation and puncture (CLP) study. (FIG. 22 A) Schematic representation of the preclinical experiment in rats, including treatments and hemodynamic assessment (BP=blood pressure, CLP=cecal ligation and puncture, PBS=phosphate-buffered saline, AB=anti-DPP3 antibody, TTE=transthoracic echocardiography). (FIG. 22 B) Flowchart of the randomization.

FIG. 23 A, 23 B, 23 C, 23 D, and 23 E: Anti-DPP3 antibody injection improved hemodynamics and cardiac function during circulatory failure induced by sepsis. (FIG. 23 A) Graphic representation of the evolution of the left ventricular shortening fraction in time in sham+PBS (blue horizontal dashed line), CLP+PBS (grey line (lower data line)), and CLP+anti-DPP3 antibody groups (black line (upper data line)) (p<0.05 using repeated measures ANOVA). Red dashed vertical line corresponds to the moment of bolus injection followed by infusion. (FIG. 23 B) Evolution of cardiac output, (FIG. 23 C) stroke volume, (FIG. 23 D) heart rate, (FIG. 23 E) mean blood pressure before randomization and 120 min after therapy with anti-DPP3 antibody or PBS injection. Comparisons were performed using Wilcoxon rank-sum test. bpm=beats per minute; CLP=cecal ligation and puncture.

FIGS. 24 A and 24 B: Anti-DPP3 antibody injection decreased DPP3 plasmatic activity. (FIG. 24 A) Plasma DPP3 activity in sham+PBS, CLP+PBS, and CLP+anti-DPP3 antibody groups. Comparisons were made using Wilcoxon rank-sum test. (FIG. 24 B) Myocardial qPCR of DPP3 mRNA of sham+PBS, CLP+PBS, and CLP+anti-DPP3 antibody rats. Comparisons were made using Wilcoxon rank-sum test.

FIGS. 25 A, 25 B, 25 C, and 25 D: Anti-DPP3 antibody injection improved myocardial oxidative stress. Representative images (FIG. 25 A) and quantification (FIG. 25 B) of DHE staining from myocardial sections of sham+PBS, CLP+PBS, and CLP+anti-DPP3 antibody rat groups. Myocardial mRNA expression of HO-1 (FIG. 25 C) and NQO-1 (FIG. 25 D) in of sham+PBS, CLP+PBS, and CLP+anti-DPP3 antibody rat groups. Comparisons were made by Wilcoxon rank-sum test. DHE=dihydroethidium; HO-1=heme oxygenase 1; NQO-1=NAD(P)H dehydrogenase (quinone 1).

FIG. 26: Protocol timeline for a pig septic shock model. Two hours after the end of the instrumentation, baseline measurements were obtained, and feces were injected into the peritoneum. Animals were allowed to develop sepsis and cardiac dysfunction until a severe hypotensive state arbitrarily set at a mean arterial pressure (MAP)<50 mmHg (corresponding to the shock time-point) was reached. According to prior randomization, anti-DPP3 antibody was started. Severe hypotension (between 45 and 50 mmHg MAP) was left untreated for one hour. Thereafter, fluid resuscitation was started for 20 minutes. Full resuscitation was then started with norepinephrine, source control, antibiotics, and abdominal wall incision. Animals were euthanized 13 hours and 20 minutes after the shock time-point.

FIGS. 27 A, 27 B, and 27 C: Hemodynamic parameters for control and anti-DPP3 antibody groups in a pig septic shock model. (FIG. 27 A) Norepinephrine dose during the resuscitation phase. Fluid balance index from shock time point to the end of the experiment. (FIG. 27 B) Mean arterial pressure, cardiac output index, heart rate and left ventricle dP/dTmax. (FIG. 27 C) Indexes of cardiac preload (pulse pressure variation and left ventricle end diastolic pressure). Values are expressed as median interquartile range. BL=baseline; S=shock time-point; R=Resuscitation time-point. P-value for interaction. *P-value<0.05 between anti-DPP3 antibody and control group for post-hoc interactions.

FIG. 28: Tissue perfusion indices and organ blood flow for anti-DPP3 antibody and control groups. Values are expressed as median & interquartile range P-value for interaction. *P-value<0.05 between anti-DPP3 antibody and control group in case of overall interaction.

FIG. 29: Evolution of Dipeptidyl Peptidase 3 (DPP3) activity and anti-DPP3 antibody concentrations over time in the septic pig model. Animals received either standard treatment (control) or anti-DPP3 antibody. Values are expressed as mean+/−standard deviation. P-value for interaction between groups for DPP3 measurements. *P-value<0.05 for time points R: Resuscitation time point, after one hour of hypoperfusion.

FIGS. 30 A and 30 B: Circulating catecholamines and equilibrium analysis of the RAAS. (FIG. 30 A) Circulating catecholamines at different time-points. (FIG. 30 B) Equilibrium analysis of the RAAS. Values are expressed as median & interquartile range. P-value is reported for overall interaction. *P-value<0.05 between anti-DPP3 antibody and control group. Ang=Angiotensin.

FIGS. 31 A, 31 B, and 31 C: Expression of IL-6 and myocardial and vascular angiotensin receptors in the control group (center bar of group), the anti-DPP3 antibody group (right bar of group), and sham group (left bar of group). (FIG. 31 A) Relative mRNA expression of IL-6 in the left ventricle, aorta, femoral, and radial arteries. Relative quantification was achieved using the comparative 2^{-ΔΔCt} method by normalization with the housekeeping gene (ActB-actin). Results are expressed as relative fold increase above the mean value of relative mRNA expression of the sham group arbitrarily fixed at 1. (FIG. 31 B) Relative mRNA expression and relative protein expression of AT₁ and AT₂ in the left ventricle. Results are expressed as relative fold increase above the mean value of the sham group arbitrarily fixed at 1. (FIG. 31 C) Relative mRNA expression and relative protein expression of AT₁ and AT₂ in the aorta, femoral, and radial arteries. Results are expressed as relative fold increase above the mean value of the sham group arbitrarily fixed at 1. Values are expressed as median & interquartile range. *P-value<0.05; **P-value<0.01 between anti-DPP3 antibody and control groups.

FIGS. 32 A and 32 B: Myocardial and vascular mRNA and protein expression of adrenergic receptors in control (grey, center), anti-DPP3 antibody (blue, right), and sham (white, left) groups. (FIG. 32 A) Alpha-1 mRNA and protein expression. (FIG. 32 B) Beta-1 mRNA and protein expression, vascular beta-2 mRNA expression and vascular alpha-2 protein expression. Values are expressed as median & interquartile range *P-value<0.05.

FIG. 33: Median (IQR) PCZ concentration and DPP3 activity in mice after repeated dosing with 37.5 mg/kg PCZ on days 1, 2, 13, and 14 (D1, D2, D13, D14), indicated by arrows. Time points of sampling for PCZ concentration and DPP3 activity are indicated on the x-axis (h for hours, min for minutes). Per timepoint, n=6 animals were analyzed. Interquartile range=IQR.

FIG. 34: Median (IQR) PCZ concentration and DPP3 activity in mice after repeated dosing with 150 mg/kg PCZ on days 1, 2, 13, and 14 (D1, D2, D13, D14), indicated by arrows. Time points of sampling for PCZ concentration and DPP3 activity are indicated on the x-axis (h for hours, min for minutes). Per timepoint, n=6 animals were analyzed. Interquartile range=IQR.

FIG. 35: Median (IQR) PCZ concentration and DPP3 activity in cynomolgus monkeys after repeated dosing with 40 mg/kg PCZ on days 1, 2, 13, and 14 (D1, D2, D13, D14), indicated by arrows. Time points of sampling for PCZ concentration and DPP3 activity are indicated on the x-axis (h for hours, min for minutes). Interquartile range=IQR.

FIG. 36: Median (IQR) PCZ concentration and DPP3 activity in cynomolgus monkeys after repeated dosing with 350 mg/kg PCZ on days 1, 2, 13, and 14 (D1, D2, D13, D14), indicated by arrows. Time points of sampling for PCZ concentration and DPP3 activity are indicated on the x-axis (h for hours, min for minutes). Interquartile range=IQR.

FIG. 37: Overview of the experimental setup. BP=blood pressure; ECG=electrocardiogram.

FIG. 38: An overview of the interventions performed on the treatment day, including the in-hospital (overnight) observation period up until 24 hours after start of IMP administration. IMP-infusion=investigation medicinal product infusion; prep-time=preparation time needed for placement of venous and arterial catheter, applying equipment necessary for patient monitoring; ECG=electrocardiogram.

FIG. 39: Mean PCZ concentrations in healthy men (n=6 per dose) after dosing with 3 mg/kg, 6 mg/kg, or 12 mg/kg PCZ, administered as a single i.v. infusion over 2 hours.

FIG. 40: Mean cDPP3 activity in healthy men (n=6 per group) after dosing with 3 mg/kg, 6 mg/kg, or 12 mg/kg PCZ, or placebo, administered as a single i.v. infusion over 2 hours.

FIG. 41: Trial scheme for Example 14, multi-center, randomized, placebo-controlled, double-blind Phase 1b trial to investigate safety, tolerability, and pharmacokinetics of procizumab (PCZ) in patients with cardiogenic shock and elevated circulating Dipeptidyl Peptidase 3 (cDPP3) concentrations.

FIGS. 42 A and 42 B: Norepinephrine dosing before (“pre-dose”) and after (“24 h and 48 h post-dose”) treatment with PCZ. Shown are mean/SD values obtained from the three patients treated with PCZ. FIG. 42 A shows the absolute dosages; FIG. 42 B shows the dosages relative to the pre-dose dosages.

FIG. 43: Blood lactate concentrations before (“pre-dose”) and after (“24 h and 48 h post-dose”) treatment with PCZ. Shown are mean/SD values obtained from the three patients treated with PCZ. Hyperlactatemia is defined as a lactate concentration above 2 mmol/L (indicated by the dotted line) (Vieira, Petrova, and Moura 2022).

FIG. 44: Blood IL-6 concentrations before (“pre-dose”) and after (“24 h and 48 h post-dose”) treatment with PCZ. Shown are mean/SD values obtained from the three patients treated with PCZ. The upper limit of the healthy normal range is indicated by a dotted line.

FIGS. 45 A and 45 B: Kidney function before (“pre-dose”) and after (“24 h and 48 h post-dose”) treatment with PCZ. Shown are mean/SD values obtained from the three patients treated with PCZ for blood creatinine concentrations (FIG. 45 A) and the Glomerular Filtration Rate (GFR) calculated by the CKD-EPI formula (FIG. 45 B). Limits of healthy normal ranges are indicated by dotted lines (between 0.55 and 1.02 mg/dL creatinine, and between 90 and 120 mL/min for the GFR).

FIG. 46: Blood NT-proBNP concentrations before (“pre-dose”) and after (“24 h and 48 h post-dose”) treatment with PCZ. Shown are mean/SD values obtained from the three patients treated with PCZ. The upper limits of the healthy normal ranges are dependent on age and sex. The applicable limits according to Welsh et al. 2022 are indicated by dotted lines and the patient individual data are indicated with arrows.

FIG. 47: Mean PCZ concentration and cDPP3 activity in extreme-critically ill patients with refractory shock and multi-organ failure (n=3) after dosing with 10 mg/kg PCZ infused over two hours.

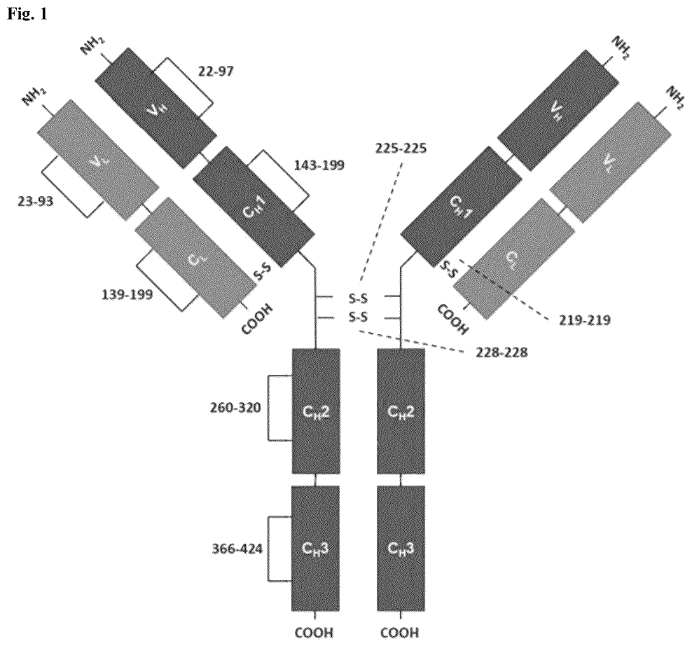

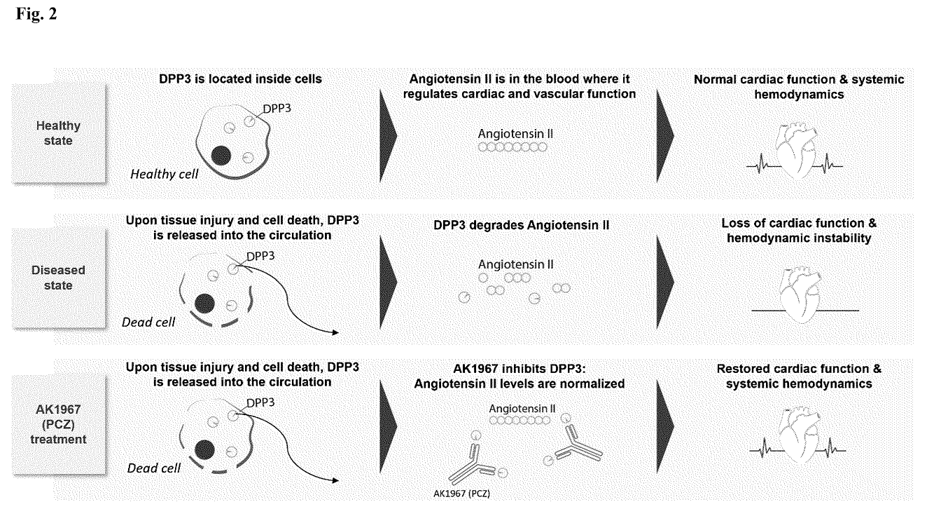

FIG. 48: Schematic representation of the development and selection of anti-DPP3 antibodies using the SELMA technology.
a) Myeloma cells are used for fusion with B-cells. The myeloma cells are stable transfected with a construct enabling the expression of a surface marker containing the biotin acceptor peptide sequence for site-specific biotinylation by biotin ligase (BirA).
b) After fusion of these myeloma cells with the B-cells derived from an immunized mouse, the pool of hybridoma cells are biotinylated in vitro at their surface via biotin ligase.
c) A separately prepared DPP3-streptavidin conjugate is added to the pool of cells, where it binds to the biotin-residues carried by the hybridoma cells.
d) During a subsequent incubation, the hybridoma cells secrete their monoclonal antibodies (mAbs). Those mAbs directed against the target protein DPP3 immediately after secretion bind to the DPP3-streptavidin conjugate nearby, whereas other mAbs from other hybridoma cells diffuse into the culture medium.
e) A fluorescence-labeled anti-mouse IgG is added and binds to mAbs. “Positive” hybridoma cells now carry a fluorescence label (complex formed by biotin covalently attached at the cell surface*Streptavidin conjugated DPP3*anti-DPP3 mAb* fluorescence-labeled anti-mouse IgG); “negative” hybridoma cells do not. By fluorescence-activated cell sorting (FACS), “positive” hybridoma cells can be easily isolated.

FIG. 49: Schematic representation of designs for assessment of DPP3 binding and influence on DPP3 enzymatic activity for anti-DPP3 antibodies.

### ILLUSTRATIVE EMBODIMENTS

The term “patient” as used herein refers to a living human or non-human organism that is receiving medical care or that should receive medical care due to a disease. This includes persons with no defined illness who are being investigated for signs of pathology. Thus, the methods and assays described herein are applicable to both human and veterinary disease.

Myocardial injury is defined by an elevation of cardiac troponin values above the 99th percentile upper reference limit. In particular, myocardial injury is a structural injury of myocardial cells and tissue (e.g. cardiomyocytes cardiofibroblasts, smooth muscle cells or endothelial cells).

It is considered a prerequisite for the diagnosis of myocardial infarction but also an entity in itself and can arise from non-ischemic or non-cardiac conditions (Thygesen et al., 2018, Eur Heart J 40(3):237-69 (Thygesen 2018); Chapman et al., 2016, Heart I 03(1):I0-8). The term ‘myocardial injury’ might be used in the setting of direct cardiac damage such as cardiac contusion, but it might also occur in diverse other clinical scenarios such as myocardial infarction, myocardial inflammation, sepsis, and iatrogenic injury.

Specifically, myocardial injury can have the following causes:
primary myocardial ischemia/myocardial infarction (atherosclerotic plaque rupture with thrombosis),
mismatch in myocardial oxygen supply and demand (coronary vasospasm, microvascular dysfunction, coronary embolism/microembolism/dissection, sustained bradyarrhythmias/tachyarrhythmias, hypovolemic shock, respiratory failure/severe anemia, left ventricular hypertrophy/hypertrophic cardiomyopathy, severe hypertension),
non-ischemic myocardial injury (heart failure, myocardial inflammation/myocarditis, cardiomyopathies/Tako-tsubo cardiomyopathy, cardiac contusion, iatrogenic (revascularization, cardiac surgery, ablation, pacing, cardioversion, defibrillation), rhabdomyolysis
multifactorial and systemic causes (sepsis/critical illness, cardiotoxicity (drugs), infiltrative disease (cardiac amyloidosis, cardiac sarcoidosis), pulmonary embolism/pulmonary hypertension, acute or chronic renal disease, stroke/subarachnoid hemorrhage)).

Presumed mechanisms of myocardial injury include direct cardiac damage with cardiomyocyte injury, myocardial strain as a result of excessive wall stress and myocardial ischemia due to myocardial oxygen supply and demand mismatch. Myocardial injury might be irreversible and is often associated with myocardial necrosis or apoptosis (Park et al., 2017, Cardiovasc Res. 113 (14):1708-18).

In the context of the present invention “blood pressure” means mean arterial pressure (MAP), which is the average arterial pressure throughout one cardiac cycle, systole, and diastole. MAP is influenced by cardiac output and systemic vascular resistance, each of which is influenced by several variables. MAP is a major determinant of the perfusion pressure seen by organs in the body. Current guidelines recommend targeting a MAP goal of 65 mm Hg or more in critically ill medical patients (Dellinger et al., 2012, Crit. Care Med. 2013; 41(2):580-637; Peberdy, et al., 2010, Circulation 122(18 Suppl 3): S768-786).

In one embodiment blood pressure decline is a MAP <65 mmHg, more preferred <60 mmHg, even more preferred <55 mmHg, most preferred <50 mmHg.

In another embodiment said blood pressure decline is a reduction in MAP of at least 5 mmHg, more preferred of at least 10 mmHg, even more preferred of at least 15 mmHg, most preferred of at least 20 mmHg.

Interleukin-6 (IL-6) is an important inflammatory mediator that is secreted to the circulatory system in response to infections and tissue injuries in the acute phases. IL-6 expression is tightly regulated, with low levels of expression in healthy individuals. Cardiomyocytes produce IL-6 under hypoxic and ischemic stress (Fuchs, et al., 2003, FASEB J 17 (14): 2118-2120). This activates the JAK/STAT cascade in these cells to exert negative inotropic and cytotoxicity. The inflammatory reaction mediates neutrophil infiltration and activation, triggering the release of further cytokines into the blood, co-stimulating vascular endothelium and inducing cardiomyocytes to express ICAM-1 to lead to myocardial fibrosis and ischemia/reperfusion injury (Gwechenberger; et al., 1999, Circulation 99(4):546-551), which, as a consequence, accelerates myocardial damage and dysfunction (Halawa, et al., 1999, Pol. Arch. Med. Wewn 101(3):197-203).

Troponins are structural proteins found in the troponin complex within skeletal and cardiac muscle thin filaments. The troponin complex consists of three subunits (I, T, and C) and along with calcium ions plays an important role in the regulation of muscle contraction (Kozinski, et al., 2017, Critical Reviews in Clinical Laboratory Sciences 54 (3):143-172). Each molecule has a specific role in the muscle contraction process: troponin T attaches the troponin complex to the actin filament, troponin C acts as the calcium binding site, and troponin I inhibits interaction with myosin heads in the absence of sufficient calcium ions (Garg, et al., 2017, Internal and Emergency Medicine 12(2):147-155). Troponin T and I are mainly localised in the myocardium, thus being referred to as cardiac troponin (cTnI and cTnT). It is generally accepted that these biomarkers possess the greatest specificity in identifying myocardial injury (Chauin, 2021, Vascular Health and Risk Management 17:299-316). Myocardial injury is ascertained if detectable cardiac troponin concentrations are found above the 99th percentile of the upper reference limit (URL) (Thygesen, et al., 2019, Eur Heart J. 40:237-269 (Thygesen 2019)).

Myocardial protection means the prevention of myocardial injury.

Prevention of myocardial injury is defined as a prevention of an increase of cardiac troponins in the circulation. In particular, prevention of myocardial injury is defined as a prevention of structural injury of myocardial cells and tissue (e.g. cardiomyocytes cardiofibroblasts, smooth muscle cells or endothelial cells) defined as an increase of cardiac troponins in the circulation.

In a specific embodiment myocardial injury is characterized by blood levels of cardial troponin above a threshold, increased myocardial expression of pro-inflammatory interleukin-6 (IL-6) and/or need of vasopressor (to maintain blood pressure and cardiac output).

Said cardial troponin is selected from the group comprising cardial troponin T (cTnT) and cardial troponin I (cTnI). Troponins may be measured with high-sensitive troponin (hs-Tn) assays. Thresholds of cardiac troponin concentrations are for example above the 99th percentile of the upper reference limit (URL) (Thygesen 2019).

The elevation of cardiac troponin values is further defined as rising of cardiac high-sensitive Troponin I (hs-cTnI) or cTnT values with at least one value above the 99th percentile of the upper reference limit.

Reference limits are sex-dependent (with higher values in men compared to women). Moreover, the reference values depend on the assay used (Sandoval, et al., 2022, Circulation 146:569-581), see Table 1 below.

TABLE 1
FDA-Cleared High-Sensitivity Cardiac Troponin Assay Thresholds

| 99th percentile, ng/L | | | |
|---|---|---|---|
| | Sex-specific | | |
| Overall | Male | Female | Assay |
| 28 | 17 | 35 | Abbott ARCHITECT hs-cTnI |
| 17.5 | 11.6 | 19.8 | Beckman Coulter Access 2 hs-cTnI (plasma) |
| 18.1 | 11.8 | 19.7 | Beckman Coulter Access 2 hs-cTnI (serum) |
| 17.9 | 14.9 | 19.8 | Beckman Coulter Dx1 Access hs-cTnI (plasma) |
| 18.1 | 13.6 | 19.8 | Beckman Coulter Dx1 Access hs-cTnI (serum) |
| 19 | 14 | 22 | Roche cobas e601, e602, E170/TnT Gen 5 STAT |
| 45.4 | 38.6 | 53.5 | Siemens ATELLICA high-sensitivity TnI (TNIH) |
| 46.5 | 39.6 | 58.0 | Siemens ADVIA Centaur XP/XPT/CP high-sensitivity TnI |
| | | | (TNIH) |
| 58.9 | 53.7 | 78.5 | Siemens Dimension VISTA high-sensitivity TnI (TNIH) |
| 60.4 | 51.4 | 76.2 | Siemens Dimension ExL high-sensitivity TnI (TNIH) |

The skilled person will readily determine suitable threshold values in view of the particular assay conditions, based on routine considerations and activities that are common knowledge in the field.

Vasopressors increase vasoconstriction, which leads to increased systemic vascular resistance (SVR). Increasing the SVR leads to increased mean arterial pressure (MAP) and increased perfusion to organs. Vasopressors are selected from the group comprising isoproterenol, dobutamine, dopamine, phenylephrine, norepinephrine, epinephrine, vasopressin or terlipressin.

Need of vasopressor is defined as a mean arterial pressure (MAP) below 65 mmHg. Said patient is a critically ill patient suffering from a disease selected from the group of severe infectious diseases, sepsis, pneumonia including community-acquired pneumonia (CAP), pulmonary embolism, myocardial infarction, any type of shock (including cardiogenic shock, septic shock or anaphylactic shock) and acute respiratory distress syndrome (ARDS).

“Critically ill” means that said patient is suffering from an acute disease or acute condition which is life-threatening and in which death is possible or imminent. In a specific embodiment said critically ill patient is an ICU patient.

Said infectious disease may be of bacterial, viral, fungal or parasitic origin. Said viral infection may be selected from infection caused by influenza virus or coronavirus.

Said coronavirus is selected from the group comprising SARS-CoV-1, SARS-CoV-2, MERS-CoV, in particular SARS-CoV-2. Coronaviruses cause diseases in mammals and birds. In humans, the viruses cause respiratory infections, including the common cold, which are typically mild, though rarer forms such as SARS, MERS and COVID-19 can be lethal. SARS-CoV-1 or -2 infection may present with mild, moderate, or severe illness; the latter includes severe pneumonia, acute respiratory distress syndrome (ARDS), sepsis and septic shock.

In a specific embodiment said viral infection is not caused by coronavirus; in other words, the infection is not an infection with a coronavirus.

Acute respiratory distress syndrome (ARDS) is a type of respiratory failure characterized by rapid onset of widespread inflammation in the lungs. Symptoms include shortness of breath, rapid breathing, and bluish skin coloration. For those who survive, a decreased quality of life is common. Causes may include sepsis, pancreatitis, trauma, pneumonia, and aspiration. The underlying mechanism involves diffuse injury to cells which form the barrier of the microscopic air sacs of the lungs, surfactant dysfunction, activation of the immune system, and dysfunction of the body's regulation of blood clotting. In effect, ARDS impairs the lungs' ability to exchange oxygen and carbon dioxide. Diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. The primary treatment involves mechanical ventilation together with treatments directed at the underlying cause. Ventilation strategies include using low volumes and low pressures. If oxygenation remains insufficient, lung recruitment maneuvers and neuromuscular blockers may be used. If this is insufficient, extracorporeal membrane oxygenation (ECMO) may be an option. The syndrome is associated with a death rate between 35 and 50%.

Acute coronary syndrome refers to a group of diseases in which blood flow to the heart is decreased and includes ST-elevation myocardial infarction (STEMI), non-ST elevation myocardial infarction (NSTEMI), and unstable angina. It is a type of coronary heart disease (CHD), which is responsible for one-third of total deaths in people older than 35 years of age. Some forms of CHD can be asymptomatic, but ACS is always symptomatic. According to the guidelines, acute myocardial infarction is defined as follows: The term acute myocardial infarction should be used when there is acute myocardial injury with clinical evidence of acute myocardial ischaemia and with detection of a rise and/or fall of cardiac troponin (cTn) values with at least one value above the 99th percentile upper reference level (URL) and at least one of symptoms of myocardial ischaemia, new ischaemic ECG changes, development of pathological Q waves, imaging evidence of new loss of viable myocardium or new regional wall motion abnormality in a pattern consistent with an ischaemic aetiology or identification of a coronary thrombus by angiography or autopsy (Thygesen 2018). Moreover, there also exists coronary procedure-related myocardial infarction as percutaneous coronary intervention (PCI) or coronary artery bypass grafting (CABG).

Pulmonary function is assessed, particularly in patients under invasive ventilation in critical care, with the Horowitz index (synonyms: oxygenation after Horowitz, Horowitz coefficient, P/F ratio). It is useful for evaluating the extent of damage to the lungs. The Horowitz index (PaO2/FiO2 ratio) is a well-known marker of acute pulmonary injury and predicts mortality in patients with acute respiratory failure. The Horowitz index is defined as the ratio of partial pressure of oxygen in blood (PaO2), in millimeters of mercury, and the fraction of oxygen in the inhaled air (FiO2)—the PaO2/FiO2 ratio. In healthy lungs the Horowitz index depends on age and usually falls between 350 and 500. A value below 300 is the threshold for mild lung injury, and 200 is indicative of a moderately severe lung injury. A value below 100 as a criterion for a severe injury. The Horowitz index plays a major role in the diagnosis of acute respiratory distress syndrome (ARDS). Three seventies of ARDS are categorized based on the degree of hypoxemia using the Horowitz index, according to the Berlin definition (Matthav, et al., 2012, J. Clin. Invest. 122(8):2731-2740).

Pulmonary dysfunction or reduction of pulmonary function is defined as a Horowitz index below 350, more preferred below 300, even more preferred below 200, most preferred below 100.

In one embodiment of the invention said patient has a reduction of pulmonary function, which is defined as a decrease in the Horowitz index of at least 10%, preferably at least 20%, more preferred at least 50%, most preferred of at least 75%.

Improvement of pulmonary function is defined as an increase in the Horowitz index of more than 5%, more preferred of more than 10%, even more preferred of more than 20%, even more preferred of more than 30%, most preferred of more than 50%.

### Ventilation

In another specific embodiment said critically ill patient is under ventilation.

The term “ventilation” includes non-invasive or invasive ventilation.

Non-invasive ventilation is the use of breathing support administered through a face mask, nasal mask, or a helmet. Air, usually with added oxygen, is given through the mask under positive pressure.

Mechanical ventilation or assisted ventilation, is the medical term for artificial ventilation where mechanical means are used to assist or replace spontaneous breathing.

This may involve a machine called a ventilator, or the breathing may be assisted manually by a suitably qualified professional, such as an anesthesiologist, respiratory therapist (RT), Registered Nurse, or paramedic, by compressing a bag valve mask device. Mechanical ventilation is termed “invasive” if it involves any instrument inside the trachea through the mouth, such as an endotracheal tube or the skin, such as a tracheostomy tube. Face or nasal masks are used for non-invasive ventilation in appropriately selected conscious patients.

Extracorporeal membrane oxygenation (ECMO), also known as extracorporeal life support (ECLS), is an extracorporeal technique for providing prolonged cardiac and respiratory support to persons whose heart and lungs are unable to provide an adequate amount of gas exchange or perfusion to sustain life. The technology for ECMO is largely derived from cardiopulmonary bypass, which provides shorter-term support with arrested native circulation. ECMO works by removing blood from the person's body and artificially removing carbon dioxide from, and adding oxygen to, the patient's red blood cells. Generally, it is used either post-cardiopulmonary bypass or in late-stage treatment of a person with profound heart and/or lung failure, although it is now seeing use as a treatment for cardiac arrest in certain centers, allowing treatment of the underlying cause of arrest while circulation and oxygenation are supported. ECMO is also used to support patients with the acute viral pneumonia associated with e.g. COVID-19 in cases where artificial ventilation is not sufficient to sustain blood oxygenation levels. In addition, venovenous ECMO could be used to provide respiratory support in patients with septic shock.

### DPP3 Inhibitor

Inhibitors are molecules that preferably significantly inhibit DPP3 activity. Those molecules can be peptides and small molecules, antibodies, antibody fragments or non-Ig scaffolds.

Significantly inhibiting means inhibiting the activity of DPP3 more than 60%, preferably more than 70%, more preferably more than 80%, preferably more than 90%, more preferably almost or actually 100% inhibition.

The activity of DPP3 can be inhibited unspecifically by different general protease inhibitors (e.g., PMSF, TPCK), sulfhydryl reagents (e.g., pHMB, DTNB) and metal chelators (EDTA, o-phenantroline). (Abramić 2000).

DPP3 activity can be further inhibited specifically by different kinds of compounds: an endogenous DPP3-inhibitor is the peptide spinorphin. Several synthetic derivatives of spinorphin, e.g., tynorphin, have been produced and shown to inhibit DPP3 activity to varying extents. (Yamamoto, et al., 2000, Life Sciences 62(19):1767-1773 (Yamamoto 2000)). Other published peptide inhibitors of DPP3 are propioxatin A and B (U.S. Pat. No. 4,804,676) and propioxatin A analogues (Inaoka, et al., 1988, J. Biochem 104(5):706-711).

A “derivative or analogue” is a chemical compound that is derived from a parent compound by a chemical reaction with the replacement of one atom or substitution of a group of atoms by a functional group. Parent and derivative compounds have similar chemical structures.

DPP3 can also be inhibited by small molecules such as fluostatins and benzimidazol derivatives. Fluostatins A and B are antibiotics produced in Streptomyces sp. TA-3391 that are non-toxic and strongly inhibit DPP3 activity. So far, 20 different derivatives of benzimidazol have been synthesized and published (Agid, et al., 2007, Bioorganic Chemistry 35(2):153-169 (Agid 2007); Rastija, et al., 2015, Acta Chimica Slovenica 62:867-878), of which the two compounds 1′ and 4′ show the strongest inhibitory effect (Agid 2007). Several dipeptidyl hydroxamic acids have been shown to inhibit DPP3 activity as well (Cviteŝić, et al., 2016, J Enzyme Inhib Med Chem 31(sup2):40-45).

A “small molecule” is in particular a low molecular weight (more particularly <1000 daltons) organic compound. Such small molecules may in particular regulate a biological process, e.g. bind a specific biological macromolecule, in the present invention in particular DPP3, and act as an effector, in particular an inhibitor, altering the activity or function of the biological macromolecule. Small molecules can be of natural origin or artificial.

Particular examples of small molecule and peptide inhibitors of DPP3 are shown in the following Table 2, see also SEQ ID NOs: 39-61. Compounds 1′ and 4′ show the strongest inhibitory effect (Agid 2007).

| Compound Type | | Full Name/Amino Acid Sequence | Reference |
|---|---|---|---|
| small molecules | fluostatins | 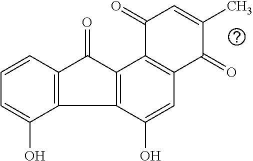 [SMILES: Cc1cc(=O)c2c(cc(O)c3c4c(O)cccc4c(=O)c32)c1=O] | Akiyama et al. 1998 |
| | | Fluostatin A | |
| | | 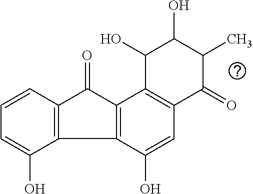 [SMILES: CC1C(=O)c2cc(O)c3c(c2C(O)C1O)C(=O)c1cccc(O)c1-3] | |
| | | Fluostatin B | |
| | benzimidazol derivatives | 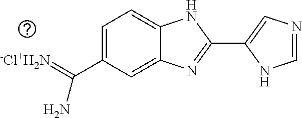 | Agic et al. 2007 |
| | | Compound 1 | |
| | | 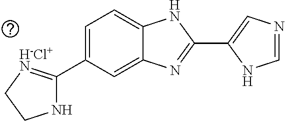 | |
| | | Compound 2 | |
| | | 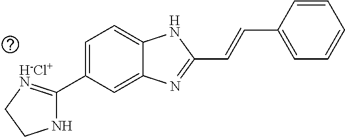 | |
| | | Compound 3 | |
| | | 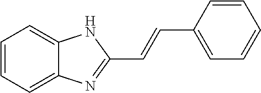 [SMILES: C(=C\c1nc2ccccc2[nH]1)/c1ccccc1] | |
| | | Compound 4 | |
| | | 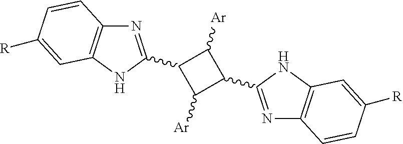 | |
| | | Compound 1′-8′ | |
| | | 1′ Ar = phenyl | |
| | | 4′ Ar = o-Cl-phenyl | |
| | | 7′ Ar = 2-furyl | |
| | | 8′ Ar = 2-thienyl | |
| | | 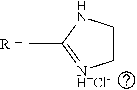 | |
| | | 2′ Ar = phenyl | |
| | | 5′ Ar = o-Cl-phenyl | |
| | | 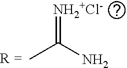 | |
| | | 3′ Ar = phenyl | |
| | | 6′ Ar = o-Cl-phenyl | |
| | | 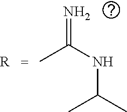 | |
| | | 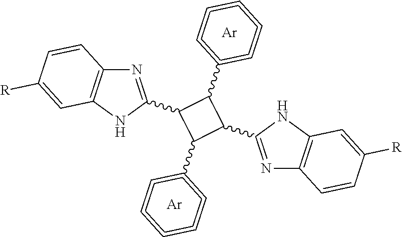 | Rastija et al. 2015 |
| | | Compound 1′-16 | |
| | | 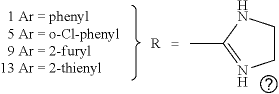 | |
| | | 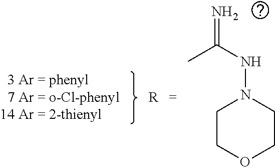 | |
| | | 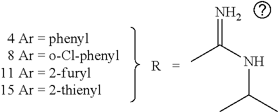 | |
| | | 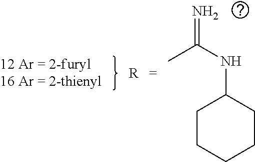 | |
| | | 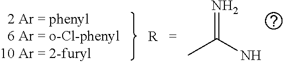 | |
| peptides | propioxatins and propioxatin A analogues | 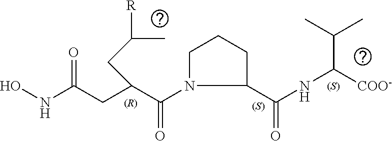 | US 4804676 A/Inaoka et al. 1986 |
| | | Propioxatin A and B | |
| | | A: R = H | |
| | | B: R = CH₃ | |
| | | Propioxatin A analogues (Compound 1-17) | Inaoka et al. |
| | | 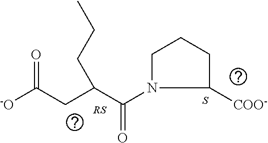 | 1988 |
| | | 1 | |
| | | 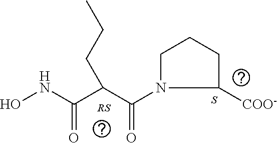 | |
| | | 2 | |
| | | 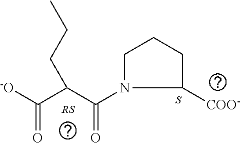 | |
| | | 3 | |
| | | 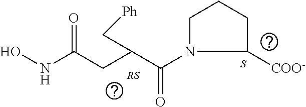 | |
| | | 4 | |
| | | 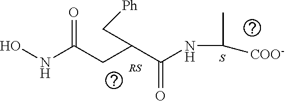 | |
| | | 5 | |
| | | 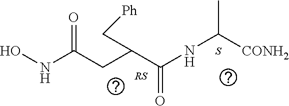 | |
| | | 6 | |
| | | 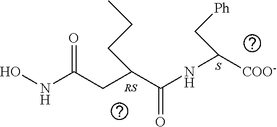 | |
| | | 7 | |
| | | 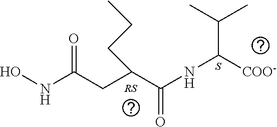 | |
| | | 8 | |
| | | 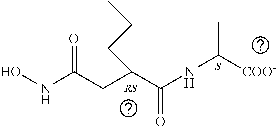 | |
| | | 9 | |
| | | 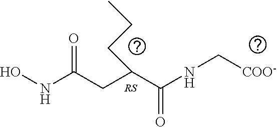 | |
| | | 10 | |
| | | 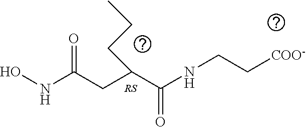 | |
| | | 11 | |
| | | 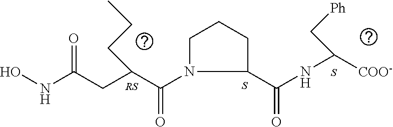 | |
| | | 12 | |
| | | 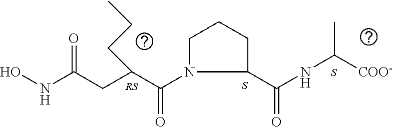 | |
| | | 13 | |
| | | 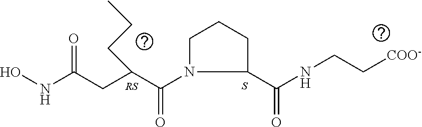 | |
| | | 14 | |
| | | 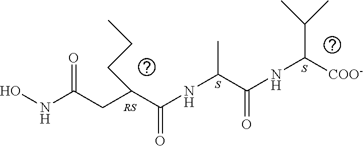 | |
| | | 15 | |
| | | 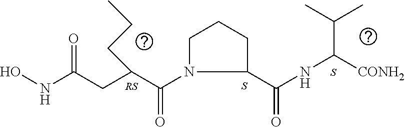 | |
| | | 16 | |
| | | 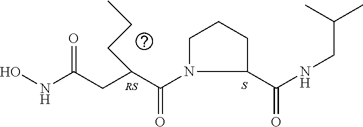 | |
| | | 17 | |
| | spinorphin | Spinorphin (LVVYPWT) | Yamamoto |
| | and | Tynorphin (VVYPW) | 2000; Chiba |
| | spinorphin | Spinorphin derivatives (AVYPW, FIVPW, FVAPW, | et al. 2003 |
| | derivatives | FVYPW, GVYPW, IVYPW, LVVPW, LVVYP, LVVYPW, | |
| | | LVYPW, PWT, SVYPW, VVYP, VVYPWT, VYP, VYPW, | |
| | | VYPWT, WVYPW, YAIPW, YPW, YPWT, YSIPW, | |
| | | YSVPW, YVYPW) | |

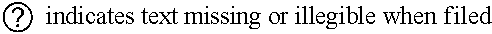

Subject-matter of the present application is an inhibitor of the activity of DPP3 for use in therapy or intervention in a critically ill patient with a decline in blood pressure for myocardial protection and/or prevention of myocardial injury, wherein the inhibitor of the activity of DPP3 is selected from the group comprising small molecules, anti-DPP3 antibody, anti-DPP3 antibody fragment or anti-DPP3 non-lg scaffold.

Subject matter of the present application is an inhibitor of the activity of DPP3 for use in therapy or intervention in a critically ill patient with a decline in blood pressure for myocardial protection and/or prevention of myocardial injury, wherein the inhibitor of the activity of DPP3 is a small molecule selected from the group comprising spinorphin, tynorphin, propioxatin A and B, fluostatin A and B, enzimidazole or derivatives or analogues thereof.

Subject matter of the present application is an inhibitor of the activity of DPP3 for use in therapy or intervention in a critically ill patient with a decline in blood pressure for myocardial protection and/or prevention of myocardial injury, wherein said inhibitor is an anti-DPP3 antibody, anti-DPP3 antibody fragment or anti-DPP3 non-lg scaffold that exhibits a minimum binding affinity to DPP3 of equal or less than 10⁻⁷ M.

Subject matter of the present application is an inhibitor of the activity of DPP3 for use in therapy or intervention in a critically ill patient with a decline in blood pressure for myocardial protection and/or prevention of myocardial injury, wherein said antibody is a monoclonal antibody or monoclonal antibody fragment.

Subject-matter of the present application is an inhibitor of the activity of DPP3 for use in therapy or intervention of pulmonary function in a critically ill patient with a reduction in pulmonary function for the improvement of pulmonary function, wherein the inhibitor of the activity of DPP3 is selected from the group comprising small molecules, anti-DPP3 antibody, anti-DPP3 antibody fragment or anti-DPP3 non-Ig scaffold.

Subject matter of the present application is an inhibitor of the activity of DPP3 for use in therapy or intervention in a critically ill patient with a reduction in pulmonary function for the improvement of pulmonary function, wherein the inhibitor of the activity of DPP3 is a small molecule selected from the group comprising spinorphin, tynorphin, propioxatin A and B, fluostatin A and B, benzimidazol or derivatives or analogues thereof.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody, anti-DPP3 antibody fragment or anti-DPP3 non-Ig scaffold that exhibits a minimum binding affinity to DPP3 of equal or less than 10⁻⁷ M.

Subject matter of the present application is an inhibitor of the activity of DPP3 for use in therapy or intervention in a critically ill patient with a reduction in pulmonary function for the improvement of pulmonary function, wherein said antibody is a monoclonal antibody or monoclonal antibody fragment.

Throughout the specification the “antibodies”, or “antibody fragments” or “non-Ig scaffolds” in accordance with the invention are capable to bind DPP3, and thus are directed against DPP3, and thus can be referred to as “anti-DPP3 antibodies”, “anti-DPP3 antibody fragments”, or “anti-DPP3 non-Ig scaffolds”.

The term “antibody” generally comprises monoclonal and polyclonal antibodies and binding fragments thereof, in particular Fc-fragments as well as so called “single-chain-antibodies” (Bird, et al., 1988, Science 242:423-426 (Bird 1988)), chimeric, humanized, in particular CDR-grafted antibodies, and dia or tetrabodies (Holliger; P, et al., 1993, Proc Nat Acad Sci USA, July 15; 90(14):6444-8). Also comprised are immunoglobulin-like proteins that are selected through techniques including, for example, phage display to specifically bind to the molecule of interest contained in a sample. In this context the term “specific binding” refers to antibodies raised against the molecule of interest or a fragment thereof. An antibody is considered to be specific if its affinity towards the molecule of interest or the aforementioned fragment thereof is at least preferably 50-fold higher, more preferably 100-fold higher, most preferably at least 1000-fold higher than towards other molecules comprised in a sample containing the molecule of interest. It is well known in the art how to make antibodies and to select antibodies with a given specificity.

In one embodiment of the invention the anti-DPP3 antibody or anti-DPP3 antibody fragment or anti-DPP3 non-Ig scaffold is monospecific.

Monospecific anti-DPP3 antibody or monospecific anti-DPP3 antibody fragment or monospecific anti-DPP3 non-Ig scaffold means that said antibody or antibody fragment or non-Ig scaffold binds to one specific region encompassing at least 5 amino acids within the target DPP3 (SEQ ID NO: 1). Monospecific anti-DPP3 antibody or monospecific anti-DPP3 antibody fragment or monospecific anti-DPP3 non-Ig scaffold are anti-DPP3 antibodies or anti-DPP3 antibody fragments or anti-DPP3 non-Ig scaffolds that all have affinity for the same antigen. Monoclonal antibodies are monospecific, but monospecific antibodies may also be produced by other means than producing them from a common germ cell.

In a specific embodiment said anti-DPP3 antibody, anti-DPP3 antibody fragment or anti-DPP3 non-Ig scaffold is an inhibiting antibody, fragment or non-Ig scaffold. Said anti-DPP3 antibody, anti-DPP3 antibody fragment or anti-DPP3 non-Ig scaffold is inhibiting the activity of DPP3 more than 50%, preferably more than 60%, preferably more than 70%, more preferably more than 80%, preferably more than 90%, even more preferred more than 95%, preferably almost or actually 100%.

An antibody or fragment according to the present invention is a protein including one or more polypeptides substantially encoded by immunoglobulin genes that specifically binds an antigen. The recognized immunoglobulin genes include the kappa, lambda, alpha (IgA), gamma (IgG₁, IgG₂, IgG₃, IgG₄), delta (IgD), epsilon (IgE) and mu (IgM) constant region genes, as well as the myriad immunoglobulin variable region genes. Full-length immunoglobulin light chains are generally about 25 Kd or 214 amino acids in length.

Full-length immunoglobulin heavy chains are generally about 50 Kd or 446 amino acid in length. Light chains are encoded by a variable region gene at the NH₂-terminus (about 110 amino acids in length) and a kappa or lambda constant region gene at the COOH-terminus. Heavy chains are similarly encoded by a variable region gene (about 116 amino acids in length) and one of the other constant region genes.

The basic structural unit of an antibody is generally a tetramer that consists of two identical pairs of immunoglobulin chains, each pair having one light and one heavy chain. In each pair, the light and heavy chain variable regions bind to an antigen, and the constant regions mediate effector functions. Immunoglobulins also exist in a variety of other forms including, for example, Fv, Fab, and (Fab′)₂, as well as bifunctional hybrid antibodies and single chains (e.g., Lanzavecchia, et al., 1987, Eur J. Immunol. 17:105; Huston, et al., 1988, Proc. Natl. Acad. Sci. U.S.A., 85:5879-5883; Bird 1988; Hood, et al., 1984, Immunology, Benjamin, N.Y., 2nd ed.; Hunkapiller and Hood, 1986, Nature 323:15-16). An immunoglobulin light or heavy chain variable region includes a framework region interrupted by three hypervariable regions, also called complementarity determining regions (CDRs) (see Sequences of Proteins of Immunological Interest, E. Kabat et al., 1983, U.S. Department of Health and Human Services). As noted above, the CDRs are primarily responsible for binding to an epitope of an antigen. An immune complex is an antibody, such as a monoclonal antibody, chimeric antibody, humanized antibody or human antibody, or functional antibody fragment, specifically bound to the antigen.

Chimeric antibodies are antibodies whose light and heavy chain genes have been constructed, typically by genetic engineering, from immunoglobulin variable and constant region genes belonging to different species. For example, the variable segments of the genes from a mouse monoclonal antibody can be joined to human constant segments, such as kappa and gamma 1 or gamma 3. In one example, a therapeutic chimeric antibody is thus a hybrid protein composed of the variable or antigen-binding domain from a mouse antibody and the constant or effector domain from a human antibody, although other mammalian species can be used, or the variable region can be produced by molecular techniques. Methods of making chimeric antibodies are well known in the art (e.g., see U.S. Pat. No. 5,807,715). A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions, which have substantially no effect on antigen binding or other immunoglobulin functions. Exemplary conservative substitutions are those such as gly, ala; val, ile, leu; asp, glu; asn, gln; ser, thr; lys, arg; and phe, tyr. Humanized immunoglobulins can be constructed by means of genetic engineering (e.g., see U.S. Pat. No. 5,585,089). Human antibodies can also be prepared by using transgenic animals carrying a human immunoglobulin gene (for example, see WO 93/12227; WO 91/10741). A human antibody is an antibody wherein the light and heavy chain genes are of human origin. Human antibodies can be generated using methods known in the art. Antibodies can be produced by immortalizing a (e.g. mammalian) B cell secreting the antibody of interest. Immortalization can be accomplished, for example, by Epstein Barr Virus EBV infection of a B-cell or by fusing a B cell with a myeloma to make a hybridoma or fusing a B cell to a hybridoma cell to produce a trioma cell. Antibodies can also be produced by phage display methods (see, e.g., WO 91/17271; WO 92/001047; WO 92/20791) or selected from a combinatorial monoclonal antibody library (see the Morphosys website). Human antibodies can also be prepared by using transgenic animals carrying a human immunoglobulin gene (for example, see WO93/12227; WO 91/10741).

Thus, the anti-DPP3 antibody may have the formats known in the art. Examples are human antibodies, monoclonal antibodies, humanized antibodies, chimeric antibodies, CDR-grafted antibodies. In a preferred embodiment antibodies according to the present invention are recombinantly produced antibodies as e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the C_{H}3 domain; bivalent Fab or multivalent Fab, e.g. formed via multimerization with the aid of a heterologous domain, e.g. via dimerization of dHLX domains, e.g. Fab-dHLX-FSx2; F(ab′)₂-fragments, scFv-fragments, multimerized multivalent and/or multi-specific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulines and numerous others.

In a preferred embodiment the anti-DPP3 antibody format is selected from the group comprising Fv fragment, scFv fragment, Fab fragment, scFab fragment, F(ab)₂ fragment and scFv-Fc Fusion protein. In another preferred embodiment the antibody format is selected from the group comprising scFab fragment, Fab fragment, scFv fragment and bioavailability optimized conjugates thereof, such as PEGylated fragments. One of the most preferred formats is the scFab format.

Non-Ig scaffolds may be protein scaffolds and may be used as antibody mimics as they are capable to bind to ligands or antigens. Non-Ig scaffolds may be selected from the group comprising tetranectin-based non-Ig scaffolds (e.g. described in US 2010/0028995), fibronectin scaffolds (e.g. described in EP 1 266 025; lipocalin-based scaffolds (e.g. described in WO 2011/154420); ubiquitin scaffolds (e.g. described in WO 2011/073214), transferrin scaffolds (e.g. described in US 2004/0023334), protein A scaffolds (e.g. described in EP 2 231 860), ankyrin repeat based scaffolds (e.g. described in WO 2010/060748), microproteins preferably microproteins forming a cysteine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1 941 867).

In one embodiment of the invention anti-DPP3 antibodies according to the present invention may be produced by synthesizing fragments of DPP3 as antigens or full-length DPP3. Thereafter, binder to said fragments are identified using the below described methods or other methods as known in the art.

In another preferred embodiment, the anti-DPP3 antibody, anti-DPP3 antibody fragment, or anti-DPP3 non-Ig scaffold is a full-length antibody, antibody fragment, or non-Ig scaffold.

Subject matter of the present application is an inhibitor of the activity of DPP3 for use in therapy or intervention in a critically ill patient with a reduction in pulmonary function for the improvement of pulmonary function, wherein the CDRs in the heavy chain comprises the sequences: SEQ ID NO: 7, SEQ ID NO: 8, and/or SEQ ID NO: 9 and the CDRs in the light chain comprises the sequences: SEQ ID NO:10, KVS, and/or SEQ ID NO: 11.

Subject matter of the present application is an inhibitor of the activity of DPP3 for use in therapy or intervention in a critically ill patient with a decline in blood pressure for myocardial protection and/or prevention of myocardial injury, wherein said monoclonal antibody or antibody fragment is a humanized monoclonal antibody or humanized monoclonal antibody fragment.

Subject matter of the present application is an inhibitor of the activity of DPP3 for use in therapy or intervention in a critically ill patient with a decline in blood pressure for myocardial protection and/or prevention of myocardial injury, wherein the heavy chain comprises the sequence SEQ ID NO: 12 and wherein the light chain comprises the sequence SEQ ID NO: 13.

Subject matter of the present application is an inhibitor of the activity of DPP3 for use in therapy or intervention in a critically ill patient with a decline in blood pressure for cardiac protection and/or prevention of cardiac damage, wherein said inhibitor is an anti-DPP3 antibody or anti-DPP3 antibody fragment or anti-DPP3 non-lg scaffold that binds an epitope of at least 4 or 5 amino acids in length comprised in SEQ ID NO: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO: 1.

Subject matter of the present application is an inhibitor of the activity of DPP3 for use in therapy or intervention in a critically ill patient with a decline in blood pressure for myocardial protection and/or prevention of myocardial injury, wherein said inhibitor is an anti-DPP3 antibody or anti-DPP3 antibody fragment or anti-DPP3 non-lg scaffold that binds an epitope of at least 4 or 5 amino acids in length comprised in SEQ ID NO: 2, and wherein the epitope is comprised in DPP3 as depicted in SEQ ID NO: 1. In one embodiment said inhibitor is an anti-antibody or anti-DPP3 antibody fragment or anti-DPP3 non-lg scaffold that binds an epitope of at least 4 amino acids in length comprised in SEQ ID NO: 2, and wherein the epitope is comprised in DPP3 as depicted in SEQ ID NO: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO: 2, and wherein the epitope is comprised in DPP3 as depicted in SEQ ID NO: 1. An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes and linear epitopes, based on their structure and interaction with the paratope. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontinuous amino acid residues. A linear or a sequential epitope is an epitope that is recognized by antibodies by its linear sequence of amino acids, or primary structure and is formed by the 3-D conformation adopted by the interaction of contiguous amino acid residues.

Subject matter of the present application is an inhibitor of the activity of DPP3 for use in therapy or intervention in a critically ill patient with a decline in blood pressure for myocardial protection and/or prevention of myocardial injury, wherein said inhibitor is an anti-DPP3 antibody or anti-DPP3 antibody fragment or anti-DPP3 non-lg scaffold that binds an epitope of at least 4 or 5 amino acids in length comprised in SEQ ID NO: 3, and wherein the epitope is comprised in DPP3 as depicted in SEQ ID NO: 1. In one embodiment said inhibitor is an anti-DPP3 antibody or anti-DPP3 antibody fragment or anti-DPP3 non-lg scaffold that binds an epitope of at least 4 amino acids in length comprised in SEQ ID NO: 3, and wherein the epitope is comprised in DPP3 as depicted in SEQ ID NO: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO: 3, and wherein the epitope is comprised in DPP3 as depicted in SEQ ID NO: 1.

Subject matter of the present application is an inhibitor of the activity of DPP3 for use in therapy or intervention in a critically ill patient with a decline in blood pressure for myocardial protection and/or prevention of myocardial injury, wherein said inhibitor is an anti-DPP3 antibody or anti-DPP3 antibody fragment or anti-DPP3 non-lg scaffold that binds an epitope of at least 4 amino acids in length comprised in SEQ ID NO: 4, and wherein the epitope is comprised in DPP3 as depicted in SEQ ID NO: 1.

Subject matter of the present application is an inhibitor of the activity of DPP3 for use in therapy or intervention in a critically ill patient with a reduction in pulmonary function for the improvement of pulmonary function, wherein said monoclonal antibody or antibody fragment is a humanized monoclonal antibody or humanized monoclonal antibody fragment.

Subject matter of the present application is an inhibitor of the activity of DPP3 for use in therapy or intervention in a critically ill patient with a reduction in pulmonary function for the improvement of pulmonary function, wherein the heavy chain comprises the sequence SEQ ID NO: 12 and wherein the light chain comprises the sequence SEQ ID NO: 13.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID NO: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO: 1.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID NO: 2, and wherein the epitope is comprised in DPP3 as depicted in SEQ ID NO: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO: 2, and wherein the epitope is comprised in DPP3 as depicted in SEQ ID NO: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO: 2, and wherein the epitope is comprised in DPP3 as depicted in SEQ ID NO: 1.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 or 5 amino acids in length comprised in SEQ ID NO: 3, and wherein the epitope is comprised in DPP3 as depicted in SEQ ID NO: 1. In one embodiment said inhibitor is an anti-DPP3 antibody or anti-DPP3 antibody fragment or anti-DPP3 non-Ig scaffold that binds an epitope of at least 4 amino acids in length comprised in SEQ ID NO: 3, and wherein the epitope is comprised in DPP3 as depicted in SEQ ID NO: 1. In another embodiment said inhibitor is an anti-DPP3 antibody or anti-DPP3 antibody fragment or anti-DPP3 non-Ig scaffold that binds an epitope of at least 5 amino acids in length comprised in SEQ ID NO: 3, and wherein the epitope is comprised in DPP3 as depicted in SEQ ID NO: 1.

Subject matter of the present application is an inhibitor of the activity of DPP3 for use in therapy or intervention in a critically ill patient with a reduction in pulmonary function for the improvement of pulmonary function, wherein said inhibitor is an anti-DPP3 antibody or anti-DPP3 antibody fragment or anti-DPP3 non-Ig scaffold that binds an epitope of at least 4 amino acids in length comprised in SEQ ID NO: 4, and wherein the epitope is comprised in DPP3 as depicted in SEQ ID NO: 1.

An epitope, also known as antigenic determinant, is the part of an antigen that is recognized by the immune system, specifically by antibodies. For example, the epitope is the specific piece of the antigen to which an antibody binds. The part of an antibody that binds to the epitope is called a paratope. The epitopes of protein antigens are divided into two categories, conformational epitopes and linear epitopes, based on their structure and interaction with the paratope. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontiguous amino acid residues. A linear or a sequential epitope is an epitope that is recognized by antibodies by its linear sequence of amino acids, or primary structure and is formed by the 3-D conformation adopted by the interaction of contiguous amino acid residues.

To assess the identity between two amino acid sequences, a pairwise alignment is performed. Identity defines the percentage of amino acids with a direct match in the alignment.

In a preferred embodiment, the treatment with an inhibitor of DPP3 activity is initiated or changed immediately upon provision of the result of the sample analysis indicating the level of DPP3 in the sample. In further embodiments, the treatment may be initiated within 12 hours, preferably 6, 4, 2, 1, 0.5, 0.25 hours or immediately after receiving the result of the sample analysis.

In some embodiments, the method comprises or consists of a single and/or multiple measurement of DPP3 in a sample from a patient in a single sample and/or multiple samples obtained at essentially the same time point, in order to guide and/or monitor and/or stratify a therapy, wherein said therapy is the administration of an inhibitor of the activity of DPP3

The term “pharmaceutical formulation” or “dosage form” means a pharmaceutical ingredient in combination with at least one pharmaceutically acceptable excipient, which is in such form as to permit the biological activity of a pharmaceutical ingredient contained therein to be effective, and which contains no additional components which are unacceptably toxic to a subject to which the formulation would be administered.

The term “pharmaceutical ingredient” means a therapeutic component (e.g., an anti-DPP3 antibody or an anti-DPP3 antibody fragment or an anti-DPP3 non-Ig scaffold) which can be combined with pharmaceutically acceptable excipients to provide a pharmaceutical formulation or dosage form.

In one embodiment the pharmaceutical formulation suitable for infusion or injection is an aqueous solution comprising a buffer. In another embodiment such solution comprises a sugar, wherein the sugar is, for example, sucrose, trehalose, or mannitol. In a further embodiment such solution comprises an amino acid, wherein the amino acid is, for example, lysine, arginine, or histidine. In one embodiment the pharmaceutical formulation suitable for injection is a dispersion comprising glycerol, liquid polyethylene glycols, triacetin, and mixtures thereof. Preferably, pharmaceutical formulation solutions are sterilized prior to administration to a patient.

In one embodiment the pharmaceutical formulation is in a freeze-dried state. Such a freeze-dried state may be a lyophilized cake that can be reconstituted to create a concentrated solution or a solution suitable for infusion or injection as described above.

The anti-DPP3 antibody or an anti-DPP3 antibody fragment or an anti-DPP3 non-Ig scaffold can be administered alone or with other pharmaceutical ingredients. For example, the anti-DPP3 antibody or an anti-DPP3 antibody fragment or an anti-DPP3 non-Ig scaffold can be administered before, substantially contemporaneous with, and/or after another pharmaceutical ingredient. The one or more pharmaceutical ingredients can be combined in a single pharmaceutical formulation. Other pharmaceutical ingredients include those considered to be the standard of care treatment for shock including but not limited to vasopressors and inotropic agents. Examples of vasopressors include vasopressin, norepinephrine, and epinephrine. Examples of inotropic agents include dopamine and dobutamine.

The pharmaceutical formulation comprising the anti-DPP3 antibody or an anti-DPP3 antibody fragment or an anti-DPP3 non-Ig scaffold may be administered to a mammalian animal, such as a human subject.

In one embodiment of the invention, the anti-DPP3 antibody or an anti-DPP3 antibody fragment or an anti-DPP3 non-Ig scaffold may be produced as outlined in Example 1 of U.S. Pat. No. 11,530,276, which is incorporated by reference for this production process, by synthesizing fragments of DPP3 as antigens or full-length DPP3. Thereafter, binders to said anti-DPP3 antibody or an anti-DPP3 antibody fragment or an anti-DPP3 non-Ig scaffold are identified using the described methods in U.S. Pat. No. 11,726,094, which is incorporated by reference for these identification methods, or other methods as known in the art.

Humanization of murine antibodies may be conducted according to the following procedure.

For humanization of an antibody of murine origin the antibody sequence is analysed for the structural interaction of framework regions (FR) with the CDR and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro and Fransson, 2008, Front Biosci.; 13:1619-33). The use of molecular modeling to achieve a humanized antibodies may involve additional conservative amino acid substitutions at the junction of the CDR-FR. Exemplary conservative substitutions are those such as gly, ala; val, ile, leu; asp, glu; asn, gln; ser, thr; lys, arg; and phe, tyr, wherein the amino acids identified in each group can be substituted with another amino acid identified within the same identified group, for example val could be substituted with either ile or leu. Additionally, back mutations may be implemented, where selection of residues outside of the region defining the specificity and thus target for back mutation (to mimic the source sequence, i.e., murine) to restore or improve the affinity of the humanized antibody. (Almagro 2008). When available, the three dimensional models of the antibodies may be used to guide the humanization process.

With the above context, the variable region can be connected to any subclass of constant regions (IgG, IgM, IgE, and IgA), or to scaffolds, Fab fragments, Fv, Fab, and F(ab)2. The variable region of murine anti-DPP3 antibody in the heavy chain is shown in SEQ ID No. 5 and the variable region of murine anti-DPP3 antibody in the light chain is shown in SEQ ID No. 6. For chimerization and humanization a human IgG1κ backbone was used.

For epitope binding the CDRs are of importance. The CDRs for the heavy chain and the light chain of the murine anti-DPP3 antibody (anti-DPP3 antibody) and humanized anti-DPP3 antibody are shown in SEQ ID NO: 7, SEQ ID NO: 8, and SEQ ID NO: 9 for the heavy chain and SEQ ID NO: 10, sequence KVS, and SEQ ID NO: 11 for the light chain, respectively.

Sequencing of the humanized anti-DPP3 antibody revealed an antibody heavy chain region (H chain) according to SEQ ID NO: 12 and an antibody light chain region (L chain) according to SEQ ID NO: 13.

In a further aspect, procizumab (“PCZ”) is a monoclonal antibody obtainable by methods known in the art, or methods described herein. PCZ comprises two heavy chain sequences comprising SEQ ID NO: 17 and two light chain sequence comprising SEQ ID NO: 18. PCZ that is administered to mice, cynomolgus monkeys, and humans is humanized AK1967.

In one embodiment, a humanized anti-DPP3 antibody was generated from an IgG2 antibody produced in mice, which were injected with a linear immunization peptide originated from human DPP3 (see U.S. Pat. No. 11,530,276, which is incorporated by reference for or the characterization of and the methods to generate PCZ). After humanization and codon optimization, the light and heavy chain sequences were inserted into an expression vector for stable production in CHO cells.

In a further aspect, a monoclonal antibody is obtainable by methods known in the art, or methods described herein. Culturing cells that express the antibody may be used to manufacture the protein at various scales. The antibody may be generated, for example, by any of the methods known in the art or disclosed herein used in the development and identification of the antibody. The SEQ ID NO: 12 for the heavy chain and SEQ ID NO: 13 for the light chain as well as SEQ ID NO: 16 for the heavy chain sequence after treatment with Papain (Fab fragment) contain their respective N-terminal signal peptide sequences that can be used to manufacture the antibody. The signal peptide sequence for the heavy chain is SEQ ID NO: 19 (MDPKGSLSWRILLFLSLAFELSYG) and for the light chain is SEQ ID NO: 20 (METDTLLLWVLLLWVPGSTG). As the protein is produced by the cell, the signal peptide sequence is cleaved off by cell before the protein is secreted. The DNA encoding the generated antibody may be isolated and identified, for example, by DNA sequencing of the gene encoding the antibody against DPP3. Recombinant DNA technology well-known in the art may be used to manipulate the DNA. For example, the DNA encoding the antibody may be modified to make antibody fragments and/or full-length antibody to be combined with, for example, expression plasmid DNA. The expression plasmid DNA encoding the antibody or a fragment thereof may then be introduced, for example, by transfecting into cells used for culturing and expressing the antibody or a fragment thereof. The culturing cells expressing the antibody or a fragment thereof may be used to manufacture the antibody or a fragment thereof at various scales. The culturing cells may be, for example, Chinese Hamster Ovary (CHO) cells (including strains K1-, DukX B11-, DG44, Lec13), Human Embryonic Kidney 293 (HEK293) cells, baby hamster kidney (BHK21) cells, murine myeloma cells (NS0 and Sp2/0), Vero cells, MRC-5 cells, PerC.6 cells, AGE1.HN, or any other cells known in the art that are used to culture therapeutic proteins for manufacturing at various scales. Other non-mammalian cell lines or other cells may be used for culturing the antibody or a fragment thereof, such as, S. cerevisiae and E. coli, or other known cells known in the art. Depending on the glycosylation status of the antibody, disulfide bond formation, and/or other post translation modifications of the protein may be taken into account for the choice of the cells for culturing.

The humanized anti-DPP3 antibody may recombinantly be produced with a CHO DG44 cell line. In a particular embodiment, the creation of a production cell, e.g., the CHO cell line encoding the DPP3 binder, is used for producing the DPP3 binder. The antibody binds to its target molecule DPP3, inhibiting its enzymatic activity as part of its mode of action. The humanized anti-DPP3 antibody is directed against a linear epitope SEQ ID NO: 3 (INPETG within the DPP3 sequence). The in silico derived molecular weights of the light and heavy chain of PCZ are 23,943 Dalton (Da) and 48,950 Da, respectively.

A unit dose in mg/kg refers to mg of antibody per kg of patient is based on a certain molecular weight (“MW”) of the administered antibody. All references to mg/kg in this application refer to the full-length anti-DPP3 antibody having a MW of 145,734 Da determined in silico. For any other MW of antibodies and fragments thereof the appropriate doses may be calculated accordingly.

Corresponding molar doses of antibodies or antibody fragments having other molecular weights can in particular be calculated according to the following explanation:

A unit of dose in mg of antibody or fragment thereof per kg of patient is based on a specific molecular weight of the administered antibody or fragment thereof. If the molecular weight of the antibody would be for instance 145,734 g/mol (as is the case for the humanized anti-DPP3 antibody AK1967 (Procizumab; PCZ)), a dose of about 18 to 22 mg/kg would correspond to about 0.12 to 0.15 μmol/kg. If, in contrast, the molecular weight of the antibody would be for instance 165,000 g/mol, the same molar doses of about 0.12 to 0.15 μmol/kg would correspond to about 20 to 25 mg/kg.

The method of how the mass of PCZ was determined was as follows: The PCZ concentration attributed to the drug substance and drug product as well as the doses described in mass per weight (mg/kg) were based on measurement of the absorption of the PCZ at 280 nm measured in a spectrophotometer with a path length of 1 cm. Conversion of A280 nm measurement into mol/L was done by employing a theoretically calculated extinction coefficient εₘₒₗₐᵣ using the formula: molar Concentration=A280 nm/εₘₒₗₐᵣ. The extinction coefficient for the PCZ was calculated as follows: The amino acid sequences of the heavy and light chain of the PCZ, as shown below, were combined, duplicated, and then pasted in a calculator program (Expasy—ProtParam; https: web.expasy.orglprotparam; Protein Identification and Analysis Tools on the Expasy Server; Gasteiger E., Hoogland C., Gattiker A., Duvaud S., Wilkins M R., Appel R. D., Bairoch A.; (In) John M Walker (ed): The Proteomics Protocols Handbook, Humana Press (2005), pp. 571-607 Full text—Copyright Humana Press.).

PCZ heavy chain amino acid sequence
(SEQ ID No. 17):
QITLKESGPTLVKPTQTLTLTCTFSGFSLSTSGMSVGWIRQPPGK
ALEWLAHIWWNDNKSYNPALKSRLTITRDTSKNQVVLTMTNMDPV
DTGTYYCARNYSYDYWGQGTLVTVSSASTKGPSVFPLAPSSKSTS
GGTAALGCLVKDYFPEPVTVSWNSGALTSGVHTFPAVLQSSGLYS
LSSVVTVPSSSLGTQTYICNVNHKPSNTKVDKKVEPKSCDKTHTC
PPCPAPELLGGPSVFLFPPKPKDTLMISRTPEVTCVVVDVSHEDP
EVKFNWYVDGVEVHNAKTKPREEQYNSTYRVVSVLTVLHQDWLNG
KEYKCKVSNKALPAPIEKTISKAKGQPREPQVYTLPPSRDELTKN
QVSLTCLVKGFYPSDIAVEWESNGQPENNYKTTPPVLDSDGSFFL
YSKLTVDKSRWQQGNVFSCSVMHEALHNHYTQKSLSLSPG
PCZ light chain amino acid sequence
(SEQ ID No. 18):
DIVMTQTPLSLSVTPGQPASISCKSSRSLVHSIGSTYLYWYLQKP
GQSPQLLIYKVSNRFSGVPDRFSGSGSGTDFTLKISRVEAEDVGV
YYCSQSTHVPWTFGGGTKVEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSFNRGEC

The resulting calculation was: Molar extinction coefficient εₘₒₗₐᵣ (at 280 nm in H₂O)=228,440 M⁻¹*cm⁻¹.

The value was calculated under the assumption that all cysteine residues form cystines.

The molar concentration of PCZ can be determined by dividing the measured absorption at 280 by the molar extinction coefficient εₘₒₗₐᵣ.

For the conversion from mol into mass of PCZ a molecular weight of 145,734 g/mol was used. This molecular weight was calculated by amino acid sequence-based in silico prediction using the same calculator program as mentioned above (Expasy ProtParam; https://web.expasy.org/protparam/): First, the amino acid sequence SEQ ID No. 17 of the heavy chain was predicted as having a molecular weight of 48,950 g/mol. Secondly, the amino acid sequence SEQ ID No. 18 of the light chain was predicted as having a molecular weight of 23,943 g/mol. The molecular weight for the full-length PCZ was then calculated as 2×48,950 Da+2×23,943 Da=145,734 g/mol (1 Da=1 g/mol).

Of note, by using the method described above for the definition of the concentration of the PCZ, all concentrations and doses of the PCZ mentioned in the current invention are defined by the amino acid composition of the antibody only and do not take into consideration post-translational modifications such as glycosylation.

In one embodiment, the anti-DPP3 antibody may be manufactured as follows: after a 10-day fermentation period in fed-batch mode, the antibody is captured via a ProteinA chromatography from the cell-free harvest followed by viral inactivation. Two polishing steps remove Host Cell Proteins (HCPs) and product-related impurities. This is followed by dead-end filtration to remove viral contaminants. The final steps of the anti-DPP3 antibody manufacturing are buffer exchange, concentration to 20.0 mg/mL, and sterile filtration. Bulk Drug Substance is stored at ≤−70° C.

### Methods for Obtaining Monoclonal Antibodies

In all of the following embodiments detailing methods for obtaining or producing antibodies, the term monoclonal antibody is meant to include monoclonal antibodies, as well as fragments of monoclonal antibodies, such as the ones detailed herein, more particularly monoclonal antibodies.

### Hybridoma

In a further aspect, the antibody according to the present invention is a monoclonal antibody obtainable by a method comprising:
fusing antibody-secreting cells from an animal previously immunized with an antigen with myeloma cells to obtain a multitude of hybridomas,
isolating from said multitude of hybridomas a hybridoma producing a desired monoclonal antibody.

In certain embodiments, the antibody according to the present invention is a monoclonal antibody obtainable by isolating from a multitude of hybridomas a hybridoma producing a desired monoclonal antibody, wherein said multitude of hybridomas were produced by fusing antibody-secreting cells from an animal previously immunized with an antigen with myeloma cells to obtain multitude of hybridomas.

A desired monoclonal antibody is in particular a monoclonal antibody binding the antigen, in particular with a binding affinity of at least 10⁷ M⁻¹, preferred 10⁸ M⁻¹, more preferred affinity is greater than 10⁹ M⁻¹, most preferred greater than 10¹⁰ M⁻¹.

In certain embodiments of the method for obtaining an antibody, in step i) the animal is a mammal, particularly a rabbit, a mouse or a rat, more particularly a mouse, more particularly a Balb/c mouse.

In certain embodiments of the method for obtaining an antibody, in step i) the antibody-secreting cell is a splenocyte, more particularly an activated B-cell.

In certain embodiments of the method for obtaining an antibody, in step i) fusing involves the use of polyethylene glycol (PEG).

In certain embodiments of the method for obtaining an antibody, in step i) the myeloma is derived from a mammal, in certain embodiments from the same species of mammal from which the multitude of antibody-secreting cells is obtained. In certain specific embodiments of the method for obtaining an antibody, in step i) the myeloma cells are of the cell line SP2/0.

In certain embodiments of the method for obtaining an antibody, said fusing in step i) comprises PEG-assisted fusion, Sendai virus-assisted fusion or electric current-assisted fusion.

In certain embodiments of the method for obtaining an antibody, said isolating in step ii) comprises performing an antibody capture assay, an antigen capture assay, and/or a functional screen.

In certain embodiments of the method for obtaining an antibody, in step ii) isolating the hybridoma producing a desired monoclonal antibody may involve cloning and re-cloning the hybridomas using the limiting-dilution technique.

In one embodiment, said antibody capture assay comprises
binding an antigen to a surface, particularly a solid phase,
allowing the produced antibodies to bind to the antigen,
removing unbound antibodies by washing,
detecting bound antibodies.

In one embodiment, said antigen capture assay comprises
binding the produced antibodies via a pre-coated antibody binder to a surface, particularly a solid phase,
allowing antigen to bind to said antibodies,
removing unbound antigen by washing,
detecting bound antigen;
   or said antigen capture assay comprises
allowing an antigen to bind the produced antibodies to form an antibody-antigen complex,
binding said antibody-antigen complex via a pre-coated antibody binder to a surface, particularly a solid phase,
removing unbound antigen by washing,
detecting bound antigen.

In one embodiment, said isolating of step ii) comprises performing an immunoassay, fluorescence-activated cell sorting, cell staining, immunoprecipitation, and/or a western blot.

In one embodiment, said detecting of the antibody or the antigen is accomplished with an immunoassay, in particular with an enzyme-linked immunosorbent assay.

In one embodiment, the animal is a transgenic animal, in particular a transgenic mouse (wherein in particular the mouse immunoglobulin (Ig) gene loci have been replaced with human loci within the transgenic animal genome), such as HuMabMouse or XenoMouse.

In one embodiment, the antigen comprises a peptide according to SEQ ID NO: 2, or according to SEQ ID NO: 1 or a fragment thereof, respectively, which in certain embodiments (in particular for immunization) may be conjugated to a protein, particularly a serum protein, more particularly a serum albumin, more particularly BSA.

For the antigen comprising the peptide or fragment of SEQ ID NO: 1, the peptide or fragment is 15 to 55 amino acids long; is 15, 20 25, 30, 35, 40, 45, 50, or 55 amino acids long; or is about 15, about 20, about 25, about 30, about 35, about 40, about 45, about 50, or about 55 amino acids long, wherein the term “about” in this context of amino acid length for the peptide can be plus or minus 2 amino acids, e.g., about 20 amino acids means the range of 23-27 amino acids long.

For the antigen comprising the peptide or fragment of SEQ ID NO: 1, the peptide or fragment preferably comprises the catalytic site from 316 to 669 amino acids, preferably includes the catalytic motifs SEQ ID NO: 14 or SEQ ID NO: 15 and/or at least one or more of the important amino acids important for substrate binding and hydrolysis chosen from the list consisting of Glu316, Tyr318, Asp366, Asn391, Asn394, His568, Arg572, Arg577, Lys666, and Arg669, and/or preferably comprises the epitopes of SEQ ID NO: 3 or more preferably SEQ ID NO: 4.

In a preferred embodiment, the antibody according to the present invention is a monoclonal antibody obtainable by a method comprising:
fusing splenocytes cells from a Balb/c mouse previously immunized with a peptide according to SEQ ID NO: 2, or according to SEQ ID NO: 1 or a fragment thereof, in particular as described herein above, with SP2/0 myeloma cells using polyethylene glycol, to obtain a multitude of hybridomas,
isolating from said multitude of hybridomas a hybridoma producing a desired monoclonal antibody;
   more preferably, the method comprises
growing hybridomas for a first period (in particular 2 weeks) in HAT medium [RPMI 1640 culture medium supplemented with 20% fetal calf serum and HAT-Supplement]
followed replacing HAT medium with HT Medium for a multitude of passages (in particular 3) followed by returning to the normal cell culture medium for a second time period, in particular until the end of three weeks after fusion
primary screening of Cell culture supernatants for antigen-specific IgG antibodies
propagating microcultures of cells that tested positive in the preceding screening step
retesting Cell culture supernatants of microcultures for antigen-specific IgG antibodies
cloning and re-cloning cultures that tested positive in the preceding retesting step, using the limiting-dilution technique
optionally determining the isotypes of clones obtained from the cloning and re-cloning
optionally propagating the cells obtained after re-cloning, and
optionally purifying antibodies via Protein A.

### Phage Display

In a further aspect, the antibody according to the present invention is a monoclonal antibody obtainable by a method comprising:
i) isolating at least one antibody having affinity to an antigen from an antibody gene library;
ii) generating at least one cell strain expressing said at least one antibody;
iii) isolating the at least one antibody from a culture of the at least one cell strain obtained in the generating step ii).

An antibody having affinity to an antigen is in particular an antibody with a binding affinity of at least 10⁷ M⁻¹, preferred 10⁸ M⁻¹, more preferred affinity is greater than 10⁹ M⁻¹, most preferred greater than 10¹⁰ M⁻¹.

In a certain embodiment, the antibody according to the present invention is a monoclonal antibody obtainable by isolating at least one antibody from a culture derived from at least one cell strain which expressed at least one antibody having affinity to an antigen from an antibody gene library.

In one embodiment, the antigen comprises a peptide according to SEQ ID NO: 2, or according to SEQ ID NO: 1 or a fragment thereof, in particular as described herein above, respectively, which in certain embodiments may be bound to a solid phase.

In certain embodiments of the method for obtaining an antibody, in step i) the antibody gene library is a naive antibody gene library, particularly a human naive antibody gene library, more particularly in said library the antibodies are presented via phage display, i.e. on phages comprising a nucleotide sequence encoding for such respective antibody; more particularly the library HAL 7, HAL 8, or HAL 9, more particularly a library comprising the human naive antibody gene libraries HAL7/8.

In certain embodiments of the method for obtaining an antibody, in step i) screening comprises the use of an antigen, particularly an antigen containing a tag, more particularly a biotin tag, linked thereto via two different spacers. In particular embodiments, such panning strategy includes a mix of panning rounds with non-specifically bound antigen and antigen bound specifically via the tag, in the case of a biotin tag, bound to streptavidin. In this way, the background of non-specific binders may be minimized.

In certain embodiments of the method for obtaining an antibody, in step i), in embodiments wherein the library is a phage display library, the antibody is isolated by isolating a phage presenting said antibody (and comprising a nucleotide sequence encoding for the antibody).

In certain embodiments of the method for obtaining an antibody, in step ii) said cell strain is generated via introduction of a nucleotide sequence encoding for the antibody, in embodiments wherein the library in step i) is a phage display library, the isolated phage from step i) may be used to produce a bacterial strain, e.g. an E. coli strain, expressing the antibody.

In certain embodiments of the method for obtaining an antibody, in step iv); in embodiments wherein the library in step i) is a phage display library and wherein a bacterial strain is produced in step ii), antibody may be isolated from the supernatant of the culture.

It is understood that, as used in describing the methods for obtaining an antibody, the term “one antibody” in the expression “at least one antibody” in particular may include more than one antibody molecule of antibodies having the same amino acid sequence. This understanding applies, mutatis mutandis, to the term “one cell strain”.

In certain embodiments of the method for obtaining an antibody, more than one antibody (referring to a multitude of antibodies having distinct amino acid sequences, respectively) is isolated in step i) and accordingly more than one cell strain is generated in step ii). Such method may involve the selection of clones that are positive for binding to the antigen, e.g. via a binding assay, e.g. an ELISA assay involving the antigen, and cells positive for binding to the antigen may be isolated to produce monoclonal cell strains.

In a preferred embodiment, the antibody according to the present invention is a monoclonal antibody obtainable by a method comprising:
i) isolating at least one antibody having affinity to an antigen from an antibody gene library comprising the human naive antibody gene libraries HAL7/8, by eluting phages carrying said antibody from the library;
ii) generating at least one E. coli cell strain expressing said at least one antibody;
iii) isolating the at least one antibody from the supernatant a culture of the at least one E. coli cell strain obtained in step ii).

In a further aspect, an antibody fragment according to the present invention is produced by a method involving enzymatic digestion of an antibody. In certain embodiments, this method produces e.g. Fab or F(ab)2 antibody fragments. In certain embodiments, this method involves digestion with pepsin or papain, which are optionally immobilized on a surface.

In certain embodiments, antibodies may be humanized by CDR-grafting, in particular by a process involving the steps:
extracting RNA from hybridomas expressing an antibody of interest (e.g. obtained by a method as described herein);
amplifying said extracted RNA via RT-PCR, in particular with primer sets specific for the heavy and light chains of the antibody of interest, to obtain a DNA product;
further amplifying the DNA product via PCR, in particular using semi-nested primer sets specific for antibody variable regions;
determining the sequence of the DNA product;
translating the DNA product to determine the sequence of the protein;
aligning the protein sequence with homologous human framework protein sequences to determine a humanized protein sequence for the variable heavy chain and the variable light chain sequences (of the desired antibody).

In certain embodiments, antibodies may be humanized by aligning the sequence of a DNA product that was obtained by amplifying RNA extracted from hybridomas expressing an antibody of interest via RT-PCR, in particular with primer sets specific for the heavy and light chains of the antibody of interest and further amplifying the DNA obtained therefrom via PCR, in particular using semi-nested primer sets specific for antibody variable regions, with homologous human framework sequences to determine a humanized sequence for the variable heavy chain and the variable light chain sequences (of the desired antibody).

In certain embodiments, antibodies may be humanized by
determining the complementary determining regions (CDR), which may be accomplished by analysing the structural interaction of framework regions (FR) with the CDR and the antigen;
transplanting said CDR sequences into a human framework region.

In certain embodiments, antibodies may be humanized by transplanting CDR sequences, which may preferably have been determined by analysing the structural interaction of framework regions (FR) with the CDR and the antigen, into a human framework region.

In certain embodiments variations in the amino acid sequence of the CDRs or FRs may be introduced to maintain structural interactions with the antigen (which may otherwise be abolished by introducing the human FR sequences), for instance by a random approach using phage display libraries or via directed approach guided by molecular modelling.

The DNA sequences encoding for antibodies determined as detailed herein can be transferred by known genetic engineering techniques into cells and used for production of the antibody.

### Producing Antibodies

In a further aspect, the antibody according to the present invention is a monoclonal antibody obtainable by the methods described herein, produced by a method comprising:
culturing a cell strain comprising a nucleotide sequence encoding for the antibody;
isolating the antibody from said culture.

In a further certain aspect, the antibody according to the present invention is a monoclonal antibody generated by the methods described herein, produced by isolating the antibody from a culture of a cell strain comprising a nucleotide sequence encoding for said antibody.

In certain embodiments of said method, the cell strain used to culture the antibody or antibody fragment thereof is produced as described herein above and may comprise bacterial cells, such as gram-negative bacteria, e.g. E. coli, Proteus mirabilis, or Pseudomonas putidas, gram-positive bacteria, e.g. Bacillus brevis, Bacillus subtilis, Bacillus megaterium, Lactobacilli such as Lactobacillus zeae/casei or Lactobacillus paracasei, or Streptomyces, such as Streptomyces lividans; eucariotic cells such as yest, e.g. Pichia pastoris, Saccharomyces cerevisiae, Hansenula polymorpha, Schizosaccharomyces pombe, Schwanniomyces occidentalis, Kluyveromyces lactis, or Yarrowia lipolytica; fugi, such as filamentous fungi, e.g. of the genus Trichoderma of Aspergillus, such as A. niger (e.g. subgenus A. awamori) and Aspergillus oryzae, Trichoderma reesei, Chrysosporium, such as C. lucknowense; protozoae, such as Leishmania, e.g. L. tarentolae; insect cells, such as insect cells transfected a Baculovirus, e.g. AcNPV, such as insect cell lines from Spodoptera frugiperda, e.g. Sf-9 or Sf-21, Drosophila melanogaster, e.g. DS2, or Trichopulsia ni, e.g. High Five cells (BTI-TN-5B1-4); mammalian cells such as hamster, e.g. Chinese hamster ovary such as K1-, DukX B11-, DG44, Lec13, or BHK, mouse, e.g. mouse myeloma such as NS0, Homo sapiens, e.g. Per.C6, AGE1.HN, HEK293.

In certain embodiments of said method, the cells may be hybridoma cells, e.g. as described herein.

In a further aspect, procizumab (“PCZ”) is a monoclonal antibody obtainable by methods known in the art, or methods described herein. PCZ comprises two heavy chain sequences comprising SEQ ID NO: 17 and two light chain sequence comprising SEQ ID NO: 18. The respective SEQ ID NO: 12 for the heavy chain and SEQ ID NO: 13 for the light chain contain their respective N-terminal signal peptide sequences that can be used to manufacture the PCZ. The signal peptide sequence for the heavy chain is SEQ ID NO: 19 (MDPKGSLSWRILLFLSLAFELSYG) and for the light chain is SEQ ID NO: 20 (METDTLLLWVLLLWVPGSTG). As the PCZ is produced by the cell, the signal peptide sequence is cleaved off by cell before the protein is secreted. Culturing cells that express PCZ may be used to manufacture the protein at various scales. PCZ may be generated, for example, by any of the methods disclosed above used in the development and identification of the antibody. The DNA encoding the generated PCZ may be isolated and identified, for example, by DNA sequencing of the gene encoding the antibody against DPP3. Recombinant DNA technology well-known in the art may be used to manipulate the DNA. For example, the DNA encoding PCZ may be modified to make PCZ fragments and/or full-length PCZ to be combined with, for example, expression plasmid DNA. Further, the DNA can be codon optimized. The expression plasmid DNA encoding the PCZ or a fragment thereof may then be introduced, for example, by transfecting into cells used for culturing and expressing the PCZ or a fragment thereof. The culturing cells expressing the PCZ or a fragment thereof may be used to manufacture the PCZ or a fragment thereof at various scales. The culturing cells may be, for example, Chinese Hamster Ovary (CHO) cells (including strains K1-, DukX B11-, DG44, Lec13), Human Embryonic Kidney 293 (HEK293) cells, baby hamster kidney (BHK21) cells, murine myeloma cells (NS0 and Sp2/0), Vero cells, MRC-5 cells, PerC.6 cells, AGE1.HN, or any other cells known in the art that are used to culture therapeutic proteins for manufacturing at various scales. Other non-mammalian cell lines or other cells may be used for culturing PCZ or a fragment thereof, such as, S. cerevisiae and E. coli and the others described above. The post translational modification of the protein should be taken into account for the choice of the cells for culturing.

In certain embodiments of said method, culturing may take place in a static suspension culture, an agitated suspension culture, a membrane-based culture, a matrix-based culture or a high cell density bioreactor; a vessel for such culturing may be selected from the group comprising a T-flask, a roller culture, a spinner culture, a stirred tank bioreactor, an airlift bioreactor, a static membrane-based or matrix-based culture system, a suspension bioreactor, a fluidized bed bioreactor, a ceramic bioreactor, a perfusion system, a hollow fiber bioreactor.

In certain embodiments of said method, the cells may be immobilized on a matrix.

A high cell density bioreactor is in particular a culture system capable of generating cell densities greater than 10⁸ cells/ml.

In a further aspect, the antibody according to the present invention is a monoclonal antibody obtainable by the methods described herein, produced by a method comprising:
generating a transgenic plant or animal comprising a nucleotide sequence encoding for the antibody;
isolating the antibody from said plant or animal or a secretion or product of said plant or animal.

In a certain further aspect, the antibody according to the present invention is a monoclonal antibody obtainable by the methods described herein, produced by isolating the antibody from a transgenic plant or transgenic animal or a secretion or product of a transgenic plant or transgenic animal having a nucleotide sequence encoding for the antibody.

Said animal may e.g. be selected from a chicken, a mouse, a rat, a rabbit, a cow, a goat, a sheep, a pig; said secretion or product may e.g. be milk or an egg. Said plant may e.g. be selected from tobacco (N. tabacum or N. benthamiana), duckweed (Lemna minor), Chlamydomonas reinhardtii, rice, Arabidopsis thaliana, alfalfa (Medicago sativa), lettuce, maize.

The antibodies can in certain embodiments be isolated by physicochemical fractionation, e.g. size exclusion chromatography, precipitation, e.g. using ammonium sulphate, ion exchange chromatography, immobilized metal chelate chromatography gel filtration, zone electrophoresis; based on their classification e.g. binding to bacterial proteins A, G, or L, jacalin; antigen-specific affinity purification via immobilized ligands/antigens; if necessary, low molecular weight components can be removed by methods like dialysis, desalting, and diafiltration.

In some embodiments the antibody is encoded by a nucleotide sequence where the nucleotide sequence is a reverse transcription of an amino acid sequence from an antibody produced by one of the processes described herein.

In one embodiment, the anti-DPP3 antibody is contained in a pharmaceutical formulation. In one embodiment, the anti-DPP3 antibody pharmaceutical formulation is a concentrated solution (i.e., to be diluted prior to administration) for infusion and contains the anti-DPP3 antibody as an active substance together with one or more pharmaceutically acceptable excipients. The pharmaceutically acceptable excipients are those that are compatible with the anti-DPP3 antibody. The pharmaceutically acceptable excipients for use with the anti-DPP3 antibody include stabilizers, antioxidants, tonicity modifiers, and buffers. Suitable stabilizers include sugars, sugar alcohols, amino acids, surfactants, polyols, and stabilizing proteins. Examples of sugar stabilizers are sucrose, glucose, lactose, trehalose, and maltose; examples of sugar alcohol stabilizers include mannitol, xylitol, and sorbitol; examples of amino acid stabilizers include arginine, histidine, glycine, proline, alanine, lysine, leucine, and methionine; and examples of surfactant stabilizers include Polysorbate 20 and Polysorbate 80 (i.e., Tween 20 and Tween 80, respectively), Poloxamers (i.e., poloxamer 188 and Pluronic F68, 127), and PEGylated surfactants; examples of stabilizing polyols include glycerol, polyethylene glycol (PEG, low MW) and examples of stabilizing proteins include transferrin, human serum albumin, and bovine serum albumin. Antioxidants useful in an anti-DPP3 concentrated solution include methionine, ascorbic acid, reduced glutathione, cysteine, Thio glycerol, sodium thiosulfate, platinum, N-acetyl-DL-tryptophan, ethylenediaminetetraacetic acid (EDTA) and diethylenetriaminepentaacetic acid (DTPA). Examples of suitable tonicity modifiers include sodium chloride, potassium chloride, calcium chloride, mannitol, sorbitol, and glycerol. Buffers suitable in a concentrated solution include histidine, citrate, phosphate (e.g., phosphate-buffered saline), acetate, succinate, MES, HEPES, and Tris (e.g. tris-buffered saline). Examples of suitable pHs are in the range of 4.8 to 8.0, preferred in the range of 5.0 to 6.5, or more preferred in the range of 5.5 to 6.8; or at a pH of about 5.0, about 5.5, about 6.0, or about 6.5; preferred at a pH of 6.5. In one embodiment the anti-DPP3 antibody is lyophilized and is formulated with bulking agents (i.e., lyoprotectants) and suitable bulking agents include sucrose, trehalose, mannitol, glycine, and dextran. The excipients can serve multiple purposes within a single formulation and are not limited to a single function. Multiple excipients can be included in the formulation to fulfill similar purposes. In one embodiment, the anti-DPP3 antibody is formulated with 10 mM Histidine-HCl, 250 mM Trehalose, and 20 mM L-Methionine pH 6.5 buffer.

In one embodiment, the anti-DPP3 antibody pharmaceutical formulation manufacturing consists of pooling and filtration of the anti-DPP3 antibody and filling into Type-1 glass vials (20R DIN Ph. Eur.). The vials are capped via coated bromobutyl stoppers and 20 mm flip-off seals in red with aluminum crimps. 20.0 mL of the filtered anti-DPP3 antibody solution, with a concentration of 20.0 mg/mL, is filled aseptically into 20R vials. This represents an extractable volume of >19 mL.

In one embodiment, the anti-DPP3 antibody pharmaceutical formulation may be stored at 2-8° C. Before use, the vials of the concentrated solution are equilibrated to room temperature and then diluted with 0.9% saline, water for injection (WFI), phosphate buffered with saline (PBS), or WFI formulated with a tonicity modifier(s) (e.g., sodium chloride, potassium chloride, calcium chloride, mannitol, sorbitol, and glycerol) prior to infusion. In one embodiment the diluent has the same or equivalent excipient composition as is used for the concentrated pharmaceutical form, albeit, without the anti-DPP3 antibody. In another embodiment the diluent has the same or equivalent excipient composition and concentrations of the excipients as used for the concentrated pharmaceutical form, albeit, without the anti-DPP3 antibody.

In one embodiment, the composition of the anti-DPP3 antibody pharmaceutical formulation is shown in Table 3.

TABLE 3
Components of the anti-DPP3 antibody
pharmaceutical formulation.

| | | Quantity | | Quantity | |
|---|---|---|---|---|---|
| Name of ingredient | Function | (per mL) | | (per vial) | |
| Humanized anti-DPP3 | API | 20.0 | mg | 400 | mg |
| antibody | | | | | |
| Trehalose Dihydrate | Stabilizer | 90.7 | mg | 1.8154 | g |
| L-Methionine | Antioxidant | 2.86 | mg | 57.2 | mg |
| L-Histidine | Buffer | 1.11 | mg | 22.2 | mg |
| L-Histidine HCl * H₂O | pH adjustment | 0.51 | mg | 10.2 | mg |
| Water for Injection | Solvent | Ad 1.0 | mL | Ad 20.0 | mL |

In one embodiment of the invention the anti-DPP3 antibody pharmaceutical formulation is administered at a dose between 1 to 350 mg/kg bodyweight, 1 to 150 mg/kg bodyweight, 1 to 30 mg/kg bodyweight, preferably 2 to 20 mg/kg bodyweight, preferably 3 to 15 mg/kg body weight, preferably 5 to 12 mg/kg bodyweight, preferably 7 to 10 mg/kg bodyweight, and most preferably 10 mg/kg or 20 mg/kg bodyweight.

In one embodiment of the invention the anti-DPP3 antibody pharmaceutical formulation is administered at a dose of 3 mg/kg bodyweight, 6 mg/kg bodyweight, or 12 mg/kg bodyweight.

In one embodiment of the invention the anti-DPP3 antibody pharmaceutical formulation is administered as an infusion over up to 10 hours, 1 to 3 hours, preferably 1.5 to 2.5 hours, and most preferred 2 hours.

In one embodiment of the invention the anti-DPP3 antibody pharmaceutical formulation is administered at a dose of 10 mg/kg or 20 mg/kg bodyweight and applied as infusion over up to 10 hours, 1 to 3 hours, preferably 1.5 to 2.5 hours, and most preferred 2 hours.

In one embodiment of the invention the anti-DPP3 antibody pharmaceutical formulation is administered as multiple infusions.

In one embodiment of the invention the anti-DPP3 antibody pharmaceutical formulation is administered as multiple infusions, each infusion being over up to 10 hours, 1 to 3 hours, preferably 1.5 to 2.5 hours, and most preferred 2 hours.

In one embodiment of the invention the anti-DPP3 antibody pharmaceutical formulation dose is administered as a bolus injection.

In one embodiment of the invention the anti-DPP3 antibody pharmaceutical formulation dose is administered as multiple bolus injections over a 2 hour period, wherein the number of injections is 2, 3, 4, 5, or 6.

In one embodiment of the invention the anti-DPP3 antibody pharmaceutical formulation dose is administered as an injection multiple times over the course of the treatment.

In one embodiment of the invention said extreme-critically ill patient is characterized by a level of lactate of 2 mmol/1 or higher and/or a level of IL-6 of 5.9 pg/ml or higher and/or a level of CRP of 1 mg/dL or higher and/or a level of PCT of 0.5 ng/ml or higher and/or a level of bio-ADM of 70 μg/ml or higher and/or serum creatinine of 1.18 mg/dL (for men) and 1.02 mg/dL (for women) and/or a supranormal level of NT-proBNP in a sample of bodily fluid of said patient.

In one embodiment of the invention the antibody is a monoclonal antibody or a fragment thereof.

In one embodiment of the invention the anti-DPP3 antibody or the anti-DPP3 antibody fragment is a human or humanized antibody or derived therefrom. In one specific embodiment one or more (murine) CDRs are grafted into a human antibody or antibody fragment.

In one embodiment of the invention the anti-DPP3 antibody or the anti-DPP3 antibody fragment is a human or humanized CDR-grafted antibody or antibody fragment thereof that binds to DPP3, wherein the human or humanized CDR-grafted antibody or antibody fragment thereof comprises an antibody heavy chain (H chain) comprising:

(SEQ ID NO: 7)
GFSLSTSGMS,
(SEQ ID NO: 8)
IWWNDNK,
(SEQ ID NO: 9)
ARNYSYDY

and/or further comprises an antibody light chain (L chain) comprising:

(SEQ ID NO: 10)
RSLVHSIGSTY,
KVS (not part of the sequencing listing),
(SEQ ID NO: 11)
SQSTHVPWT

or a sequence wherein one or two amino acids are exchanged from the original CDR sequences in at least one of the CDR sequences provided that the binding affinity to DPP3 is diminished by not more than 20%.

In one specific embodiment of the invention the anti-DPP3 antibody or the anti-DPP3 antibody fragment is a human or humanized monoclonal antibody that binds to DPP3, wherein the heavy chain comprises at least one CDR selected from the group comprising:

(SEQ ID NO: 7)
GFSLSTSGMS,
(SEQ ID NO: 8)
IWWNDNK,
(SEQ ID NO: 9)
ARNYSYDY

and wherein the light chain comprises at least one CDR selected from the group comprising:

(SEQ ID NO: 10)
RSLVHSIGSTY,
KVS
(not part of the sequencing listing),
(SEQ ID NO: 11)
SQSTHVPWT

or a sequence wherein one or two amino acids are exchanged from the original CDR sequences in at least one of the CDR sequences provided that the binding affinity to DPP3 is diminished by not more than 20%.

The anti-DPP3 antibody or anti-DPP3 antibody fragment or anti-DPP3 non-Ig scaffold according to the present invention exhibits an affinity towards human DPP3 such that affinity constant is greater than 10⁻⁷ M, preferred 10⁻⁸ M, preferred affinity is greater than 10⁻⁹ M, most preferred higher than 10⁻¹⁰ M. A person skilled in the art knows that it may be considered to compensate for lower affinity by applying a higher dose of compounds and this measure would not lead out-of-the-scope of the invention. The affinity constants may be determined according to the method as described in Example 2 of U.S. Pat. No. 11,530,276, which is incorporated by reference for determining the affinity constant.

In one specific embodiment of the invention the anti-DPP3 antibody or the anti-DPP3 antibody fragment is a human or humanized monoclonal antibody that binds to DPP3, wherein said antibody or fragment comprises the following sequence as a variable heavy chain:

SEQ ID NO: 5
QVTLKESGPGILQPSQTLSLTCSFSGFSLSTSGMSVGWIRQPSGKGLEWL
AHIWWNDNKSYNPALKSRLTISRDTSNNQVFLKIASVVTADTGTYFCARN
YSYDYWGQGTTLTVSS

and comprises the following sequence as a variable light chain:

SEQ ID NO: 6
DVVVTQTPLSLSVSLGDPASISCRSSRSLVHSIGSTYLHWYLQKPGQSPK
LLIYKVSNRFSGVPDRFSGSGSGTDFTLKISRVEAEDLGVYFCSQSTHVP
WTFGGGTKLEIK.

In one specific embodiment of the invention the anti-DPP3 antibody or the anti-DPP3 antibody fragment is a human or humanized monoclonal antibody that binds to DPP3, wherein said antibody or fragment comprises the following sequence as a heavy chain:

SEQ ID NO: 12
MDPKGSLSWRILLFLSLAFELSYGQITLKESGPTLVKPTQTLTLTCTFSG
FSLSTSGMSVGWIRQPPGKALEWLAHIWWNDNKSYNPALKSRLTITRDTS
KNQVVLTMTNMDPVDTGTYYCARNYSYDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPG

and comprises the following sequence as a light chain:

SEQ ID NO: 13
METDTLLLWVLLLWVPGSTGDIVMTQTPLSLSVTPGQPASISCKSSRSLV
HSIGSTYLYWYLQKPGQSPQLLIYKVSNRFSGVPDRFSGSGSGTDFTLKI
SRVEAEDVGVYYCSQSTHVPWTFGGGTKVEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC.

In a specific embodiment of the invention the antibody comprises the following sequence as a heavy chain: SEQ ID NO: 12 or a sequence that is >80% identical to it, >90% identical to it, >95% identical to it, preferably >98%, or preferably >99% and comprises the following sequence as a light chain: SEQ ID NO: 13 or a sequence that is >80% identical to it, >90% identical to it, >95% identical to it, preferably >98%, or preferably >99%, provided that the binding affinity to DPP3 is diminished by not more than 20%.

In one specific embodiment of the invention the anti-DPP3 antibody or the anti-DPP3 antibody fragment is a human or humanized monoclonal antibody that binds to DPP3, wherein said antibody is a fragment comprises the following sequence as a heavy chain fragment:

SEQ ID NO: 16
MDPKGSLSWRILLFLSLAFELSYGQITLKESGPTLVKPTQTLTLTCTFSG
FSLSTSGMSVGWIRQPPGKALEWLAHIWWNDNKSYNPALKSRLTITRDTS
KNQVVLTMTNMDPVDTGTYYCARNYSYDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH

and comprises the following sequence as a light chain:

SEQ ID NO: 13
METDTLLLWVLLLWVPGSTGDIVMTQTPLSLSVTPGQPASISCKSSRSLV
HSIGSTYLYWYLQKPGQSPQLLIYKVSNRFSGVPDRFSGSGSGTDFTLKI
SRVEAEDVGVYYCSQSTHVPWTFGGGTKVEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC.

In a specific embodiment of the invention the antibody fragment comprises the following sequence as a heavy chain fragment: SEQ ID NO: 21 or a sequence that is >80% identical to it, >90% identical to it, >95% identical to it, preferably >98%, or preferably >99% and comprises the following sequence as a light chain: SEQ ID NO: 18 or a sequence that is >80% identical to it, >90% identical to it, >95% identical to it, preferably >98%, or preferably >99% provided that the binding affinity to DPP3 is diminished by not more than 20%.

In one specific embodiment of the invention the anti-DPP3 antibody or the anti-DPP3 antibody fragment is a human or humanized monoclonal antibody that binds to DPP3, wherein said antibody is a fragment comprises the portion of the heavy chain fragment that is the variable region of the following sequence:

SEQ ID NO: 16
MDPKGSLSWRILLFLSLAFELSYGQITLKESGPTLVKPTQTLTLTCTFSG
FSLSTSGMSVGWIRQPPGKALEWLAHIWWNDNKSYNPALKSRLTITRDTS
KNQVVLTMTNMDPVDTGTYYCARNYSYDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH

and comprises the portion of the light chain fragment that is the variable region of the following sequence:

SEQ ID NO: 13
METDTLLLWVLLLWVPGSTGDIVMTQTPLSLSVTPGQPASISCKSSRSLV
HSIGSTYLYWYLQKPGQSPQLLIYKVSNRFSGVPDRFSGSGSGTDFTLKI
SRVEAEDVGVYYCSQSTHVPWTFGGGTKVEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC.

In a specific embodiment of the invention the antibody fragment comprises the portion of the heavy chain fragment that is the variable region of the following sequence: SEQ ID NO: 21 or a sequence that is >80% identical to it, >90% identical to it, >95% identical to it, preferably >98%, or preferably >99% and comprises the portion of the light chain fragment that is the variable region of the following sequence: SEQ ID NO: 18 or a sequence that is >80% identical to it, >90% identical to it, >95% identical to it, preferably >98%, or preferably >99%. Additionally, the described invention above is a Fv embodiment and may comprise an additional linker sequence used to connect the heavy and light chain Fv regions together.

In a more specific embodiment, the anti-DPP3 antibody or fragment thereof is a humanized anti-DPP3 antibody or fragment thereof.

In one specific embodiment of the invention the anti-DPP3 antibody or the anti-DPP3 antibody fragment is a human or humanized monoclonal antibody that binds to DPP3, wherein said antibody or fragment comprises the following sequence as a heavy chain:

SEQ ID NO: 17
QITLKESGPTLVKPTQTLTLTCTFSGFSLSTSGMSVGWIRQPPGKALEWL
AHIWWNDNKSYNPALKSRLTITRDTSKNQVVLTMTNMDPVDTGTYYCARN
YSYDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG

and comprises the following sequence as a light chain:

SEQ ID NO: 18
DIVMTQTPLSLSVTPGQPASISCKSSRSLVHSIGSTYLYWYLQKPGQSPQ
LLIYKVSNRFSGVPDRFSGSGSGTDFTLKISRVEAEDVGVYYCSQSTHVP
WTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC.

In a specific embodiment of the invention the antibody comprises the following sequence as a heavy chain: SEQ ID NO: 17 or a sequence that is >80% identical to it, >90% identical to it, >95% identical to it, preferably >98%, or preferably >99% and comprises the following sequence as a light chain: SEQ ID NO: 18 or a sequence that is >80% identical to it, >90% identical to it, >95% identical to it, preferably >98%, or preferably >99% wherein in more particular embodiments the heavy chain comprises at least one CDR selected from the group comprising:

(SEQ ID NO: 7)
GFSLSTSGMS,
(SEQ ID NO: 8)
IWWNDNK,
(SEQ ID NO: 9)
ARNYSYDY

and the light chain comprises at least one CDR selected from the group comprising:

(SEQ ID NO: 10)
RSLVHSIGSTY,
KVS
(not part of the sequencing listing),
(SEQ ID NO: 11)
SQSTHVPWT

or a sequence wherein one or two amino acids are exchanged from the original CDR sequences in at least one of the CDR sequences, provided that the binding affinity to DPP3 is diminished by not more than 20%.

### EXAMPLES

In the examples, the antibody is in particular a humanized antibody. In the examples “murine AK1967” means the mouse form and the humanized form may be referred to as “humanized AK1967,” “the anti-DPP3 antibody,” “Procizumab,” or “PCZ”.

DPP3 activity and cDPP3 activity are generically described herein as DPP3 activity. In general, if DPP3 activity is measured in a bodily fluid, such as the blood, this means that cDPP3 activity is being measured.

DPP3 concentration and cDPP3 concentration are generically described herein as DPP3 concentration. In general, if DPP3 concentration is measured in a bodily fluid, such as the blood, this means that cDPP3 concentration is being measured.

### Example 1—Development of Humanized Anti-DPP3 Antibody

Murine antibodies raised against SEQ ID NO: 2 were characterized in more detail (epitope mapping, binding affinities, specificity, inhibitory potential). Here the results for murine clone 1967 of SEQ ID NO: 2 (murine anti-DPP3 antibody) are shown as an example.

### Determination of Murine Anti-DPP3 Antibody Epitope on DPP3:

For epitope mapping of murine an anti-DPP3 antibody a number of N- or C-terminally biotinylated peptides were synthesized (peptides & elephants GmbH, Hennigsdorf, Germany). These peptides include the sequence of the full immunization peptide (SEQ ID NO: 2) or fragments thereof, with stepwise removal of one amino acid from either C- or N-terminus (see Table 5 for a complete list of peptides).

High binding 96 well plates were coated with 2 pg Avidin per well (Greiner Bio-One international AG, Austria) in coupling buffer (500 mM Tris-HCl, pH 7.8, 100 mM NaCl). Afterwards plates were washed and filled with specific solutions of biotinylated peptides (10 ng/well; buffer—1×PBS with 0.5% BSA).

Murine Anti-DPP3 Antibody was Labelled with a Chemiluminescence Label.

The plates were filled with 200 μl of labelled and diluted detection antibody (tracer) and incubated for 4 h at room temperature. Unbound tracer was removed by washing 4 times with 350 μl washing solution (20 mM PBS, pH 7.4, 0.1% Triton X-100). Well-bound chemiluminescence was measured by using the Centro LB 960 luminometer (Berthold Technologies GmbH & Co. KG). Binding of murine anti-DPP3 antibody to the respective peptides is determined by evaluation of the relative light units (RLU). Any peptide that shows a significantly higher RLU signal than the unspecific binding of murine anti-DPP3 antibody is defined as murine anti-DPP3 antibody binder. The combinatorial analysis of binding and non-binding peptides reveals the specific DPP3 epitope of murine anti-DPP3 antibody.

### Determination of Binding Affinities Using Octet:

The experiment was performed using Octet Red96 (ForteBio). Murine Anti-DPP3 antibody was captured on kinetic grade anti-human Fc (AHC) biosensors. The loaded biosensors were then dipped into a dilution series of recombinant GST-tagged human DPP3 (100, 33.3, 11.1, 3.7 nM). Association was observed for 120 seconds followed by 180 seconds of dissociation. The buffers used for the experiment are depicted in Table 4. Kinetic analysis was performed using a 1:1 binding model and global fitting.

TABLE 4
Buffers used for Octet measurements

| Buffer | Composition |
|---|---|
| Assay Buffer | PBS with 0.1% BSA, 0.02% Tween-21 |
| Regeneration Buffer | 10 mM Glycine buffer (pH 1.7) |
| Neutralization Buffer | PBS with 0.1% BSA, 0.02% Tween-21 |

### Western Blot Analysis of Binding Specificity of a Murine Anti-DPP3 Antibody:

Blood cells from human EDTA-blood were washed (3× in PBS), diluted in PBS and lysed by repeated freeze-thaw-cycles. The blood cell lysate had a total protein concentration of 250 μg/ml, and a DPP3 concentration of 10 μg/ml. Dilutions of blood cell lysate (1:40, 1:80, 1:160 and 1:320) and of purified recombinant human His-DPP3 (31.25-500 ng/ml) were subjected to SDS-PAGE and Western Blot. The blots were incubated in (1) blocking buffer (1×PBS-T with 5% skim milk powder), (2) primary antibody solution (murine anti-DPP3 antibody 1:2.000 in blocking buffer), and (3) HRP labelled secondary antibody (goat anti mouse IgG, 1:1.000 in blocking buffer). Bound secondary antibody was detected using the Amersham ECL Western Blotting Detection Reagent and the Amersham Imager 600 UV (both from GE Healthcare).

### DPP3 Inhibition Assay:

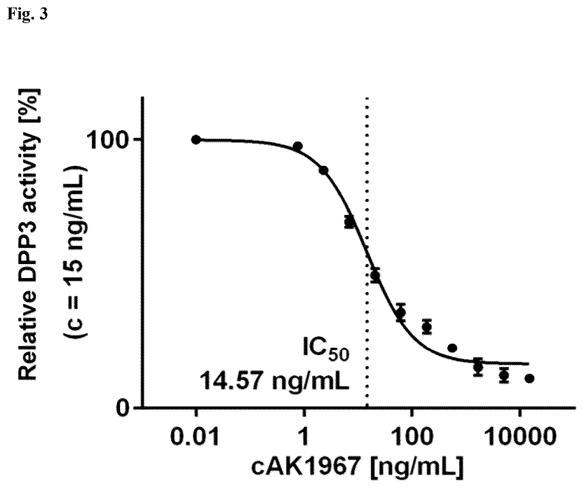

To analyse the capability of DPP3 inhibition by murine anti-DPP3 antibody a DPP3 activity assay with known procedure (Jones & Kapralou 1982) was performed. The inhibitory ability murine anti-DPP3 antibody is defined as the decrease of GST-hDPP3 activity by incubation with said antibody in percent. The resulting lowered DPP3 activities are shown in an inhibition curve in FIG. 3.

### Epitope Mapping:

The analysis of peptides that murine anti-DPP3 antibody binds to and does not bind to revealed the DPP3 sequence DPETG (SEQ ID NO: 3) as necessary epitope for murine anti-DPP3 antibody binding (see Table 5). Similar results were obtained for the humanized anti-hDPP3 antibody.

### Binding Affinity:

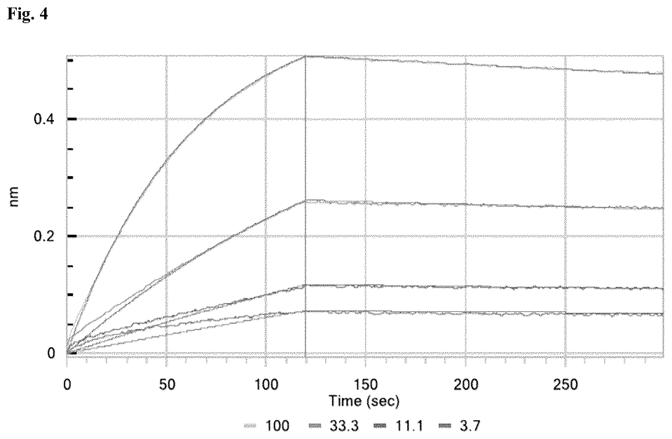

Murine anti-DPP3 antibody binds with an affinity of 2.2*10⁻⁹ M to recombinant GST-hDPP3 (kinetic curves see FIG. 4).

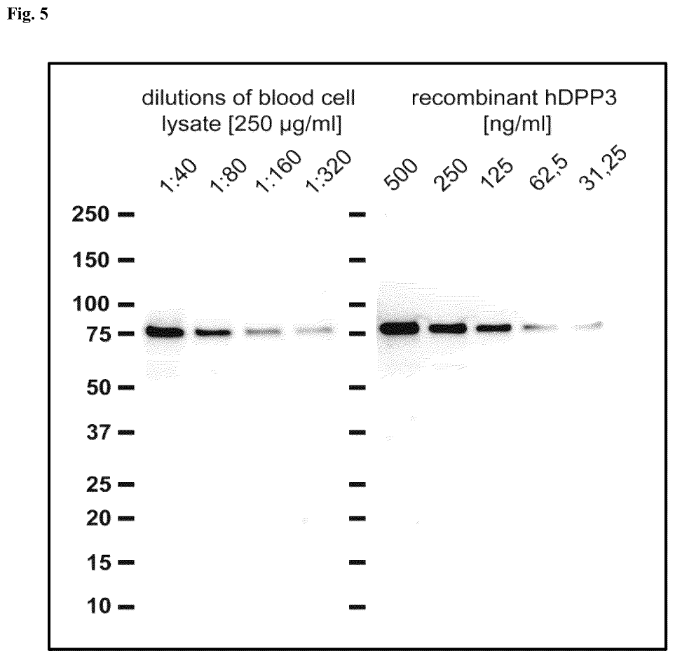

The only protein detected with murine anti-DPP3 antibody as primary antibody in lysate of blood cells was DPP3 at 80 kDa (FIG. 5). The total protein concentration of the lysate was 250 μg/ml whereas the estimated DPP3 concentration is about 10 μg/ml. Even though there is 25 times more unspecific protein in the lysate, murine anti-DPP3 antibody binds and detects specifically DPP3 and no other unspecific binding takes place.

The murine anti-DPP3 antibody inhibits 15 ng/ml DPP3 in a DPP3 activity assay with an IC50 of about 15 ng/ml (FIG. 3).

### Chimerization/Humanization:

The murine monoclonal antibody anti-DPP3, with the ability of inhibiting DPP3 activity by 70%, was chosen as possible therapeutic antibody and was also used as template for chimerization and humanization.

### Humanization of Murine Antibodies May be Conducted According to the Following Procedure:

For humanization of an antibody of murine origin the antibody sequence is analysed for the structural interaction of framework regions (FR) with the CDR and the antigen. Based on structural modelling an appropriate FR of human origin is selected and the murine CDR sequences are transplanted into the human FR. Variations in the amino acid sequence of the CDRs or FRs may be introduced to regain structural interactions, which were abolished by the species switch for the FR sequences. This recovery of structural interactions may be achieved by random approach using phage display libraries or via directed approach guided by molecular modelling (Almagro 2008).

With the above context, the variable region can be connected to any subclass of constant regions (IgG, IgM, IgE. IgA), or only scaffolds, Fab fragments, Fv, Fab and F(ab)2. For chimerization and humanization a human IgG1κ backbone was used.

For epitope binding only the CDRs are of importance. The CDRs for the heavy chain and the light chain of the murine anti-DPP3 antibody as well as for the humanized anti-hDPP3 antibody are shown in SEQ ID NO: 7, SEQ ID NO: 8, and SEQ ID NO: 9 for the heavy chain and SEQ ID NO: 10, sequence KVS, and SEQ ID NO: 11 for the light chain, respectively.

Sequencing of the humanized anti-DPP3 antibody revealed an antibody heavy chain region (H chain) according to SEQ ID NO: 12 and an antibody light chain region (L chain) according to SEQ ID NO: 13.

### Example 2—DPP3 and Organ Dysfunction in Sepsis

The AdrenOSS-1 study was used to assess the association between circulating DPP3 (cDPP3), organ dysfunction (e.g. cardiovascular and renal dysfunction) in patients admitted for sepsis and septic shock. The AdrenOSS-1 is a European prospective, observational, multinational study (ClinicalTrials.gov NCT02393781) including 583 patients admitted to the ICU with sepsis or septic shock. The primary outcome was 28-day mortality. Secondary outcomes included organ failure defined by SOFA score, organ support with focus on vasopressor use and need for renal replacement therapy. Blood for the central laboratory was sampled within 24 hours after ICU admission and on day 2.

For the quantification of DPP3 protein concentrations (DPP3-LIA) an assay as recently described was used (Rehfeld 2019).

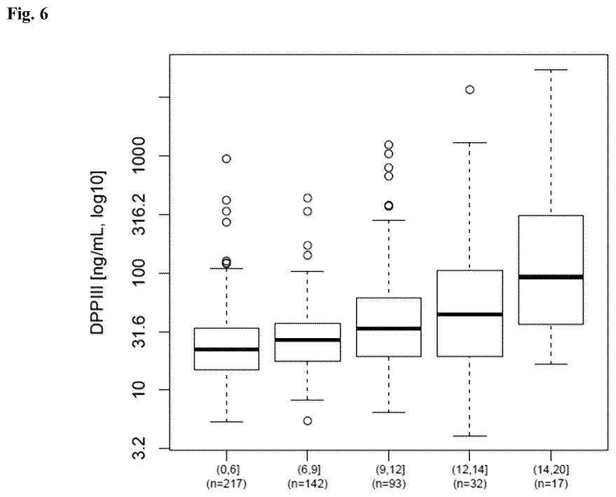

Median cDPP3 measured at admission in all AdrenOSS-1 patients was 45.1 ng/mL (inter quartile range 27.5-68.6). High DPP3 levels measured at admission were associated with worse metabolic parameters, renal and cardiac function, and SOFA score: patients with DPP3 levels below the median had a median SOFA score (points) of 6 (interquartile range (IQR) 4-9) compared to a median SOFA score of 8 (IQR 5-11) for patients with DPP3 levels above the median of 45.1 ng/mL (FIG. 6).

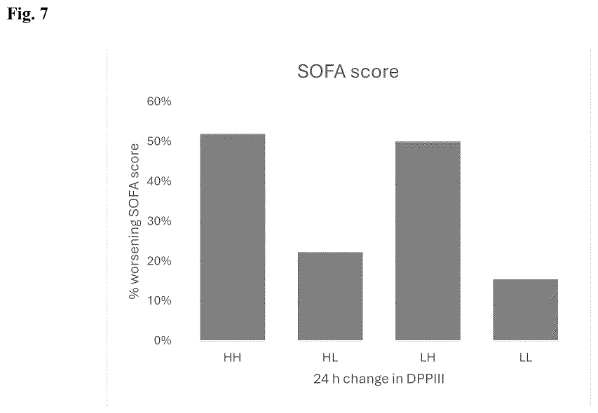

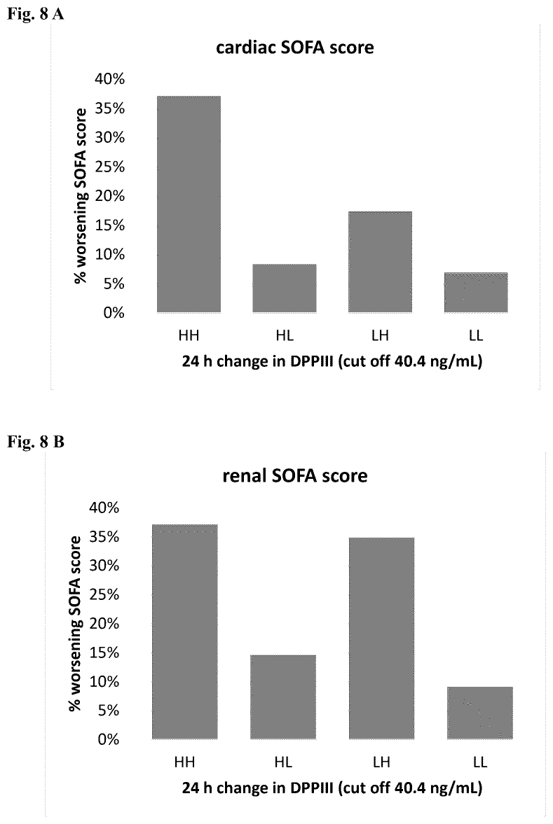

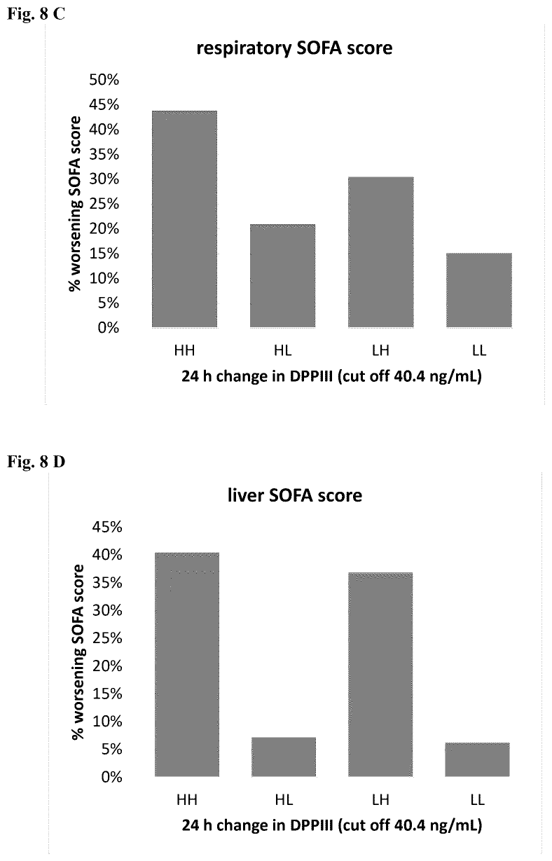

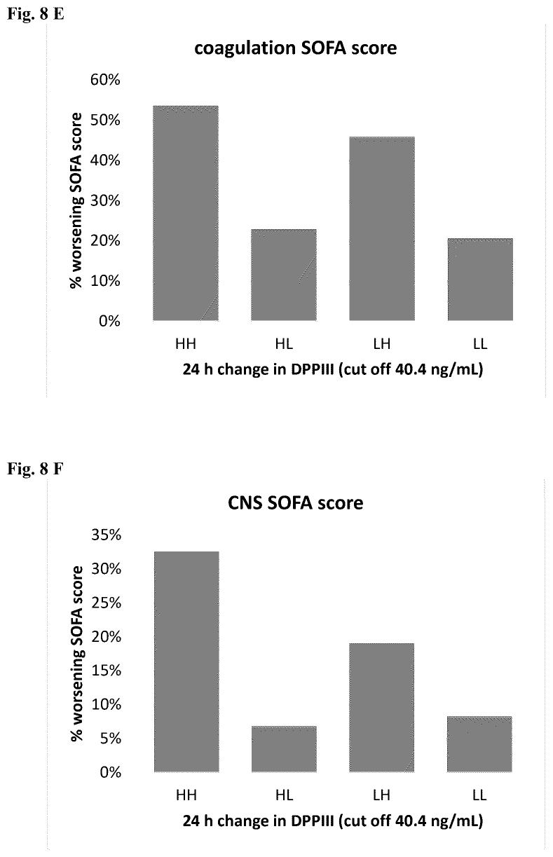

Whatever levels of cDPP3 at admission, high concentrations of cDPP3 levels 24 hours later were associated with worst SOFA scores whether global FIG. 7 or by organ (FIG. 8A-F).

In summary these data showed that high levels of cDPP3 were associated with survival and the extent of organ dysfunction in a large international cohort septic or septic shock patients. The study found marked association between cDPP3<45.1 ng/ml at admission and short-term survival as well as the prognostic cut-off value of 45.1 pg/ml in both sepsis and septic shock. Concerning organ dysfunction, there was a positive relationship between cDPP3 and SOFA score at ICU admission. More importantly, the relationship between cPDPP3 levels and extent of organ dysfunction, seen at ICU admission, was also true during the recovery phase. Indeed, patients with high cDPP3 levels at admission who showed a decline towards normal cDPP3 values at day 2 were more likely to recover all organ function including cardiovascular, kidney, lung, liver.

### Example 3—DPP3 in Ventilated ICU All-Corners

To assess the association between circulating DPP3 (cDPP3) and 28-day mortality in mechanically ventilated patients, n=390 patients admitted to the ICU and in need of mechanical ventilation on day of admission were included. The cohort includes, among others, patients admitted with Covid-19 infection, sepsis, septic shock, or cardiogenic shock.

For the quantification of DPP3 protein concentrations (DPP3-LIA) an assay as recently described was used (Rehfeld 2019).

Median cDPP3 measured at admission in all patients was 35.6 ng/mL (inter quartile range 19.9-63.4). High DPP3 levels measured at admission were associated with 28-day mortality: patients with DPP3 levels below 40.0 ng/mL had a survival rate of 74.2%, while patients with cDPP3 above 40.0 ng/mL had a mortality rate of only 55.5% (HR 2.1, 95-% CI 1.5-2.9, p<0.0001, FIG. 9).

In summary these data showed that high levels of cDPP3 were associated with survival in a large international cohort of ventilated ICU patients. The study found marked association between cDPP3 <40.0 ng/ml at admission and short-term survival.

### Example 4—DPP3 in Non-Invasive and Invasive Ventilated Patients with Septic Shock

The same study as described in Example 2 was used to assess the association between circulating DPP3 (cDPP3) and 28-day mortality in invasive and non-invasive ventilated patients on admission. From 583 patients admitted to the ICU with sepsis or septic shock, n=217 were in need of invasive ventilation and n=131 in need of non-invasive ventilation upon admission. The primary outcome was 28-day mortality. For the quantification of DPP3 protein concentrations (DPP3-LIA) an assay as recently described was used (Rehfeld 2019).

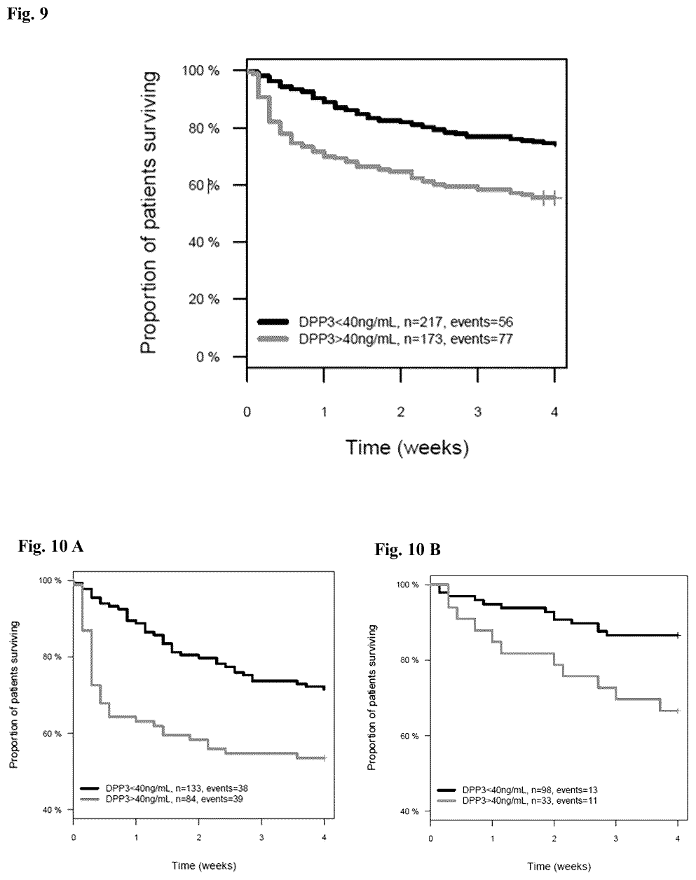

Median cDPP3 measured at admission in invasively ventilated patients (n=217) was 31.9 ng/mL (inter-quartile range 17.5-57.1). High DPP3 levels measured at admission were associated with 28-day mortality: patients with DPP3 levels below 40.0 ng/mL had a survival rate of 71.4%, while patients with cDPP3 above 40.0 ng/mL had a mortality rate of only 53.6% (HR 2.1, 95-% CI 1.3-3.3, p=0.0013, FIG. 10 A).

Median cDPP3 measured at admission in non-invasively ventilated patients (n=131) was 26.7 ng/mL (inter-quartile range 17.6-40.1). High DPP3 levels measured at admission were associated with 28-day mortality: patients with DPP3 levels below 40.0 ng/mL had a survival rate of 86.6%, while patients with cDPP3 above 40.0 ng/mL had a mortality rate of only 66.7% (HR 2.8, 95-% CI 1.2-6.2, p=0.0097, FIG. 10 B).

In summary these data showed that high levels of cDPP3 were associated with survival in a large international cohort of both invasively and non-invasively ventilated ICU patients with sepsis or septic shock. The study found marked association between cDPP3<40.0 ng/ml at admission and short-term survival.

### Example 5—DPP3 Activity and Concentration Levels in Healthy Mouse, Cynomolgus Monkey, Pig, and Human Samples

A study was performed to compare the DPP3 activity, concentration, and specific activity across species (human, mouse, cynomolgus monkey (a Non-Human Primate (“NHP”)), and pig).

Mouse, NHP (cynomolgus monkey), and pig samples were analysed via DPP3 activity assays validated or qualified for the serum or plasma of each species. Human DPP3 activity was tested using the DPP3 activity assay qualified for human serum or plasma. Similarly, mouse, cynomolgus monkey, and pig samples were analysed via DPP3 concentration assays qualified for the serum or plasma of each species. Human DPP3 concentration has been tested using the DPP3 concentration assay developed, qualified for human serum and CE-marked in vitro diagnostic (IVD) for human EDTA plasma. Specific activity was calculated by dividing the DPP3 activity by the DPP3 concentration in each sample.

DPP3 concentration levels in samples from healthy cynomolgus monkey, pig, mouse, and humans were comparable, with mouse and pig samples having slightly higher DPP3 concentration in comparison to human and cynomolgus monkey samples (Table 6). While mouse and human DPP3 activity was comparable, the pig and cynomolgus monkey samples exhibited a higher DPP3 activity in sera or plasma, leading to a higher specific activity (Table 6).

TABLE 6
Median and IQR of DPP3 concentration, activity,
and specific activity in relevant species.

| | DPP3 median | | DPP3 median | | DPP3 median specific | |
|---|---|---|---|---|---|---|
| | concentration | | activity in | | activity in | |
| Species | in ng/mL (IQR) | | U/L (IQR) | | μmol/(min*mg) (IQR) | |
| Human | 14 | (11.0-19.0) | 74.0 | (58.7-106.0) | 6.8 | (6.1-7.6) |
| | (n = 5021) | | (n = 578) | | (n = 577) | |
| Mouse | 22.6 | (18.5-28.5) | 94.7 | (74.9-124.8) | 3.8 | (3.0-4.4) |
| | (n = 127) | | (n = 434) | | (n = 127) | |
| Pig | 32.7 | (23.5-52.0) | 278.0 | (190.3-384.4) | 12.6 | (9.3-15.4) |
| | (n = 114) | | (n = 174) | | (n = 91) | |
| Cynomolgus | 15.8 | (12.1-23.2) | 467.2 | (380.6-527.1) | 28.4 | (24.3-34.0) |

| | | | |
|---|---|---|---|
| monkey | (n = 88) | (n = 60) | (n = 60) |

n = Number; IQR = inter-quartile range.

This study demonstrated that the DPP3 concentration levels across all of the species that were studied are comparable. Higher DPP3 activities were observed in pig and cynomolgus monkey samples, resulting in higher DPP3 specific activity compared to human samples.

### Sphingotest® DPP3 Immunoluminometric Assay (ILMA) for the Quantitative Measurement of Dipeptidyl Peptidase 3 in Human EDTA Plasma:

The protein concentration of DPP3 from human EDTA plasma samples is measured using the Sphingotest® DPP3 (sphingotec: the Biomarker Company, Germany) assay kit. It is a non-automated immunoluminometric assay (ILMA) for the in vitro diagnostic quantitative measurement of DPP3 protein concentration in human EDTA plasma. The instructions from the manufacturer were followed (IFU-IVD-DPP V06 released October 2022). In brief, two antigen-specific monoclonal antibodies are used, which bind DPP3 (antigen) at two different epitopes. One of the antibodies is luminescent labelled (tracer antibody), the other is immobilized on the inside of the microtiter plate wells (capture antibody). During incubation, both antibodies react with the DPP3 in the plasma sample building a sandwich complex. In this way, the tracer antibody is indirectly bound to the surface of the microtiter plate. Afterwards, the residual excess tracer is completely removed by careful washing. The amount of captured tracer antibody is determined by measuring the luminescence in a suitable luminometer, using the supplied Sphingotest® Lightning reagents. The luminescence signal (relative light units, RLU) is directly proportional to the DPP3 concentration of the respective plasma sample. Parallel measurement of the included calibrators, with known concentrations of native human DPP3, enables the generation of a calibration curve, through which the unknown DPP3 concentration in the sample can be deduced.

### Example 6—Anti-DPP3 Antibody Inhibition Studies in Mouse, Cynomolgus Monkey, Pig, and Human Serum

A study was performed to test the ability of the anti-DPP3 antibody to inhibit native and recombinant mouse, pig, and cynomolgus monkey DPP3 and compare the anti-DPP3 antibody inhibition potential in the different species to the inhibition of human DPP3.

Mouse and cynomolgus monkey sera and pig plasma samples were analysed via DPP3 activity assays qualified for the matrix of each species. Human DPP3 inhibition has been tested using the DPP3 activity assay qualified for human sera and plasma (Similarly, recombinant mouse (Uniprot No. Q99KK7), cynomolgus monkey (Uniprot No. AOA7N9C8Y6) and pig (Uniprot No. AOA5G2RLJ6) DPP3 were expressed in E. coil and purified via a N-terminal His-tag. Recombinant proteins were analyzed in the respective assays described above in Example 5. Human DPP3 was purified from commercially available human blood cell lysate as described by Kaufmann et al. (Kaufmann, P et al., PLoS One 14, e0220866 (2019)).

The study demonstrated that the anti-DPP3 antibody inhibits DPP3 in native serum from cynomolgus monkey, pig, and mouse in a similar fashion as it inhibits human DPP3 (Table 7). In addition, the anti-DPP3 antibody inhibits recombinantly produced mouse, cynomolgus monkey, and pig DPP3 (Table 8).

TABLE 7
Inhibition of native DPP3 from different species
by the anti-DPP3 antibody in native serum/plasma
of the different species as compared to humans.

| | Mean +/− SD | Mean +/− SD |
|---|---|---|
| Species serum/plasma | IC₅₀ [μg/mL] | Iₘₐₓ [%] |
| Human | 5.8 +/− 1.7 | 85.2 +/− 9.1 |
| Mouse | 2.3 +/− 0.5 | 83.2 +/− 1.0 |
| Pig | 4.0 +/− 0.2 | 80.8 +/− 1.7 |
| Cynomolgus monkey | 10.1 +/− 2.5 | 57.2 +/− 3.4 |

Iₘₐₓ = maximal inhibition; IC₅₀ = half-maximum inhibitory concentration; SD = Standard deviation.

TABLE 8
Inhibition of recombinant DPP3 from different species
by the anti-DPP3 antibody as compared to humans.

| | IC₅₀ [μg/mL] | IC₅₀ [μg/mL] | |
|---|---|---|---|
| | at 15 DPP3 | at 200 DPP3 | Iₘₐₓ [%] |
| DPP3 | ng/mL | ng/mL | mean |
| Human native | 6.1 | 8.9 | 82.6 |
| Mouse recombinant | 2.5 | 3.1 | 86.6 |
| Pig recombinant | 4.2 | 4.8 | 83.6 |
| Cynomolgus monkey | 5.0 | 7.6 | 73.7 |
| recombinant | | | |

Imax = maximal inhibition; IC50 = half-maximum inhibitory concentration.

### Example 7—DPP3 and the Anti-DPP3 Antibody Mode of Action on Systemic and Renal Hemodynamics in Mice

A study was performed to investigate the effects of DPP3 and the anti-DPP3 antibody when given as i.v. administration on the modulation of systemic and renal hemodynamics as well as the renin-angiotensin system in healthy mice.

The study design is provided in Table 9 and Table 10.

TABLE 9
Overview of Study Design

| | | | Number of |
|---|---|---|---|
| Group (Description) | Pre-treatment | Treatment | Animals |
| 1 (PBS) | No | PBS; i.v. | 91 |
| 2 (anti-DPP3 | No | anti-DPP3 antibody; | 91 |
| antibody) | | 10 mg/kg; i.v. | |
| 3 (DPP3) | No | DPP3; 0.55 mg/kg; i.v. | 91 |
| 4 (DPP3) | Yes, valsartan | DPP3; 0.55 mg/kg; i.v. | 6 |
| 5 (DPP3) | Yes, labetalol | DPP3; 0.55 mg/kg; i.v. | 5 |
| 6 (DPP3) | Yes, naloxone | DPP3; 0.55 mg/kg; i.v. | 3 |

PBS = Phosphate buffered saline.

TABLE 10
Overview of Study Design for pharmacological treatment
before and after DPP3 or anti-DPP3 antibody injection.

| | Pharmacological test before | | |
|---|---|---|---|
| Group | and after DPP3/anti-DPP3 | | |
| (Description) | antibody | Treatment | Number of Animals |
| 7 (anti-DPP3 | Yes, Ang II/norepinephrine | anti-DPP3 antibody; | 5 (3 norepinephrine |
| antibody) | | 10 mg/kg; i.v. | sub-group) |
| Group | Pharmacological test before | Treatment | Number of Animals |
| (Description) | and after DPP3/anti-DPP3 | | |
| | antibody | | |
| 8 (DPP3) | Yes, Ang II/norepinephrine | DPP3; 0.55 mg/kg; i.v. | 6 (4 norepinephrine |
| | | | sub-group) |

Ang II = Angiotensin II

Ten-week-old male C57Bl/6JRj mice were administered i.v. bolus injection (4 mL/kg) of 0.55 mg/kg DPP3 (n=91), 10 mg/kg the anti-DPP3 antibody (n=91), or phosphate-buffered saline (PBS) (n=91). Continuous measurements of hemodynamic variables (mean arterial pressure (MAP) and renal blood flow (RBF)) were performed in anesthetized mice. Sodium chloride (0.9%) was i.v. infused for fluid maintenance. RBF and MAP were measured before and after i.v. bolus injections of 0.55 mg/kg DPP3 (n=6), 10 mg/kg anti-DPP3 antibody (n=5), 0.25 to 2 ng Ang II, and 20 ng norepinephrine. In some experiments, 0.8 mg/kg valsartan (Ang II type 1 receptor antagonist) (n=6), 1.6 mg/kg naloxone (pan-opioid receptor antagonist) (n=3), or 10 mg/kg labetalol (alpha- and beta-adrenergic receptor blocker) (n=5) was administered as i.v. bolus injection, 5 min before DPP3 (0.55 mg/kg) administration in order to single out the receptor(s) responsible for the DPP3 effects on renal and systemic hemodynamics. Untreated animals received a bolus of 100 μL 0.9% sodium chloride. Renovascular resistance (RVR) was estimated as the ratio of MAP/RBF.

Following 5 min, 15 min, or 60 min after administration of DPP3, PBS, or the anti-DPP3 antibody, the animals were sacrificed. DPP3 activity was assessed via a soluble activity assay. The anti-DPP3 antibody concentration was measured for quality control via an ELISA assay. Angiotensin peptides were determined by mass spectrometry. Epinephrine, norepinephrine, and dopamine were measured using an isocratic high performance liquid chromatography system with electrochemical detection.

DPP3-injected mice exhibited a transient and massive increase of RBF, while MAP remained constant, reflecting decreased RVR. The maximal effect of a single bolus injection of DPP3 was reached within ˜1 min and lasted 15 to 30 min. Conversely, the anti-DPP3 antibody administration produced a progressive, small but significant reduction of RBF without change in MAP, thus reflecting increased RVR (FIGS. 11 A, 11 B, and 11 C).

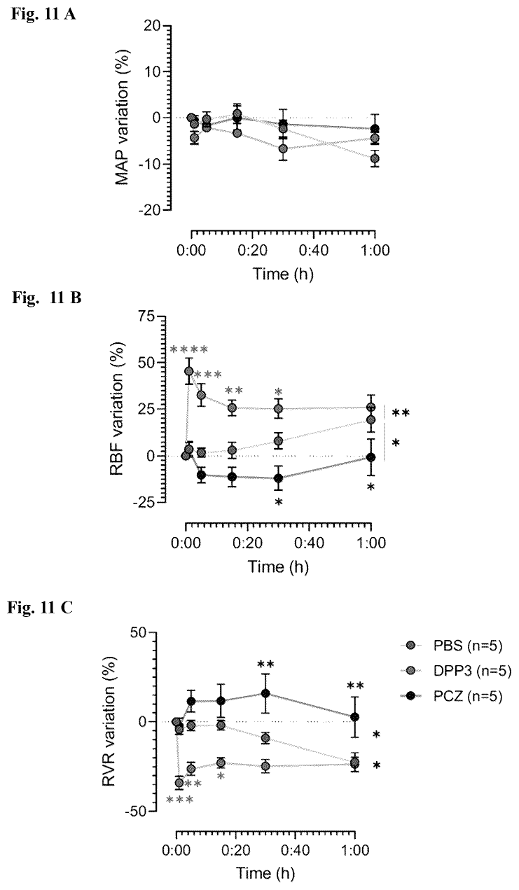

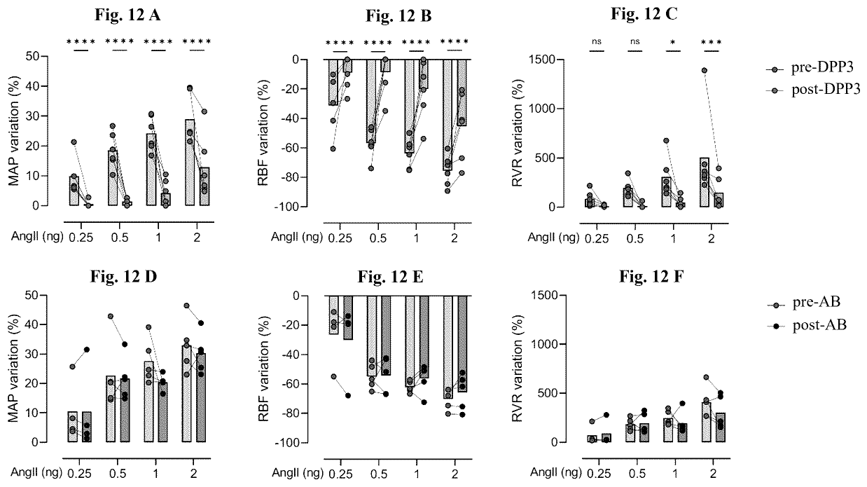

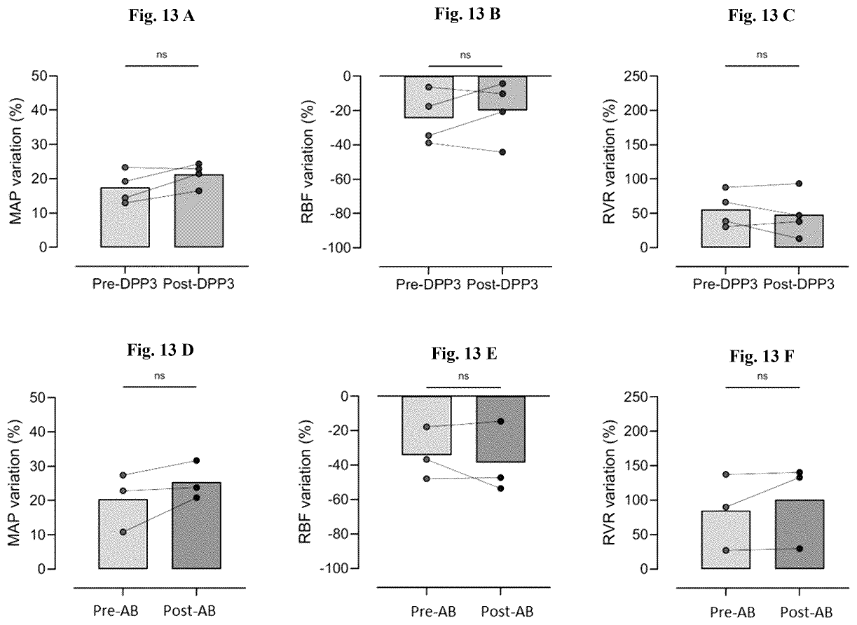

As Ang II is a substrate of DPP3, the effects of DPP3 or the anti-DPP3 antibody administration on systemic or renal vasoreactivity to Ang II were tested. Prior to DPP3 injection, repeated i.v. bolus of increasing doses of Ang II induced a dose-dependent increase in MAP concomitantly with a dose-dependent decrease in RBF reflecting increased RVR (FIGS. 12 A, 12 B, and 12 C). After DPP3 administration, the systemic (MAP) and renal (RBF, RVR) vasoreactivity to Ang II was systematically decreased. In contrast, DPP3 or the anti-DPP3 antibody administration did not affect the vasoconstrictive effect of norepinephrine, a vasopressor which metabolism is DPP3-independent (FIGS. 13 A, 13 B, 13 C, 13 D, 13 E, and 13F). Conversely, the anti-DPP3 antibody administration did not affect the systemic or renal vasoreactivity of Ang II (FIG. 12 D, 12 E, and 12 F).

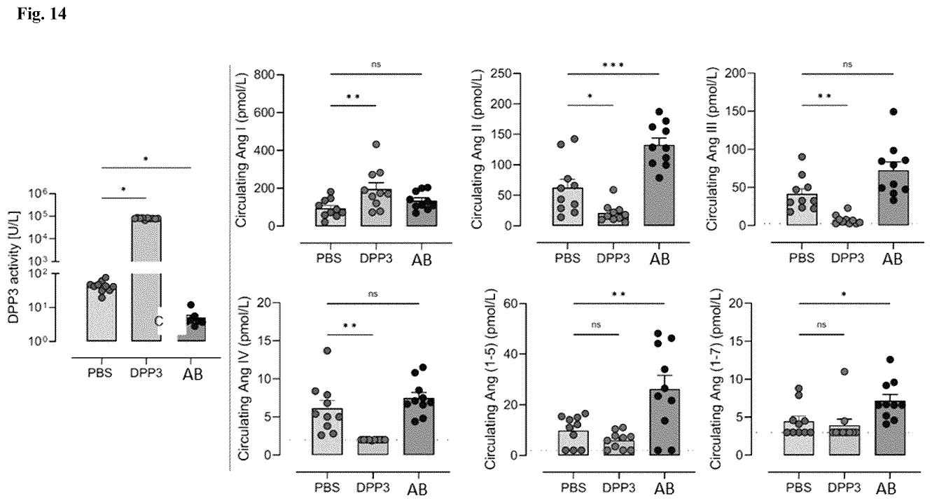

Compared to PBS-injected animals, DPP3 administration was associated with increased circulating Ang I and decreased concentrations of Ang II, III, and IV, 15 min after injection. Conversely, the anti-DPP3 antibody administration led to increased circulating concentrations of Ang II and (1-5) (FIG. 14). Effect of DPP3 but not anti-DPP3 antibody on circulating angiotensin concentrations persisted 60 min after injection (FIG. 14).

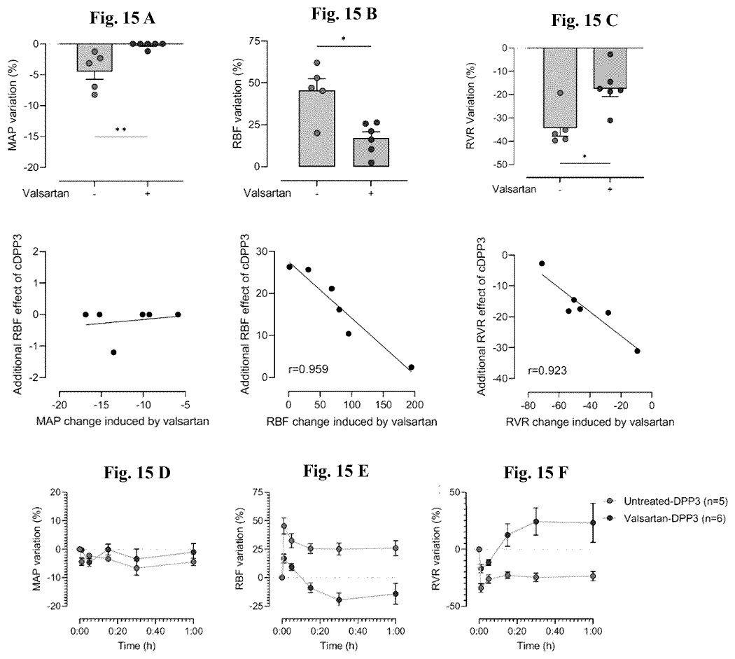

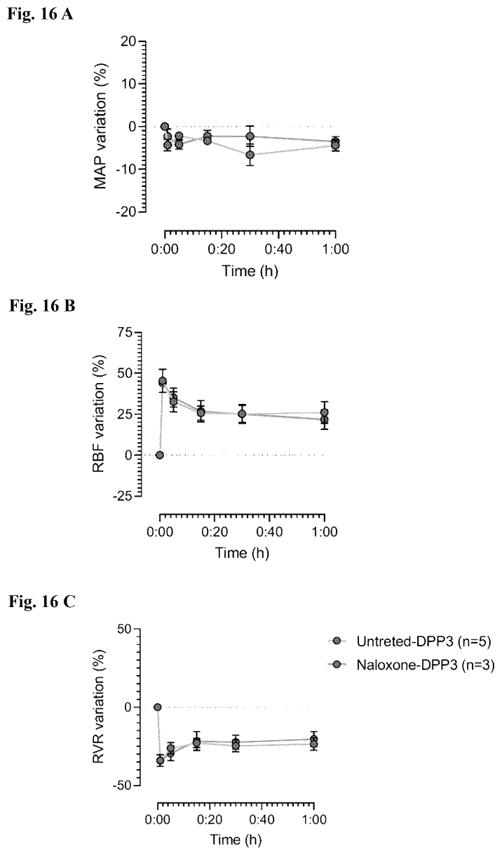

Compared to untreated mice, valsartan-pretreated mice (an Ang II type 1 receptor antagonist mediating the vasoconstrictive effect of Ang II) exhibited decreased response to DPP3 (FIGS. 15 A, 15 B, 15 C, 15 D, 15 E, and 15 F). A strong negative correlation between the effect induced by valsartan on RBF and the added effect of subsequent DPP3 injection was observed, suggesting that the more effective AT1R blockade is, the less additive DPP3 effect is observed. In contrast, a pre-treatment by the pan-opioid receptor antagonist naloxone produced a marginal hemodynamic effect and did not alter the response to subsequent DPP3 administration (FIGS. 16 A, 16 B, and 16 C). Therefore, DPP3 mediates its effect on renal hemodynamics through decreased ATIR signaling.

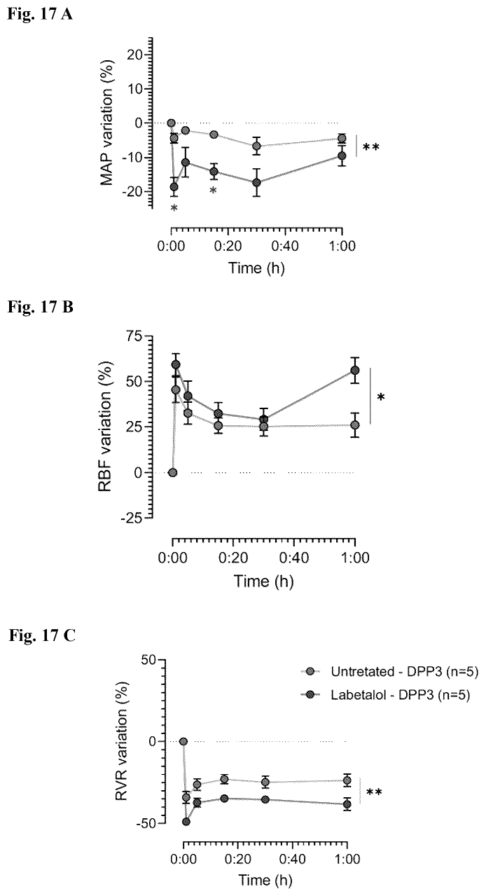

In comparison with PBS-injected mice, DPP3-injected mice exhibited a progressive release of catecholamines, reaching maximal concentrations at 1h while the anti-DPP3 antibody-injected mice tend to exhibit the opposite effect (Table 11). To confirm that catecholamine release prevents decrease of blood pressure post-hDPP3 injection, mice were pre-treated with the alpha- and beta-adrenergic receptor blocker, labetalol, before DPP3 administration. As expected, labetalol pre-treatment induced a sustained decrease of MAP, without significant changes of nor RVR. Upon DPP3 administration, an abrupt fall of blood pressure was observed in labetalol-pretreated mice, contrasting with untreated mice. Additionally, pre-treatment by labetalol was associated with enhanced DPP3 effects on renal hemodynamics (FIGS. 17 A, 17 B, and 17 C). Taken together, these findings confirm that endogenous catecholamine release compensates for the hemodynamic changes induced by DPP3-mediated angiotensin cleavage in healthy mice.

TABLE 11
Mean endogenous catecholamine concentrations at 5, 15, and 60
min after PBS, DPP3, or anti-DPP3 antibody administration.

| | | | |
|---|---|---|---|
| | | anti-DPP3 | p |
| PBS | DPP3 | antibody | ANOVA or |
| Mean ± SD | Mean ± SD | Mean ± SD | KW |

Norepinephrine (pg/mL)

| | | | | |
|---|---|---|---|---|
| T5 | 10321 ± 2402 | 12342 ± 2331 | 8734 ± 2392 | 0.008 |
| T15 | 10800 ± 2221 | 10378 ± 2863 | 7327 ± 2953 * | 0.0201 |
| T60 | 9411 ± 1352 | 17744 ± 3993 **** | 7500 ± 1094 | <0.0001 |

Epinephrine (pg/mL)

| | | | | |
|---|---|---|---|---|
| T5 | 1503 ± 485.2 | 1961 ± 494.9 | 1195 ± 282.4 | 0.0035 |
| T15 | 1349 ± 575.7 | 2436 ± 824.1 | 873.2 ± 223.0 | 0.0002 |
| T60 | 1282 ± 174.8 | 3325 ± 1337 * | 765.4 ± 152.3 * | <0.0001 |

Dopamine (pg/mL)

| | | | | |
|---|---|---|---|---|
| T5 | 2484 ± 519.3 | 2718 ± 683.4 | 2471 ± 586.1 | 0.5916 |
| T15 | 1961 ± 723.3 | 2624 ± 698.8 * | 1780 ± 352.8 | 0.0122 |
| T60 | 1720 ± 505.1 | 2834 ± 596.5 **** | 1234 ± 250.8 | <0.0001 |

Post-hoc comparisons with PBS group: * p < 0.05, ** p < 0.01, *** p < 0.001, **** p < 0.0001 in comparison with the PBS group at the same timepoint.
N = 8-10 per group at each timepoint.
DPP3 = Dipeptidyl Peptidase 3;
KW = Kruskal-Wallis test;
PBS = Phosphate buffered saline;
SD = Standard deviation;
T = Timepoint.

DPP3 administration to mice induces a transient drop in RVR caused by enhanced degradation of Ang II leading to increased RBF whereas blood pressure is maintained by means of increased circulating endogenous catecholamines. The anti-DPP3 antibody-injected mice tend to exhibit the opposite effect. Therefore, DPP3 is an actor of circulatory failure and contributes to RAAS alterations and associated consequences in shock. The anti-DPP3 antibody counteracts the negative effects of DPP3 in this model.

### Analytical Method for Anti-DPP3 Antibody Measurement in Human Serum Using Immunoassays:

The objective of this method will be to measure the anti-DPP3 antibody in human serum, using an ELISA. In brief, the anti-DPP3 antibody will bind to a tagged Biotin-DPP3 peptide bound to a precoated Streptavidin clear microplate. The biotin-DPP3-peptide is biotin—Trioxatridecan—Succinamic Acid (Ttds)—ETVINPETGEQIQSWYR-NH2, where SEQ ID NO: 62 ETVINPETGEQIQSWYR. The biotin-DPP3 peptide/anti-DPP3 antibody complex will be detected by a Mouse anti-human IgG1 Fc horse radish peroxidase (HRP) conjugated antibody. A TMB Sureblue substrate solution will be added to the wells and a color will develop in proportion to the amount of analyte (anti-DPP3 antibody) bound in the initial step. The color development will be stopped with stop solution and the absorbance will be read at 450 nm in a microplate reader.

The precoated streptavidin plate (ThermoFisher Cat. No. 436014) will be blocked with 300 μl of Superblock Blocking Buffer in PBS (Thermo Cat. No. 37515), covered, incubated for 1 hour and then the block buffer will be removed. The plate will be washed one time with wash buffer (PBS 1×+0.05% Tween 20). The washed microplate will be coated with 100 μl of Biotin-DPP3 peptide diluted to 50 ng/ml with coating buffer (PBS 1×+Bovine Serum Albumin (BSA) 0.1%+Tween 20 0.05%) covered, protected from light and incubated at room temperature for 1 hour while shaking at 600 rpms. The coated microplates will be washed four times with 300 μl of the wash buffer. After the last wash buffer has been removed, a series of serum samples will be diluted with dilution buffer (PBS 1×+BSA 1%+0.1 M CaCl₂-2 H₂O) and will be deposited onto the washed plates at 100 μl and incubated for 2 hours at room temperature, protected from light and shaking at 600 rpms. The coated microplates will be washed again for four times with 300 μl of the wash buffer. The secondary antibody Mouse anti-human IgG1 Fc horse radish peroxidase (HRP) conjugated antibody (Invitrogen, Cat. No. A10648) will be diluted with the coating buffer (20:12,000) and 100 μl will be added and then incubated for 1 hour at room temperature, protected from light and shaking at 600 rpms. The coated microplates will be washed five times with 300 μl of the wash buffer. The substrate TMB (3,3′,5,5′-tetramethylbenzidine) Sureblue (Eurobio, Cat. No. 5120-0077) will be added at 100 μl for ten minutes at room temperature and protected from light. The reaction will be stopped at ten minutes by the addition 100 μl of stop solution (H₂SO₄ 2N 1M). The plate will be read (endpoint) within 30 minutes via microplate reader (SpectraMax i3x) at 450 nm while plate shaking for 5 seconds. All volume steps are provided per well. The samples will be measured against known concentrations of anti-DPP3 antibody calibration standards to create the standard curve for the assay.

### Example 8—Isoproterenol-Induced Cardiac Dysfunction in Mice

This study tested the efficacy of the anti-DPP3 antibody in a mouse model of acute cardiac stress. The study also included a limited set of safety-relevant endpoints.

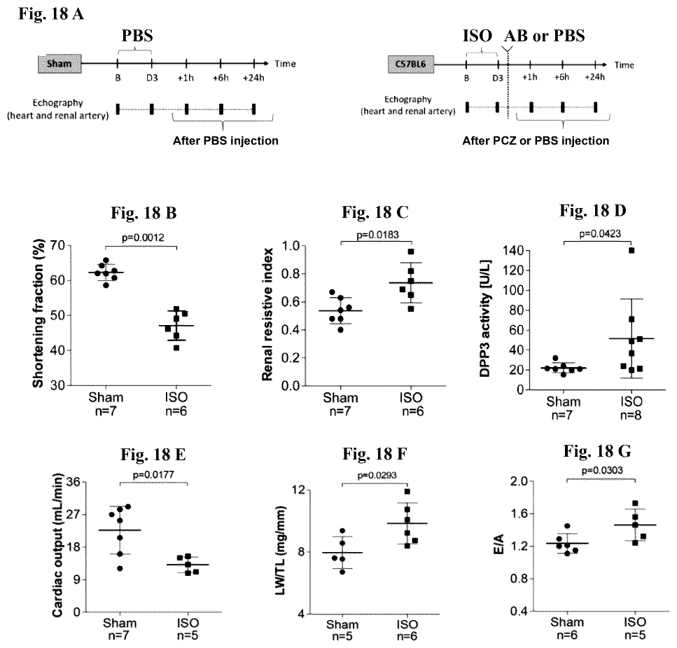

The study design is provided in Table 12 and FIG. 18A.

TABLE 12
Overview of Study Design

| | HF | | Number |
|---|---|---|---|
| | Induction | | of |
| Group (Description) | by ISO | Treatment | Animals |
| 1 (Sham) | No | PBS; i.v. | 20 |
| 2 (Sham + anti-DPP3 antibody) | No | anti-DPP3 antibody; 10 mg/kg i.v. | 3 |
| 3 (ISO-HF + PBS) | Yes | PBS; i.v. | 14 |
| 4 (ISO-HF + anti-DPP3 | Yes | anti-DPP3 antibody; 10 mg/kg; i.v. | 21 |
| antibody) | | | |
| 5 (ISO-HF + IgG) | Yes | IgGI isotype control; 10 mg/kg; i.v. | 3 |

HF = Heart Failure; ISO = Isoproterenol; i.v. = Intravenous; PBS = Phosphate Buffered Saline.

Acute cardiac stress was provoked in 3-month-old C57BL/6 male mice by subcutaneous injections of 300 mg/kg of isoproterenol (ISO) in 0.9% NaCl (saline) twice daily for 2 days. (Vergaro, G., et al., 2016, Hypertension 67, 606-612 (Vergaro 2016)). Sham mice (n=20) only received saline. Anti-DPP3 antibody (10 mg/kg; n=14), phosphate-buffered saline (PBS) (n=21) or isotype control antibody (10 mg/kg; n=3) were i.v. injected in mice and isoproterenol-induced cardiac dysfunction was assessed by a significant decrease in fractional shortening 12 h after the last ISO injection. Mice were sacrificed 1, 6, or 24 h after injection (FIG. 18A). For the analysis of long-term effects, isoproterenol-induced heart failure (HF) (ISO-HF)+PBS (n=5) and ISO-HF+anti-DPP3 antibody (n=5) mice were observed for 14 days. Heart and kidney function were monitored by echocardiography and kidney ultrasound, respectively. Afterwards, the animals were sacrificed. Body, heart, and lung weights and tibia length were recorded. Lung weight variations were expressed relative to the tibia length. The heart was transversally divided into two parts: the base part was embedded into Tissue-Tek optimal cutting temperature (OCT) compound and frozen into isopentane pre-cooled in liquid nitrogen for oxidative stress analysis; the apex was flash-frozen in liquid nitrogen and used for mRNA analysis. DPP3 activity was assessed via the soluble activity assay. Anti-DPP3 antibody concentration was measured via an ELISA assay. Safety-relevant endpoints included in the study were monitoring of clinical signs, and macropathology.

The i.v. treatment with 10 mg/kg anti-DPP3 antibody was well tolerated in mice with no apparent adverse test item-related clinical signs or findings at macropathology.

The ISO treatment of mice led to heart failure indicated by a significant decrease in left ventricular shortening fraction (LVSF) (FIG. 18B), significant increase in renal resistive index (RRI) (FIG. 18C), and significant decrease in cardiac output (CO) compared to the sham-treated ISO animals (FIG. 18E). Furthermore, a significant increase in lung congestion and in E/A wave mitral ratio was observed (FIG. 18F and FIG. 18G, respectively). DPP3 activity was significantly higher in the ISO-HF group compared to the sham control group, which received only PBS without ISO treatment (FIG. 18D). Bolus administration of the anti-DPP3 antibody in mice was shown to follow the expected profile for i.v. administered drugs. Cₘₐₓ was detected early after administration and followed by a rapid decrease in exposure.

Furthermore, cardiac DPP3 mRNA and protein levels were significantly higher in ISO-HF mice when compared with sham mice.

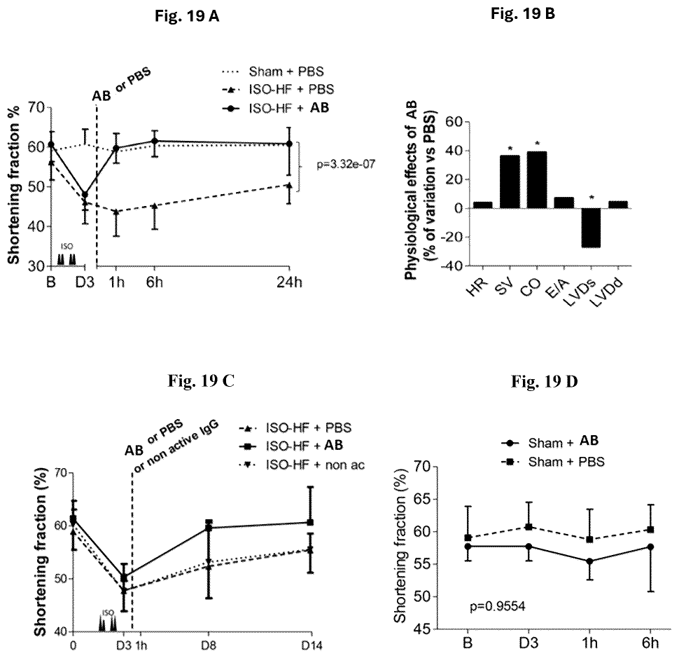

The anti-DPP3 antibody injection rapidly normalized the LVSF, and improved stroke volume (SV) as well as CO of ISO-HF+anti-DPP3 antibody mice within 1 h and 6 h, respectively after injection when compared with ISO-HF+PBS group; LVSF remained normal at 24 h and 14 days after the single initial anti-DPP3 antibody injection in ISO-HF+anti-DPP3 antibody mice. The injection of a non-active, isotype control IgG had no effect on LVSF in ISO-HF mice and values were similar to those of the ISO-HF+PBS at day 8 and day 14 (FIG. 19 C). LVSF was not modified in the Sham+anti-DPP3 antibody group compared with Sham+PBS, 1 h and 6 h after anti-DPP3 antibody injection (FIG. 19D).

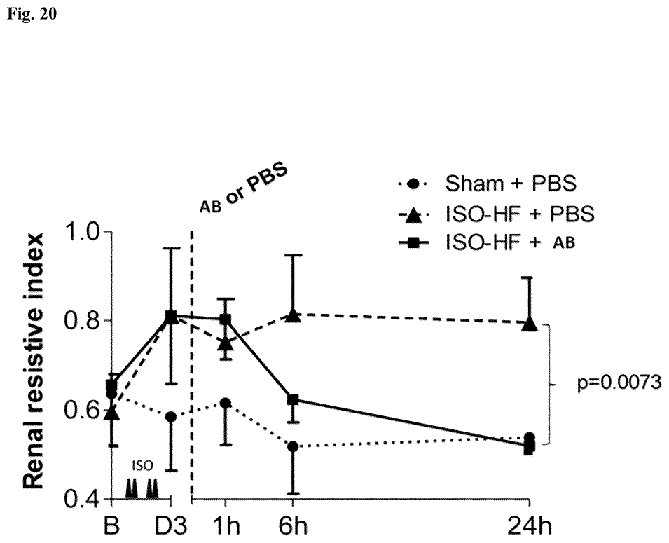

Renal hemodynamics measured by renal resistance index were restored in ISO-HF+anti-DPP3 antibody mice within 24 h to levels of the sham control (PBS) and where significantly lower than those of ISO-HF+PBS treated mice (FIG. 20).

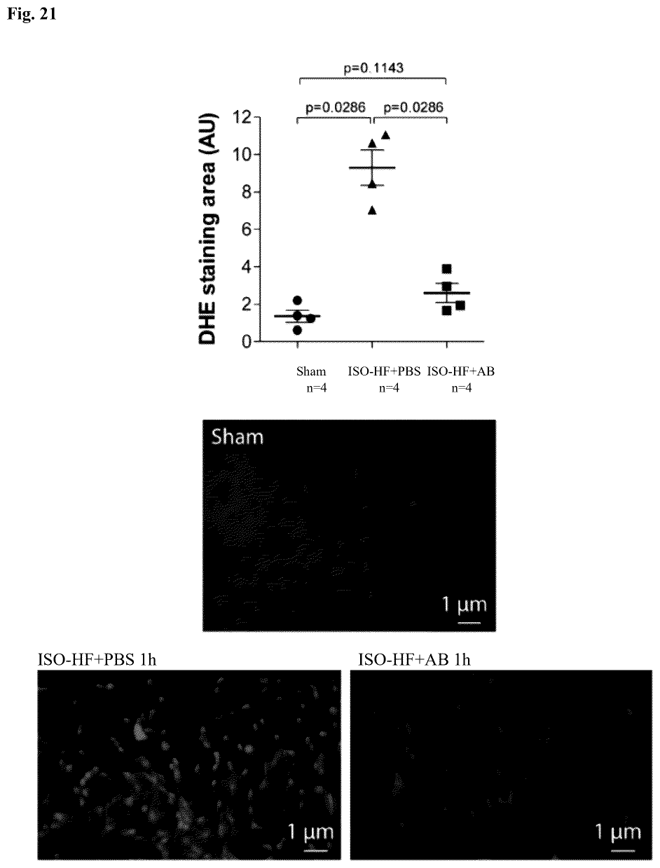

In addition to the improvement in hemodynamics, the anti-DPP3 antibody rapidly reduced oxidative stress in the heart of ISO-HF+anti-DPP3 antibody mice when compared with those of ISO-HF+PBS animals as can be seen by reduced levels of dihydroethidium (DHE) labelling (FIG. 21).

The study demonstrated that 10 mg/kg anti-DPP3 antibody when i.v. injected as a bolus is safe and well tolerated in mice under the conditions of the study. The ISO-HF model was found suitable for efficacy testing of anti-DPP3 antibody since it led to an increase in the target cDPP3 accompanied by signs of acute heart failure. Administration of anti-DPP3 antibody improved renal hemodynamics and cardiac function, as well as reduced oxidative stress in the heart of ISO-HF mice compared to the control vehicle.

### Example 9—Anti-DPP3 Antibody Efficacy Dose Range Finding in Mice

A study was performed to investigate the efficacy dose range (therapeutic window) of the anti-DPP3 antibody, when i.v. administered in mice. Efficacy was defined as improvement of cardiac dysfunction in an isoproterenol-induced acute heart failure (AHF) mouse model. In addition, a correlation of the largest improvement in shortening fraction with the most prominent DPP3 inhibition was performed. The study also included a limited set of safety-relevant endpoints.

The study design is provided in Table 13.

TABLE 13
Overview of Study Design

| | HF | | Number |
|---|---|---|---|
| | Induction | | of |
| Group (Description) | by ISO | Treatment | Animals |
| 1 (ISO-HF + PBS) | Yes | PBS; i.v. | 3 |
| 2 (ISO-HF + anti- | Yes | anti-DPP3 antibody | 4 |
| DPP3 antibody) | | 1 mg/kg; i.v. | |
| 3 (ISO-HF + anti- | Yes | anti-DPP3 antibody | 4 |
| DPP3 antibody) | | 5 mg/kg; i.v. | |
| 4 (ISO-HF + anti- | Yes | anti-DPP3 antibody | 9 |
| DPP3 antibody) | | 10 mg/kg; i.v. | |
| 5 (ISO-HF + anti- | Yes | anti-DPP3 antibody | 9 |
| DPP3 antibody) | | 50 mg/kg; i.v. | |
| 6 (ISO-HF + anti- | Yes | anti-DPP3 antibody | 3 |
| DPP3 antibody) | | 100 mg/kg; i.v. | |

HF = Heart Failure; ISO = Isoproterenol; i.v. = Intravenous; PBS = Phosphate Buffered Saline.

Acute cardiac stress was provoked in 3-month-old C57BL/6 male mice by subcutaneous injections of 300 mg/kg of isoproterenol (ISO) in 0.9% NaCl (saline) twice daily for 2 days (Vergaro2016). Twelve hours after the last isoproterenol injection, animals were anesthetized by intraperitoneal ketamine injection and an echocardiography was performed for 5-10 min (before injection (BI)). Mice presenting with AHF (i.e., decrease of 10% in left ventricular shortening fraction—LVSF) received the anti-DPP3 antibody or PBS bolus injection in a volume of 100 μL via the retro-orbital vein. The mice were divided into 6 groups: ISO-HF+PBS (n=3), ISO-HF+anti-DPP3 antibody 1 mg/kg (n=4), ISO-HF+anti-DPP3 antibody 5 mg/kg (n=4), ISO-HF+anti-DPP3 antibody 10 mg/kg (n=9), ISO-HF+anti-DPP3 antibody 50 mg/kg (n=9), and ISO-HF+anti-DPP3 antibody 100 mg/kg (n=3). Cardiac function in each group was monitored by echocardiography for a period of 15 minutes, and reported at time-points T 5 min, T 30 min, and T 60 min. The animals were re-injected with ketamine 5 min before each timepoint, if required. Due to the negative inotrope and chronotope effect of overstimulation of adrenergic receptors via ISO injection, animals with a heart rate below 300 bpm were excluded from the cardiac function (LVSF) analyses. At the end of cardiac monitoring, a blood draw was performed, and the mice were sacrificed. At necropsy, gross pathology was performed. Baseline (Baseline or BL) echocardiography under ketamine anaesthesia and a retro-orbital blood draw were performed a week before the experiment start. DPP3 activity was assessed via a soluble activity assay. Anti-DPP3 antibody concentration was measured for quality control via an ELISA assay. Safety-relevant endpoints included in the study were monitoring of clinical signs and macropathology.

The i.v. treatment with anti-DPP3 antibody at all doses was well tolerated in mice with no obvious adverse test item-related clinical signs or findings at macropathology.

As expected, the ISO treatment of mice led to heart failure indicated by a significant decrease in left ventricular shortening fraction (LVSF) (Table 14). The animals included in the different groups had a mean decrease in LVSF of 14.79% after ISO administration (BI timepoint). PBS injection did not modify the LVSF of ISO-HF mice. Anti-DPP3 antibody administration led to a numerical improvement of LVSF at doses of 5, 10, and 50 mg/kg, with significance achieved only for the 10 and 50 mg/kg doses. Improvement of cardiac function was most pronounced at 30 and 60 mins. The i.v. administration of the anti-DPP3 antibody at 100 mg/kg did not modify the LVSF of ISO-HF mice.

DPP3 measurements at one hour (T60 mins) after PBS dosing (ISO-HF+PBS) was used as the mean baseline to compare changes in DPP3 activity in the anti-DPP3 antibody treatment groups. The ISO-HF mice that received 10, 50, and 100 mg/kg doses displayed 67%, 60%, and 68% DPP3 activity inhibition, respectively, one-hour after anti-DPP3 antibody i.v. administration. No DPP3 inhibition was observed at the 1 mg/kg dose and a 36% inhibition was observed for the 5 mg/kg dose in ISO-HF mice (Table 14). Anti-DPP3 antibody concentration was also measured in all samples for all groups at 120 min to confirm either its absence or the correlation between DPP3 activity inhibition and concentration of the anti-DPP3 antibody in the blood.

TABLE 14
Changes in left ventricular shortening fraction (LVSF) upon injection of different doses
of the anti-DPP3 antibody in mice with isoproterenol-induced cardiac dysfunction.

| | | | | | | DPP3 |
|---|---|---|---|---|---|---|
| | LVSF | | | | | inhibition at |
| | | | | 30 min | 60 min | 60 min after |
| | | BI (after ISO, | 5 min after | after PBS | after PBS | PBS or anti- |
| | | before PBS | PBS or | or anti- | or anti- | DPP3 |
| Group | | or anti-DPP3 | anti-DPP3 | DPP3 | DPP3 | antibody |
| (Description) | BL | antibody) | antibody | antibody | antibody | (%) |
| 1 (ISO-HF + PBS) | 55.3 | 44.4 | 45.6 | 44.5 | 44.3 | 0 |
| Change to BI (%) | 24 | 0 | 3 | 0 | 0 | |
| 2 (ISO-HF + anti- | 54.2 | 46.1 | 48.8 | 48.6 | 46.5 | 0 |
| DPP3 antibody) | | | | | | |
| 1 mg/kg | | | | | | |
| Change to BI (%) | 18 | 0 | 6 | 5 | 1 | |
| 3 (ISO-HF + anti- | 54.6 | 46.8 | 50.0 | 49.0 | 49.8 | 36 |
| DPP3 antibody) | | | | | | |
| 5 mg/kg | | | | | | |
| Change to BI (%) | 17 | 0 | 7 | 5 | 6 | |
| 4 (ISO-HF + anti- | 53.5 | 46.9 | 50.0 | 53.4 | 53.3 | 67 |
| DPP3 antibody) | | | | | | |
| 10 mg/kg | | | | | | |
| Change to BI (%) | 14 | 0 | 7 | 14 | 14 | |
| 5 (ISO-HF + anti- | 54.6 | 45.2 | 49.1 | 48.5 | 49.1 | 60 |
| DPP3 antibody) | | | | | | |
| 50 mg/kg | | | | | | |
| Change to BI (%) | 21 | 0 | 9 | 7 | 9 | |
| 6 (ISO-HF + anti- | 54.2 | 48.3 | 47.1 | 46.8 | 46.5 | 68 |
| DPP3 antibody) | | | | | | |
| 100 mg/kg | | | | | | |
| Change to BI (%) | 12 | 0 | −3 | −3 | −4 | |

BL = baseline; BI = Before injection of anti-DPP3 antibody or PBS; HF = Heart Failure; ISO = Isoproterenol; LVSF = left ventricular shortening fraction; PBS = Phosphate Buffered Saline.

This study demonstrated that dose ranges from 1 to 100 mg/kg anti-DPP3 antibody when i.v. injected as a bolus is safe and well tolerated in mice under the conditions of the study. Administration of anti-DPP3 antibody at 5, 10, and 50 mg/kg led to improved cardiac function. At these doses, inhibition of DPP3 was also observed. No or minimal improvement in cardiac function was observed at 1 and 100 mg/kg.

### Example 10—CLP Induced Cardiac Dysfunction in Rats

This study was conducted to test the efficacy of the anti-DPP3 antibody in a rat model of cardiac dysfunction caused by sepsis. The study also included a limited set of safety-relevant endpoints. Parts of the study were also published by Deniau et. al. (Deniau 2020 Inhibition).

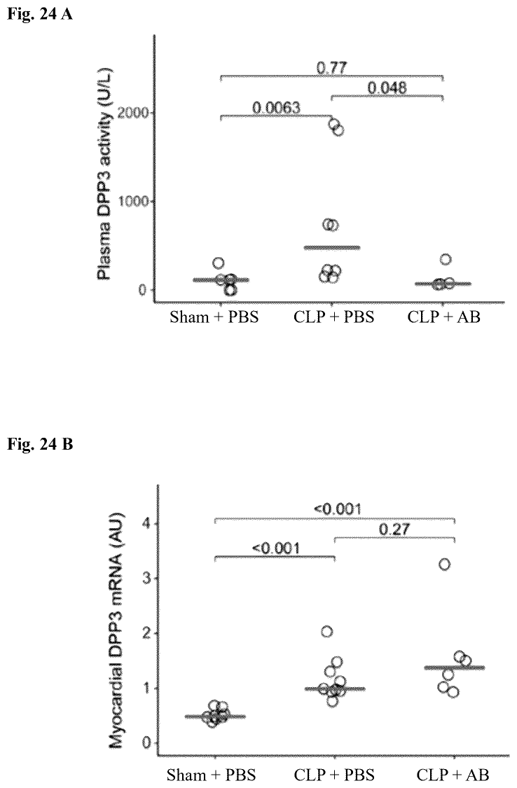

The study design is provided in Table 15 and FIGS. 24 A and 24 B.

TABLE 15
Overview of Study Design

| Group | Cardiac dysfunction | | Number |
|---|---|---|---|
| (Description) | induced by CLP | Treatment | of Animals |
| 1 (Sham + PBS) | No | PBS | 12 |
| 2 (CLP + anti- | Yes | anti-DPP3 | 6 |
| DPP3 antibody) | | antibody | |
| 3 (CLP + PBS) | Yes | PBS | 11 |

CLP = cecal ligation and puncture; PBS = Phosphate buffered saline.

Three-month-old male Wistar rats weighing 350-450 g were used for polymicrobial sepsis induction by cecal ligation and puncture (CLP). (See Rittirsch, D., et al., 2009, Immunodesign of experimental sepsis by cecal ligation and puncture, Nat. Protoc. 4, 31-36). A ventral midline incision (1 cm) was made to allow exteriorization of the caecum. The caecum was then ligated just below the ileocecal valve and punctured once with an 18-gauge needle. The abdominal cavity was closed in two layers, and rats were given fluid resuscitation (3 mL/100 g of body weight) of saline, injected subcutaneously. A sham operation was carried out by isolating the caecum with neither ligation nor puncture (sham+PBS). 16 hours later, rats were placed in the supine position and intubated. Catheters were inserted into (a) the left jugular vein to administer the anti-DPP3 antibody or placebo and (b) into the right carotid artery for invasive blood pressure monitoring. Based on the SEPSIS-3 consensus definition of septic shock (Singer 2016), only rats with mean blood pressure (MBP) below 65 mmHg were included. To ensure cardiac dysfunction, only rats with a left ventricular shortening fraction (LVSF) below 45% were included, as assessed by transthoracic echocardiography. Overall, twenty-nine rats were enrolled in the study: 11 in the CLP+PBS group, 6 in the CLP+anti-DPP3 antibody group, and 12 in the sham+PBS group (FIG. 22A and FIG. 22B). After a bolus injection of 1.5 mL PBS or anti-DPP3 antibody (concentration of 1 mg/mL) through the jugular vein in 5 min, which is equivalent as a fluid resuscitation, PBS (2 mL) and anti-DPP3 antibody (2 mL at the concentration of 0.42 mg/mL) were continuously infused with an electric syringe during the 120 minutes of follow-up. At the end of the protocol, the rats were sacrificed by lethal anaesthesia with an injection of pentobarbital solution. The heart was transversally divided into two parts: the base part was embedded into Tissue-Tek optimal cutting temperature (OCT) compound and frozen into liquid nitrogen pre-cooled isopentane for oxidative stress analysis; the apex was flash-frozen in liquid nitrogen and used for mRNA analysis. Heparin blood was collected and DPP3 activity was assessed in heparin plasma samples via a soluble activity assay. Anti-DPP3 antibody concentration was measured for quality control via an ELISA assay. Safety-relevant endpoints included in the study were monitoring of clinical signs, and macropathology.

The i.v. treatment with 10 mg/kg anti-DPP3 antibody was well tolerated in rats with no obvious adverse test item-related clinical signs or findings at macropathology.

Sixteen hours after the CLP or sham procedure, clinical signs of sepsis (reduced motor activity, lethargy, shivering, piloerection, and hunched posture) were only present in CLP and not in sham+PBS rats (Table 16). Furthermore, post-mortem examination of the abdominal cavity of all CLP rats showed varying degrees of peritonitis with a grey-black dilated caecum and purulent and malodorous peritoneal fluid. Baseline characteristics of the sham and CLP rats after randomization are summarized in Table 16.

TABLE 16
Hemodynamics and cardiac parameters in sham and CLP rats before
randomization (i.e., before PBS or anti-DPP3 antibody injection).

| | | | CLP before | | |
|---|---|---|---|---|---|
| | Sham | | randomization | | |
| Parameter | N = 12 | | N = 17 | | P value |
| LVSF (mean ± SD) | 54 ± 7% | | 39 ± 4% | | <0.001 |
| SBP (mean ± SD) | 99 ± 11 | mmHg | 79 ± 10 | mmHg | <0.001 |
| DBP (mean ± SD) | 70 ± 12 | mmHg | 46 ± 10 | mmHg | <0.001 |
| MBP (mean ± SD) | 79 ± 11 | mmHg | 57 ± 8 | mmHg | <0.001 |
| sHR (mean ± SD) | 321 ± 77 | bpm | 341 ± 62 | bpm | 0.428 |
| CO (mean ± SD) | 113 ± 33 | mL/min | 99 ± 26 | mL/min | 0.231 |
| SV (mean ± SD) | 0.4 ± 0.1 | mL | 0.3 ± 0.1 | mL | 0.064 |

Values are expressed as mean ± SD.
Comparisons were done by using the Wilcoxon rank-sum test.
CLP = cecal ligation and puncture; CO = cardiac output; DBP = diastolic blood pressure; HR = heart rate; LVSF = left ventricular shortening fraction; MBP = mean blood pressure; SBP = systolic blood pressure; SV = stroke volume.

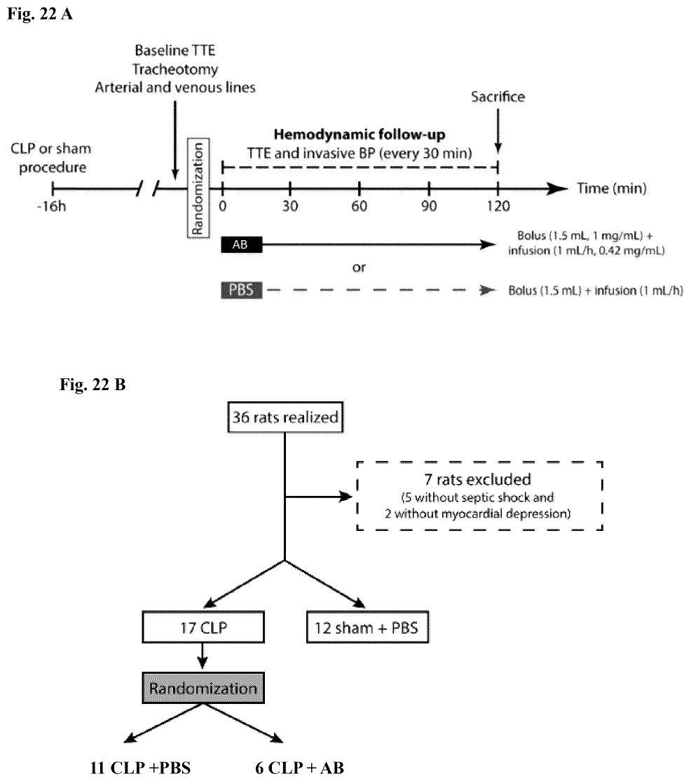

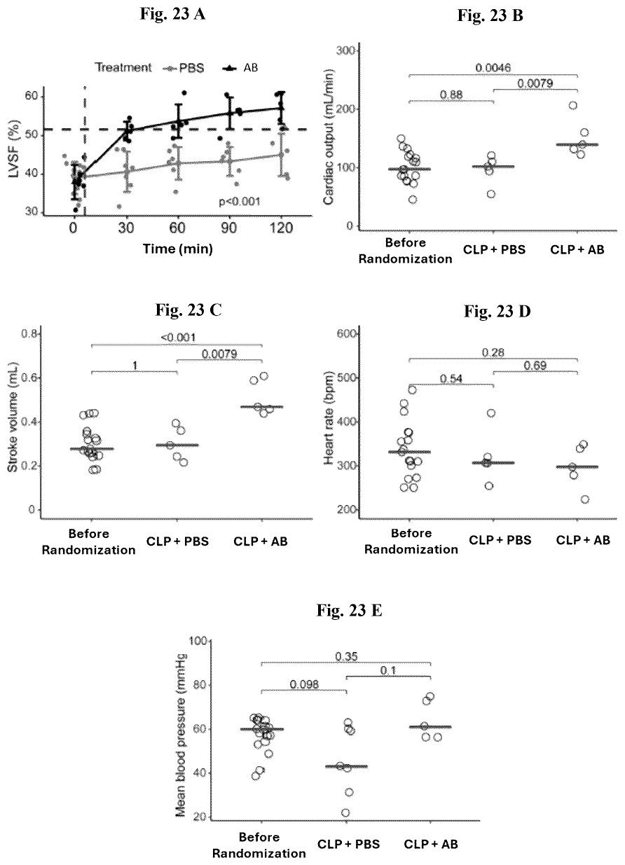

Anti-DPP3 antibody administration rapidly restored LVSF within 30 min of anti-DPP3 antibody initiation. LVSF was higher in CLP+anti-DPP3 antibody group when compared to CLP+PBS group (FIG. 23 A). The LVSF of the CLP+PBS rats remained unchanged (FIG. 23 A). At the end of the protocol, 120 minutes after the initiation of anti-DPP3 antibody or PBS, administration of anti-DPP3 antibody was associated with a greater cardiac output (CO) (FIG. 23 B) and stroke volume (SV) (FIG. 23C) compared to the rats receiving PBS. In contrast, heart rate (HR) and mean blood pressure (MBP) measured at 120 minutes were similar in all CLP groups (FIG. 23 D and FIG. 23 E). Finally, the survival rate was higher at 120 min in the CLP+anti-DPP3 antibody group compared to the CLP+PBS group. Of note, LVSF and other hemodynamic parameters remained stable throughout the duration of the experiment in the sham+PBS group.

In addition, CLP+PBS rats had a higher cDPP3 plasma activity than the sham+PBS rats at 120 min (FIG. 24 A). Similarly, DPP3 transcripts were up regulated in the heart of the CLP+PBS rats compared to the sham+PBS group (FIG. 24 B). Plasma cDPP3 activity was lower at 120 min in the CLP+anti-DPP3 antibody group when compared to the CLP+PBS (FIG. 24 A), without accompanying changes in myocardial transcription of DPP3 (FIG. 24 B). The DPP3 activity inhibition at 120 min in the CLP+anti-DPP3 antibody group is consistent with the anti-DPP3 antibody concentration in the blood.

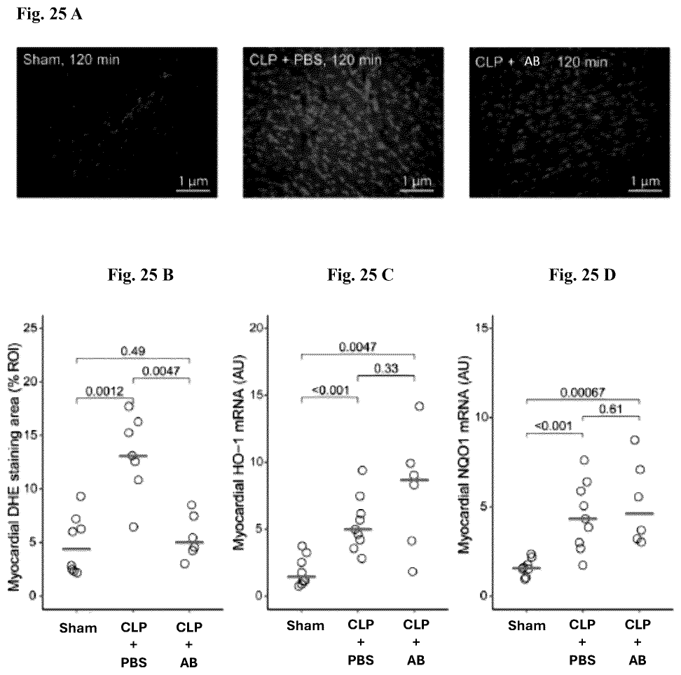

At the cardiac level, anti-DPP3 antibody administration rapidly reduced oxidative stress in the heart, measured by DHE staining, compared to PBS administration 120 min after PBS or anti-DPP3 antibody injection, respectively (FIG. 25 A and FIG. 25 B). However, the anti-DPP3 antibody effect was not accompanied by any change in the myocardial HO-1 and NQO1 expression, two oxidative stress-induced genes (FIG. 25C and FIG. 25D).

This study demonstrated that 7.8 mg/kg anti-DPP3 antibody when i.v. injected as a bolus followed by infusion is safe and well tolerated in rats under the conditions of the study. The CLP model was found suitable for efficacy testing of the anti-DPP3 antibody since it led to an increase in the target cDPP3 accompanied by signs of sepsis-induced cardiac dysfunction. Administration of the anti-DPP3 antibody inhibited cDPP3, and improved cardiac contractility and survival compared to the control vehicle. In addition, the anti-DPP3 antibody reduced oxidative stress in the heart of CLP rats compared to the control vehicle.

### Example 11—Anti-DPP3 Antibody Efficacy in Septic Swine Model

This study was conducted to test the efficacy of the anti-DPP3 antibody in a pig model of cardiac dysfunction caused by sepsis. The study also included a limited set of safety-relevant endpoints.

The study design is provided in Table 17.

TABLE 17
Overview of Study Design

| | Cardiac | | |
|---|---|---|---|
| Group | dysfunction | | Number of |
| (Description) | induced by sepsis | Treatment | Animals |
| 1 (sham) | no | Resuscitation with fluids | 4 |
| 2 (anti-DPP3 | yes | anti-DPP3 antibody; | 8 |
| antibody) | | 9.3 mg/kg; i.v. + | |
| | | Standard therapy* | |
| 3 (control) | yes | Standard therapy* | 8 |

*Resuscitation with fluids, antimicrobial therapy, peritoneal lavage, and norepinephrine.

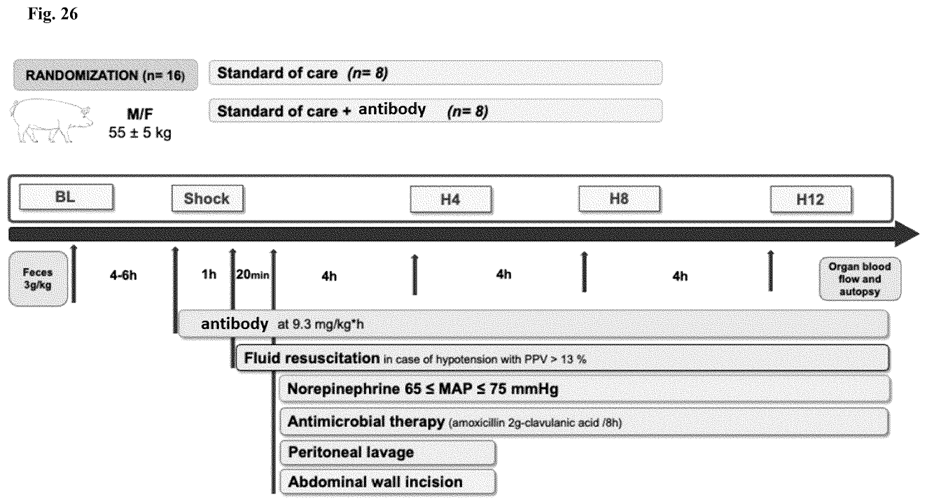

Cardiovascular dysfunction was induced in 16 male and female pigs weighing 55+/−5 kg by an intraperitoneal instillation of 3 g/kg of autologous feces, previously collected from the animal's cage via two peritoneal drains. (Garcia, B., et al., 2022, Crit. Care 26, 1-13). Sham pigs (n=4) underwent only anesthesia and surgical preparation, were observed for 4 hours after baseline until euthanasia, and were used as a reference for tissue analysis. 16 anesthetized and mechanically ventilated pigs with peritonitis were randomized to receive an infusion of the anti-DPP3 antibody (9.3 mg/kg for 13.3 hours; n=8) on top of standard treatment or only standard treatment (control; n=8) when the mean arterial pressure reached 50 mmHg (shock timepoint). Resuscitation with fluids, antimicrobial therapy, peritoneal lavage, and norepinephrine (standard of care) was then initiated and continued for 12 hours (FIG. 26). Afterwards, the animals were sacrificed with a lethal dose of potassium chloride i.v. administered under deep anesthesia, and tissues were collected for mRNA and protein expression analyses.

Hemodynamic parameters, tissue oxygenation indices, and measures of organ failure and myocardial injury were collected. Organ blood flow was assessed using isotopic assessment (^{99m}technetium albumin, ^{99m}Tc). Circulating catecholamines were assessed in EDTA plasma via solid phase extraction using an isocratic high performance liquid chromatography system with electrochemical detection. Equilibrium analysis of the renin-angiotensin system was performed by mass spectrometry via LC-MS/MS using heparin plasma samples. DPP3 activity was assessed in EDTA-plasma samples via a soluble activity assay. The anti-DPP3 antibody concentration was measured via an ELISA assay. Tissue mRNA expression of inflammatory cytokines and downregulation of adrenergic and angiotensin receptors were assessed on vascular and myocardial samples. Safety-relevant endpoints included in the study were monitoring of clinical signs.

The i.v. treatment with 9.3 mg/kg anti-DPP3 antibody was well tolerated in pigs with no obvious adverse test item-related clinical signs.

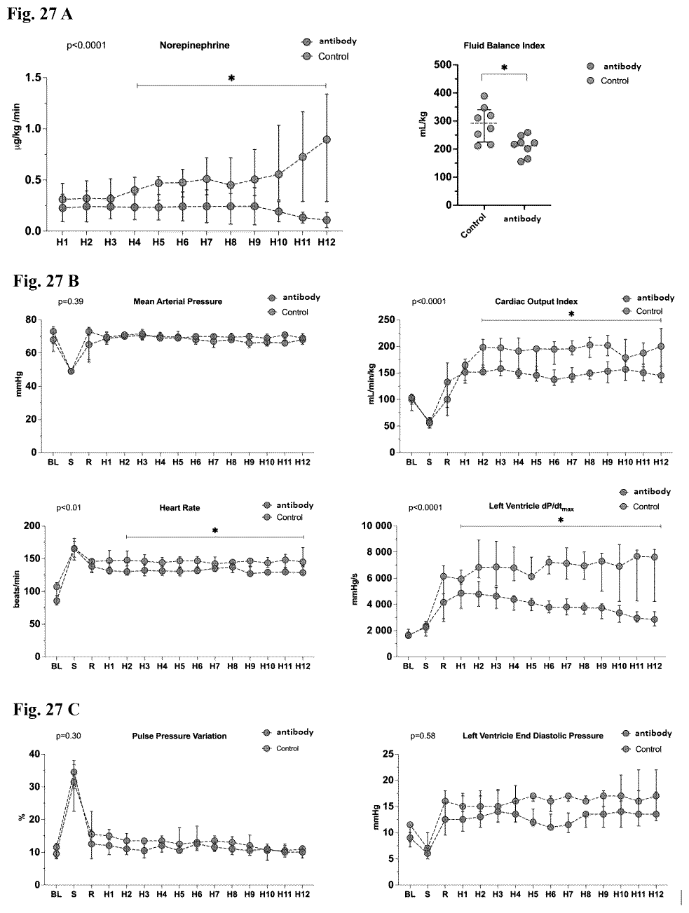

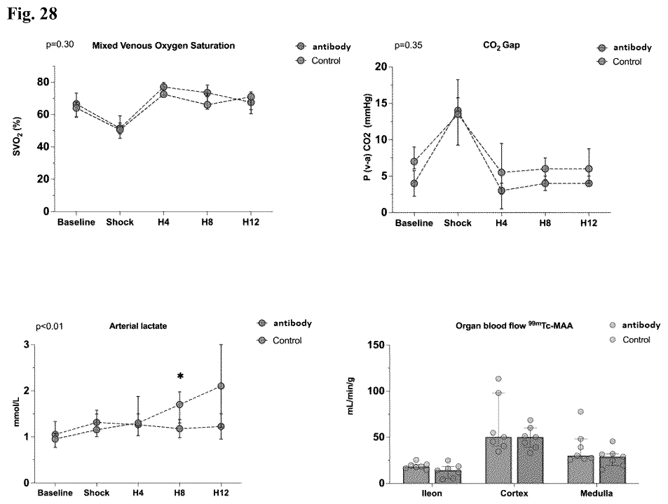

All 16 animals of Group 2 and 3 developed severe hypotension and tachycardia, with decreased mixed venous oxygen saturation (SvO₂) and increased veno-arterial CO₂ partial pressure difference (P(v-a)-CO₂) (CO₂ Gap) at the shock timepoint (MAP below 50 mmHg) compared to baseline (FIG. 27 B and FIG. 28). The mean time to reach the shock timepoint was similar in the two groups. There were no statistically significant differences in hemodynamic parameters between the treatment groups until the shock timepoint (FIG. 27 C and Table 18).

After fluid resuscitation and during vasopressor therapy, MAP was maintained between 65 and 75 mmHg in all animals (FIG. 27 B). The median dose of norepinephrine required to maintain MAP was significantly lower in the anti-DPP3 antibody group than the control group, from the 4th hour of vasopressor therapy onwards until the end of the experiment (interaction p<0.0001, FIG. 27 A). Of note, norepinephrine could even be weaned in one animal in the anti-DPP3 antibody group before the end of the experiment. The total cumulative fluid balance (indexed to body weight) between shock time-point and H12 time-point was lower in the anti-DPP3 antibody group (FIG. 27 A).

Heart rate and cardiac index were significantly lower in the anti-DPP3 antibody group than in the control group from H3 to H12 (interaction p<0.01 and p<0.0001, respectively, FIG. 27 B). One episode of atrial fibrillation occurred in the control group. Left ventricular contractility, assessed by the maximal slope of left ventricle rise in pressure during systolic upstroke (LV dP/dTmax), increased in both groups with the vasopressor infusion, and was significantly lower in the anti-DPP3 antibody group than in the control group from H1 to H12 (interaction p<0.0001, FIG. 27 B). No statistical differences were observed in the left ventricle end-diastolic pressure (LVEDP) (interaction p=0.60), or in the PPV (interaction, p=0.30), between groups throughout the experiment (FIG. 27 C).

P-(v-a)-CO₂ (CO₂ Gap) and SvO₂ were restored to and remained at normal values from 4 h after the start of resuscitation; without differences between groups (FIG. 28). Arterial lactate was significantly lower in the anti-DPP3 antibody group than in the control group at H8 (interaction p<0.01, FIG. 26). There was no significant difference between groups in regional blood flow, as assessed using ^{99m}Tc-macro aggregated albumin in the ileum, the kidney cortex, and the medulla (FIG. 28).

The PaO₂/FiO₂ ratio was significantly higher in the anti-DPP3 antibody group than in the control group from H4 to H12 (interaction p=0.01, Table 18). Creatinine clearance and creatinine levels were similar between groups (Table 18). There was significantly less myocardial injury, as shown by reduced levels of high sensitivity cardiac troponin I, at H12 in animals receiving the anti-DPP3 antibody than in control animals (Table 18). There were no statistically significant differences between groups in aspartate transaminase, alkaline phosphatase, albumin, haematocrit, or platelet count. There were no statistically significant differences between the groups in plasma levels of interleukin (IL)-6, IL-10, or Tumor Necrosis Factor (TNF)-α (Table 18).

TABLE 18
Biological and oxygenation values in the anti-DPP3 antibody and control groups at the different study
timepoints. Values are expressed as median-interquartile range. P-value refers to interaction.

| VARIABLES | | | | | | | P-value |
|---|---|---|---|---|---|---|---|
| Median [IQR] | | Baseline | Shock | H4 | H8 | H12 | Interaction |
| Hematocrit | anti-DPP3 | 27 | 45 | 24 | 24 | 22 | 0.74 |
| (%) | antibody | [24-29] | [41-48] | [19-26] | [20-28] | [18-23] | |
| | Control | 27 | 43 | 22 | 22 | 20 | |
| | | [25-28] | [41-47] | [20-30] | [19-28] | [19-25] | |
| Platelets | anti-DPP3 | 114 | 100 | 105 | 88 | 85 | 0.88 |
| (g/L) | antibody | [99-152] | [53-122] | [65-121] | [79-104] | [69-105] | |
| | Control | 135 | 113 | 96 | 94 | 85 | |
| | | [127-147] | [94-170] | [64-143] | [81-99] | [69-146] | |
| Albumin | anti-DPP3 | 26 | 26 | 9 | 9 | 8 | 0.94 |
| (g/L) | antibody | [24-31] | [23-29] | [7-11] | [7-10] | [7-9] | |
| | Control | 27 | 24 | 7 | 7 | 7 | |
| | | [20-30] | [20-28] | [7-10] | [6-7] | [6-7] | |
| Creatinine | anti-DPP3 | 1.1 | 2.0 | 1.6 | 1.6 | 1.4 | 0.83 |
| (mg/dL) | antibody | [1.1-1.4] | [1.7-2.2] | [1.3-1.7] | [1.3-1.7] | [1.1-1.7] | |
| | Control | 0.9 | 1.7 | 1.2 | 1.2 | 1.2 | |
| | | [0.8-1] | [1.6-2] | [1.1-1.6] | [1.1-1.6] | [1-1.3] | |
| CC | anti-DPP3 | 201 | 61 | 96 | 122 | 121 | 0.32 |
| (mL/min) | antibody | [100-346] | [44-119] | [68-137] | [98-130] | [109-164] | |
| | Control | 239 | 62 | 80 | 178 | 96 | |
| | | [132-516] | [32-74] | [53-104] | [103-258] | [80-135] | |
| PaO₂/FiO₂ | anti-DPP3 | 390 | 352 | 366 * | 310 * | 314 * | P < 0.01 |
| | antibody | [355-423] | [310-377] | [333-380] | [298-353] | [274-337] | |
| | Control | 333 | 270 | 243 | 193 | 173 | |
| | | [280-383] | [259-320] | [213-293] | [178-201] | [128-218] | |
| RSC | anti-DPP3 | 37 | 34 | 32 | 29 | 24 | 0.27 |
| (mL/cmH₂O) | antibody | [34-40] | [29-37] | [28-37] | [23-34] | [22-31] | |
| | Control | 29 | 27 | 22 | 20 | 20 | |
| | | [26-32] | [23-32] | [20-26] | [17-23] | [15-23] | |
| Troponin I | anti-DPP3 | 293 | NA | NA | NA | 1294 * | NA |
| (ng/L) | antibody | [131-1571] | | | | [131-1571] | |
| | Control | 824 | | | | 3677 | |
| | | [513-1252] | | | | [1866-4904] | |
| IL-6 | anti-DPP3 | 6 | 1129 | 540 | 570 | 466 | 0.63 |
| (pg/mL) | antibody | [3-9] | [795-1157] | [325-1025] | [326-861] | [309-466] | |
| | Control | 6 | 1146 | 696 | 677 | 710 | |
| | | [4-12] | [981-1161] | [406-1046] | [424-1067] | [377-1090] | |
| IL-10 | anti-DPP3 | 2 | 53 | 35 | 37 | 28 | 0.97 |
| (pg/mL) | antibody | [1-8] | [16-98] | [20-44] | [16-62] | [11-57] | |
| | Control | 2 | 48 | 46 | 63 | 68 | |
| | | [1-7] | [23-92] | [23-59] | [36-63] | [37-81] | |
| TNF | anti-DPP3 | 84 | 184 | 142 | 126 | 109 | 0.24 |
| (pg/mL) | antibody | [75-101] | [182-206] | [116-165] | [99-143] | [94-109] | |
| | Control | 104 | 257 | 127 | 129 | 128 | |
| | | [84-115] | [187-302] | [115-172] | [114-169] | [116-160] | |

* post-hoc P-value < 0.05 between anti-DPP3 antibody and control group.
RSC = Respiratory system compliance; CC = Creatine Clearance, IL-6 = interleukin 6; IL-10 = interleukin 10; TNF = tumor necrosis factor; NA = not applicable since no repetitive measurements available.

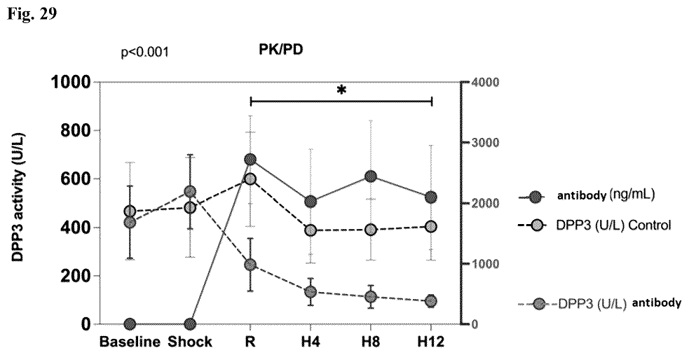

DPP3 plasma activity increased similarly in the two groups from baseline to the shock time-point and decreased significantly after anti-DPP3 antibody administration compared to the control group (FIG. 29). The DPP3 activity inhibition follows the anti-DPP3 antibody concentration in the blood. The anti-DPP3 antibody Cₘₐₓ was detected early (resuscitation timepoint) and anti-DPP3 antibody concentration remained constant throughout the infusion period until the last timepoint (H12) (FIG. 29).

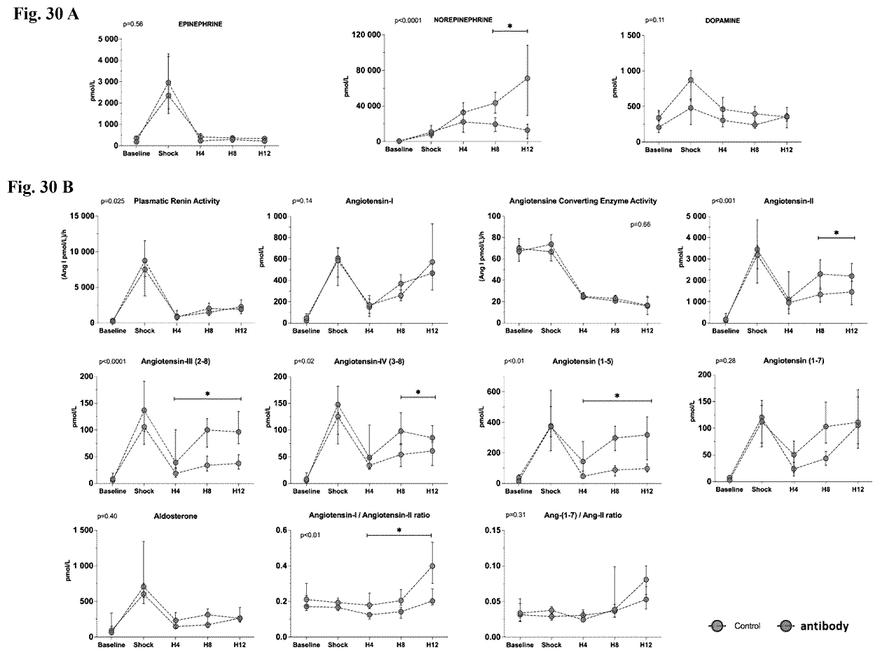

Circulating norepinephrine levels were significantly lower in the anti-DPP3 antibody group than in the control group after resuscitation (interaction p<0.0001); there were no significant differences in circulating epinephrine or dopamine levels (interaction p=0.56 and 0.11, respectively (FIG. 30 A).

There were no differences between the groups at any time-point in plasma renin activity, ACE activity, or Ang I concentrations. Anti-DPP3 antibody administration was associated with higher Ang II concentrations compared to the control group (interaction p<0.001). The Ang I/Ang II ratio remained stable over time in the anti-DPP3 antibody group but increased significantly in the control group from H4 to H12 (interaction p=0.01). Anti-DPP3 antibody administration was associated with significantly higher Ang III (interaction p<0.0001), Ang IV (interaction p=0.02), and Ang-(1-5) (interaction p<0.01) levels compared to the control group (FIG. 30 B).

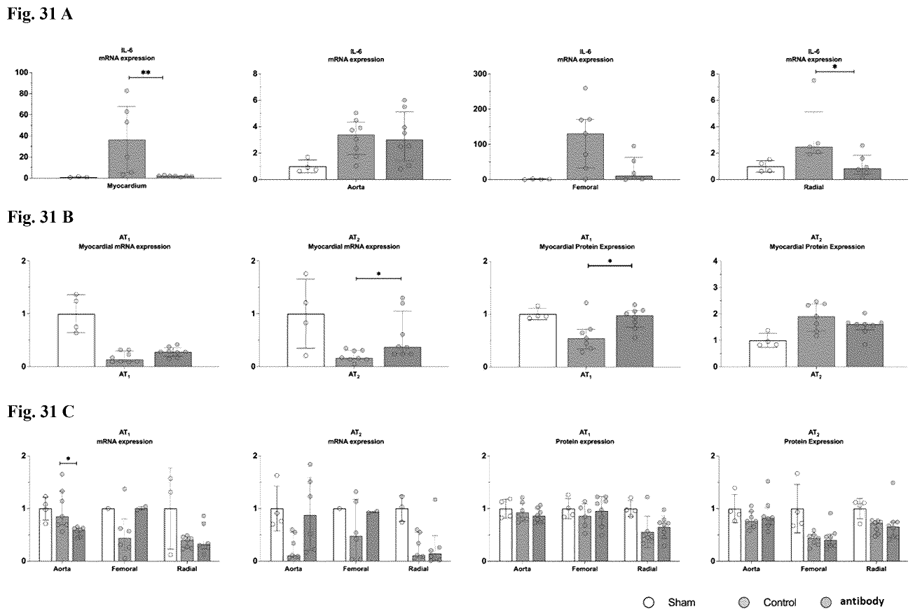

Inflammatory activity, evaluated by upregulation of IL-6 mRNA levels, was significantly reduced in the myocardium and radial artery in the anti-DPP3 antibody group. Accordingly, myocardial AT₁ receptor protein downregulation was attenuated in the anti-DPP3 antibody group compared to the control group. No significant differences were observed in the other vascular samples regarding angiotensin receptor expression, with exception for lower aortic AT₁ mRNA expression in the anti-DPP3 antibody group (FIGS. 31 A, 31 B, and 31 C).

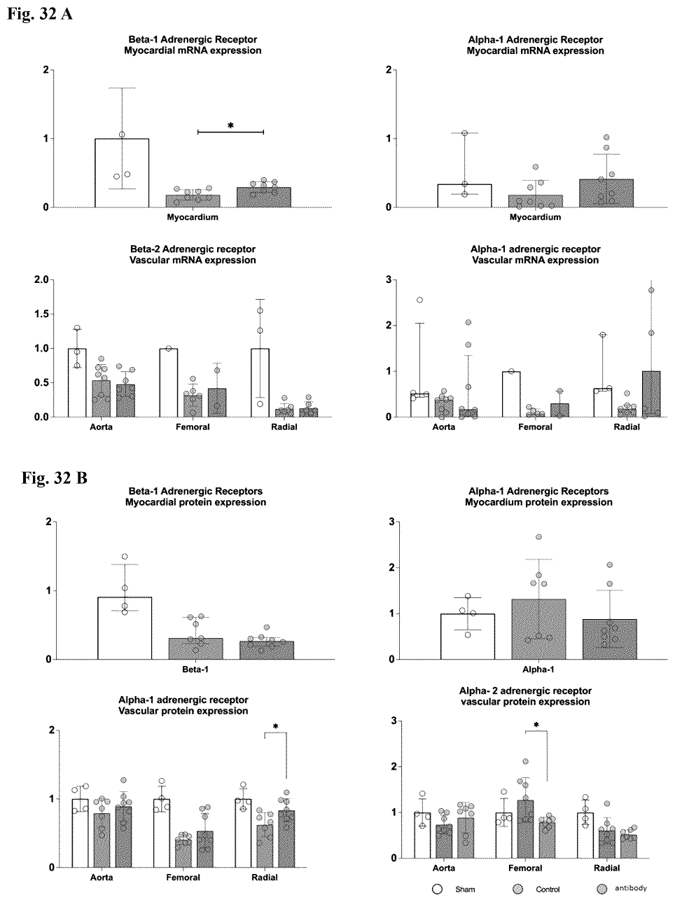

Alpha-1 adrenergic receptor protein expression was higher in the radial artery of anti-DPP3 antibody group (FIG. 32 B), whereas no differences were observed in the other vascular samples. Myocardial beta-1 adrenergic receptor mRNA expression was higher in the anti-DPP3 antibody group, while protein expression was similar between groups. No other significant differences were observed, with the exception for higher levels of beta-2 adrenergic protein expression in the femoral artery of the anti-DPP3 antibody group, compared to the control groups (FIGS. 32 A and 32 B).

In the pig study of cardiovascular dysfunction induced by sepsis, a treatment with anti-DPP3 antibody in addition to the recommended resuscitation treatment resulted in reduced catecholamine requirement, along with a reduced fluid balance, although adequate tissue perfusion was maintained. These effects were associated with an increase in circulating Ang II concentrations, a preserved Ang I/Ang II ratio, prevention of AT₁ downregulation in the left ventricle and higher alpha-1 adrenergic receptor expression in the radial artery, higher myocardial beta-1 adrenergic receptor mRNA expression, and higher levels of beta-2 adrenergic receptor expression in the femoral artery. Moreover, the reduction in catecholamine exposure and local tissue inflammation with anti-DPP3 antibody administration had additional benefits for the pulmonary and cardiovascular systems.

### Example 12—2-Day Dose Range Finding Toxicity Study with Anti-DPP3 Antibody by Intravenous Injection Administration to Mice and Cynomolgus Monkeys

Anti-DPP3 antibody was tested in two species, mouse and cynomolgus monkeys. In both species, a 2-day dose range finding study was conducted followed by a GLP-compliant 2-week repeat-dose toxicity study. These studies also included analysis of toxicokinetic, recovery, and pharmacological activity of anti-DPP3 antibody (inhibition of cDPP3 activity). The selection of the species for toxicity testing was based on the following considerations. The protein sequence of the target DPP3 is well conserved in mice (93%) and in cynomolgus monkeys (98%). In addition, the DPP3 binding site for the anti-DPP3 antibody is 100% conserved in both species indicating that anti-DPP3 antibody can inhibit the species-specific DPP3 forms comparable to the human target. To support this statement, in vitro investigations (anti-DPP3 antibody inhibition studies with mouse and cynomolgus monkey DPP3) and results confirmed that the antibody inhibits cDPP3 activity in these species. Further, a set of pharmacology studies was conducted in mice, which provides additional evidence for the pharmacological relevance of this species. Finally, mice were preferred over rats since after i.v. administration clinical signs (i.e., edema formation) have been observed immediately after first dosing in rats indicating an unspecific immunological effect likely triggered by the systemic administration of a foreign protein. This has not been observed in any other animal species and was thus considered rat-specific. Accordingly, rats were not considered a suitable representative species for anti-DPP3 antibody toxicity testing.

Although a considerable amount of data has been collected on the target DPP3 in humans, rodents, and pigs to support the proposed mode of action, there is still quite some uncertainty related to the biological function of DPP3 in the human body. It is assumed that DPP3 is involved in more biological processes than currently identified.

To investigate the DPP3 mode of action and to improve safety assessment prior to administration of anti-DPP3 antibody to humans, toxicological testing in cynomolgus monkeys was performed. DPP3 might exert a comparable biological profile in non-human primates (NHPs) (being closely related to humans), and thus studies in NHPs may provide an assessment of potential safety issues related to binding of the antibody to its target and inhibition of the enzyme's activity. Further, the potential risks related to binding to those off-targets were considered best covered by a closely related species. Lastly, as binding of anti-DPP3 antibody to Fc-receptors (FcR) is confirmed (as expected for IgG1), the cynomolgus monkey allows for monitoring of adverse effects related to FcR-binding. This is because validated assays are available only for NHPs to measure complement activation and a broad panel of cytokines. In addition, the FcR and classical downstream immune reactions (e.g., antibody dependent cell-mediated cytotoxicity (ADCC), antibody dependent cellular phagocytosis (ADCP), complement-dependent cytotoxicity (CDC)) are well described for this species and are potentially comparable to humans in NHPs.

In the repeat-dose toxicity studies, PCZ was applied intravenously as planned for the clinical phase. Due to the complexity of an i.v. infusion setting in small species as mouse, PCZ was administered via slow (injection lasting about 15 seconds) bolus injection in mice. This is considered to cover the worst case in terms of maximum plasma concentration (Cₘₐₓ). In the cynomolgus monkeys, an infusion duration of 2 hours using PCZ was chosen, which is comparable to the clinical setting.

The duration of the repeat-dose toxicity studies was 2-weeks with a total of 4 doses applied (Days 1, 2 and Days 13, 14). In the clinical trial discussed in Example 13, human subjects received a single dose of the humanized anti-DPP3 antibody. Thus, the frequency applied in the GLP toxicity studies exceeds the dosing frequency in the Example 13 clinical trial (i.e., single dose) thereby increasing the chances to detect adverse effects.

2-Day Dose Range Finding Toxicity Study with Anti-DPP3 Antibody by Intravenous Injection Administration to Mice

This study evaluated the toxicity and determines the toxicokinetics of anti-DPP3 antibody when given as escalating doses via i.v. administration on Days 1 and 2 of the dosing phase to mice. Data obtained was also used to establish dose levels for subsequent pivotal studies.

Male and female C57BL/6J (C57BL/6JCrl) mice were assigned to four groups, and anti-DPP3 antibody doses (Batch V1802D, V1829D) were administered as indicated in Table 19. Animals were dosed by i.v. (slow bolus) injection once daily at a volume of 7.5 or 5 mL/kg for Groups 1 and 4 or Groups 2 and 3, respectively. The vehicle control article was 10 mM His, 250 mM Trehalose, 20 mM L-methionine, pH 6.5.

TABLE 19
Overview of Study Design.

| | Dose | Concentration | Dose Volume | |
|---|---|---|---|---|
| Group | (mg/kg) | (mg/mL) | (mL/kg) | Number of Animals |
| 1* | 0 | 0 | 7.5 | Main: 6 M + 6 F |
| | | | | TK: 6 M + 6 F |
| | | | | Cytokine: 6 M + 6 F |
| 2 | 50 | 10 | 5 | Main: 6 M + 6 F |
| | | | | TK 30 M + 30 F |
| | | | | Cytokine: 6 M + 6 F |
| 3 | 94.9 | 18.98 | 5 | Main: 6 M + 6 F |
| | | | | TK: 30 M + 30 F |
| | | | | Cytokine: 6 M + 6 F |
| 4 | 142.35 | 18.98 | 7.5 | Main: 6 M + 6 F |
| | | | | TK: 30 M + 30 F |
| | | | | Cytokine: 6 M + 6 F |

*Control animals received vehicle (10 mM His, 250 mM Trehalose, 20 mM L-methionine, pH 6.5); F = Females; M = Males; TK = Toxicokinetic.

Assessment of toxicity was based on mortality, clinical, post-dose, and cage-side observations, as well as body weights, food consumption, clinical pathology, organ weights, and macroscopic observations. Blood samples were collected for toxicokinetic (TK) and cytokine evaluations. TK samples were also used to measure cDPP3 activity.

The treatment with anti-DPP3 antibody was well tolerated up the highest tested dose level of 142.35 mg/kg (highest technically feasible dose based on dosing volume and batch concentration). There were no test article-related findings observed in body weight, food consumption, hematology, blood chemistry, or macropathology.

On Day 1, one control male and one female given 50 mg/kg were found dead, one female given 50 mg/kg died during the 0.5-hour post dose blood sampling, and one male given 142.35 mg/kg died following dosing. Due to lack of relevant findings, and no other tissues examined, the cause of death could not be established, however, these deaths were not considered to be related to treatment, but presumably related to stress.

Cytokine assessment of mice revealed statistically significant decreases in keratinocyte chemoattractant/human growth regulated oncogene (KC/GRO), monocyte chemoattractant protein-1 (MCP-1), and Tumor necrosis factor-alpha (TNF-α) mouse plasma concentrations observed 2 hours post-dose on Day 1 in both sexes administered up to 142.35 mg/kg anti-DPP3 antibody compared to control, although the decrease was more evident in females.

Test item-related findings observed during microscopic examination of the liver included minimal to slight rarefaction of hepatocytes, minimal necrosis at a low incidence in both sexes administered 94.9 mg/kg or 142.35 mg/kg and an increased incidence and severity of mixed inflammatory cell infiltrate in males administered 94.9 mg/kg or 142.35 mg/kg and in females at all dose levels with no dose relationship. While the changes correlated with higher absolute body weight and brain weight adjusted for liver weights in the females at all doses, the liver findings in both sexes are considered non-adverse.

Results of the TK analysis are summarized in Table 20.

TABLE 20
Overview of TK Results in Mice.

| Occasion | | Dose | | Cₘₐₓ | Tₘₐₓ | AUC₀₋₈ | AUC₀₋₁₈ |
|---|---|---|---|---|---|---|---|
| (Day) | Group | (mg/kg) | Sex | (ng/mL) | (h) | (h*ng/mL) | (h*ng/mL) |
| 1 | 2 | 50 | M | 1860000 | 0.0833 | 4650000 | 5150000 |
| | | | F | 1710000 | 0.250 | 2320000 | 2640000 |
| | | | MF | 1680000 | 0.0833 | 3480000 | 3890000 |
| | 3 | 94.9 | M | 5060000 | 0.250 | 13800000 | 15900000 |
| | | | F | 5320000 | 0.0833 | 13900000 | 16000000 |
| | | | MF | 4500000 | 0.0833 | 13900000 | 16000000 |
| | 4 | 142.35 | M | 6040000 | 0.0833 | 18300000 | 23300000 |
| | | | F | 4380000 | 0.500 | 19000000 | 21900000 |
| | | | MF | 4360000 | 0.500 | 18500000 | 22300000 |
| 2 | 2 | 50 | M | 636000 | 0.0833 | 1610000 | 2500000 |
| | | | F | 2460000 | 0.0833 | 5110000 | 5700000 |
| | | | MF | 1550000 | 0.0833 | 3230000 | 3970000 |
| | 3 | 94.9 | M | 3470000 | 0.250 | 9270000 | 11300000 |
| | | | F | 2320000 | 0.0833 | 6190000 | 7180000 |
| | | | MF | 2730000 | 0.250 | 7720000 | 9230000 |
| | 4 | 142.35 | M | 3450000 | 0.0833 | 13000000 | 18300000 |
| | | | F | 3240000 | 0.0833 | 12700000 | 15500000 |
| | | | MF | 3340000 | 0.0833 | 12900000 | 16900000 |

AUC = Area under the Curve; Cₘₐₓ = Maximum Serum Concentration; F = Female; M = Male; MF = Combined Male and Female Values; Tₘₐₓ = Timepoint of Maximum Serum Concentration.

In general, sex differences in anti-DPP3 antibody Cmax, AUC0-8, and AUC0-18 values were less than 2-fold, except at the 50 mg/kg dose level, with higher exposure observed in males compared to females on Day 1 and higher exposure observed in females compared to males on Day 2. Exposure, as assessed by the anti-DPP3 antibody Cmax, AUC0-8, and AUC0-18 values, generally increased with the increase in dose level from 50 mg/kg to 142.35 mg/kg. The increases in the anti-DPP3 antibody Cmax, AUC0-8, and AUC0-18 values were generally greater than dose proportional over the dose range 50 mg/kg to 94.5 mg/kg and approximately dose proportional over the dose range 94.5 mg/kg to 142.35 mg/kg; however, in females on Day 2 these increases were approximately dose proportional over the full dose range (50 mg/kg to 142.35 mg/kg). Cmax, AUC0-8, and AUC0-18 values were generally lower on Day 2 than on Day 1, with no accumulation of the anti-DPP3 antibody observed after multiple doses in mice, apart from Group 2 females (50 mg/kg), where the AUC0-8 and AUC0-18 values were higher on Day 2 than on Day 1.

Measurement of cDPP3 activity revealed that anti-DPP3 antibody at all tested dose levels inhibited the enzyme to 90% and above, already at the first measured timepoint (5 min after dosing). The effect resolved over time, with around 70-80% inhibition seen 24 h after the last dose and approximately 60-70% at 96 h (last timepoint collected).

It is concluded that IV bolus administration of anti-DPP3 antibody to C57BL/6J mice on two subsequent days at dosages up to 142.35 mg/kg is well tolerated. With the exception of the non-adverse findings in the liver and lower cytokine concentrations, no clear test item-related findings were noted for any treated animal and, consequently, dosages up to 142.35 mg/kg (Cmax 3,450,000 ng/mL in males and 3,240,000 in females, AUC0-8 130,000,000 h*ng/mL in males, and 127,000,000 h*ng/mL in females) were considered to be suitable for studies of a longer duration. Further, anti-DPP3 antibody was found to strongly inhibit the activity of cDPP3 at all dose levels tested.

Anti-DPP3 Antibody: 2-Week Intermittent Intravenous Toxicity and Toxicokinetic Study in Mice with a 2-Week Recovery

This repeat-dose toxicity study was to investigate the toxicity and toxicokinetics of the anti-DPP3 antibody when administered via i.v. bolus to mice on Days 1, 2, 13, and 14. The reversibility of any finding was investigated as part of a 2-week recovery period.

A total number of 213 male and 213 female C57BL/6J mice (63-90 days of age) were divided into 4 groups and treated as outlined below in Table 21.

TABLE 21
Overview of Study Design

| | | | Dose | |
|---|---|---|---|---|
| | Dose | Concentration | Volume | |
| Group | (mg/kg) | (mg/mL) | (mL/kg) | Number of Animals |
| 1* | 0 | 0 | 7.5 | Main: 12 M + 12 F |
| | | | | Recovery: 6 M + 6 F |
| | | | | TK + DPP3: 6 M + 6 F |
| | | | | Cytokine: 6 M + 6 F |
| | | | | ADA: 6 M + 6 F |
| 2 | 37.5 | 5 | 7.5 | Main: 12 M + 12 F |
| | | | | TK + DPP3: 33 M + 33 F |
| | | | | Cytokine: 6 M + 6 F |
| | | | | ADA: 6 M + 6 F |
| 3 | 75 | 10 | 7.5 | Main: 12 M + 12 F |
| | | | | TK + DPP3: 33 M + 33 F |
| | | | | Cytokine: 6 M + 6 F |
| | | | | ADA: 6 M + 6 F |
| 4 | 150 | 20 | 7.5 | Main: 12 M + 12 F |
| | | | | Recovery: 6 M + 6 F |
| | | | | TK + DPP3: 33 M + 33 F |
| | | | | Cytokine: 6 M + 6 F |
| | | | | ADA: 6 M + 6 F |

*Control animals received vehicle (10 mM His, 250 mM Trehalose, 20 mM L-methionine, pH 6.5); ADA = Antidrug Antibody; DPP3 = Dipeptidyl Peptidase 3; F = Females; M = Males; TK = Toxicokinetic.

The anti-DPP3 antibody was administered via i.v. bolus administration into the tail vein to the animals on Days 1, 2, 13, and 14 at dose levels of 37.5 mg/kg, 75 mg/kg, and 150 mg/kg (Groups 2, 3, and 4, respectively). The vehicle group received 10 mM His, 250 mM Trehalose, 20 mM L-methionine, pH 6.5. In-life monitoring included monitoring of clinical signs (including detailed post-dose observations), ophthalmoscopy, body weight assessment (weekly), and monitoring of food consumption (weekly). Necropsy, including macropathology and organ weight assessment, was performed on Day 15 for all main study animals and on Day 28 for all recovery animals. Blood was collected on these days as well to investigate changes in hematology and clinical chemistry. Histopathology, including a full set of tissues, was conducted for all high dose and control animals at the end of the main study and at the end of the recovery period. In addition, potential target organs of toxicity (liver, spleen, kidney, and macroscopic lesions) were assessed for the low dose group and the mid dose group.

Due to the limited blood volume in mice, additional satellite animals (“ADA” animals identified in Table 21) were included in the study to evaluate changes in cytokine levels (Days 1 and 14, 2 h after dosing), and cDPP3 activity (Day 1: 0.5 h and 24 h post dose; Day 14: 0.5 h and 336 h post dose; non-GLP investigation). Sampling for putative ADA analysis was conducted pre-dose and on 336 h post last dose (not performed). TK sampling was performed on Day 1 and Day 14.

A Modified Irwin Screen was conducted on Day 1 and Day 13 at assumed maximum exposure (Cₘₐₓ) (approx. 0.5 h and 6 h after dosing) for the first five male and female main study animals.

A total of 7 non-test item-related deaths occurred during the study: 1 control male (died on Day 14), 2 low dose group males (died on Day 13, Day 14), 1 mid dose male (died Day 13), 2 high dose males (both died on Day 1). These decedents were either found dead, euthanized due to moribund condition, or died during bleeding or dosing procedures. No adverse clinical signs were observed prior to death for any of the early decedents. At necropsy, no adverse macroscopic or microscopic findings were noted. No cause of death could be established. Nevertheless, these early deaths are not considered to be related to treatment with the anti-DPP3 antibody, but likely related to stress.

No anti-DPP3 antibody-related clinical signs were noted at any time during the study in any group. No test article-related alterations in body weight or body weight gain, food consumption, or ophthalmology were noted for all groups. Further, there were no test item-related effects observed on hematology or clinical chemistry.

Cytokine analysis did not show any clear test item-related effects for any group at any of the tested dose levels. Most samples for IFN-γ, IL-1β, IL-2, IL-4, IL-5, and IL-6 were below LOQ in all groups at all timepoints. For IL-10, KC/GRO, and TNF-α measurable levels were detected in either only one sex and/or without any dose-response and/or with an overall small magnitude of change compared to the control. Those changes were thus not considered being of biological relevance.

Intravenous bolus administration of the anti-DPP3 antibody at up to 150 mg/kg in male and female mice produced no effects on the behavioral and physiological state of the animals as assessed by Irwin Screen. In addition, no effects on body temperature were recorded.

Necropsy revealed no test article-related macroscopic findings or effects of the anti-DPP3 antibody treatment on organ weight except for a lower absolute and adjusted to brain and body weight to the liver weight in male animals of Group 4 (150 mg/kg) at the end of the main study period when compared to the control group. This difference was not observed at the end of the recovery period (Table 22).

TABLE 22
Liver Weight for End of Main Study and End of Recovery Period

| | Males | | | | Females | | | |
|---|---|---|---|---|---|---|---|---|
| Group | 1 | 2 | 3 | 4 | 1 | 2 | 3 | 4 |
| Dose Level (mg/kg) | 0 | 37.5 | 75 | 150 | 0 | 37.5 | 75 | 150 |
| Absolute liver weight (g) | 1.43 | 97 | 94 | 88* | 1.03 | 96 | 101 | 94 |
| % Body weight | 5.42 | 99 | 97 | 92* | 5.07 | 98 | 101 | 95* |
| % Brain Weight | 325.69 | 98 | 94 | 90* | 236.34 | 98 | 101 | 95 |

*p ≤ 0.05 (compared with respective control group); Values for treatment groups are expressed as percentage of control mean values.

Histopathological analysis revealed the following finding in the liver of males treated with 75 mg/kg or 150 mg/kg anti-DPP3 antibody at the end of the main study: decreased rarefaction of the cytoplasm of hepatocytes (Table 23). As it correlated with the reduced absolute and relative liver weight in males of the high dose group, the finding was considered test item-related. However, the finding was of minimal to slight in severity and was not associated with necrotic or clinical pathology changes (e.g., increase in AST or ALT or bilirubin) indicative for impaired hepatic function. Furthermore, it was not present in animals at the end of the recovery period. Accordingly, it was not considered adverse.

TABLE 23
Histopathological Liver Findings at the End of the Main Study

| | Males | | | | Females | | | |
|---|---|---|---|---|---|---|---|---|
| Group | 1 | 2 | 3 | 4 | 1 | 2 | 3 | 4 |
| No. Examined | 12 | 11 | 12 | 12 | 12 | 12 | 12 | 12 |

Decreased rarefaction, cytoplasm, hepatocyte

| | | | | | | | | |
|---|---|---|---|---|---|---|---|---|
| Minimal | 1 | 0 | 5 | 3 | 0 | 1 | 0 | 2 |
| Slight | 0 | 0 | 1 | 7 | 1 | 0 | 0 | 0 |

Minimal = 1-2 foci; slight = 3-6 foci.

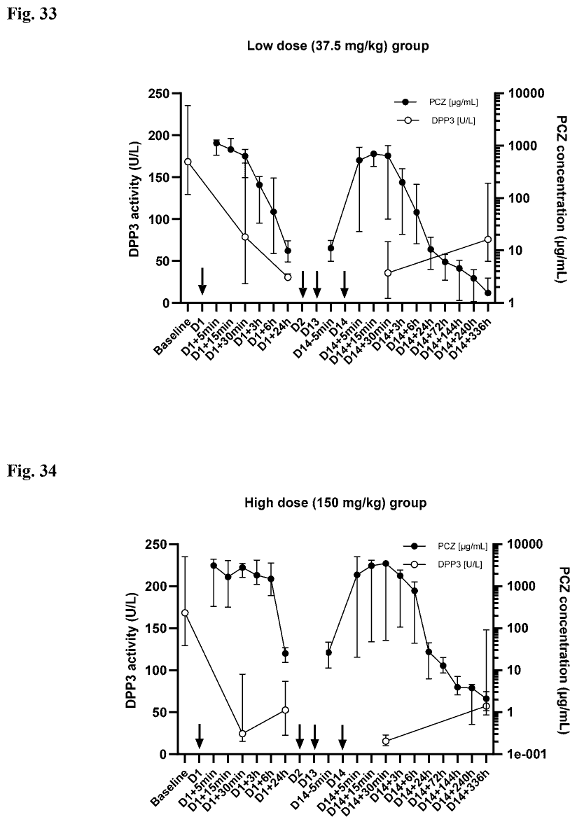

Results of the TK noncompartmental analysis are summarized in Table 24. The time courses of PCZ concentration and cDPP3 activity obtained for the low dose (37.5 mg/kg) and high dose (150 mg/kg) are shown in FIG. 33 and FIG. 34, respectively.

TABLE 24
Overview of TK Results in Mice

| Occasion | | Dose | | Cₘₐₓ | Tₘₐₓ | AUC₀₋₂₄ | AUC₀₋₃₃₆ | t_{1/2} |
|---|---|---|---|---|---|---|---|---|
| (Day) | Group | (mg/kg) | Sex | (μg/mL) | (h) | (h*μg/mL) | (h*μg/mL) | (h) |
| 1 | 2 | 37.5 | M | 1070 | 0.0830 | 2980 | 2980 | 5.68 |
| | | | F | 1070 | 0.0830 | 2300 | 2300 | 5.40 |
| | 3 | 75 | M | 2250 | 0.250 | 13500 | 13500 | 3.90 |
| | | | F | 1510 | 0.0830 | 4810 | 4810 | 3.77 |
| | 4 | 150 | M | 2850 | 6.00 | 40000 | 40000 | ND |
| | | | F | 3660 | 0.0830 | 19800 | 19800 | 3.50 |
| 14 | 2 | 37.5 | M | 788 | 0.500 | 3240 | 4790 | 113 |
| | | | F | 643 | 0.250 | 1520 | 2640 | 152 |
| | 3 | 75 | M | 1620 | 0.0830 | 4970 | 7160 | 133 |
| | | | F | 1490 | 0.500 | 4620 | 6450 | 161 |
| | 4 | 150 | M | 2930 | 0.0830 | 16200 | 18600 | 107 |
| | | | F | 3550 | 0.250 | 18300 | 20300 | 165 |

AUC = Area under the Curve; Cₘₐₓ = Maximum Serum Concentration; F = Females; M = Males; Tₘₐₓ = Timepoint of Maximum Serum Concentration; t_{1/2} = Half-life; ND = not determinable as acceptance criteria for calculation were not met.

All concentration values of the anti-DPP3 antibody in the control group were below the lower limit of quantitation (<20.0 ng/mL). Because sex ratios for AUC₀₋₂₄ were above 2 on a total of three occasions, the TK evaluation is discussed per sex and no mean calculation is performed. Cₘₐₓ sex ratios were below 2 on all occasions. Values generally show high variation; thus the data does not allow for a clear interpretation of a sex-specific pharmacokinetic profile. As expected for an i.v. bolus administration, Cₘₐₓ was observed at early timepoints (between 5 min and 30 min after injection) at the majority of timepoints. On Day 1 in high dose males, Cₘₐₓ was measured at 6 h. After reaching Cₘₐₓ, anti-DPP3 antibody concentrations readily declined, with t_{1/2} values ranging from 3.90 hours to 5.68 hours in males and from 3.50 hours to 5.40 hours in females on Day 1, and from 107 hours to 133 hours in males, and from 152 hours to 165 hours in females on Day 14. The marked difference between t_{1/2} values on Days 1 and 14 is a result of the sampling regimen, whereby samples were taken up to 24 hours post-dose on Day 1 and up to 336 hours post-dose on Day 14. Mean concentration values for the anti-DPP3 antibody were measurable through 24 hours post-dose on Day 1 and through 336 hours post-dose on Day 14. Exposure, as assessed by the anti-DPP3 antibody Cₘₐₓ and AUC₀₋₂₄ values, increased with the increase in dose level from 37.5 mg/kg to 150 mg/kg. The increases in the anti-DPP3 antibody Cₘₐₓ values were generally dose proportional, while AUC values were generally greater than dose proportional (between 4.99 fold and 13.4-fold when comparing low and high dose with a 4-fold increase between dose levels). The anti-DPP3 antibody Cₘₐₓ and AUC₀₋₂₄ values were generally similar on Days 1 and 14, indicating no accumulation of the anti-DPP3 antibody after multiple doses in mice.

Upon administration of PCZ, cDPP3 activity decreased at all dose levels tested (FIG. 33 and FIG. 34). Only low dose and high dose (i.e., 37.5 mg/kg and 150 mg/kg, respectively) data is presented here since the mid-dose followed similar kinetics. When AK1967 concentrations fell below about 5 μg/mL, cDPP3 activity started to rise again. This confirms that the anti-DPP3 antibody was pharmacologically active in the animals. All dosing Groups showed an inhibition over 40% after first dosage which increased to over 90% after fourth dosage for Group 3. In Group 4, more than 50% of samples, and more than in the other dosing Groups, were found below the limit of quantification indicating a very strong inhibition effect.

A clear dose-dependent trend could not be observed. The inhibition was measured until the last timepoint tested (336 hours) and was between 17 to 60%. Overall, the inhibition effect was comparable for male and female mice; minor variations are likely caused by the very few control samples representing the baseline per sex.

Based on the results of the study, an i.v. treatment on Days 1, 2, 13, and 14 with the anti-DPP3 antibody at dose levels up to 150 mg/kg (maximum feasible dose) was well tolerated. The only test item-related finding was non-adverse and reversible rarefaction in liver hepatocytes. As no adverse test item-related effects were observed up to the highest tested dose level, 150 mg/kg (maximum technically feasible dose) was determined as the NOAEL (AUC₀₋₃₃₆=18,600 and 20,300 h*μg/mL for males and females on Day 14, respectively; Cₘₐₓ=2,930 and 3,350 μg/mL for males and females on Day 14, respectively).

### The Anti-DPP3 Antibody: Dose Range Finding Study by Intravenous Infusion Administration to Cynomolgus Monkeys on 2 Subsequent Occasions

This study was to determine the tolerability and toxicity of the anti-DPP3 antibody when administered as i.v. infusion over 2 h to cynomolgus monkeys on two subsequent days.

1 male and 1 female naive cynomolgus monkey were assigned to each of the four Groups of the study and administered anti-DPP3 antibody on Days 1 and 2 of the study as outlined in Table 25. After dosing, the animals were observed for 5 additional days.

TABLE 25
Overview of Study Design

| | Dose | Concentration | Dose Volume | Number of |
|---|---|---|---|---|
| Group | (mg/kg) | (mg/mL) | (mL/kg) | Animals |
| 1 | 50 | 2.5 | 20 | 1 M + 1 F |
| 2 | 150 | 7.5 | 20 | 1 M + 1 F |
| 3 | 200 | 10 | 20 | 1 M + 1 F |
| 4 | 400 | 20 | 20 | 1 M + 1 F |

F = Females; M = Males.

During the study, TK, clinical condition, body weight, electrocardiography, blood pressure, hematology (peripheral blood), blood chemistry, cytokine and complement analysis, Dipeptidyl Peptidase 3 (DPP3) analysis, organ weight, macropathology, and histopathology investigations were undertaken.

There were no deaths during the study.

Clinical signs included transient piloerection and muscle fasciculation observed on Day 2 of dosing in animals receiving 200 mg/kg and on both days of dosing in animals receiving 400 mg/kg. These signs were resolved by the end of the working day. Muscle fasciculation was also noticed in the female receiving 400 mg/kg the day after the last dosing occasion. There were no findings at the injection sites that were clearly attributed to the test item although transient, warm to touch was observed in both animals receiving 400 mg/kg.

There was no effect on body weight in either sex at any dose level. There were no test item-related hematology changes, with all changes considered to be procedural.

The blood chemistry investigations revealed clear anti-DPP3 antibody-induced effect on enzyme activities which consisted of an increase in alanine aminotransferase (ALT), aspartate aminotransferase (AST), and creatinine kinase (CK) activities on Day 3. These findings showed clear evidence of recovery on Day 7, with CK activity being lower than pre-treatment values in some animals. Alkaline phosphatase (ALP) and gamma-glutamyl transpeptidase (γGT) activities were lower than pre-treatment values on Day 3 and for some animals also on Day 7 (Table 26). Overall, those findings were not considered adverse.

TABLE 26
Overview of Clinical Chemistry Changes

| | | | ALP | ALT | AST | CK | g GT |
|---|---|---|---|---|---|---|---|
| Group | Sex | Day | (U/L) | (U/L) | (U/L) | (U/L) | (U/L) |
| 1 | M | PD | 1128 | 70 | 86 | 584 | 175 |
| (50 mg/kg) | | 3 | 861 | 123 | 438 | 6433 | 124 |
| | | 7 | 868 | 81 | 58 | 287 | 116 |
| 2 | M | PD | 1089 | 46 | 45 | 240 | 395 |
| (150 mg/kg) | | 3 | 807 | 66 | 348 | 1926 | 273 |
| | | 7 | 785 | 48 | 51 | 119 | 248 |
| 3 | M | PD | 440 | 24 | 64 | 561 | 84 |
| (200 mg/kg) | | 3 | 409 | 57 | 296 | 357 | 71 |
| | | 7 | 424 | 39 | 35 | 119 | 73 |
| 4 | M | PD | 780 | 124 | 64 | 368 | 167 |
| (400 mg/kg) | | 3 | 585 | 158 | 1133 | 6231 | 121 |
| | | 7 | 508 | 112 | 79 | 174 | 122 |
| 1 | F | PD | 914 | 40 | 68 | 729 | 125 |
| (50 mg/kg) | | 3 | 624 | 135 | 447 | 880 | 92 |
| | | 7 | 619 | 72 | 48 | 125 | 91 |
| 2 | F | PD | 580 | 44 | 50 | 246 | 99 |
| (150 mg/kg) | | 3 | 450 | 99 | 439 | 1242 | 77 |
| | | 7 | 503 | 54 | 41 | 823 | 80 |
| 3 | F | PD | 778 | 24 | 51 | 1211 | 101 |
| (200 mg/kg) | | 3 | 669 | 117 | 518 | 593 | 76 |
| | | 7 | 719 | 58 | 37 | 195 | 86 |
| 4 | F | PD | 707 | 35 | 44 | 338 | 110 |
| (400 mg/kg) | | 3 | 580 | 113 | 729 | 1587 | 88 |
| | | 7 | 507 | 60 | 40 | 96 | 89 |

ALP = Alkaline Phosphatase; ALT = Alanine Aminotransferase; AST = Aspartate Aminotransferase; CK = Creatinine Kinase; F = Female; γGT = Gamma-glutamyl transpeptidase; M = Male; PD = Pre-dose.

The electrocardiographic (ECG) and blood pressure investigations performed in this study during Day 2 did not reveal any test item-related effects on heart rate, ECG intervals (PR, QRS, QT), QTcB, waveform morphology, or blood pressure (systolic, diastolic, mean arterial) or pulse rate at any dose level.

On Day 1, plasma concentrations in the male animal administered 400 mg/kg showed an increase in granulocyte colony-stimulating factor (G-CSF), interferon-gamma (IFN-γ), interleukin (IL)-2, IL-4 and TNF-α levels. Generally, on Day 2, levels for these cytokines were decreased compared to Day 1 in this animal. In females, all or most of these cytokines were below the limit of quantification (BLQ), except for G-CSF levels, where a small increase in animals administered 150 mg/kg, 200 mg/kg, or 400 mg/kg was noted on Day 1 with G-CSF levels maintained or increased on Day 2. IL-6 plasma concentrations in male animals administered 150 mg/kg or 400 mg/kg was increased on Day 1, with a further increase on Day 2. All treated females showed increased IL-6 concentrations on Day 1 with a further increase seen on Day 2 in most animals, except for the female administered 50 mg/kg where the IL-6 concentration declined almost to pre-dose value.

Increases in complement Bb fragment (Bb) concentrations were noted on Day 1 in all animals administered up to 400 mg/kg; these concentrations were maintained or showed a further increase on Day 2 in both sexes.

At the end of the observation period, there were no treatment-related organ weight changes, macropathology, or micropathology findings.

The results of the TK analysis conducted on Day 1 and Day 14 are summarized in Table 27.

TABLE 27
Overview of TK Results in Cynomolgus Monkeys

| | | Dose | | | | | | |
|---|---|---|---|---|---|---|---|---|
| Occasion | | (mg/kg/ | | | Cₘₐₓ | Tₘₐₓ | AUC₀₋₂₄ | t_{1/2} |
| (Day) | Group | day) | Sex | Animal | (ng/mL) | (h) | (h*ng/mL) | (h) |
| 1 | 1 | 50 | M | 157 | 2560000 | 3.00 | 22700000 | ND |
| | | | F | 161 | 2520000 | 3.00 | 19700000 | ND |
| | | | MF | | 2540000 | 3.00 | 21200000 | ND |
| | 2 | 150 | M | 158 | 3560000 | 3.00 | 49900000 | ND |
| | | | F | 162 | 5400000 | 3.00 | 54200000 | ND |
| | | | MF | | 4480000 | 3.00 | 52100000 | ND |
| | 3 | 200 | M | 159 | 7100000 | 2.00 | 93500000 | ND |
| | | | F | 163 | 7680000 | 2.00 | 77700000 | ND |
| | | | MF | | 7390000 | 2.00 | 85600000 | ND |
| | 4 | 400 | M | 160 | 13200000 | 6.00 | 213000000 | ND |
| | | | F | 164 | 14900000 | 3.00 | 193000000 | ND |
| | | | MF | | 14100000 | 4.50 | 203000000 | ND |
| 2 | 1 | 50 | M | 157 | 1690000 | 2.00 | 18100000 | 30.1 |
| | | | F | 161 | 1330000 | 2.00 | 14800000 | 27.3 |
| | | | MF | | 1510000 | 2.00 | 16500000 | 28.7 |
| | 2 | 150 | M | 158 | 5190000 | 2.00 | 75400000 | 16.3 |
| | | | F | 162 | 6280000 | 2.00 | 74000000 | 18.4 |
| | | | MF | | 5730000 | 2.00 | 74700000 | 17.4 |
| | 3 | 200 | M | 159 | 6340000 | 2.00 | 94900000 | 20.2 |
| | | | F | 163 | 4650000 | 2.00 | 67700000 | 32.2 |
| | | | MF | | 5500000 | 2.00 | 81300000 | 26.2 |
| | 4 | 400 | M | 160 | 16500000 | 8.00 | 315000000 | 8.88 |
| | | | F | 164 | 15200000 | 8.00 | 266000000 | 13.6 |

| | | | | |
|---|---|---|---|---|
| MF | 15900000 | 8.00 | 291000000 | 11.2 |

AUC = Area under the Curve; Cₘₐₓ = Maximum Serum Concentration; F = Females; M = Males; MF = Combined Male and Female Values; Tₘₐₓ = Timepoint of Maximum Serum Concentration; t_{1/2} = Half-life; ND = not determinable as acceptance criteria for calculation were not met.

Analysis of cDPP3 activity showed an inhibition by the anti-DPP3 antibody in all dosing groups on Day 1 and Day 2. Generally, inhibition could be seen already 5 min after start of infusion on Day 1 in all groups. At that timepoint, a dose-dependent effect was obvious with the lower dose groups (50 mg/kg, 150 mg/kg) showing a mean inhibition of 7% and 32%, respectively, while an inhibition of 41% was observed at 200 mg/kg. At 400 mg/kg, an inhibition of 38% was observed. At the remaining timepoints, all groups showed an inhibition of >50%, which remained at around this level until the last timepoint (24 h). In all groups, the maximum cDPP3 activity inhibition was approximately 70%.

It was concluded that administration of the anti-DPP3 antibody to cynomolgus monkeys via i.v. infusion (2 h) on two occasions at doses up to 400 mg/kg followed by a 5 day off dose period was well tolerated. The findings in this study indicated that the dose of up to 400 mg/kg could be a suitable high dose level for long-term administration. Further, the anti-DPP3 antibody was shown to inhibit its target cDPP3 at all tested dose levels.

The Anti-DPP3 Antibody: 2-Week Intravenous Infusion Toxicity and Toxicokinetic Study in Cynomolgus Monkeys with a 2-Week Recovery Period

This repeat-dose toxicity study was to investigate the toxicity and toxicokinetics of the anti-DPP3 antibody when administered as 2 hour-i.v. infusion to cynomolgus monkeys on Days 1, 2 and Days 13, 14. The reversibility of any finding was investigated as part of a 2-week recovery period.

A total of 16 male and 16 female naive cynomolgus monkeys were allocated to 4 Groups as outlined in Table 28. During the pre-dose phase, ECG findings were detected in three animals (1 male of the control Group, 1 male of the high dose Group, 1 female of the control Group) and the animals were found unsuitable for test article or vehicle administration. No data was thus collected for those animals.

TABLE 28
Overview of Study Design

| | | | Dose | |
|---|---|---|---|---|
| | Dose | Concentration | Volume | |
| Group | (mg/kg) | (mg/mL) | (mL/kg) | Number of Animals |
| 1^{#} | 0 | 0 | 20 | Main Study: 2 M* + 2 F* |
| | | | | Recovery: 2 M + 2 F |
| 2 | 40 | 2 | 20 | Main Study: 3 M + 3 F |
| | | | | Recovery: / |
| 3 | 120 | 6 | 20 | Main Study: 3 M + 3 F |
| | | | | Recovery: / |
| 4 | 350 | 17.5 | 20 | Main Study: 2 M* + 3 F |
| | | | | Recovery: 2 M + 2 F |

*Reduced number of animals in the group since animals had to be excluded from study due to pre-treatment findings;
^{#}Control animals received vehicle (mixture of formulation buffer (10 mM His, 250 mM Trehalose, 20 mM L-methionine, pH 6.5) and 0.9% saline); F = Females; M = Males.

The anti-DPP3 antibody was administered via i.v. infusion (2 h) to animals on Days 1, 2, 13, and 14 at dose levels of 40 mg/kg, 120 mg/kg, and 350 mg/kg (Groups 2, 3, and 4, respectively). The vehicle group received a mixture of formulation buffer (10 mM His, 250 mM Trehalose, 20 mM L-methionine, pH 6.5) and 0.9% saline, comparable to the vehicle/test item ratio of the high dose Group. In-life monitoring included monitoring of clinical signs (including detailed post-dose observations), ophthalmoscopy, body weight assessment (weekly), and monitoring of food consumption (weekly). Necropsy, including macropathology and organ weight assessment, was performed on Day 15 for all main study animals and on Day 28 for all recovery animals. Blood was collected on these days as well to investigate changes in hematology, and clinical chemistry. Urine collection was performed pre-dose, at the end of Week 2 and of the recovery period. Histopathology, including a full set of tissues, was conducted for all treated and control animals at the end of the main study and at the end of the recovery period. In addition, the study included electrocardiogram examinations (pre-dose, Day 2, and Day 13), respiratory inductive plethysmography (pre-dose, Day 2, Day 13, Week 2 of recovery), neurobehavioral assessment (pre-dose, Day 3), body temperature measurement (pre-dose, Days 1, 2, 13, and 14 after completion of dosing), and measurement of blood pressure (pre-dose, Day 1, and Day 14, Week 2 of recovery). Bioanalytical samples were collected for TK evaluation on Day 1 and Day 14. Additional blood samples were collected pre-dose, Day 1, and Day 14 for analysis of complement (complement component 3a fragment (C3a) and Bb), cytokine analysis (IFN-γ, TNF-α, IL-1p, IL-2, IL-4, IL-5, IL-6, IL-8) as well as cDPP3 activity measurement. For putative anti-drug antibody (ADA) analysis, blood was collected (pre-dose, Day 14, and end of recovery).

There were no unscheduled deaths during the study. Post-dose observations included transient decreased activity, piloerection, cold extremities, and pale appearance (gingiva) observed on Day 14 of dosing in one female receiving 350 mg/kg. These signs were resolved by the next day.

Injection site observations included bruising/abnormal purple color of the skin, swelling, and thickening of skin at dose sites. These findings were observed in animals across all groups, suggesting these were attributed to the method of dose administration (i.v. infusion).

No test item-related alterations in body weight or body weight gain or food consumption were noted for animals in any of the groups.

No test item-related ophthalmic observations were noted.

Hematology and coagulation evaluation revealed an increase in reticulocyte counts and fibrinogen when comparing pre-dose values on Day 15 in males and females in all Groups including control Group (see Table 29). As this trend was observed in all Groups, it is not considered test item-related, but could be a result of the blood sampling (i.e., blood loss) over the course of the study. These changes improved at the end of the recovery period, demonstrating recovery. All other hematology differences observed were either minimal, inconsistent between sexes, or reflected trends that were present at the pre-treatment investigation and therefore were considered to be normal biological variations.

TABLE 29
Overview of Hematology Changes in Cynomolgus Monkeys

| | | RETIC (10⁹/L) | | RETIC (%) | | Fibrinogen (g/L) | |
|---|---|---|---|---|---|---|---|
| Group | Day | M | F | M | F | M | F |
| 1 | PD | 41.2 | 25.8 | 0.6 | 0.4 | 2.68 | 1.70 |
| (vehicle) | 15 | 46.1 | 34.5 | 0.8 | 0.6 | 3.05 | 2.16 |
| | Recovery | 45.3 | 60.2 | 0.8 | 1.1 | 2.30 | 1.74 |
| 2 | PD | 30.3 | 42.2 | 0.5 | 0.6 | 2.21 | 2.05 |
| (40 | 15 | 46.5 | 70.3 | 0.7 | 1.3 | 3.40 | 2.87 |
| mg/kg) | Recovery | NA | NA | NA | NA | NA | NA |
| 3 | PD | 39.4 | 36.4 | 0.6 | 0.6 | 2.17 | 2.01 |
| (120 | 15 | 49.8 | 71.0 | 0.9 | 1.2 | 3.46 | 3.21* |
| mg/kg) | Recovery | NA | NA | NA | NA | NA | NA |
| 4 | PD | 39.3 | 42.5 | 0.6 | 0.7 | 2.41 | 2.02 |
| (350 | 15 | 57.2 | 61.0 | 1.0 | 1.0 | 3.31 | 3.03* |
| mg/kg) | Recovery | 34.9 | 55.5 | 0.55 | 0.85 | 2.58 | 2.19 |

F = Female; M = Male; NA = Not applicable; PD = pre-dose; RETIC = Reticulocytes;
*<P ≤ 0.05 (ANOVA followed by Dunnett's).

Clinical chemistry investigation on Day 15 showed an increase in alanine aminotransferase (ALT), aspartate aminotransferase (AST), and creatinine kinase (CK) in all Groups in both sexes compared to pre-dose values (see Table 30). The increase was also observed in control Groups, but to a lesser extent than in the treated animals. There was no clear dose-dependency of the changes. All these findings showed clear recovery with mean activities similar to pre-treatment values in high dose animals at the end of recovery period. Alkaline phosphatase (ALP) and gamma-glutamyl transpeptidase (γGT) activities were lower than pre-dose values on Day 15, but mean values were comparable to pre-dose values at end of the recovery period. Total bilirubin concentration (TBIL) was slightly increased in all test item-treated animals and control males, but not in control females. TBIL values improved in 2/4 animals at the end of recovery period, demonstrating at least partial recovery.

TABLE 30
Overview of Clinical Chemistry Changes in Cynomolgus Monkeys

| | | ALT | | AST | | CK | | TBIL | |
|---|---|---|---|---|---|---|---|---|---|
| | | (U/L) | | (U/L) | | (U/L) | | (μmol/L) | |
| Group | Day | M | F | M | F | M | F | M | F |
| 1 | PD | 58 | 70 | 38 | 44 | 86 | 143 | 1.5 | 3.0 |
| (ve- | 15 | 67 | 74 | 111 | 85 | 941 | 569 | 2.6 | 3.1 |
| hicle) | RV | 29 | 53 | 39 | 33 | 141 | 169 | 1.6 | 3.9 |
| 2 | PD | 43 | 42 | 56 | 42 | 227 | 230 | 2.0 | 3.0 |
| (40 | 15 | 79 | 76 | 349 | 224 | 677 | 1718 | 2.8 | 4.3 |
| mg/kg) | RV | NA | NA | NA | NA | NA | NA | NA | NA |
| 3 | PD | 34 | 60 | 42 | 36 | 197 | 181 | 2.3 | 2.5 |
| (120 | 15 | 72 | 100 | 407* | 398* | 967 | 493 | 4.1 | 4.1 |
| mg/kg) | RV | NA | NA | NA | NA | NA | NA | NA | NA |
| 4 | PD | 46 | 57 | 50 | 45 | 242 | 209 | 2.0 | 2.5 |
| (350 | 15 | 61 | 103 | 321 | 476* | 570 | 1132 | 2.1 | 3.7 |
| mg/kg) | RV | 29 | 63 | 44 | 51 | 73 | 102 | 1.8 | 2.5 |

ALT = Alanine Aminotransferase; AST = Aspartate Aminotransferase; CK = Creatinine Kinase; F = Female; γGT = Gamma-glutamyl transpeptidase; M = Male; PD = Pre-dose; RV = Recovery; TBIL = Total Bilirubin;
*<P ≤ 0.05 (ANOVA followed by Dunnett's).

No test article-related urinalysis effects were observed.

Analysis of DPP3 activity showed no inhibition of cDPP3 activity in the vehicle-treated animals. In the anti-DPP3 antibody-treated animals, inhibition of enzyme activity could be shown for every timepoint (Day 1: 4 and 24 h; Day 14: 4, 24, and 197 h) when comparing to individual baseline-values. Generally, the inter-individual variation of cDPP3 activity at baseline and after treatment was high, while the number of animals per Group was low. Therefore, data does not allow for interpretation in terms of dose-dependency. The general range of activity inhibition was between 8 and 60% in Group 2, between 7 and 54% in Group 3, and between 5 and 37% in Group 4. It is noteworthy that an increase in cDPP3 activity compared to baseline was observed in the control animals for Day 1 and Day 14 samples. This might indicate a stress-related reaction, where cDPP3 levels increase as consequence of manipulation of the animals (e.g., administration and monitoring procedures). Overall, this data confirms that the anti-DPP3 antibody was pharmacologically active in the study animals.

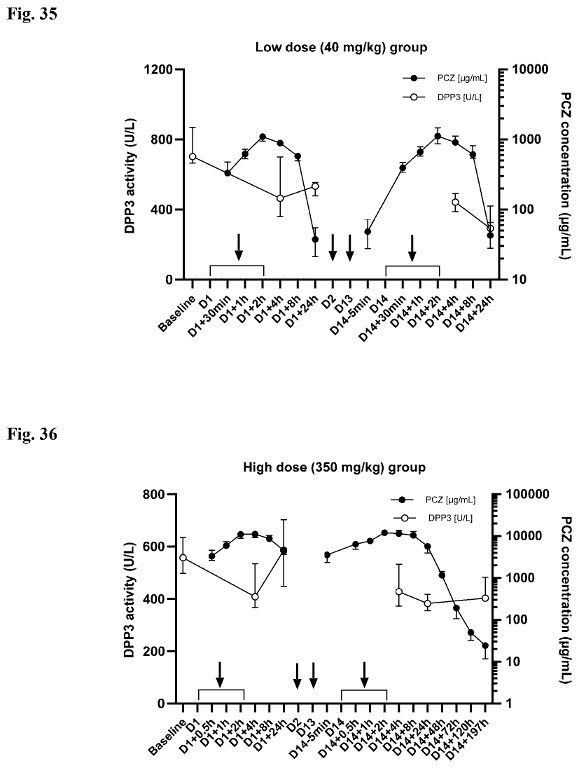

The results of the TK analysis are summarized in Table 31. The time courses of PCZ concentration as well as cDPP3 activity obtained for the low dose (40 mg/kg) and high dose (350 mg/kg) are shown in FIG. 35 and FIG. 36, respectively.

TABLE 31
Overview of TK Results in Cynomolgus Monkeys

| | | Dose | | | | | |
|---|---|---|---|---|---|---|---|
| Occasion | | (mg/kg/ | | Cₘₐₓ | Tₘₐₓ | AUC₀₋₂₄ | t_{1/2} |
| (Day) | Group | day) | Sex | (μg/mL) | (h) | (h*μg/mL) | (h) |
| 1 | 2 | 40 | M | 1000 | 2.00 | 10700 | 4.51 |
| | | | F | 1160 | 2.00 | 11400 | 3.98 |
| | | | MF | 1080 | 2.00 | 11000 | 4.25 |
| | 3 | 120 | M | 2920 | 2.00 | 38600 | 9.10 |
| | | | F | 2820 | 2.00 | 40500 | 7.69 |
| | | | MF | 2870 | 2.00 | 39600 | 8.39 |
| | 4 | 350 | M | 12200 | 4.00 | 181000 | ND |
| | | | F | 11300 | 2.00 | 175000 | ND |
| | | | MF | 11700 | 4.00 | 178000 | ND |
| 14 | 2 | 40 | M | 945 | 4.00 | 11000 | 5.46 |
| | | | F | 1390 | 2.00 | 14000 | 4.13 |
| | | | MF | 1170 | 2.00 | 12500 | 4.46 |
| | 3 | 120 | M | 3540 | 2.00 | 46600 | 7.30 |
| | | | F | 3550 | 2.00 | 47700 | 7.55 |
| | | | MF | 3550 | 2.00 | 47200 | 7.49 |
| | 4 | 350 | M | 13800 | 4.00 | 233000 | 40.3 |
| | | | F | 12600 | 2.00 | 211000 | 45.4 |
| | | | MF | 13100 | 4.00 | 219000 | 42.8 |

AUC = Area under the Curve; Cₘₐₓ = Maximum Serum Concentration; F = Females; M = Males; MF = Combined Male and Female Values; Tₘₐₓ = Timepoint of Maximum Serum Concentration; t½ = Half-life; ND = not determinable as acceptance criteria for calculation were not met.

All concentrations for the anti-DPP3 antibody for the control Group were below the limit of quantification (i.e., <320 ng/mL).

For the low dose (40 mg/kg), Cₘₐₓ was reached at the end of the infusion of PCZ. This was true for the first infusion (D1) as well as after repeated dosing (D14), with similar concentrations at both timepoints. For the low dose (40 mg/kg), 24 hours after start of the first PCZ infusion, the PCZ concentrations accounted only for 3.4% of the Cₘₐₓ concentrations, and 24 hours after the repeated dosing of PCZ at D14, the PCZ concentrations accounted only for 3.9% of the Cₘₐₓ concentrations.

For the high dose (350 mg/kg), Cₘₐₓ was reached at the end of the infusion of AK1967, as observed for the low dose (40 mg/kg). However, decrease of PCZ concentrations proportionally took longer: 24 hours after start of the first PCZ infusion, the PCZ concentrations accounted for 40% of the Cₘₐₓ concentrations, and 24 hours after the repeated dosing of PCZ at D14, the PCZ concentrations accounted for 51.1% of the Cₘₐₓ concentrations.

The calculated sex ratios were less than 2 for mean Cₘₐₓ and AUC and thus, no sex difference was found in the TK profile of the anti-DPP3 antibody. Generally, after i.v. infusion, the concentrations of the anti-DPP3 antibody readily declined with mean t₁₂ values of 4.25 and 8.39 hours for Groups 2 and 3, respectively, on Day 1, and 4.46, 7.49, and 42.8 hours for Groups 2, 3, and 4, respectively, on Day 14. Due to an inability to appropriately define the elimination phase, t_{1/2} was not reportable for Group 4 on Day 1. Mean t₁i2 values for the anti-DPP3 antibody were longer for Group 4 on Day 14 since, for this Group, samples could be collected at later timepoints in the recovery animals, which were absent for the groups 2 and 3. Mean concentration values for the anti-DPP3 antibody were measurable through 24 hours post start of infusion on Days 1 and 14, and through 197 hours post start of infusion on Day 14 for the Group 4 recovery animals.

Exposure, as assessed by the anti-DPP3 antibody mean C_{mw}, and AUC₀₋₂₄ values, increased with the increase in dose level from 40 to 350 mg/kg in a dose-proportional manner for the low and mid dose. For the high dose, where a slight trend to higher than dose-proportional increase (i.e., >8.75) has been observed (ratios for low to high dose were between 9.07 and 21.1). No accumulation potential has been detected when comparing exposure on Day 1 and Day 14. For Group 4 recovery animals, AUC_{0-197 h} could be calculated due to additional, later sampling timepoints and was 344,750 μg*h/mL.

Upon administration of PCZ, cDPP3 activity decreased at all dose levels tested (see FIG. 35 and FIG. 36; only low dose and high dose are presented since mid-dose followed similar kinetics). This cDPP3 drop was more pronounced in the high dose (350 mg/kg) than in the low dose (40 mg/kg) Group. The general range of activity inhibition was between 8 and 60% in Group 2, between 7 and 54% in Group 3 and between 5 and 37% in Group 4.

The electrocardiographic and respiration investigations performed during Day 2 and Day 13 and the blood pressure investigations performed during Day 1 and Day 14 did not reveal any effects that were attributable to treatment with the anti-DPP3 antibody at 40 mg/kg, 120 mg/kg, or 350 mg/kg.

Neurobehavioral investigations did not reveal any remarkable test item-related effects up to the highest tested dose of 350 mg/kg.

Individual body temperatures were variable between animals across all groups and on different days of dosing but were generally similar to pre-treatment levels and within normal biological range.

No test item-related effects on any of the investigated cytokines was noted up to a dose of 350 mg/kg. There was no clear effect of the treatment with the anti-DPP3 antibody on the complement component C3a. A slight tendency of increased Bb fragments was observed on Days 1 and 14 in controls and with a more pronounced effects in treated animals. However, the modulation was considered within biological variation and not test item-related.

Statistically significant differences in organ weights compared with controls were limited to lower absolute, body weight-relative, and brain weight-relative thymus weights in females administered 40 or 350 mg/kg, and higher body weight-relative uterus/cervix weights in females administered 40 mg/kg. Given the lack of a dose-relationship or microscopic correlates, and the inherent variability in organ weight of these tissues in non-human primates, they were considered to be within normal background variation and, therefore, not test article-related.

Macroscopic findings were limited to findings at the injection site and included dark red perivenous discoloration and thickened perivascular tissue in control and treated animals.

Histopathology revealed no test item-related findings. Haemorrhages and mixed inflammatory cell infiltrates were observed at the end of the main study in control and treated animals at the injection site and were, therefore, considered procedure-related.

It is concluded that administration of the anti-DPP3 antibody to cynomolgus monkeys via i.v. infusion on four occasions (on Days 1, 2, 13, and 14) at doses up to 350 mg/kg was well tolerated, with only minor, transient clinical signs in one female at 350 mg/kg observed on Day 14. There were slight alterations in hematology and clinical chemistry parameters, but they showed clear recovery and were not accompanied by histopathological changes in any organ. Accordingly, there were no adverse test item-related effects. Consequently, the No Observable Adverse Effect Level (NOAEL) was considered to be 350 mg/kg, equivalent to a mean systemic exposure in terms of AUC₀₋₁₉₇ of 344,750 μg*h/mL and in terms of Cₘₐₓ value of 13,100 μg/mL on Day 14.

### Example 13—A Randomized Double-Blind Placebo-Controlled Phase I Study on the Safety, Tolerability, and Pharmacokinetics/Pharmacodynamics of Escalating Single Intravenous Doses of the Anti-DPP3 Antibody Procizumab (PCZ) in Healthy Male Volunteers

This was a randomized, double-blind, placebo-controlled, escalating single-dose Phase I study in healthy male subjects. The double-blind, placebo-controlled, single-dose design was used to reduce expectancy and bias in all study procedures. A placebo control was used to establish the frequency and magnitude of changes in study endpoints that may have occurred in the absence of active treatment.

This study was the first-in-human study of PCZ in healthy male subjects. Prior to this study there was no information about any adverse events of PCZ in healthy humans. This study demonstrated the safety and tolerability of PCZ as determined by the differences between the placebo group and the PCZ groups.

Based on the aforementioned Example 12, the starting dose for this human study was 3 mg/kg PCZ, administered as a single infusion over 2 hours. The maximum dose was 12 mg/kg to provide a sufficient safety margin for the intended therapeutic dose of 5-10 mg/kg. As the actual body weight might be difficult to acquire in critically ill patients, and a slightly higher dose for a later treatment may turn out to be more beneficial, 12 mg/kg provided a sufficient safety window. The intended maximum dose of 12 mg/kg still provided a 13- and 29-fold safety margin to the observed NOAELs of the Example 12 studies (Table 32).

TABLE 32
NOAELs of the mouse and NHP studies, and associated
safety margins compared to the minimal and
maximal doses in the Phase I study.

| | NOAEL | Safety Margin to | Safety Margin to max. |
|---|---|---|---|
| Species | (mg/kg) | starting dose (3 mg/kg) | clinical dose (12 mg/kg) |
| Mouse | 150 | 50 | 13 |
| NHP | 350 | 116 | 29 |

The main inclusion criteria for this human study were:
Written informed consent to participate in the trial prior to any study-mandated procedure;
Male subjects aged 18 to 35 years, inclusive;
Agreement to use a reliable way of contraception with their partners from study entry until one month after study drug administration;
BMI between 18 and 30 kg/m², with a lower limit of body weight of 50 kg and an upper limit of 100 kg; and
Healthy as determined by medical history, physical examination, vital signs, 12-lead electrocardiogram, and clinical laboratory parameters.

A total of 24 healthy male subjects were recruited and divided over 3 groups of 8 subjects each. PCZ (or placebo) was administered as an i.v. infusion over 2 hours in 3 sequential groups of 8 healthy male subjects (1st group=3 mg/kg, 2nd group=6 mg/kg, 3rd group=12 mg/kg) (n=6 active, n=2 placebo for each group). Blood samples for quantification of PCZ concentration and cDPP3 activity were collected immediately prior to the start of PCZ/placebo infusion (Baseline, TO), and thereafter at 0.5 hours (T30), 1 hour (T60), 1.5 hours (T90), 2 hours (T120), 3 hours (T180), 4 hours (T240), 5 hours (T300), 6 hours (T360), 7 hours (T420), 8 hours (T480), 9 hours (T540), 10 hours (T600), 24 hours (Day 1), 48 hours (Day 2), 72 hours (Day 3), 168 hours (Day 7), 336 hours (Day 14), 504 hours (Day 21), and 672 hours (Day 28).

### Investigational Medicinal Product(s) (IMP(s))

The investigational medicinal product was a solution for intravenous administration presented in Ph.Eur. type I glass vials with fluoropolymer coated bromobutyl rubber stoppers and tear-off plain aluminum over seals. 20R vials (25 mL total container volume) were aseptically filled with 20 mL PCZ to allow for an extractable volume of 18.5 mL The PCZ composition is listed in Table 33.

TABLE 33
Composition of the PCZ drug product. Manufacturer:
BioConnection, Oss, the Netherlands

| | | | Quantity | | Quantity | |
|---|---|---|---|---|---|---|
| Name of Ingredient | Function | Reference | (per mL) | | (per vial) | |
| PCZ | Active | IMPD | 20 | mg | 400 | mg |
| Trehalose Dihydrate | Stabilizer | Ph. Eur/USP | 90.7 | mg | 1.8154 | g |
| L-Methionine | Antioxidant | Ph. Eur/USP | 2.86 | mg | 57.2 | mg |
| L-Histidine | Buffer | Ph. Eur/USP-NF | 1.11 | mg | 22.2 | mg |
| L-Histidine HCl * H2O | pH adjustment | Ph. Eur | 0.51 | mg | 10.2 | mg |

| | | | | |
|---|---|---|---|---|
| Water for Injection | Solvent | Ph. Eur/USP/JP | Ad 1 ml | Ad 20 ml |

The clinical trial medication was diluted from the Drug Product stock solution with sterile NaCl solution 0.9% to the required dosing (3.0 mg/kg, 6.0 mg/kg, or 12.0 mg/kg) with an infusion volume of 1.0-1.2 mL/kg depending on the required dose.

The placebo was a solution for intravenous administration presented in Ph.Eur. type I glass vials with fluorpolymer coated bromobutyl rubber stoppers and tear-off plain aluminum over seals. 20R vials (25 mL total container volume) were aseptically filled with 20 mL placebo to allow for an extractable volume of 18.5 mL. The placebo composition is listed in Table 34.

TABLE 34
Composition of the placebo drug product. Manufacturer: BioConnection, Oss, the Netherlands.

| Name of Ingredient | Function | Reference | Quantity (per mL) | | Quantity (per vial) | |
|---|---|---|---|---|---|---|
| Trehalose Dihydrate | Stabilizer | Ph. Eur/USP | 90.7 | mg | 1.8154 | g |
| L-Methionine | Antioxidant | Ph. Eur/USP | 2.86 | mg | 57.2 | mg |
| L-Histidine | Buffer | Ph. Eur/USP-NF | 1.11 | mg | 22.2 | mg |
| L-Histidine HCl * H2O | pH adjustment | Ph. Eur | 0.51 | mg | 10.2 | mg |

| | | | | |
|---|---|---|---|---|
| Water for Injection | Solvent | Ph. Eur/USP/JP | Ad 1 ml | Ad 20 ml |

The safety and tolerability of PCZ was determined by:
Reported number of adverse events from baseline (start of PCZ administration) up until the last follow-up visit 28 days after the PCZ administration;
Vital signs (e.g., heart rate, blood pressure, peripheral oxygen saturation and body temperature) during the first 24 hours after start of the PCZ administration, as well as during the six follow-up visits;
Local tolerability at site of i.v. infusion of PCZ;
Safety laboratory parameters (e.g., Hb, Ht, leukocytes, thrombocytes, leukocyte differential blood count, sodium, potassium, creatinine, urea, alkaline phosphatase, ALT, AST, bilirubin, GGT, CK, CRP, PT, APTT, fibrinogen) from baseline (just prior to the start of the PCZ administration) up until the last follow-up visit 28 days after PCZ administration; and
12-lead electrocardiogram (ECG) at baseline (screening), compared to ECG's performed 2 hours, 9 hours, and 7 days after start of the PCZ administration.

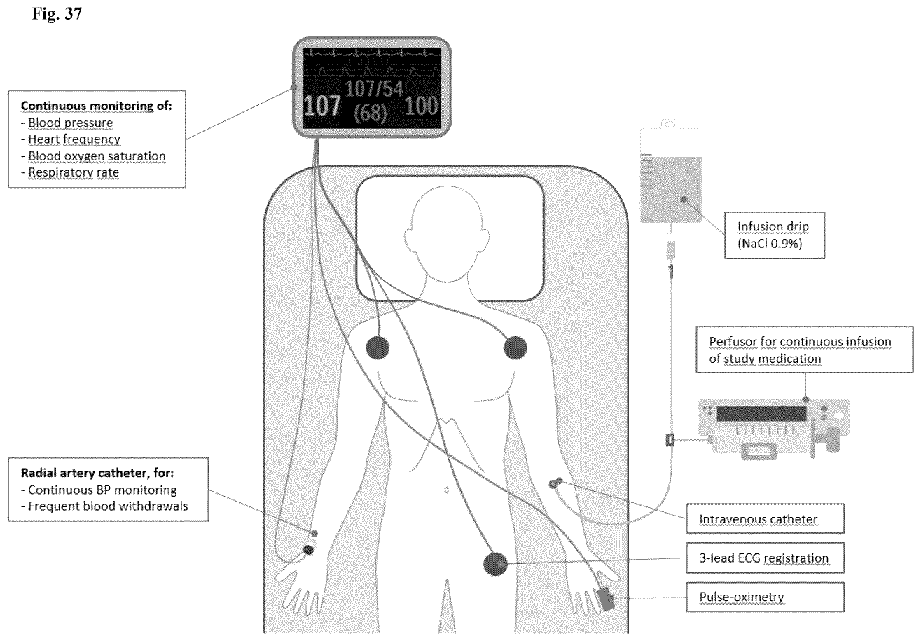

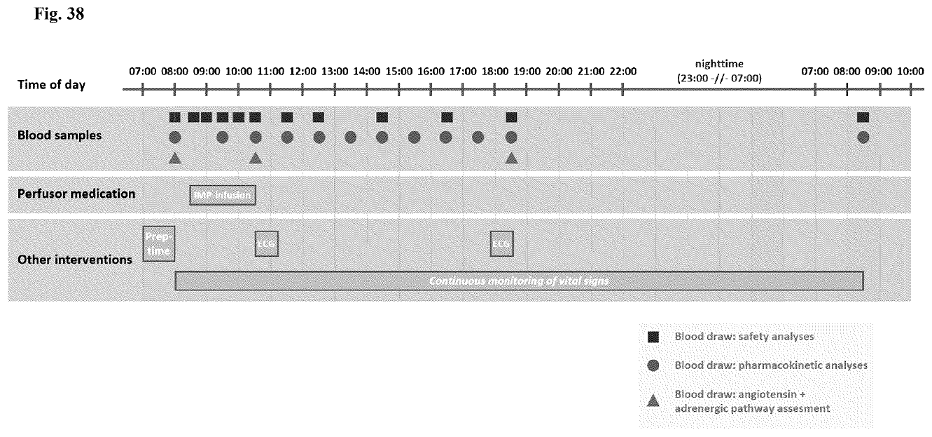

On the treatment day, subjects were hospitalized for approximately 26 hours (taking preparation time prior to administration of PCZ into account). There was continuous monitoring of symptoms, (intra-arterial) blood pressure, SpO2(%), and heart rate (ECG). Subjects received one venous catheter in the forearm. An arterial cannula was placed in the radial artery, following local anesthesia using a lidocaine 1% solution for injection. Subjects then received a single dose of study medication (PCZ—3 mg/kg, 6 mg/kg, or 12 mg/kg, or placebo), administered as an intravenous infusion over a 2-hour period. During the treatment day, serial blood samples were drawn from the arterial cannula. A 12-lead electrocardiogram was performed 2 and 9 hours after start of the PCZ administration. Approximately 12 hours after initiation of PCZ administration, cannulas were removed, and a pressure bandage was applied to the site of arterial cannulation (see FIG. 37 for an overview of the experimental setup). Subjects stayed overnight at the research Intensive Care Unit for continuous monitoring of vital signs. In the morning, a venous blood withdrawal was performed approximately 24 hours after start of IMP administration. The subjects returned an additional 6 times for follow-up visits over the next 28 days (subjects were checked for adverse events and blood samples were obtained via venipuncture) (see FIG. 38 for an overview).

The pharmacodynamic effects of PCZ were monitored by performing an assessment of both systemic angiotensin responses and systemic adrenergic responses at key timepoints just prior to and after PCZ administration, serving as additional (secondary) measures of the main (molecular) pharmacodynamic properties. To allow for the accurate quantification of these metabolites, assays implementing liquid chromatography mass spectrometry (LC-MS) were used. Prior to performing preclinical experiments with PCZ, luminometric immunoassays (LIA) (also known as, Immunoluminometric assay (ILMA)) for cDPP3 concentration quantification and fluorescence assays for cDPP3 activity quantification were developed. These assays allowed for the accurate quantification of cDPP3 enzyme activity and cDPP3 concentration, both under baseline circumstances as well as during different critical illness etiologies. The availability of enzyme activity assays allowed for an additional method to monitor PCZ pharmacokinetic (and pharmacodynamic) effects, because the dose-dependency of its enzyme-inhibitory potential can be quantified. Thus, these assays served as additional (secondary) measures of PCZ pharmacokinetic properties.

The study demonstrated various pharmacokinetic/pharmacodynamic effects of PCZ.

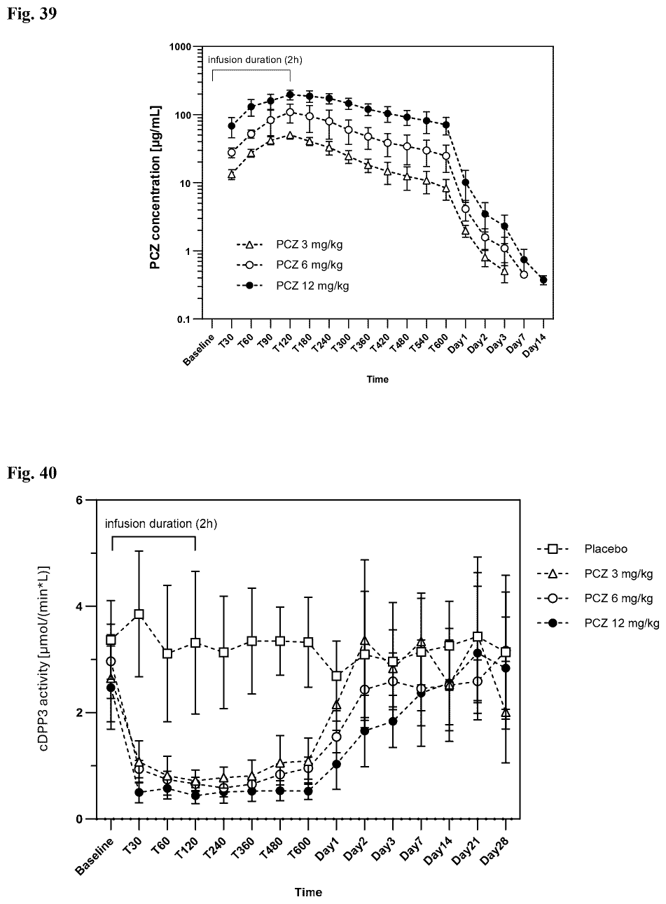

Pharmacokinetic Results: The time course of PCZ concentrations observed after administration of the three doses of PCZ is shown in FIG. 39. Pharmacokinetic parameters obtained after non-compartmental analysis of plasma samples are summarized in Table 35. In all three dose groups, the Cₘₐₓ was attained at or shortly after termination of infusion. Dose-proportional increases in Cₘₐₓ and AUC were observed. A small VD (˜297-422 ml/kg) indicates that PCZ predominantly remains within the circulation. The terminal elimination T_{1/2} of PCZ following administration of 12 mg/kg was ˜53 hours. The terminal elimination T_{1/2} of PCZ could not be calculated for the 3 mg/kg and 6 mg/kg doses due to limited observation time in which PCZ levels could be quantified.

TABLE 35
Pharmacokinetic parameters of PCZ in the first-in-human trial after single dose.

| Dose Group/ | 3 mg/kg | 6 mg/kg | 12 mg/kg |
|---|---|---|---|
| PK Parameter | (n = 6) | (n = 6) | (n = 6) |
| Cₘₐₓ (μg/ml) | 50.4 ± 6.5 | 109 ± 34.7 | 199 ± 31.5 |
| DN_Cₘₐₓ (μg/ml/mg) | 16.8 ± 2.17 | 18.2 ± 5.79 | 16.6 ± 2.62 |
| AUC_{0-∞} (μg*h/ml) | 363 ± 74.7 | 877 ± 339 | 2110 ± 590 |
| DN_AUC_{0-∞} (μg*h/ml/mg) | 121 ± 24.9 | 146 ± 56.4 | 176 ± 49.2 |
| VD (ml/kg) | 297 ± 59.5 | 344 ± 107 | 422 ± 82.7 |
| Terminal T_{1/2} (h) | n.d. due to limited | n.d. due to limited | 53.1 ± 21.5 |
| | observation time | observation time | |
| Cl (ml/h/kg) | 8.54 ± 1.63 | 7.67 ± 2.68 | 6.14 ± 2.06 |

Data are expressed as mean ± standard deviation.
AUC_{0-∞}, area under the plasma concentration-time curve from time zero to infinity; Cₘₐₓ, highest observed plasma concentration; Cl, total clearance calculated; DN_AUC_{0-∞}, dose-normalized AUC_{0-∞}; DN_Cₘₐₓ, dose-normalized Cₘₐₓ; Terminal T_{1/2}, elimination half-life; VD, apparent volume of distribution characterized by the terminal phase.

Pharmacodynamic Results: From preclinical animal studies, it is known that the anti-DPP3 antibody has a short half-life compared to other IgG1 antibodies. This shorter half-life results in a reduced time-range of relevant pharmacodynamic effects compared to other antibodies, with animal disease models demonstrating therapeutic effects in a 24-48 hour time-window.

For the three doses tested, the plasma concentration of PCZ dropped as follows:
3 mg/kg: from a mean Cₘₐₓ of 50.4 μg/mL at the end of PCZ infusions by 96% to a mean Cₘₐₓ of 2.0 μg/mL within 24 hours after start of administration of PCZ, and by 99% to a mean Cₘₐₓ of 0.5 μg/mL within 72 hours after start of administration of PCZ.
6 mg/kg: from a mean Cₘₐₓ of 109 μg/mL at the end of PCZ infusions by 96.2% to a mean Cₘₐₓ of 4.1 μg/mL within 24 hours after start of administration of PCZ, and by 99% to a mean Cₘₐₓ of 1.1 μg/mL within 72 hours after start of administration of PCZ.
12 mg/kg: from a mean Cₘₐₓ of 196 μg/mL at the end of PCZ infusions by 94.8% to a mean Cₘₐₓ of 10.2 μg/mL within 24 hours after start of administration of PCZ, and by 98.8% to a mean Cₘₐₓ of 2.3 μg/mL within 72 hours after start of administration of PCZ.

The time course of cDPP3 activity observed after administration of three doses of PCZ is shown in FIG. 40. PCZ elicited a dose-dependent inhibition of cDPP3 activity in plasma. For the three doses tested, the plasma cDPP3 activity dropped as follows:
3 mg/kg: from a mean of 2.7 U/L immediately prior to PCZ infusion by 73% to a minimum of a mean of 0.72 U/L at the end of PCZ infusion, and an increase by 8% to a mean of 2.8 U/L compared to the pre-dose activity within 72 hours after the start of administration of PCZ.
6 mg/kg: from a mean of 3.0 U/L immediately prior to PCZ infusion by 78% to a minimum of a mean of 0.66 U/L achieved at the end of PCZ infusion, and a decrease by 13% to a mean of 2.6 U/L compared to the pre-dose activity within 72 hours after the start of administration of PCZ.
12 mg/kg: from a mean of 2.5 U/L immediately prior to PCZ infusion by 82% to a minimum mean of 0.44 U/L achieved at the end of PCZ infusion, and a decrease by 28% to a mean of 1.8 U/L compared to pre-dose activity within 72 hours after the start of administration of PCZ.

### Example 14—Multi-Center, Randomized, Placebo-Controlled, Double-Blind Phase 1b Trial to Investigate Safety, Tolerability, and Pharmacokinetics of Procizumab (PCZ) in Patients with Cardiogenic Shock and Elevated Circulating Dipeptidyl Peptidase 3 (cDPP3) Concentrations

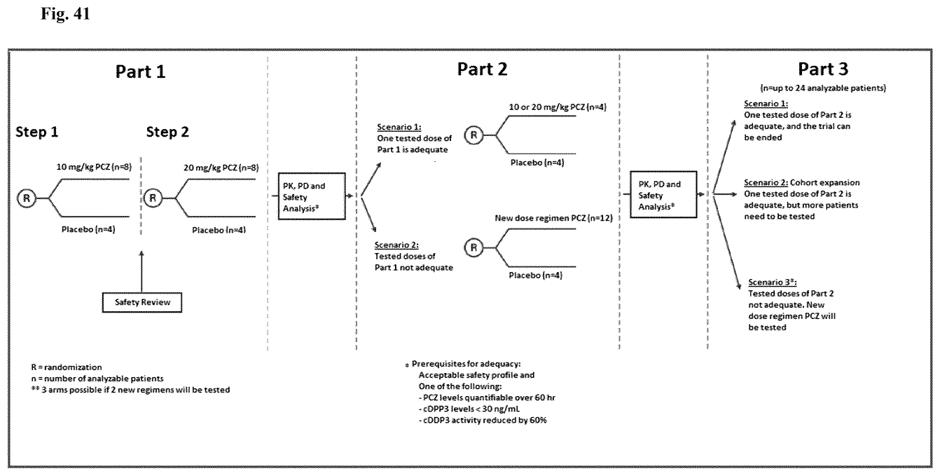

This trial will investigate the safety, pharmacokinetics (PK), and pharmacodynamics (PD) of procizumab in patients presenting with cardiogenic shock (CS) due to acute coronary syndrome (ACS) or CS with septic origin. Each treatment arm to be investigated will consist of 50% CS with ACS and 50% CS with septic origin. The trial scheme is depicted in FIG. 41.

The trial consists of 2 parts. Part 1 will be a randomized, double-blind, placebo-controlled investigation of 10 mg/kg and 20 mg/kg of procizumab. The IMPs—procizumab and placebo—will be administered as a single intravenous infusion over 2 hours. For safety reasons, Part 1 will be done in 2 steps. In the first step of Part 1, patients will be randomized to either 10 mg/kg procizumab (n=8) or placebo (n=4). After Step 1, safety data will be reviewed to determine if Step 2 of Part 1 may be conducted. More specifically, at the end of Part 1 when the last patient will have been observed over 30 days, PK, PD, and safety data will be analyzed. In Step 2 of Part 1, patients will be randomized to 20 mg/kg procizumab (n=8) or placebo (n=4); procizumab and placebo will be administered as a single intravenous infusion over 2 hours.

The design of Part 2 will depend on the safety, PK, and PD results in Part 1. Two scenarios will be considered for the design of Part 2. In Scenario 1, if one of the tested dose regimens in Part 1 fulfills the following prerequisites, the respective dose will be further investigated in a dose expansion cohort: The dose regimen has an acceptable safety profile and one of the following applies: the dose of procizumab is sufficient to maintain plasma levels of procizumab above IC₅₀ (3,850 ng/mL) up to at least 60 hours after start of infusion or the dose of procizumab is sufficient to keep the cDPP3 level below 30 ng/mL up to 60 hours after the start of IMP infusion or the dose of procizumab is sufficient to reduce the cDPP3 activity by 50% from the screening cDPP3 activity value up to 60 hours after the start of IMP infusion. If both tested doses fulfill the prerequisites, an independent recommendation will be followed.

Scenario 2 will apply if none of the tested dose regimens in Part 1 fulfills the prerequisites described above. In Scenario 2 the data of Part 1 will be used to model 1 or 2 optimized dose regimen(s). The dose levels will not be increased and will remain either 10 or 20 mg/kg, however the dosing regimen may be modified, e.g., repeated doses (up to a maximum of 3 times) or prolonged infusion times. Scenario 2 will be a randomized, placebo-controlled investigation with the target to treat 12 patients in each of the dose regimens selected. Only 4 patients will be treated with placebo, since 8 placebo patients will have already been treated in Part 1. Therefore, the randomization ratio will be 3:1 for procizumab to placebo, respectively, if 1 procizumab dose regimen will be investigated and, 3:3:1 if 2 procizumab dose regimens will be investigated.

At the end of Part 2, if it is determined that data from additional patients will be needed to identify the recommended dose for the next phase of a procizumab trial, e.g. if existing data are not sufficient in terms of safety, PK, and PD, then either additional data will be generated for one of the investigated doses in Part 2, i.e. in an expansion cohort, or that another dose regimen will be investigated in a further Part 3 of the trial. In the latter case, the same principles will be used as described for Part 2. Up to 24 analyzable patients may be treated, including a placebo arm.

To be eligible to participate in this trial, a patient will be an adult (aged 18-75 years) and will meet all the following criteria:
1. Signed informed consent.
2. Diagnosis of CS due to ACS or CS of septic origin based on the following entry criteria:
   a. Need for ongoing vasopressors (any dose of vasopressors with or without inotropes) by the time of injecting the trial intervention to maintain a MAP ≥65 mm≥or SBP ≥90 mmHg. Start of treatment intervention to be done within a maximum of 16 hours after the start of vasopressors.
   b. Serum lactate >2.0 mmol/L.
   c. High cDPP3 concentration >30 ng/mL.

The primary endpoints for all parts of the study will be:
Incidence of treatment-emergent adverse events (TEAEs);
Incidence of treatment-emergent serious adverse events (TESAEs);
Changes in vital signs;
Changes in clinical laboratory assessments;
Incidence of adverse events of special interest (AESIs); and
Clinically significant arrhythmias identified on Holter monitoring.

The secondary endpoints for all parts of the study will be:

### Pharmacokinetics (Procizumab):

Cₘₐₓ;
AUC (AUC₀₋ₗₐₛₜ, AUC_{0-inf}, AUCₜ);
Tₛₛ (if applicable);
Tₘₐₓ;
Cₘᵢₙ at the end of the dosing interval (if applicable);
T_{1/2};
CL;
VD; and
Kₑₗ.
   Pharmacodynamics (cDPP3):
Change in cDPP3 concentration and activity; and
Correlation between cDPP3 concentration and activity.

The exploratory endpoints for all parts of the study will be:
Cumulative dose of vasopressors/inotropes per day;
Hours on vasopressors/inotropes;
Change in VIS;
Rate and duration of mechanical respiratory support, RRT and MCS;
Change in SOFA score;
Changes in hemodynamic measures including CO, wedge pressure, SVO2, pulmonary pressures, right atrial pressure, and calculated measures such as cardiac power, pulmonary and SVR;
Change in P/F ratio;
Cumulative fluid balance;
Renin-angiotensin system response (measured via RAAS peptides, aldosterone, renin, and ACE);
Dynamics of cardiometabolic and inflammatory biomarkers; and
All-cause and cardiovascular mortality.

The Procizumab formulation will be 20 mg/mL procizumab protein solution in 10 mM His/HCl, 250 mM Trehalose, 20 mM Methionine, at pH 6.5 for an extractable volume of >19 mL. The placebo formulation will be 10 mM His/HCl, 250 mM Trehalose, 20 mM Methionine, at pH 6.5.

### Example 15—Quantification of Procizumab (PCZ) from Various Human and Animal Serums Quantification of PCZ in Human Serum Samples (Pharmacokinetic Assay)

The method used an antigen-specific binding phase to capture PCZ from the human serum sample. Streptavidin precoated microplates (Thermo Scientific #436014) were blocked with 300 μL per well using Superblock Blocking Buffer (Thermo #37515) in PBS for 1 hour. The wells were washed once with PBS+0.05% Tween 20 followed by the addition of 100 μL per well of a Biotin-DPP3 peptide, Biotin-Trioxatridecan-Succinamic Acid (Ttds)-ETVINPETGEQIQSWYR-NH₂ (Peptides & Elephants, Hennigsdorf, Germany) at 50 ng/mL in 1×PBS with 0.1% BSA and 0.05% Tween 20 for an hour at room temperature and 600 rpm on an orbital shaker. Calibration standards and quality control samples were prepared by diluting PCZ at defined concentrations in neat matrix (normal human serum) (Zenbio), validated, and stored at −80° C. until use. Clinical serum samples were prepared by allowing the blood to clot after 15-30 mins undisturbed at room temp and then centrifuging for 5 minutes at 2,000 g at 4° C. The serum supernatant was collected into tubes. Clinical serum samples were diluted in neat matrix to allow recovery within the calibration standard's measuring range. After washing 4 times with PBS+0.05% Tween 20, 100 μL each of the calibration standards, the quality control samples, and the clinical serum samples were applied into separate wells with a minimum required dilution (MRD) of 1/20 in sample dilution buffer containing PBS, 1% BSA, and 0.1 M CaCl₂. The microplate was incubated for 2 hours at room temperature and at 600 rpm on an orbital shaker. Wells were washed 4 times with washing solution (PBS+0.05% Tween 20). Then, 100 μL of detector antibody solution (mouse anti-human IgG1 (Fc specific)-peroxidase-linked detector antibody in PBS, 1% BSA) was added per well and incubated for 1 hour at room temperature at 600 rpm on an orbital shaker to bind to the solid-phase-bound PCZ. The wells were washed 5 times with PBS+0.05% Tween 20. Finally, 100 μL of the 3,3′,5,5′-Tetramethylbenzidine (TMB) substrate was added to each well and converted by the peroxidase linked to the detector antibody. The TMB stock solution was prepared by dissolving 1 mg/ml in DMSO. The working solution was made by diluting 1 ml of the DMSO stock with 9 ml phosphate-citrate buffer, pH 5.0, and then adding 2 μl of fresh 30% hydrogen peroxide per 10 ml of substrate/buffer solution immediately prior to use. After a 10 minute incubation with the substrate under 600 rpm on an orbital shaker agitation, 100 μL of the stopping solution, 1 M H₂SO₄, was added to the wells with mixing at room temp. Using a photometer, the colorimetric signal was measured at 450 nm that is directly proportional to the concentration of PCZ. A 4-parameter-logistic regression model was used to read out the sample concentration of PCZ from the calibration standard curve.

The described method was demonstrated to be not affected by the presence of DPP3 in the sample.

Quantification of PCZ in Serum Samples from Cynomolgus Monkeys (Pharmacokinetic Assay)

The method used an antigen-specific binding phase to capture PCZ from the monkey serum sample. Streptavidin precoated microplates (Thermo Scientific #436014) were blocked with 300 μL per well using Superblock Blocking Buffer (Thermo #37515) in PBS for 1 hour. The wells were washed once followed by the addition of 100 μL per well of a Biotin-DPP3 peptide, Biotin-Trioxatridecan-Succinamic Acid (Ttds)-ETVINPETGEQIQSWYR-NH₂ (Peptides & Elephants, Hennigsdorf, Germany) at 50 ng/mL in 1×PBS with 0.1% BSA and 0.05% Tween 20 for an hour at room temperature and 600 rpm on an orbital shaker. Calibration standards and quality control samples were prepared fresh on the day of running the assay by diluting PCZ at defined concentrations in neat matrix (normal serum from cynomolgus monkey of Mauritius origin (Silabe)). Clinical serum samples were prepared by allowing the blood to clot after 15-30 mins undisturbed at room temp and then centrifuging for 5 minutes at 2,000 g at 4° C. The serum supernatant was collected into tubes. Clinical serum samples were appropriately diluted in neat matrix to allow recovery within the calibration standard's measuring range. After washing 4 times with PBS+0.05% Tween 20, 100 μL of the calibration standards, the quality control samples, and the clinical serum samples were applied into separate wells with a minimum required dilution (MRD) of 1/20 in sample dilution buffer containing PBS, 1% BSA, and 0.1 M CaCl₂. The microplate was incubated for 2 hours at room temperature and at 600 rpm on an orbital shaker. Wells were washed 4 times with washing solution (PBS+0.05% Tween 20). Then, 100 μL of a detector antibody solution (mouse anti-human IgG1 (Fc specific)-peroxidase-linked detector antibody in PBS with 1% BSA were added per well, and incubated at room temperature at 600 rpm on an orbital shaker to bind to the solid-phase-bound PCZ. The wells were washed 5 times with PBS+0.05% Tween 20. Finally, 100 μL of the 3,3′,5,5′-Tetramethylbenzidine (TMB) substrate was added to each well with shaking on an orbital shaker at 600 rpms at room temp. The TMB stock solution was prepared by dissolving 1 mg/ml in DMSO. The working solution was made by diluting 1 ml of the DMSO stock with 9 ml phosphate-citrate buffer, pH 5.0, and then adding 2 μl of fresh 30% hydrogen peroxide per 10 ml of substrate/buffer solution immediately prior to use. After a 5-15 minute incubation with the substrate under 600 rpm on an orbital shaker agitation, 100 μL of the stopping solution, 1 M H₂SO₄, was added with mixing at room temp. Using a photometer, the colorimetric signal was measured at 450 nm that is directly proportional to the concentration of PCZ. A 4-parameter-logistic regression model was used to read out the sample concentration of PCZ from the calibration standard curve.

The described method was not affected by the presence of cDPP3 in the sample.

Quantification of PCZ in Serum Samples from Mice (Pharmacokinetic Assay)

A polyclonal anti-PCZ antibody was raised in rabbits by immunization with PCZ. After development of antibodies in the rabbit, the rabbit antiserum was purified as follows:
Protein A chromatography to isolate whole IgG;
Immunoaffinity purification against PCZ for positive selection; and
Immunoaffinity purification against human IgG1, kappa, from human plasma (Sigma #5154) for negative selection.

Using this purified polyclonal rabbit anti-PCZ antibody, a binding phase assay was developed to capture PCZ in mouse serum and it is described as follows. A solution of 0.5 μg/mL of the rabbit antibody was prepared in 1×PBS, and 100 μL were added to wells of a 96 well microplate (Nunc Immuno plate, Thermo Scientific #439454) to coat overnight at 4° C. The plates were washed 4 times with 1×PBS+0.05% Tween 20 and blocked with 300 μL per well using Superblock Blocking Buffer (Thermo #37515) in 1×PBS for 1 hour at room temperature. Calibration standards and quality control samples were prepared in blank matrix (pooled mouse serum of individual batches, Janvier Labs) by spiking with PCZ. Serum samples were prepared by allowing the blood to clot after 15-30 mins undisturbed at room temp and then centrifuging for 5 minutes at 2,000 g at 4° C. The serum supernatant was collected into tubes and frozen at −20° C. The frozen serum study samples were thawed at room temperature and centrifuged 5 minutes at 2,000 g at 4° C. just before analysis. Samples were appropriately diluted in blank matrix to allow recovery within the measuring range. After washing 4 times with PBS+0.05% Tween 20, 100 μL of the calibration standards, the quality control samples, and the study samples were applied to separate wells with a minimum required dilution (MRD) of 1/10 in Low Cross buffer (Candor Bioscience GmbH #100500). The microplate was covered and incubated for 2 hours at room temperature. Wells were washed 4 times with PBS+0.05% Tween 20. Then, 100 μL of the detector antibody solution (0.1 μg/mL of anti-human IgG (Fc specific)-peroxidase-linked antibody produced in goat (Sigma #A0170)) in 1×PBS and 1% BSA was added to the wells and incubated for 1 hour at room temperature to bind to the solid-phase-bound PCZ. The wells were washed 4 times with PBS+0.05% Tween 20. Finally, 100 μL of the 3,3′,5,5′-Tetramethylbenzidine (TMB) substrate was added and converted by the peroxidase linked to the detector antibody. The TMB stock solution was prepared by dissolving 1 mg/ml in DMSO. The working solution was made by diluting 1 ml of the DMSO stock with 9 ml phosphate-citrate buffer, pH 5.0, and then adding 2 μl of fresh 30% hydrogen peroxide per 10 ml of substrate/buffer solution immediately prior to use. After a 30 minute incubation with the substrate under 600 rpm on an orbital shaker agitation, 100 μL of the stopping solution (1 M H₂SO₄) was added to the wells with mixing at room temp. Using a photometer, the colorimetric signal was measured at 450 nm that is directly proportional to the concentration of PCZ. A 4-parameter-logistic regression model was used to read out the sample concentration of PCZ from the calibration standard curve.

The described method was not affected by the presence of cDPP3 in the sample.

### Example 16—Administration of Humanized Anti-DPP3 Antibody (Procizumab, PCZ) in Extreme-Critically Ill Patients with Refractory Shock and Multi-Organ Failure

PCZ was administered in three extreme-critically ill patients with refractory shock and multi-organ failure.

### Patient Description:

The patients included in this study constitute a specific patient population, for which treatment with PCZ and resulting outcome have not been investigated in the art so far. This population can be termed “extreme-critically ill patients”. This term is defined by the following features: Extreme-critically ill patient according to the present invention is defined as a patient who has a 24-hour probability of mortality of at least 70% and/or has organ dysfunction requiring mechanical or pharmacological support of at least two organs.

The patient has a 24-hour probability of mortality of at least 70%, preferably of at least 80%, more preferably of at least 90%, most preferably of at least 95%, and/or characterized by organ dysfunction requiring mechanical or pharmacological support of at least two organs. Two women (patients PCZ_01, 64 years old, and PCZ 03, 80 years old) and one man (patient PCZ_02, 84 years old) were treated. All three patients had pre-existing conditions and were in utmost critical condition with signs of multi-organ failure, with patient PCZ_01 and PCZ_02 suffering from combined septic and cardiogenic shock (AMI in patient 01 was untreated due to hemodynamic instability) and patient PCZ_03 from right-sided cardiogenic shock. All three were suffering from renal failure, in need of renal replacement therapy, two out of three (patients PCZ_01 and PCZ 02) were intubated and under invasive mechanical ventilation. All three patients were assessed and approved for treatment with PCZ by an independent international patient selection board, which considered patient PCZ_01 as having a 24-hour probability of mortality of >90%, and patients PCZ_02 and PCZ_03 as already having entered the process of dying.

### Intervention:

Patients received a single dose of PCZ infusion at a dose of 10 mg/kg body weight over a 2-hour period.

PCZ was administered using a dedicated lumen of a central venous catheter, separately from any concomitant drugs, between 1 and 3 days after transfer to intensive care unit (ICU).

### Follow-Up:

The patients were observed for a period of 6 days after administration of PCZ, and several blood samples were collected in this period for the assessment of PCZ concentration and cDPP3 activity.

### Results:

The therapy was well tolerated in all three patients, no immediate adverse reactions were noted, and no adverse events were associated with PCZ.

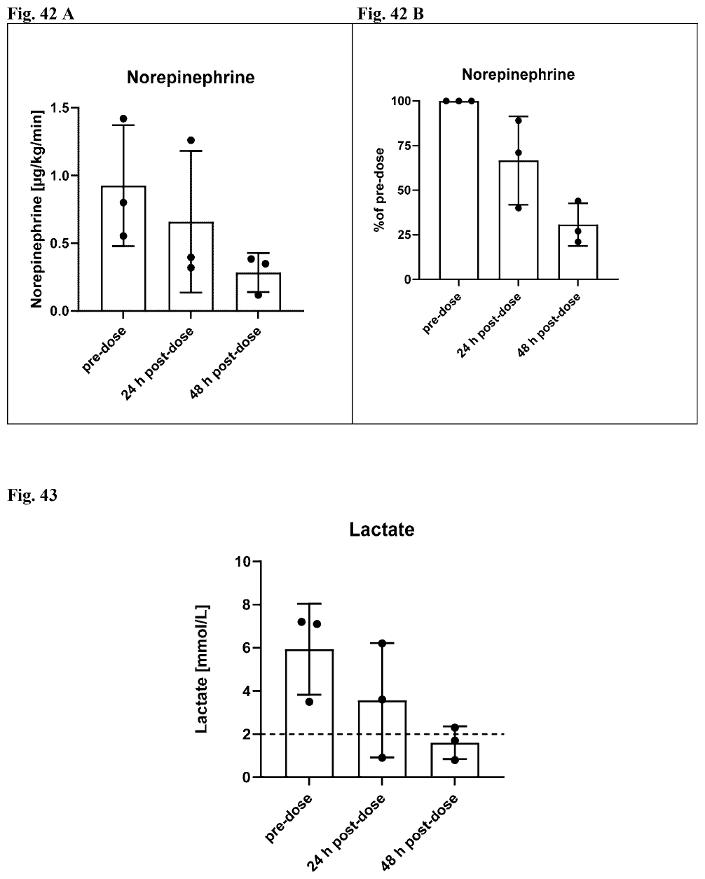

Clinical and laboratory data recorded for the patients immediately prior to the administration of PCZ as well as after 24 and 48 hours thereafter are summarized in Table 36, FIG. 42 A, FIG. 42 B, and FIG. 43.

Prior to administration of PCZ all patients suffered from multi-organ failure: Affected organs included the cardiovascular system (mean norepinephrine dosing 0.9 μg/kg/min), the lung (mean Horovitz-Index (P/F ratio) 118 mmHg), as well as the kidney (mean creatinine concentration 2.3 mg/dL) and required multi-organ support.

TABLE 36
Clinical and laboratory patient characteristics prior to and after treatment with PCZ.

| | Patient PCZ_01 | | | Patient PCZ_02 | | | Patient PCZ_03 | | |
|---|---|---|---|---|---|---|---|---|---|
| Variable | Before | Day 1 | Day 2 | Before | Day 1 | Day 2 | Before | Day 1 | Day 2 |
| Norepinephrine | 0.8 | 0.32 | 0.349 | 1.42 | 1.26 | 0.384 | 0.554 | 0.396 | 0.119 |
| [μg/kg/min] | | | | | | | | | |
| Lactate | 3.5 | 0.9 | 0.8 | 7.1 | 6.2 | 2.3 | 7.2 | 3.6 | 1.7 |
| [mmol/L] | | | | | | | | | |
| P/F ratio | 94.4 | 295 | 228 | 83.5 | 192 | 182 | 176 | 211 | 257 |
| [mmHg] | | | | | | | | | |
| Interleukin-6 | 40.9 | 11.3 | 8.5 | 241,306 | 5,016 | 408 | 893.5 | 67.8 | 27.2 |
| [ng/L] | | | | | | | | | |
| C-reactive protein | 298 | 273 | 179 | 166 | 331 | 332 | 341 | 351 | 252 |
| [mg/L] | | | | | | | | | |
| Procalcitonin | 2.33 | n/a | n/a | 30.41 | 35.94 | 21.79 | 42.94 | 31 | 18.03 |
| [μg/L] | | | | | | | | | |
| NT-proBNP | 12,811 | 10,554 | 8,188 | 11,152 | 9,469 | 7,650 | 4,602 | 4,645 | 3,981 |
| [ng/L] | | | | | | | | | |
| Creatinine | 3.26 | 2.28 | 1.67 | 2.29 | 1.66 | 0.86 | 1.53 | 0.91 | 0.74 |
| [mg/dL] | | | | | | | | | |
| Urea | 25.5 | 29.8 | 35.7 | 32.8 | 21.4 | 12.9 | 19.1 | 13.8 | 20.1 |
| [mg/dL] | | | | | | | | | |
| GFR (CKD-EPI, | 14 | 22 | 32 | 25 | 37 | 79 | 32 | 60 | 77 |
| [mL/min]) | | | | | | | | | |
| AST/GOT | 123 | 437 | 327 | 52 | 616 | 386 | 81 | 120 | 97 |
| [U/L] | | | | | | | | | |
| ALT/GPT | 63 | 284 | 298 | 30 | 232 | 185 | 42 | 68 | 53 |
| [U/L] | | | | | | | | | |
| Gamma-GT | 137 | 126 | 141 | n/a | 206 | 247 | 112 | 102 | 102 |
| [U/L] | | | | | | | | | |
| Total bilirubin | 1.5 | 1.2 | 1.1 | 0.3 | 0.4 | 0.7 | 1.1 | 1.8 | 1.9 |
| [mg/dL] | | | | | | | | | |
| Lipase [U/L] | 28 | 42 | 64 | 36 | 33 | 20 | 20 | 18 | 28 |
| Troponin I | 2,107 | 2,317 | 1,211 | 1,516 | 2,229 | 4,476 | n/a | n/a | n/a |
| [pg/mL] | | | | | | | | | |
| Potassium | 4.6 | 4.4 | 4.6 | 4.9 | 3.7 | 4.1 | 4.2 | 4.2 | 4.3 |
| [mmol/L] | | | | | | | | | |
| Sodium | 139 | 141 | 142 | 146 | 143 | 146 | 142 | 144 | 143 |
| [mmol/L] | | | | | | | | | |
| Calcium | 1.11 | 1.19 | 1.21 | 1.14 | 1.19 | 1.16 | 1.33 | 1.15 | 1.19 |
| [mmol/L] | | | | | | | | | |
| Chloride | 107 | 107 | 107 | 112 | 108 | 110 | 106 | 106 | 105 |
| [mmol/L] | | | | | | | | | |
| Glucose | 185 | 180 | 180 | 87 | 126 | 156 | 137 | 207 | 147 |
| [mg/dL] | | | | | | | | | |
| pH | 7.38 | 7.39 | 7.33 | 7.24 | 7.28 | 7.44 | 7.46 | 7.49 | 7.44 |
| Base excess | −3.6 | 1.5 | 1.8 | −6 | −4 | 4.3 | −1.5 | 4.8 | 5.9 |
| [mmol/L] | | | | | | | | | |
| Quick | 102 | n/a | n/a | 85 | n/a | n/a | 46 | 48 | 62 |
| [%] | | | | | | | | | |
| PTT | 34 | n/a | n/a | 34 | n/a | n/a | 42 | 42 | 38 |
| [sec] | | | | | | | | | |
| Thrombin time | 14.6 | n/a | n/a | 17.6 | n/a | n/a | 16.9 | 17 | 19.7 |
| [sec] | | | | | | | | | |
| Fibrinogen | >3,70 | n/a | n/a | >3,70 | n/a | n/a | >3,70 | >3,70 | >3,70 |
| [g/L] | | | | | | | | | |
| Hemoglobin | 10.8 | 9.5 | 10 | 8.2 | 10 | 8.9 | 9.8 | 9.3 | 9 |
| [g/dL] | | | | | | | | | |
| Erythrocytes | 3.32 | 2.92 | 3.07 | 2.69 | 3.29 | 2.93 | 3.04 | 2.87 | 2.94 |
| [10⁹/μL] | | | | | | | | | |
| Hematocrit | 32.5 | 28.5 | 30.4 | 27.8 | 33.2 | 28.7 | 28.6 | 27.1 | 27.2 |
| [%] | | | | | | | | | |
| MCV | 98 | 98 | 99 | 103 | 101 | 98 | 94 | 94 | 93 |
| [fL] | | | | | | | | | |
| MCH | 32.5 | 32.5 | 32.6 | 30.5 | 30.4 | 30.4 | 32.2 | 32.4 | 30.6 |
| [pg] | | | | | | | | | |
| MCHC | 33.2 | 33.3 | 32.9 | 29.5 | 30.2 | 31 | 34.3 | 34.3 | 33.1 |
| [g/dL] | | | | | | | | | |
| RDW | 14.1 | 14.3 | 15.1 | 17.1 | 17.3 | 17.3 | 16.2 | 16.4 | 16.7 |
| [%] | | | | | | | | | |
| Leukocytes | 17.5 | 14.3 | 18.2 | 5 | 22.1 | 33.3 | 35.8 | 63 | 55 |
| [10⁹/L] | | | | | | | | | |
| Thrombocytes | 178,000 | 134,000 | 151,000 | 598,000 | 410,000 | 333,000 | 113,000 | 115,000 | 92,000 |
| [n/μL] | | | | | | | | | |

Surprisingly, despite the patients being extreme-critically ill, treatment with PCZ resulted in a rapid improvement with respect to several relevant clinical features.

Administration of PCZ led to a successful treatment of shock, e.g., a significant reduction of required dosing of the vasopressor norepinephrine. Prior to the treatment with PCZ, patient PCZ 01, patient PCZ 02, and patient PCZ 03 received norepinephrine doses of 0.8, 1.4, and 0.6 μg/kg/min (mean: 0.9 μg/kg/min), respectively. One day after the PCZ treatment, the required doses of norepinephrine dropped to 67%, and after 48 hours down to 31%, compared to the starting dose.

Lactate is an established biomarker in critical care. Blood lactate levels are increased due to inadequate tissue oxygenation and other processes (Bakker, Nijsten, and Jansen 2013). Increased lactate levels reflect increased morbidity and high mortality risk, and decrease of lactate levels overtime has been shown to be associated with a favorable outcome in diverse critically ill populations including post cardiac surgery, trauma patients, sepsis and septic shock, cardiogenic shock, cardiac arrest, respiratory failure, and others. (Vincent et al. 2016).

In the present study, all three patients presented with highly elevated levels of lactate, e.g., above the established clinical cut-off of 2 mmol/L. (Vieira, Petrova, and Moura 2022). Upon treatment with PCZ, levels steadily declined and reached non-critical concentrations within 48 hours after start of treatment (FIG. 43). Median levels of lactate in healthy subjects have been reported to be 1.03 mmol/L. (Sung et al. 2016).

Interleukin-6 (IL-6) is a biomarker of systemic inflammation which has been shown in a diverse population of critically ill patients to reflect the extent of organ dysfunction, need for organ-support therapies, hemodynamic dysfunction characterized by reduced systolic and diastolic arterial pressure, coagulopathy, and hepatic, and renal dysfunction. (Picod et al. 2022).

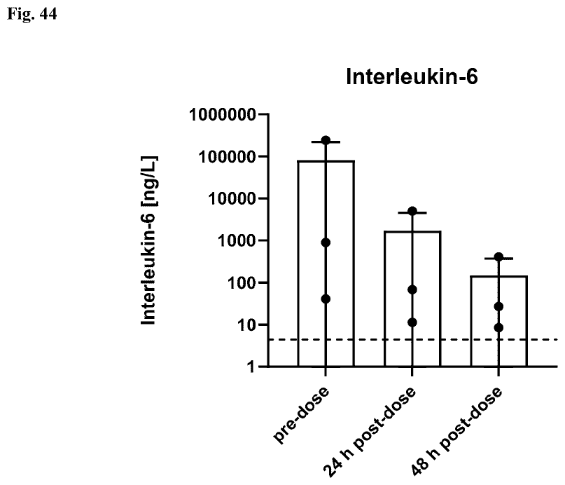

The extreme-critically patients of the present study exhibited significantly elevated IL-6 levels prior to treatment with PCZ. These levels steadily declined over the observation period of 48 hours (FIG. 44). For the general adult population, a median of 2.1 μg/mL and an upper reference range of 5.9 μg/mL have been reported earlier. (Alende-Castro, V, Alonso-Sampedro, M, Gude, F, Gonzalez-Quintela 2021).

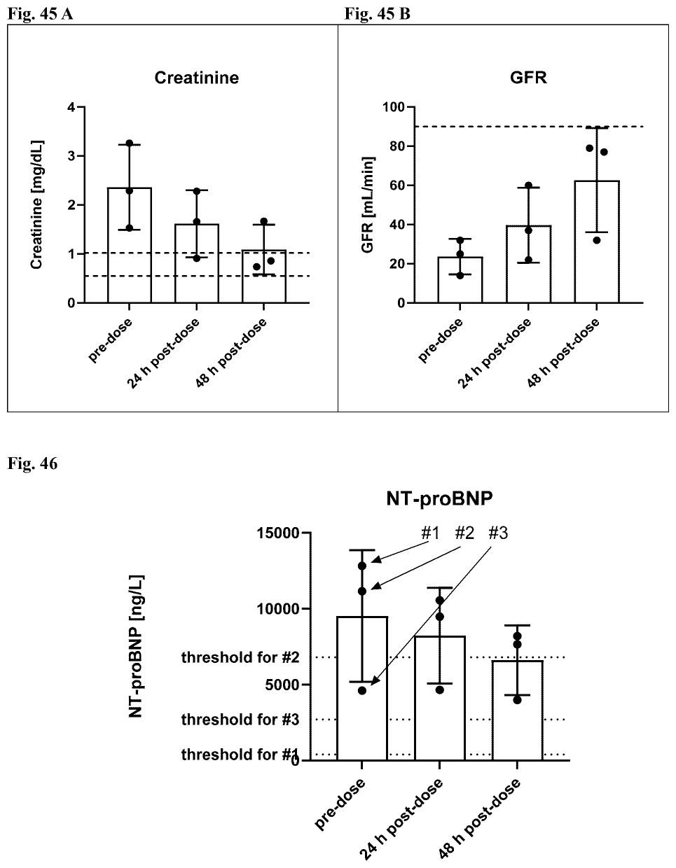

The effect of PCZ on the kidney function was assessed. All patients suffered from severely impaired kidney function, likely due to acute kidney injury, as reflected by high levels of blood creatinine and a very low glomerular filtration rate (GFR). They all required renal replacement therapy (RRT). Upon treatment with PCZ, both measures of kidney function improved steadily (FIG. 45 A and FIG. 45 B). Kidney function is considered impaired when the GFR is below 90 mL/min/1.73 m² (Levey et al. 2005). Reference intervals for serum creatinine concentrations have been reported as 0.72-1.18 mg/dL (2.5^{th} to 97^{th} percentile) for adult men and 0.55-1.02 mg/dL (2.5^{th} to 97^{th} percentile) for adult women (Ceriotti et al. 2008).

Additionally, the effect of PCZ on the heart function was assessed by measuring N-terminal prohormone of brain natriuretic peptide (NT-proBNP). NT-proBNP is an inactive prohormone fragment stemming from the same precursor peptide as the active B-type natriuretic peptide (BNP). Both BNP and NT-proBNP levels in the blood are used for screening, diagnosis of acute congestive heart failure (CHF) and may be useful to establish prognosis in heart failure, as both markers are typically higher in patients with worse outcome. (Samad, Malempati, and Restini 2023). The plasma concentrations of both BNP and NT-proBNP are also typically increased in patients with asymptomatic or symptomatic left ventricular dysfunction and are associated with coronary artery disease, myocardial ischemia, and severity of aortic valve stenosis. (Samad, Malempati, and Restini 2023). All three patients presented with supranormal levels of NT-proBNP, indicating an impaired heart function. Upon treatment with PCZ, levels of NT-proBNP declined steadily (FIG. 46). Normal reference ranges of NT-proBNP are dependent on age and sex and have been reported in detail by Welsh et al. 2022, see Table 37.

TABLE 37
Reference values for the 99^{th} percentile of NT-proBNP among
18,356 participants with no cardiovascular disease.

| | NT-proBNP [pg/L] |
|---|---|
| Females | |

| | | |
|---|---|---|
| <30 | y | 270 |
| 30-39 | y | 274 |
| 40-49 | y | 310 |
| 50-59 | y | 414 |
| 60-69 | y | 602 |
| 70-79 | y | 1353 |
| ≥80 | y | 3523 |

Males

| | | |
|---|---|---|
| <30 | y | 148 |
| 30-39 | y | 146 |
| 40-49 | y | 213 |
| 50-59 | y | 288 |
| 60-69 | y | 536 |
| 70-79 | y | 1695 |
| ≥80 | y | 6792 |

In particular embodiments of the present invention, one or more of said cut-offs for NT-proBNP are applied. The person skilled in the art would be aware of how and when to apply the respective cut-offs and how to determine the age and sex of a patient. The term “supranormal level of NT-proBNP” means that the respective level of a patient is above a respective cut-off as described above.

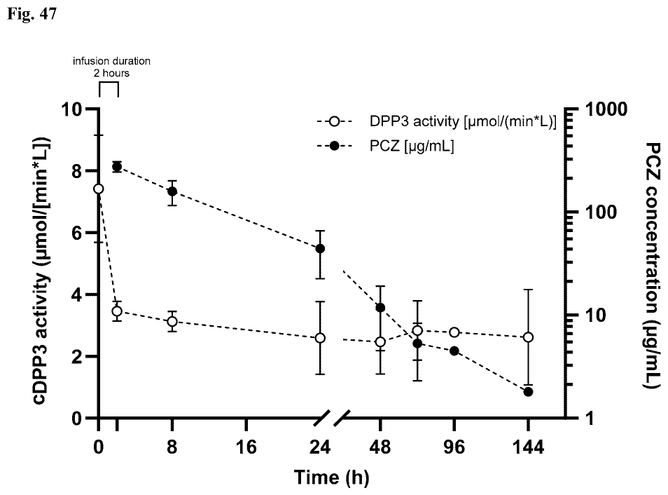

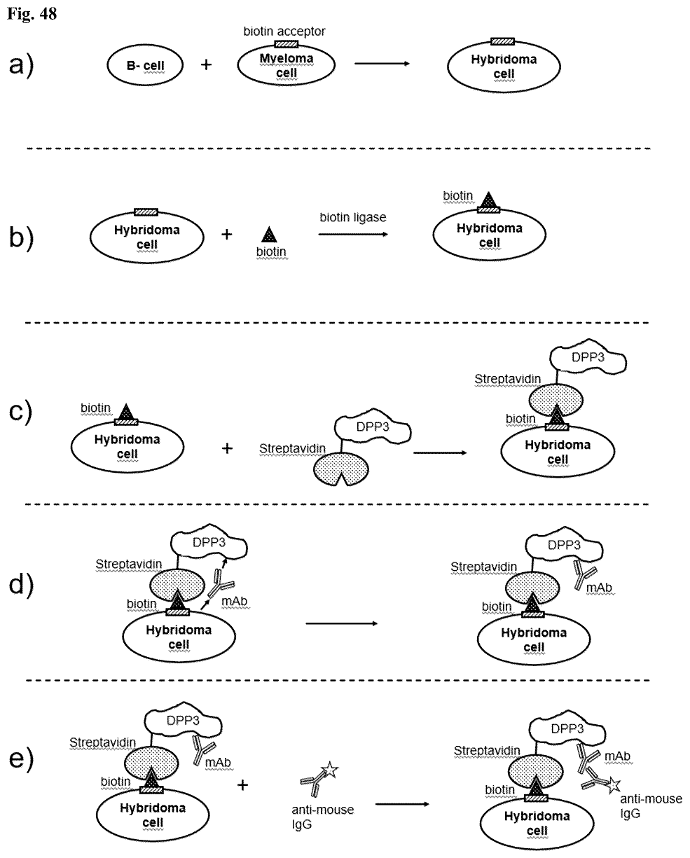

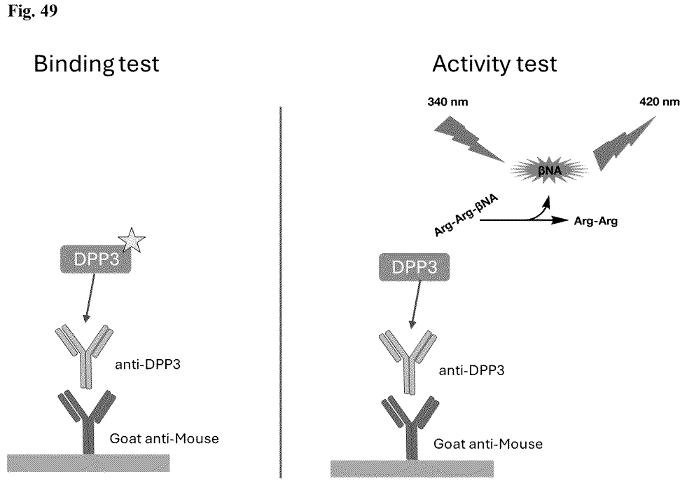

Finally, time courses of mean PCZ concentration and mean cDPP3 activity after administration of PCZ are shown in FIG. 47 and Table 38.

Cₘₐₓ of PCZ was observed immediately after the end of PCZ infusion (mean=276 μg/mL), and mean PCZ concentrations decreased to 44.3 μg/mL (16.1% of the Cₘₐₓ concentration) 24 hours after start of PCZ infusion, to 11.8 μg/mL (4.3% of the Cₘₐₓ concentration) 48 hours after start of PCZ infusion, and to 5.3 μg/mL (1.9% of the Cₘₐₓ concentration) 72 hours after start of PCZ infusion. At 48 hours individual PCZ concentrations were 18.6, 4.1, and 12.8 μg/mL, respectively, and at 72 hours individual PCZ concentrations were 7.9, 2.0, and 6.1 μg/mL, respectively.

The cDPP3 activity was significantly lower after the administration of PCZ compared to pre-dose cDPP3 activity: The minimum of decrease of cDPP3 activity was observed at 48 hours after start of PCZ infusion (33.3% compared pre-dose cDPP3 activity). After 48 hours, the cDPP3 activity started to rise again.

TABLE 38
Mean PCZ concentration and cDPP3 activity in extreme-critically
ill patients with refractory shock and multi-organ failure
(n = 3) after dosing with 10 mg/kg PCZ infused over
the first two hours (n.d. = not detectable).

| Time | PCZ | cDPP3 activity |
|---|---|---|
| [h] | [μg/mL] | [μmol/(min*L)] |
| 0 (pre-dose) | n.d. | 7.4 |
| 2 | 275.8 | 3.5 |
| 8 | 158.7 | 3.1 |
| 24 | 44.3 | 2.6 |
| 48 | 11.8 | 2.5 |
| 72 | 5.3 | 2.8 |
| 96 | 4.5 | 2.8 |
| 144 | 1.8 | 2.6 |

### Example 17—Development of Anti-DPP3 Antibodies with a High Success Rate Using SELMA

One method for the development of monoclonal antibodies against a specific antigen is the primary screening of hybridoma cells, wherein those cells that produce the antibodies of interest must be identified, selected, and separated from the other cells.

In this Example 17, an alternative method is described that will capture the released antibody on the cell surface of the corresponding hybridoma cell. It is expected that doing will enable the rapid identification and selection of the desired hybridomas while discarding the others, thereby resulting in a high success rate. SELMA™ technology (Single-cell ELISA-linked MACS/Affinity) has been developed and shown to achieve a high success rate (see WO2015161835, which is incorporated by reference; Listek, M, et al., 2020, Scientific Reports, 10(1), 1664; Listek, M, et al., 2023, ACS Applied Materials & Interfaces, 15(37), 43219-43222). In this Example 17, the SELMA method is utilized to identify, select, and isolate hybridoma cell lines producing monoclonal anti-DPP3 antibodies with a high success rate.

### Overview

A schematic overview is provided in FIG. 48. The SELMA method uses genetically engineered myeloma cells for the generation of hybridoma cells that express the specific engineered protein on the surface of the cell (the features of this protein are described in more detail below). These modified myeloma cells are fused with the B-cells of immunized mice to create a new line of transgenic hybridoma cells possessing the new artificial surface protein. The overall effect of these changes is to allow the new hybridomas to “capture” the antibodies they produce. The captured antibody and the corresponding cell may be labelled with a fluorescent dye and thus be sorted from the pool of hybridoma cells. This allows the identification of the secreted antibodies and the corresponding mother cell in one simple screening step. By an automated flow cytometry-based cell sort, the desired antibody producing cells may be selected with a high success rate that reduces experimentation and timelines, minimizing trial and error. Using the SELMA method, the antigen attaches to the marker on the cell surface. In turn, the antibody binds to the immobilized antigen-thus physically connecting the antibody to its producing cell.

### Method:

To induce antigen-specific B lymphocytes in mice, the mice are immunized with purified native human DPP3 (e.g., SEQ ID NO: 1) or with a DPP3 peptide (e.g. SEQ ID NO: 2) (4TEEN4 Pharmaceuticals, Hennigsdorf, Germany). The primary immunization contains 100 g of DPP3 and is administered intraperitoneally together with an adjuvant. After 4-8 weeks, a second immunization without adjuvant is performed, and 7 days later the spleen cells of the mice are isolated and counted. At the same time, cells of the modified myeloma cell line intended for the fusion are also prepared and used in the appropriate amount for the fusion, as described below.

The genetic modification of the myeloma cells comprises the transfection with a vector encoding a specific protein expressed at the cell surface: The genetic construct contains a truncated variant of the human immature epidermal growth factor (EGF)-receptor (EGF-R), a hemagglutinin epitope (HA) and a biotin acceptor peptide (AP; “AviTag”). To express the HA-AP-EGF-receptor fusion protein on the surface of myeloma cells, the signal peptide of the immature human EGF-receptor is inserted at the N-terminus of the cloned hemagglutinin epitope (HA) containing a biotin acceptor peptide (AP) sequence, and a truncated variant of the mature humane EGF-receptor (aa 1-651) at its C-terminus. The construct is controlled by the human elongation factor 1-alpha (EF1α)-promoter.

Spleen cells and myeloma cells are used in a ratio of 3:1 for a fusion. For this, the cells are washed and mixed together in fusion buffer (125 mM NaCl, 5 mM KCl, 4 mM CaCl₂, 2.5 mM MgCl₂, and 5 mM Tris-HCl, pH 7.4, adjusted). The cell pellet is resuspended in 200 μl fusion buffer and mixed with 200 μl PEG8000 solution. The cell solution is then transferred to an electroporation cuvette with an electrode spacing of 0.2 cm and the electroporation is carried out. The cells are fused by electroporation at a DC voltage of 500-700 V and a pulse duration of 20με. The cells then incubate for another 3 min in the cuvette and are then added to selection medium: DMEM, with 20% fetal calf serum, 2 mM glutamine, 50 M beta-mercaptoethanol, 200 M hypoxanthine, 12 M azaserine, 32 M 2′ deoxythymidine, and 5 g/mL puromycin, and are transferred and cultured until sorted.

In preparation of the in vitro biotinylation of the cells via the transgenically mediated acceptor peptide, the cell supernatant is discarded and the cells are washed twice with PBS supplemented with 5 mM MgCl₂. Following that, the cells are incubated with a mixture consisting of 5 mM MgCl₂, 1 mM ATP, 10 M biotin, and 0.789 g per 10⁶ cells BirA biotin ligase (GeneCopoeia™ Source BioScience LifeSciences, Nottingham UK) for 30 min. To get rid of the reaction mixture, the cells are washed four times with PBS containing 5 mM MgCl₂. Thereafter, the cells are ready for being loaded with the DPP3-streptavidin conjugate.

The DPP3-streptavidin conjugate is prepared by coupling purified native human DPP3 (e.g., SEQ ID NO: 1) to streptavidin via the homobifunctional crosslinker glutaraldehyde. 3 mg of DPP3 are mixed with 2 mg of streptavidin and then mixed with 0.25% glutaraldehyde in 1 mL 1×PBS for 2 hours at 4° C. The reaction mixture is then dialyzed against 5 L of 1×PBS for 1 hour.

For the coupling of the DPP3-streptavidin conjugate to the cells, 33 g of the DPP3-streptavidin conjugate are added to the previously biotinylated 10⁶ cells and are incubated for 20 minutes at 22° C. The cells are then centrifuged and taken up in fresh cell culture medium. The cells are incubated for four hours at 37° C. and 8% CO₂ to start production of antibodies. The cells are transferred to a reaction vessel and pelleted at 200 g for 8 min with a 1% bovine serum albumin solution to which 2 mM ethylenediaminetetraacetic acid (EDTA) has been added. The cell pellet is again resuspended in blocking solution (1×PBS, 5% neonatal calf serum) and labeled with fluorescein isothiocyanate goat anti-mouse antibody (Jackson ImmunoResearch Europe Ltd., Ely, UK) in a concentration of 1 g per 10⁶ cells; incubation time is for 1 hour on ice in the dark.

This is followed by another washing step and the cell pellets are taken up in 300 μl buffer for a fluorescence-activated cell sorter, namely 0.5% bovine serum albumin, 0.01% NaN₃ in PBS. For the fluorescence-activated cell sorting, a BD FACS Aria III and Flow Jo software are used. The positively labeled cells that produce the desired antibody that has bound its antigen DPP3 on the cell are then sorted out by the sorting function of the flow cytometer. The fraction obtained will be pelleted by centrifugation.

The cell pellet is then taken up in complete medium, RPMI 1640, 10% fetal calf serum, 2 mM glutamine, 50 M beta-mercaptoethanol, and seeded onto 96-well cell culture plates. The cell culture plates already contain a feeder cell population that is isolated from the mouse peritoneum, which supports the growth of the freshly fused and sorted hybridoma cells.

The antibodies harvested from the cell culture supernatants are later re-tested for binding to DPP3. Antibodies from the vast majority of cell cultures will test positive for binding to the DPP3 antigen.

### Example 18: Development of Anti-DPP3 Antibodies and Selection Based on their Inhibitory Activity

One method for the development and selection of monoclonal antibodies against inter alia a conjugated peptide derived from the DPP3 amino acid sequence SEQ ID NO: 2 is described herein (see, e.g., Paragraph [0255], above) and additionally described in WO2017182561, which is incorporated by reference.

In this Example, a new method is described that will allow for the selection of inhibitory antibodies directly based on functional features. By utilizing this method, it is expected that almost any monoclonal antibody generated using SEQ ID NO: 2 as the immunogen, which is nested within the catalytic site, will have the ability to strongly inhibit DPP3 enzymatic activity in vitro.

### Peptide/Conjugate for Immunization:

The SEQ ID NO: 2 DPP3 peptide for immunization (peptides & elephants, Hennigsdorf, Germany) was synthesized with an additional N-terminal cysteine residue—as compared to the corresponding amino acid sequence in human DPP3—for conjugation of the peptide to bovine thyroglobulin (BTG). The peptide was covalently linked to BTG by using Sulfolink-coupling gel (Perbio-science, Bonn, Germany). The coupling procedure was performed according to the instruction manual of Perbio-science. (Thermo Scientific, Cat. No. 20401 (MAN0011276_SulfoLnk_Coupling_Resin_UG.pdf)) Procedure for Immobilizing a Peptide or Protein Having Free Sulfhydryls

### A. Additional Materials Required

Column: Choose a glass or plastic column size appropriate for the volume of SulfoLink Resin to be used. The Disposable Column Trial Pack (Product No. 29925) contains accessories plus two each of three different column sizes, appropriate for 0.5-10 mL resin bed volumes. Alternatively, several centrifuge-ready Thermo Scientific Pierce Columns are available for resin bed volumes from 25 μl to 10 mL.
Coupling Buffer: 50 mM Tris, 5 mM EDTA-Na; pH 8.5. Prepare a volume equal to 20 times the volume of SulfoLink Resin to be used.
Quenching Reagent: L-cysteine-HCl (Product No. 44889)
Wash Solution: 1M sodium chloride (NaCl)
Storage Buffer: Phosphate-buffered saline (PBS) or other suitable buffer containing 0.05% sodium azide (NaN 3)

### B. Prepare SulfoLink Resin Column

1. Equilibrate SulfoLink Coupling Resin and all other reagents to room temperature. 2. Stir or swirl bottle to evenly suspend the resin, and then use a wide-bore pipette to transfer an
appropriate volume of the 50% resin slurry to an empty column. For example, transfer 2 mL of resin slurry to obtain a 1 mL resin bed.
3. Equilibrate column with four resin-bed volumes of Coupling Buffer. Replace the bottom column cap. Note: When using gravity-flow columns, do not allow the resin bed to become dry at any time throughout the procedure. Add more solution or replace the bottom cap on the column whenever the buffer drains down to the top of the resin bed.

### C. Couple Peptide/Protein to Resin

1. Dissolve prepared (i.e., reduced) peptide/protein in Coupling Buffer and add it to the column. Use 1-2 mL of peptide or protein solution per milliliter of SulfoLink Coupling Resin. If desired, retain a small amount of the peptide or protein solution for later comparison to the coupling reaction flow-through fraction to estimate coupling efficiency.
2. Replace the top cap and mix column (by rocking or end-over-end mixing) at room temperature for 15 minutes.
3. Stand the column upright and incubate the column at room temperature for an additional 30 minutes without mixing.
4. Sequentially remove top and bottom column caps and allow the solution to drain from the column into a clean tube.
5. Place the column over a new collection tube and wash column with three resin-bed volumes of Coupling Buffer.
6. Determine the coupling efficiency by comparing the protein/peptide concentrations (e.g., by absorbance at 280 nm) of the noncoupled fraction (Step 4) to the starting sample (Step 1).

### D. Block Nonspecific Binding Sites on Resin

1. Replace the bottom cap on column. Prepare a solution of 50 mM L-Cysteine-HCl in Coupling Buffer.
2. Add one resin-bed volume of 50 mM cysteine solution to the column. Mix for 15 minutes at room temperature, and incubate the reaction without mixing for an additional 30 minutes.

### E. Washing the Column

1. Sequentially remove the top and bottom caps and allow the column to drain.
2. Wash the column with at least six resin-bed volumes of Wash Solution (1M NaCl).
3. Wash the column with two resin-bed volumes of degassed Storage Buffer.

### Immunization of Mice, Immune Cell Fusion and Screening:

Five Balb/c mice were intraperitoneally (i.p.) injected with 100 μg DPP3-peptide-BTG-conjugate at day 0 (emulsified in TiterMax® Gold adjuvant (Sigma-Aldrich, Taufkirchen, Germany), 100 μg at day 14 (emulsified in complete Freund's adjuvant), and 50 μg at days 21 and 28 (emulsified in incomplete Freund's adjuvant). On day 49 the animals received an intravenous (i.v.) injection of 50 μg DPP3-peptide-BTG-conjugate dissolved in saline. Three days later, the mice were sacrificed and the immune cell fusion was performed.

For the immune cell fusion, splenocytes from the immunized mice and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium (RPMI 1640 culture medium supplemented with 20% fetal calf serum and HAT-Supplement). After one week, the HAT medium was replaced with HT medium for three passages followed by returning to the normal cell culture medium.

For the primary screening of cell culture supernatants a dual test procedure was applied as follows, which comprised measurement of binding to DPP3, and, in parallel, measurement of inhibitory enzymatic activity: The available total amount of 200 μL cell culture supernatant per hybridoma cell line candidate was split into half and subjected to a binding test and an inhibition test, respectively, as shown in FIG. 49. Two previously described anti-DPP3 antibodies were used as controls (murine antibodies #2555 and #1967 mentioned in Table 3 in WO2017182561; 4TEEN4 Pharmaceuticals, Henningsdorf, Germany).

### Binding Test:

96-well microtiter plates (white; high binding; Greiner Bio-One International GmbH, Austria) were coated with goat anti-mouse IgG (Arista, Fortis Life Science, Boston, Massachusetts, USA), as follows: 100 μl of goat anti-mouse IgG diluted at 5 μg/mL in coating buffer (71.5 mM NaHCO₃, 28.5 mM Na₂CO₃) were added per well, and plates were incubated at 4° C. overnight. Plates were rinsed once with 300 μL/well blocking buffer (6.5 mM KH₂PO₄, 3.5 mM Na₂HPO₄, 3% Karion, 0.5% BSA), and another 300 μL of blocking buffer were added, and plates were incubated for one hour at 22° C. Blocking solution was discarded, and plates were washed once with wash buffer (20 mM PBS, pH 7.4, 0.1% Tween 20).

100 μL of each undiluted cell culture supernatant were added and incubated for 90 minutes at 22° C. under agitation at 600 rpm to capture the IgG from the supernatant.

Due to the limited and defined binding capacity of the goat anti-mouse IgG solid phase, this step ensured that the same amounts of antibodies per supernatant were immobilized for further testing, as excess amounts beyond the binding capacity were discarded, making further analysis independent from the varying original antibody concentrations in the supernatants.

After washing of the plates with wash buffer (4×350 μL per well), human native DPP3, labeled with acridinium NHS-ester was added. Purified human native DPP3 (SEQ ID NO: 1; 4TEEN4 Pharmaceuticals, Hennigsdorf, Germany) was labeled with acridinium NHS-ester as follows: 100 μg (100 μl) of a purified human native DPP3 (1 mg/ml in PBS, pH 7.4) were mixed with 10 μl acridinium NHS-ester (1 mg/ml in acetonitrile, InVent GmbH, Germany; EP 0 353 971) and incubated for 30 min at room temperature. Labelled DPP3 was purified by gel-filtration HPLC on Shodex Protein 5 μm KW-803 (Showa Denko, Japan). The purified labeled DPP3 was diluted in assay buffer (PBS, 5 g/L bovine serum albumin, 0.075 g/L murine IgG, 1 g/L bovine IgG, 0.1% Tween 20, pH 7.4). The final concentration was approximately 7*10⁶ relative light units (RLU) of labeled compound (approximately 20 ng labeled DPP3) per 100 μl. The labeled acridinium ester chemiluminescence was measured as described here. Labeled DPP3 was added to the wells. After incubation for 90 minutes at 22° C. under agitation at 600 rpm, the plates were washed with wash buffer (4×350 μL per well), and solid phase-bound acridinium ester chemiluminescence was measured by using a Centro LB 960 luminometer (Berthold Technologies GmbH & Co. KG).

### Inhibition Test:

96-well microtiter plates (black; high binding; Greiner Bio-One International GmbH, Austria) were coated with goat anti-mouse IgG (Arista, Fortis Life Science, Boston, Massachusetts, USA), as follows: 100 μl of goat anti-mouse IgG diluted at 5 μg/mL in coating buffer (71.5 mM NaHCO₃, 28.5 mM Na₂CO₃) were added per well, and plates were incubated at 4° C. overnight. Plates were rinsed once with 300 μL/well blocking buffer (6.5 mM KH₂PO₄, 3.5 mM Na₂HPO₄, 3% Karion, 0.5% BSA), and another 300 μL of blocking buffer were added, and plates were incubated for one hour at 22° C. The blocking buffer solution was discarded, and plates were washed once with wash buffer (20 mM PBS, pH 7.4, 0.1% Tween 20).

100 μL of each undiluted cell culture supernatant were added and incubated for 90 minutes at 22° C. under agitation at 600 rpm to capture the IgG from the supernatant.

Due to the limited and defined binding capacity of the goat anti-mouse IgG solid phase, this step ensured that the same amounts of antibodies per supernatant were immobilized for further testing, as excess amounts beyond the binding capacity were discarded, making further analysis independent from the varying original antibody concentrations in the supernatants.

After washing of the plates with wash buffer (4×350 μL per well), 100 μl of purified human native DPP3 was added per well (1 μg/mL in PBS, 1 g/L bovine serum albumin, 0.075 g/L murine IgG, 1 g/L bovine IgG, pH 7.4), and plates were incubated for 90 minutes at 22° C. under agitation at 600 rpm. Plates were washed with wash buffer (4×350 μL per well), and 100 μL per well of substrate solution was added (100 μM Arg-Arg-B-naphthylamide in 50 mM Tris, 50 μM CoCl₂, 0.125% Triton-X, pH 7.8). After adding substrate, the increase of fluorescence was monitored for 1 h at 37° C. in Twinkle LB 970 microplate fluorometer (Berthold Technologies GmbH & Co. KG, Germany) using excitation wavelength of 340 nm and emission wavelength of 410 nm. Slopes of increasing fluorescence, determined as relative fluorescence units (RFU) per minute, represent the enzymatic activity of immobilized DPP3, and the RFU are directly proportional with molar amount of the B-naphthylamine generated (76,994,123 RFU/μmol).

Relating Inhibition Test Results with Binding Test Results:

Cell culture supernatants with no binding test signal above background were discarded for further analysis, as these do not contain detectable anti-DPP3 antibodies. For all others, the enzymatic DPP3 activity determined in the inhibition test (RFU/min) was divided by the binding test signal (RLU). This ratio represents the relative inhibitory activity of an anti-DPP3 antibody: A non-inhibitory or weakly inhibiting antibody would give a binding signal and a high DPP3 enzymatic activity signal. In contrast, a strongly inhibiting antibody would give a binding signal and a low DPP3 enzymatic activity signal. As discussed below, known weakly inhibiting as well as strong inhibiting anti-DPP3 antibodies have been run in parallel to the cell culture supernatants to facilitate interpretation of the test results obtained for cell culture supernatants.

### Results of Testing:

For the validation of the binding test and the inhibition test described above, available strongly inhibiting (AK1967) and a weakly inhibiting (AK2555; CDRs are SEQ ID NO: 63 to SEQ ID NO: 68 as shown herein below; heavy and light chain are SEQ ID NO: 69 to SEQ ID NO: 70 as shown herein below) mouse monoclonal anti-DPP3 antibodies were assessed in both tests at several dilutions in cell culture medium (see Table 39). For both antibodies it was evident that results in both tests were essentially independent from the antibody concentration used: Signals obtained for 100 ng/mL and 10,000 ng/mL differed by maximally a factor of approximately 2. Antibody AK2555 was generated using full-length DPP3 (SEQ ID NO: 1) as the immunogen in mice and is a murine antibody. (See, e.g., the incorporated by reference U.S. Pat. No. 11,726,094, Example 3.)

For the strongly inhibiting antibody (AK1967) the ratio of activity divided by binding was approximately 0.0006, whereas for the weakly inhibiting antibody (AK2555), it was approximately 1,000-fold higher (Table 39). These results demonstrated that the design of both tests and the evaluation fulfill the needs of the analysis in this Example.

TABLE 39
Results of binding test and inhibition test obtained
for a strongly inhibiting (AK1967) and a weakly
inhibiting (AK2555) monoclonal anti-DPP3 antibody.

| | B | Activity | Activity |
|---|---|---|---|
| conc. (ng/mL) | [RLU] | [RFU/min] | [RFU/min]/B[RLU] |
| | AK1967 | | |
| 10,000 | 259,961 | 199 | 0.00076 |
| 1,000 | 265,486 | 172 | 0.00065 |
| 100 | 277,933 | 92 | 0.00033 |
| 0 | 5,558 | <10 | |
| | AK2555 | | |
| 10,000 | 25,200 | 19,129 | 0.75908 |
| 1,000 | 19,895 | 17,979 | 0.90374 |
| 100 | 15,288 | 11,912 | 0.77918 |
| 0 | 4,521 | <10 | |

Results of the binding test and the inhibition test obtained for the cell culture supernatants are shown in Table 40. Only cell culture supernatants exhibiting more than 3-fold the binding observed in the absence of the cell culture supernatant, e.g. the buffer control providing the unspecific binding (UB), were considered, and ranked by their binding test signal. From the 768 cell culture supernatants obtained from the cell fusion, 30 exhibited a binding signal that was at least 3-fold higher than the UB. Of note, all of these, without exception, showed a very low activity signal, and the ratio of activity divided by binding was similarly low as observed for the strongly inhibiting reference antibody PCZ, which had been developed earlier by the same immunization technique as described in the example.

TABLE 40
Results of binding test and inhibition test obtained for the cell
culture supernatants in the primary screening of the example.

| # | B [RLU] | Activitiy [RFU/min] | Activity [RFU/min]/B[RLU] |
|---|---|---|---|
| 1 | 167,266 | 30.8 | 0.00018 |
| 2 | 157,202 | 18.1 | 0.00012 |
| 3 | 136,222 | 10.5 | <0.0001 |
| 4 | 117,209 | <10 | <0.0001 |
| 5 | 116,590 | <10 | <0.0001 |
| 6 | 113,650 | 16.0 | 0.00014 |
| 7 | 106,625 | <10 | <0.0001 |
| 8 | 99,530 | <10 | <0.0001 |
| 9 | 97,849 | <10 | <0.0001 |
| 10 | 88,388 | <10 | <0.0001 |
| 11 | 72,938 | <10 | <0.0001 |
| 12 | 70,060 | <10 | <0.0001 |
| 13 | 67,268 | <10 | <0.0001 |
| 14 | 66,322 | <10 | <0.0001 |
| 15 | 39,150 | <10 | <0.0001 |
| 16 | 38,655 | <10 | <0.0001 |
| 17 | 35,348 | <10 | <0.0001 |
| 18 | 35,260 | <10 | <0.0001 |
| 19 | 34,270 | <10 | <0.0001 |
| 20 | 30,061 | <10 | <0.0001 |
| 21 | 29,907 | <10 | <0.0001 |
| 22 | 29,556 | <10 | <0.0001 |
| 23 | 29,325 | <10 | <0.0001 |
| 24 | 27,335 | <10 | <0.0001 |
| 25 | 25,282 | <10 | <0.0001 |
| 26 | 22,634 | <10 | <0.0001 |
| 27 | 21,624 | <10 | <0.0001 |
| 28 | 21,250 | <10 | <0.0001 |
| 29 | 20,866 | <10 | <0.0001 |
| 30 | 19,526 | <10 | <0.0001 |

The results demonstrated that by application of the test designs described in this Example, monoclonal anti-DPP3 antibodies may be selected directly based on the functional feature that is their ability to inhibit DPP3 enzymatic activity, and secondly, that any monoclonal antibody raised against SEQ ID NO: 2 has the ability to strongly inhibit DPP3 enzymatic activity in vitro.

List of SEQ ID NOs
hDPP3 aa 1-737
SEQ ID No. 1
MADTQYILPNDIGVSSLDCREAFRLLSPTERLYAYHLSRAAWYGGLAVLL
QTSPEAPYIYALLSRLFRAQDPDQLRQHALAEGLTEEEYQAFLVYAAGVY
SNMGNYKSFGDTKFVPNLPKEKLERVILGSEAAQQHPEEVRGLWQTCGEL
MFSLEPRLRHLGLGKEGITTYFSGNCTMEDAKLAQDFLDSQNLSAYNTRL
FKEVDGEGKPYYEVRLASVLGSEPSLDSEVTSKLKSYEFRGSPFQVTRGD
YAPILQKVVEQLEKAKAYAANSHQGQMLAQYIESFTQGSIEAHKRGSRFW
IQDKGPIVESYIGFIESYRDPFGSRGEFEGFVAVVNKAMSAKFERLVASA
EQLLKELPWPPTFEKDKFLTPDFTSLDVLTFAGSGIPAGINIPNYDDLRQ
TEGFKNVSLGNVLAVAYATQREKLTFLEEDDKDLYILWKGPSFDVQVGLH
ELLGHGSGKLFVQDEKGAFNFDQETVINPETGEQIQSWYRSGETWDSKFS
TIASSYEECRAESVGLYLCLHPQVLEIFGFEGADAEDVIYVNWLNMVRAG
LLALEFYTPEAFNWRQAHMQARFVILRVLLEAGEGLVTITPTTGSDGRPD
ARVRLDRSKIRSVGKPALERFLRRLQVLKSTGDVAGGRALYEGYATVTDA
PPECFLTLRDTVLLRKESRKLIVQPNTRLEGSDVQLLEYEASAAGLIRSF
SERFPEDGPELEEILTQLATADARFWKGPSEAPSGQA
hDPP3 aa 474-493 (N-Cys) - immunization peptide
with additional N-terminal Cystein
SEQ ID No. 2
CETVINPETGEQIQSWYRSGE
hDPP3 aa 477-482 -an epitope of anti-DPP3 antibody
SEQ ID No. 3
INPETG
hDPP3 aa 480-483
SEQ ID No. 4
ETGE
variable region of murine
anti-DPP3 antibody in heavy chain
SEQ ID No. 5
QVTLKESGPGILQPSQTLSLTCSFSGFSLSTSGMSVGWIRQPSGKGLEWL
AHIWWNDNKSYNPALKSRLTISRDTSNNQVFLKIASVVTADTGTYFCARN
YSYDYWGQGTTLTVSS
variable region of murine anti-DPP3 antibody
in light chain
SEQ ID No. 6
DVVVTQTPLSLSVSLGDPASISCRSSRSLVHSIGSTYLHWYLQKPGQSPK
LLIYKVSNRFSGVPDRFSGSGSGTDFTLKISRVEAEDLGVYFCSQSTHVP
WTFGGGTKLEIK
CDR1 of anti-DPP3 antibody in heavy chain
SEQ ID No. 7
GFSLSTSGMS
CDR2 of anti-DPP3 antibody in heavy chain
SEQ ID No. 8
IWWNDNK
CDR 3 of anti-DPP3 antibody in heavy chain
SEQ ID No. 9
ARNSYDY
CDR1 of anti-DPP3 antibody in light chain
SEQ ID No. 10
RSLVHSIGSTY
CDR2 of anti-DPP3 antibody in light chain
KVS
CDR3 of anti-DPP3 antibody in light chain
SEQ ID No. 11
SQSTHVPWT
humanized anti-DPP3 antibody - heavy chain
sequence (IgG1K backbone)
SEQ ID No. 12
MDPKGSLSWRILLFLSLAFELSYGQITLKESGPTLVKPTQTLTLTCTFSG
FSLSTSGMSVGWIRQPPGKALEWLAHIWWNDNKSYNPALKSRLTITRDTS
KNQVVLTMTNMDPVDTGTYYCARNYSYDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSDI
AVEWESNGQPENNYKTTPPVLDSDGSFFLYSKLTVDKSRWQQGNVFSCSV
MHEALHNHYTQKSLSLSPG
humanized anti-DPP3 antibody - light chain
sequence (IgG1K backbone)
SEQ ID No. 13
METDTLLLWVLLLWVPGSTGDIVMTQTPLSLSVTPGQPASISCKSSRSLV
HSIGSTYLYWYLQKPGQSPQLLIYKVSNRFSGVPDRFSGSGSGTDFTLKI
SRVEAEDVGVYYCSQSTHVPWTFGGGTKVEIKRTVAAPSVFIFPPSDEQL
KSGTASVVCLLNNFYPREAKVQWKVDNALQSGNSQESVTEQDSKDSTYSL
SSTLTLSKADYEKHKVYACEVTHQGLSSPVTKSFNRGEC
human DPP3 amino acid 450-455
SEQ ID No. 14
HELLGH
human DPP3 507-512
SEQ ID No. 15
EECRAE
humanized anti-DPP3 antibody - heavy chain
sequence (IgG1K backbone) after
treatment with Papain (Fab fragment)
SEQ ID NO: 16
MDPKGSLSWRILLFLSLAFELSYGQITLKESGPTLVKPTQTLTLTCTFSG
FSLSTSGMSVGWIRQPPGKALEWLAHIWWNDNKSYNPALKSRLTITRDTS
KNQVVLTMTNMDPVDTGTYYCARNYSYDYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTH
humanized anti-DPP3 antibody (PCZ) - heavy
chain sequence (IgG1K backbone)
SEQ ID NO: 17
QITLKESGPTLVKPTQTLTLTCTFSGFSLSTSGMSVGWIRQPPGKALEWL
AHIWWNDNKSYNPALKSRLTITRDTSKNQVVLTMTNMDPVDTGTYYCARN
YSYDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG
humanized anti-DPP3 antibody (PCZ)- light
chain sequence (IgG1K backbone)
SEQ ID NO: 18
DIVMTQTPLSLSVTPGQPASISCKSSRSLVHSIGSTYLYWYLQKPGQSPQL
LIYKVSNRFSGVPDRFSGSGSGTDFTLKISRVEAEDVGVYYCSQSTHVPWT
FGGGTKVEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQ
GLSSPVTKSFNRGEC
humanized anti-DPP3 antibody - heavy chain
N-terminal signal peptide sequence
SEQ ID NO: 19
MDPKGSLSWRILLFLSLAFELSYG
humanized anti-DPP3 antibody - light chain
N-terminal signal peptide sequence
SEQ ID NO: 20
METDTLLLWVLLLWVPGSTG
humanized anti-DPP3 antibody (PCZ) - heavy chain
sequence (IgG1K backbone)
after treatment with Papain (Fab fragment)
SEQ ID NO: 21
QITLKESGPTLVKPTQTLTLTCTFSGFSLSTSGMSVGWIRQPPGKALEWL
AHIWWNDNKSYNPALKSRLTITRDTSKNQVVLTMTNMDPVDTGTYYCARN
YSYDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTH
Epitope mapping peptide 1
SEQ ID NO: 22
bio-AFNFDQETVINPETGEQIQSWYRSG
Epitope mapping peptide 2
SEQ ID NO: 23
bio-AFNFDQETVINPETGEQIQ
Epitope mapping peptide 3
SEQ ID NO: 24
bio-AFNFDQETVINPETGEQI
Epitope mapping peptide 4
SEQ ID NO: 25
bio-AFNFDQETVINPETGEQ
Epitope mapping peptide 5
SEQ ID NO: 26
bio-AFNFDQETVINPETGE
Epitope mapping peptide 6
SEQ ID NO: 27
bio-AFNFDQETVINPETG
Epitope mapping peptide 7
SEQ ID NO: 28
bio-AFNFDQETVINPET
Epitope mapping peptide 8
SEQ ID NO: 29
bio-AFNFDQETVINPE
Epitope mapping peptide 9
SEQ ID NO: 30
bio-AFNFDQETVINP
Epitope mapping peptide 10
SEQ ID NO: 31
bio-AFNFDQETVIN
Epitope mapping peptide 11
SEQ ID NO: 32
ETGEQIQSWYK-bio
Epitope mapping peptide 12
SEQ ID NO: 33
PETGEQIQSWYK-bio
Epitope mapping peptide 13
SEQ ID NO: 34
NPETGEQIQSWYK-bio
Epitope mapping peptide 14
SEQ ID NO: 35
INPETGEQIQSWYK-bio
Epitope mapping peptide 15
SEQ ID NO: 36
VINPETGEQIQSWYK-bio
Epitope mapping peptide 16
SEQ ID NO: 37
TVINPETGEQIQSWYK-bio
Epitope mapping peptide 17
SEQ ID NO: 38
ETVINPETGEQIQSWYK-bio
Spinorphin
SEQ ID NO: 39
LVVYPWT
Tynorphin
SEQ ID NO: 40
VVYPW
Spinorphin Derivative
SEQ ID NO: 41
AVYPW
Spinorphin Derivative
SEQ ID NO: 42
FIVPW
Spinorphin Derivative
SEQ ID NO: 43
FVAPW
Spinorphin Derivative
SEQ ID NO: 44
FVYPW
Spinorphin Derivative
SEQ ID NO: 45
GVYPW
Spinorphin Derivative
SEQ ID NO: 46
IVYPW
Spinorphin Derivative
SEQ ID NO: 47
LVVPW
Spinorphin Derivative
SEQ ID NO: 48
LVVYP
Spinorphin Derivative
SEQ ID NO: 49
LVVYPW
Spinorphin Derivative
SEQ ID NO: 50
LVYPW
Spinorphin Derivative
SEQ ID NO: 51
SVYPW
Spinorphin Derivative
SEQ ID NO: 52
VVYP
Spinorphin Derivative
SEQ ID NO: 53
VVYPWT
Spinorphin Derivative
SEQ ID NO: 54
VYPW
Spinorphin Derivative
SEQ ID NO: 55
VYPWT
Spinorphin Derivative
SEQ ID NO: 56
WVYPW
Spinorphin Derivative
SEQ ID NO: 57
YAIPW
Spinorphin Derivative
SEQ ID NO: 58
YPWT
Spinorphin Derivative
SEQ ID NO: 59
YSIPW
Spinorphin Derivative
SEQ ID NO: 60
YSVPW
Spinorphin Derivative
SEQ ID NO: 61
YVYPW
DPP3-peptide
SEQ ID NO: 62
ETVINPETGEQIQSWYR
CDR1 of AK2555 heavy chain
SEQ ID NO: 63
GYYIH
CDR2 of AK2555 heavy chain
SEQ ID No. 64
EINPSTGGTNYNQKFKG
CDR 3 of AK2555 heavy chain
SEQ ID No. 65
SYYGIYSWFAY
CDR1 of AK2555 light chain
SEQ ID No. 66
KASQDIKSYLS
CDR2 of of AK2555 light chain
SEQ ID No. 67
YATSLAD
CDR3 of AK2555 light chain
SEQ ID No. 68
LQHGESPRT
AK2555 - heavy chain sequence
SEQ ID No. 69
EVQLQQSGPELVKPGASVKISCKASGYSFTGYYIHWVKQSPENSLEWIGE
INPSTGGTNYNQKFKGKATLTVDKSSSTAYMQLKSLTSEESAVYYCTRSY
YGIYSWFAYWGQGTLVTVSA
AK2555 - light chain sequence
SEQ ID No. 70
DIKMTQSPSSMYASLGERVTITCKASQDIKSYLSWYQQKPWKSPKTLIYY
ATSLADGVPSRFSGSGSGQDYSLTISSLESDDTATYYCLQHGESPRTFGG
GTKLEIK

### REFERENCES CITED

Abramid, M., et al., 2000, Human and Rat Dipeptidy Peptidase III: Biochemical and Mass Spectrometric Arguments for Similarities and Differences, Biological Chemistry 381 (12): 1233-43. https://doi.org/10.1515/BC.2000.151.
Abramid, M. & Agid, D., 2022, Survey of Dipeptidyl Peptidase III Inhibitors: From Small Molecules of Microbial or Synthetic Origin to Aprotinin, Molecules 27 (9), https://doi.org/10.3390/molecules27093006.
Aissaoui, N., et al., 2020. “Trends in Cardiogenic Shock Complicating Acute Myocardial Infarction.” European Journal of Heart Failure 22 (4): 664-72.
Almagro & Fransson, 2008, Humanization of antibodies. Front Biosci. 2008 Jan. 1; 13:1619-33.
Arrigo, M. & Mebazaa, A., 2015, Understanding the differences among inotropes, Intensive Care Med. 41, 912-915.
Arrigo, M., et al., 2021, “Optimising Clinical Trials in Acute Myocardial Infarction Complicated by Cardiogenic Shock: A Statement from the 2020 Critical Care Clinical Trialists Workshop.” The Lancet Respiratory Medicine 9 (10): 1192-1202.
Arrigo, M., et al., 2023, “Current and Future Trial Design in Refractory Cardiogenic Shock.” European Journal of Heart Failure 25 (5): 609-15.
Barsun, M., et al., 2007, Human dipeptidyl peptidase III acts as a post-proline-cleaving enzyme on endomorphins. Biol. Chem. 388, 343-348.
Becher, P. M., et al., 2018, Venoarterial Extracorporeal Membrane Oxygenation for Cardiopulmonary Support: Insights from a German Registry. Circulation 138, 2298-2300.
Bellomo, R., et al., 2020, Renin and survival in patients given angiotensin II for catecholamine-resistant vasodilatory shock a clinical trial, Am. J. Respir. Crit. Care Med. 202, 1253-1261.
Berg, D. D., et al., 2019, Epidemiology of Shock in Contemporary Cardiac Intensive Care Units: Data from the Critical Care Cardiology Trials Network Registry. Circ. Cardiovasc. Qual. Outcomes 12, 1-10.
Blet, A., et al., 2021, Monitoring Circulating Dipeptidyl Peptidase 3 (DPP3) Predicts Improvement of Organ Failure and Survival in Sepsis: A Prospective Observational Multinational Study, Critical Care 25 (1): 1-10.
Chawla, L. S., et al., 2014, Intravenous angiotensin II for the treatment of high-output shock (ATHOS trial): A pilot study, Crit. Care 18, 1-9.
Chioncel, O., et al., 2020, Epidemiology, Pathophysiology and Contemporary Management of Cardiogenic Shock—a Position Statement from the Heart Failure Association of the European Society of Cardiology, European Journal of Heart Failure 22 (8): 1315-41.
Corréa, T. D., et al., 2015, Angiotensin II in septic shock, Crit. Care 19, 1-6.
Cruz-Diaz, N., et al., 2016, Identification of dipeptidyl peptidase 3 as the Angiotensin-(1-7) degrading peptidase in human HK-2 renal epithelial cells, Peptides 83, 29-37.
Cuinet, J., et al., 2020, Cardiogenic shock elicits acute inflammation, delayed eosinophilia, and depletion of immune cells in most severe cases, Sci. Rep. 10, 1-9.
De Backer, Daniel, et al., 2010, Comparison of Dopamine and Norepinephrine in the Treatment of Shock. N. Engl. J. Med. 362, 779-789.
Deniau, B. et al., 2020, Circulating dipeptidyl peptidase 3 is a myocardial depressant factor: dipeptidyl peptidase 3 inhibition rapidly and sustainably improves haemodynamics. Eur. J. Heart Fail. 1-10 (Note: this article was electronically published in 2019, so to avoid confusion with the other Deniau article, this article will be referred to as “Deniau 2019”).
Deniau, B., et al., 2020, Circulating Dipeptidyl Peptidase 3 Is a Myocardial Depressant Factor: Dipeptidyl Peptidase 3 Inhibition Rapidly and Sustainably Improves Haemodynamics, European J. of Heart Failure 22 (2): 290-99, https://doi.org/10.1002/ejhf.1601 (“Deniau 2020 Circulating DPP3”).
Deniau, B., et al., 2020, Inhibition of circulating dipeptidyl-peptidase 3 restores cardiac function in a sepsis-induced model in rats: A proof of concept study. PLoS One 15, 1-12 (“Deniau 2020 Inhibition”).
Dhanda, S., et al., 2008, Hydrolysis of Various Bioactive Peptides by Goat Brain Dipeptidylpeptidase-III Homologue, Cell Biochemistry and Function: Cellular Biochemistry and Its Modulation by Active Agents or Disease 26 (3): 339-45.
Diakos, N. A., et al., 2021, Systemic Inflammatory Burden Correlates with Severity and Predicts Outcomes in Patients with Cardiogenic Shock Supported by a Percutaneous Mechanical Assist Device, J. Cardiovasc. Transl. Res. 14, 476-483.
Dostal, D. E., et al., 1997, Molecular Mechanisms of Angiotensin II in Modulating Cardiac Function: Intracardiac Effects and Signal Transduction Pathways, J. of Molecular and Cellular Cardiology 29 (11): 2893-2902.
Ellis, S. & Nuenke, J. M., 1967, Dipeptidyl Arylamidase III of the Pituitary. Purification and Characterization, J. of Biological Chemistry 242 (20): 4623-29.
Ferrario, C. M. Role of Angiotensin II in Cardiovascular Disease—Therapeutic Implications of More Than a Century of Research. J. Renin-Angiotensin-Aldosterone Syst. 7, 3-14 (2006).
Garcia, B., et al., 2022, Myocardial effects of angiotensin II compared to norepinephrine in an animal model of septic shock, Crit. Care 26, 1-13.
Garcia, B., et al., 2024, Inhibition of Circulating Dipeptidyl-Peptidase 3 by Procizumab in Experimental Septic Shock Reduces Catecholamine Exposure and Myocardial Injury, Intensive Care Medicine Experimental 12 (1): 53.
Geevarghese, M., et al., 2023, Role of adrenergic receptors in shock, Front. Physiol. 14, 1-9.
Goldberg, M. R., et al., 1993, Effects of losartan on blood pressure, plasma renin activity, and angiotensin II in volunteers, Hypertension 21, 704-713.
Hochman, J. S., et al., 1999, Early revascularization in acute myocardial infarction complicated by cardiogenic shock. N. Engl. J. Med. 341, 625-634.
Hochman, J. S., et al., 2000, J. Am. Coll. Cardiol. 36: 1063-1070.
Hochman, J. S., 2003, Cardiogenic shock complicating acute myocardial infarction: Expanding the paradigm, Circulation 107, 2998-3002.
Hochman, J. S., et al., 2006, Early Revascularization Improves Long-Term Survival for Cardiogenic Shock Complicating Acute Myocardial Infarction. JAMA—J. Am. Med. Assoc. 295, 2511-2515.
Iborra-Egea, O., et al., 2020, An outlook on biomarkers in cardiogenic shock, Curr. Opin. Crit. Care 26 , 392-397.
Jentzer, J. C., et al., 2020, Systemic Inflammatory Response Syndrome Is Associated With Increased Mortality Across the Spectrum of Shock Severity in Cardiac Intensive Care Patients, Circ. Cardiovasc. Qual. Outcomes 13, E006956.
Jha, S. et al. Dipeptidyl Peptidase 3 modulates the renin-Angiotensin system in mice. J. Biol. Chem. 295 , 13711-13723 (2020).
Jones, T. H. D. & Kapralou, A., 1982, A Rapid Assay for Dipeptidyl Aminopeptidase III in Human Erythrocytes, Analytical Biochemistry 119 (2): 418-23 (Jones & Kapralou 1982).
Kaufmann, P. et al. A novel and highly efficient purification procedure for native human dipeptidyl peptidase 3 from human blood cell lysate. PLoS One 14, e0220866 (2019).
Khanna, A., et al., 2017, Angiotensin II for the treatment of vasodilatory shock, N. Engl. J. Med. 377 , 419-430.
Kono, T., et al., 1979, Effects of Angiotensin in Normal Enzyme Men Inhibitor, tensin I-converting enzyme inhibitor, SQ men was studied previously by other in-used and angiotensin I (AI) was given as multiple intravenous injections. Moreover, they mostly examined changes. Endocrinol. Jpn. 26, 411-418.
Koya & Paul 2018. Shock. StatPearls [Internet]. Treasure Island (FL): StatPearls Publishing; 2019-2018 Oct. 27.
Kumar, P., et al., 2016, Substrate Complexes of Human Dipeptidyl Peptidase III Reveal the Mechanism of Enzyme Inhibition, Scientific Reports 6 (December 2015): 1-10. https://doi.org/10.1038/srep23787.
Lee, C. M. & Snyder, S. H., 1982, Dipeptidyl-aminopeptidase III of rat brain. Selective affinity for enkephalin and angiotensin, J. Biol. Chem. 257, 12043-12050.
Listek, M., et al., 2020, A novel selection strategy for antibody producing hybridoma cells based on a new transgenic fusion cell line, Scientific Reports, 10(1), 1664.
Listek, M., et al., 2023, Comment on “Monoclonal Antibody Discovery based on precise selection of single transgenic hybridomas with an On-Cell-Surface and Antigen-Specific Anchor,” ACS Applied Materials & Interfaces, 15(37), 43219-43222.
Malovan, G., et al., 2023, The Emerging Role of Dipeptidyl Peptidase 3 in Pathophysiology, FEBS Journal 290 (9): 2246-62, https://doi.org/10.1111/febs.16429.
Mazzocco, C., et al., 2006, Identification and Characterization of Two Dipeptidyl-Peptidase III Isoforms in Drosophila Melanogaster, FEBS Journal 273 (5): 1056-64, https://doi.org/10.1111/j.1742-4658.2006.05132.x.
Mebazaa, A., et al., 2018, Management of cardiogenic shock complicating myocardial infarction, Intensive Care Med. 44, 760-773.
Menale, C., et al., 2019, Absence of Dipeptidyl Peptidase 3 increases oxidative stress and causes bone loss, J. Bone Miner. Res. jbmr.3829, doi: 10.1002/jbmr.3829.
Méndez H., et al., 2023, F. Biomarkers as Prognostic Predictors and Therapeutic Guide in Critically Ill Patients: Clinical Evidence, J. Pers. Med. 13.
Montero, S. & Bayes-Genis, A., 2022, The overlooked tsunami of systemic inflammation in post-myocardial infarction cardiogenic shock, Eur. J. Prev. Cardiol. 29, 2052-2054.
Muzafarova, T., et al., 2023, The Prognosis of Cardiogenic Shock Following Acute Myocardial Infarction—an Analysis of 2693 Cases From a Prospective Multicenter Registry,” Deutsches Arzteblatt International 120 (31-32): 538.
Ohkubo, I., et al., 1999, Dipeptidyl Peptidase III from Rat Liver Cytosol: Purification, Molecular Cloning and Immunohistochemical Localization.
Ozden, O., et al., 2022, A Novel Indicator of Myocardial Injury after Acute Myocardial Infarction: ‘DPP-3’, Clin. Appl. Thromb. 28, 1-4.
Pang, X. et al. Novel Therapeutic Role for Dipeptidyl Peptidase III in the Treatment of Hypertension. Hypertension 68, 630-641 (2016).
Picod, A., et al., 2021, Alteration of the Renin-Angiotensin-Aldosterone System in Shock: Role of the Dipeptidyl, Am J Respir Crit Care Med. 203, 526-527.
Pöss, J., et al., 2023, Circulating dipeptidyl peptidase 3: new hope for a specific treatment to improve prognosis in cardiogenic shock?Eur. Heart J. 44, 3872-3874.
Prajapati, S. C. & Chauhan, S. S., 2011, Dipeptidyl Peptidase III: A Multifaceted Oligopeptide N-End Cutter, FEBS Journal 278 (18): 3256-76. https://doi.org/10.1111/j.1742-4658.2011.08275.x.
Rehfeld, L., et al., 2019, Novel Methods for the Quantification of Dipeptidyl Peptidase 3 (DPP3) Concentration and Activity in Human Blood Samples. J. Appl. Lab. Med. 3, 943-953.
Reynolds, H. R. & Hochman, J. S., 2008, Cardiogenic shock current concepts and improving outcomes. Circulation 117, 686-697.
Rittirsch, D., Huber-Lang, M. S., Flierl, M. A. & Ward, P. A. Immunodesign of experimental sepsis by cecal ligation and puncture. Nat. Protoc. 4, 31-36 (2009).
Roks, A., et al., 1997, The Renin-Angiotensin System and Vascular Function. The Role of Angiotensin II, Angiotensin-Converting Enzyme, and Alternative Conversion ofAngiotensin I, Heart and Vessels, 119-24.
Sato, H., et al., 2003, Activity of DPP III in human cerebrospinal fluid derived from patients with pain, Masui 52, 257-63.
Shimamori, Y., et al., 1986, Purification and Characterization of Dipeptidyl Aminopeptidase III from Human Placenta, Chem. Pharm. Bull. (Tokyo). 34, 3333-3340.
Singer, M., et al., 2016, The third international concesus definitions for Sepsis and Septic shock. JAMA 315 (8): 801-810.
Soussi, S. et al. Distinct host-response signatures in circulatory shock: a narrative review. Intensive Care Med. Exp. 11, (2023)
Takagi, K., et al., 2019, Circulating dipeptidyl peptidase 3 and alteration in haemodynamics in cardiogenic shock: results from the OptimaCC trial, Eur. J. Heart Fail. 1-8, doi:10.1002/ejhf.1600.
Thiele, H., et al., 2010, Shock in acute myocardial infarction: The Cape Horn for trials?Eur. Heart J. 31 , 1828-1835.
Thiele, H., et al., 2012, Intraaortic balloon counterpulsation in acute myocardial infarction complicated by cardiogenic shock: Design and rationale of the Intraaortic Balloon Pump in Cardiogenic Shock II (IABP-SHOCK II) trial. Am. Heart J. 163, 938-945.
Thiele, H., et al., 2012, Intraaortic Balloon Support for Myocardial Infarction with Cardiogenic Shock, New England Journal of Medicine 367 (14): 1287-96.
Thiele, H., et al., 2015, Management of cardiogenic shock, Eur. Heart J. 36, 1223-1230.
Thiele, H., et al., 2017, PCI Strategies in Patients with Acute Myocardial Infarction and Cardiogenic Shock. N. Engl. J. Med. 377, 2419-2432.
Thiele, H., et al., 2017, Percutaneous short-term active mechanical support devices in cardiogenic shock: A systematic review and collaborative meta-analysis of randomized trials, Eur. Heart J. 38, 3523-3531.
Thiele, H., et al., 2023, Extracorporeal Life Support in Infarct-Related Cardiogenic Shock. N. Engl. J. Med. 1286-1297 doi:10.1056/nejmoa2307227.
Udupa & Shetty, 2018, Indian J Respir Care 7: 67-72.
Van Diepen, S., et al., 2017. Contemporary Management of Cardiogenic Shock: A Scientific Statement from the American Heart Association, Circulation Vol. 136.
Van Lier, D. & Pickkers, P., 2021, Circulating biomarkers to assess cardiovascular function in critically ill, Curr. Opin. Crit. Care 27, 261-268.
Van Lier, D., et al., 2022, Circulating dipeptidyl peptidase 3 and bio-adrenomedullin levels are associated with impaired outcomes in critically ill COVID-19 patients: a prospective international multicentre study, ERJ Open Res 9.
Van Lier, D., et al., 2023, Associations of dipeptidyl-peptidase 3 with short-term outcome in a mixed admission ICU-cohort, J. Crit. Care 78, 154383.
Vandenberg, J. I., et al., 1985, Enkephalin Degradation by Human Erythrocytes and Hemolysates Studied Using 1H NMR Spectroscopy, Archives of Biochemistry and Biophysics 242 (2): 515-22, https://doi.org/10.1016/0003-9861(85)90238-3.
Vanha-Perttula, T., 1988, Dipeptidyl Peptidase III and alanyl aminopeptidase in the human seminal plasma: Origin and biochemical properties, Clin. Chim. Acta 177, 179-195.
Vergaro, G. et al., 2016, Inhibition of Galectin-3 Pathway Prevents Isoproterenol-Induced Left Ventricular Dysfunction and Fibrosis in Mice, Hypertension 67, 606-612.
Vincent, J. L. & De Backer, D., 2014, N. Engl. J. Med. 370(6): 583.
Vincent, J. L. & De Backer, D., 2013, Circulatory shock. N. Engl. J. Med. 369, 1726-1734.
Wenzl, F. A., et al., 2023, Dipeptidyl Peptidase 3 plasma levels predict cardiogenic shock and mortality in acute coronary syndromes, Eur. Heart J. 44, 3859-3871.
Ye, P., et al., 2022, DPP3: From biomarker to therapeutic target of cardiovascular diseases, Front. Cardiovasc. Med. 9.
Zhang, W., et al., 2014, Severe sepsis: Low expression of the renin-angiotensin system is associated with poor prognosis, Exp. Ther. Med. 7, 1342-1348.

What is claimed:

1. A formulation for administering an anti-DPP3 antibody or fragment thereof to a human, wherein the formulation comprises:
(a) the anti-DPP3 antibody or fragment thereof as an active ingredient;
(b) a stabilizer;
(c) an antioxidant; and
(d) a buffer;
wherein the formulation is a concentrated solution suitable for intravenous administration upon dilution.

2. The formulation of claim 1, wherein the stabilizer is selected from the group consisting of sugars, sugar alcohols, amino acids, surfactants, and stabilizing proteins.

3. The formulation of claim 2, wherein the stabilizer is sucrose, trehalose, or maltose.

4. The formulation of claim 2, wherein the stabilizer is mannitol or sorbitol.

5. The formulation of claim 2, wherein the stabilizer is arginine, histidine, glycine, proline, or methionine.

6. The formulation of claim 2, wherein the stabilizer is Polysorbate 20 (Tween 20), Polysorbate 80 (Tween 80) or Poloxamer 188.

7. The formulation of claim 2, wherein the stabilizer is human serum albumin or bovine serum albumin.

8. The formulation of claim 1, wherein the antioxidant is selected from the group consisting of methionine, sodium thiosulfate, platinum, N-acetyl-DL-tryptophan, ethylenediaminetetraacetic acid (EDTA) and diethylenetriaminepentaacetic acid (DTPA).

9. The formulation of claim 1, wherein the buffer is selected from the group consisting of histidine, phosphate-buffered saline, and tris-buffered saline.

10. The formulation of claim 1, wherein when the concentrated solution is diluted, such diluted solution upon intravenous administration provides a dose of the active ingredient of from about 3.0 mg/kg to about 12 mg/kg bodyweight.

11. The formulation of claim 1, wherein when the concentrated solution is diluted, such diluted solution upon intravenous administration provides a dose of the active ingredient of about 3.0 mg/kg, about 6.0 mg/kg, or about 12/mg/kg bodyweight.

12. The formulation of claim 1, wherein when the concentrated solution is diluted, such diluted solution upon intravenous administration provides a dose of the active ingredient from about 10 mg/kg to about 20 mg/kg bodyweight.

13. The formulation of claim 1, wherein when the concentrated solution is diluted, such diluted solution upon intravenous administration provides a dose of the active ingredient of about 10 mg/kg or about 20 mg/kg bodyweight.

14. The formulation of claim 1, wherein when the concentrated solution is diluted, such diluted solution upon intravenous administration provides for an infusion volume of about 1.0 to 1.2 mL/kg.

15. The formulation of claim 1, wherein the concentrated solution comprises:
(a) about 20 mg/mL of the active ingredient;
(b) about 90.7 mg/mL of the stabilizer;
(c) about 2.86 mg/mL of the antioxidant; and
(d) about 1.11 mg/mL of the buffer.

16. The formulation of claim 1, wherein anti-DPP3 antibody or fragment thereof has:
a heavy chain that comprises at least one CDR selected from the group comprising SEQ ID NO: 7, SEQ ID NO: 8, and SEQ ID NO: 9, or a sequence that is >95% identical to it; and
a light chain that comprises at least one CDR selected from the group comprising SEQ ID NO: 10, CDR2 comprising KVS, and SEQ ID NO: 11, or a sequence that is >95% identical to it.

17. The formulation of claim 1, wherein the anti-DPP3 antibody or fragment thereof comprises SEQ ID No. 18 and SEQ ID No. 21.

18. The formulation of claim 1, wherein the anti-DPP3 antibody or fragment thereof is a humanized anti-DPP3 antibody or fragment thereof.

19. The formulation of claim 1, wherein the anti-DPP3 antibody or fragment thereof comprises SEQ ID No. 17 and SEQ ID No. 18.


SEQUENCE LISTING (70 sequences)

SEQ ID NO: 1 - protein, 737 aa, Homo sapiens
MADTQYILPNDIGVSSLDCREAFRLLSPTERLYAYHLSRAAWYGGLAVLLQTSPEAPYIYALLSRLFRAQDPDQLRQHALAEGLTEEEYQAFLVYAAGVYSNMGNYKSFGDTKFVPNLPKEKLERVILGSEAAQQHPEEVRGLWQTCGELMFSLEPRLRHLGLGKEGITTYFSGNCTMEDAKLAQDFLDSQNLSAYNTRLFKEVDGEGKPYYEVRLASVLGSEPSLDSEVTSKLKSYEFRGSPFQVTRGDYAPILQKVVEQLEKAKAYAANSHQGQMLAQYIESFTQGSIEAHKRGSRFWIQDKGPIVESYIGFIESYRDPFGSRGEFEGFVAVVNKAMSAKFERLVASAEQLLKELPWPPTFEKDKFLTPDFTSLDVLTFAGSGIPAGINIPNYDDLRQTEGFKNVSLGNVLAVAYATQREKLTFLEEDDKDLYILWKGPSFDVQVGLHELLGHGSGKLFVQDEKGAFNFDQETVINPETGEQIQSWYRSGETWDSKFSTIASSYEECRAESVGLYLCLHPQVLEIFGFEGADAEDVIYVNWLNMVRAGLLALEFYTPEAFNWRQAHMQARFVILRVLLEAGEGLVTITPTTGSDGRPDARVRLDRSKIRSVGKPALERFLRRLQVLKSTGDVAGGRALYEGYATVTDAPPECFLTLRDTVLLRKESRKLIVQPNTRLEGSDVQLLEYEASAAGLIRSFSERFPEDGPELEEILTQLATADARFWKGPSEAPSGQA

SEQ ID NO: 2 - protein, 21 aa, Homo sapiens
CETVINPETGEQIQSWYRSGE

SEQ ID NO: 3 - protein, 6 aa, Homo sapiens
INPETG

SEQ ID NO: 4 - protein, 4 aa, Homo sapiens
ETGE

SEQ ID NO: 5 - protein, 116 aa, Synthetic construct
QVTLKESGPGILQPSQTLSLTCSFSGFSLSTSGMSVGWIRQPSGKGLEWLAHIWWNDNKSYNPALKSRLTISRDTSNNQVFLKIASVVTADTGTYFCARNYSYDYWGQGTTLTVSS

SEQ ID NO: 6 - protein, 112 aa, Synthetic construct
DVVVTQTPLSLSVSLGDPASISCRSSRSLVHSIGSTYLHWYLQKPGQSPKLLIYKVSNRFSGVPDRFSGSGSGTDFTLKISRVEAEDLGVYFCSQSTHVPWTFGGGTKLEIK

SEQ ID NO: 7 - protein, 10 aa, Synthetic construct
GFSLSTSGMS

SEQ ID NO: 8 - protein, 7 aa, Synthetic construct
IWWNDNK

SEQ ID NO: 9 - protein, 7 aa, Synthetic construct
ARNSYDY

SEQ ID NO: 10 - protein, 11 aa, Synthetic construct
RSLVHSIGSTY

SEQ ID NO: 11 - protein, 9 aa, Synthetic construct
SQSTHVPWT

SEQ ID NO: 12 - protein, 469 aa, Synthetic construct
MDPKGSLSWRILLFLSLAFELSYGQITLKESGPTLVKPTQTLTLTCTFSGFSLSTSGMSVGWIRQPPGKALEWLAHIWWNDNKSYNPALKSRLTITRDTSKNQVVLTMTNMDPVDTGTYYCARNYSYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 239 aa, Synthetic construct
METDTLLLWVLLLWVPGSTGDIVMTQTPLSLSVTPGQPASISCKSSRSLVHSIGSTYLYWYLQKPGQSPQLLIYKVSNRFSGVPDRFSGSGSGTDFTLKISRVEAEDVGVYYCSQSTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 6 aa, Homo sapiens
HELLGH

SEQ ID NO: 15 - protein, 6 aa, Homo sapiens
EECRAE

SEQ ID NO: 16 - protein, 247 aa, Synthetic construct
MDPKGSLSWRILLFLSLAFELSYGQITLKESGPTLVKPTQTLTLTCTFSGFSLSTSGMSVGWIRQPPGKALEWLAHIWWNDNKSYNPALKSRLTITRDTSKNQVVLTMTNMDPVDTGTYYCARNYSYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 17 - protein, 445 aa, Synthetic construct
QITLKESGPTLVKPTQTLTLTCTFSGFSLSTSGMSVGWIRQPPGKALEWLAHIWWNDNKSYNPALKSRLTITRDTSKNQVVLTMTNMDPVDTGTYYCARNYSYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 219 aa, Synthetic construct
DIVMTQTPLSLSVTPGQPASISCKSSRSLVHSIGSTYLYWYLQKPGQSPQLLIYKVSNRFSGVPDRFSGSGSGTDFTLKISRVEAEDVGVYYCSQSTHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 24 aa, Synthetic construct
MDPKGSLSWRILLFLSLAFELSYG

SEQ ID NO: 20 - protein, 20 aa, Synthetic construct
METDTLLLWVLLLWVPGSTG

SEQ ID NO: 21 - protein, 223 aa, Synthetic construct
QITLKESGPTLVKPTQTLTLTCTFSGFSLSTSGMSVGWIRQPPGKALEWLAHIWWNDNKSYNPALKSRLTITRDTSKNQVVLTMTNMDPVDTGTYYCARNYSYDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 22 - protein, 25 aa, Synthetic construct
  MOD_RES 1: note = N-terminal biotinylation
AFNFDQETVINPETGEQIQSWYRSG

SEQ ID NO: 23 - protein, 19 aa, Synthetic construct
  MOD_RES 1: note = N-terminal biotinylation
AFNFDQETVINPETGEQIQ

SEQ ID NO: 24 - protein, 18 aa, Synthetic construct
  MOD_RES 1: note = N-terminal biotinylation
AFNFDQETVINPETGEQI

SEQ ID NO: 25 - protein, 17 aa, Synthetic construct
  MOD_RES 1: note = N-terminal biotinylation
AFNFDQETVINPETGEQ

SEQ ID NO: 26 - protein, 16 aa, Synthetic construct
  MOD_RES 1: note = N-terminal biotinylation
AFNFDQETVINPETGE

SEQ ID NO: 27 - protein, 15 aa, Synthetic construct
  MOD_RES 1: note = N-terminal biotinylation
AFNFDQETVINPETG

SEQ ID NO: 28 - protein, 14 aa, Synthetic construct
  MOD_RES 1: note = N-terminal biotinylation
AFNFDQETVINPET

SEQ ID NO: 29 - protein, 13 aa, Synthetic construct
  MOD_RES 1: note = N-terminal biotinylation
AFNFDQETVINPE

SEQ ID NO: 30 - protein, 12 aa, Synthetic construct
  MOD_RES 1: note = N-terminal biotinylation
AFNFDQETVINP

SEQ ID NO: 31 - protein, 11 aa, Synthetic construct
  MOD_RES 1: note = N-terminal biotinylation
AFNFDQETVIN

SEQ ID NO: 32 - protein, 11 aa, Synthetic construct
  MOD_RES 11: note = C-terminal biotinylation
ETGEQIQSWYK

SEQ ID NO: 33 - protein, 12 aa, Synthetic construct
  MOD_RES 12: note = C-terminal biotinylation
PETGEQIQSWYK

SEQ ID NO: 34 - protein, 13 aa, Synthetic construct
  MOD_RES 13: note = C-terminal biotinylation
NPETGEQIQSWYK

SEQ ID NO: 35 - protein, 14 aa, Synthetic construct
  MOD_RES 14: note = C-terminal biotinylation
INPETGEQIQSWYK

SEQ ID NO: 36 - protein, 15 aa, Synthetic construct
  MOD_RES 15: note = C-terminal biotinylation
VINPETGEQIQSWYK

SEQ ID NO: 37 - protein, 16 aa, Synthetic construct
  MOD_RES 16: note = C-terminal biotinylation
TVINPETGEQIQSWYK

SEQ ID NO: 38 - protein, 17 aa, Synthetic construct
  MOD_RES 17: note = C-terminal biotinylation
ETVINPETGEQIQSWYK

SEQ ID NO: 39 - protein, 7 aa, Homo sapiens
LVVYPWT

SEQ ID NO: 40 - protein, 5 aa, Homo sapiens
VVYPW

SEQ ID NO: 41 - protein, 5 aa, Synthetic construct
AVYPW

SEQ ID NO: 42 - protein, 5 aa, Synthetic construct
FIVPW

SEQ ID NO: 43 - protein, 5 aa, Synthetic construct
FVAPW

SEQ ID NO: 44 - protein, 5 aa, Synthetic construct
FVYPW

SEQ ID NO: 45 - protein, 5 aa, Synthetic construct
GVYPW

SEQ ID NO: 46 - protein, 5 aa, Synthetic construct
IVYPW

SEQ ID NO: 47 - protein, 5 aa, Synthetic construct
LVVPW

SEQ ID NO: 48 - protein, 5 aa, Synthetic construct
LVVYP

SEQ ID NO: 49 - protein, 6 aa, Synthetic construct
LVVYPW

SEQ ID NO: 50 - protein, 5 aa, Synthetic construct
LVYPW

SEQ ID NO: 51 - protein, 5 aa, Synthetic construct
SVYPW

SEQ ID NO: 52 - protein, 4 aa, Synthetic construct
VVYP

SEQ ID NO: 53 - protein, 6 aa, Synthetic construct
VVYPWT

SEQ ID NO: 54 - protein, 4 aa, Synthetic construct
VYPW

SEQ ID NO: 55 - protein, 5 aa, Synthetic construct
VYPWT

SEQ ID NO: 56 - protein, 5 aa, Synthetic construct
WVYPW

SEQ ID NO: 57 - protein, 5 aa, Synthetic construct
YAIPW

SEQ ID NO: 58 - protein, 4 aa, Synthetic construct
YPWT

SEQ ID NO: 59 - protein, 5 aa, Synthetic construct
YSIPW

SEQ ID NO: 60 - protein, 5 aa, Synthetic construct
YSVPW

SEQ ID NO: 61 - protein, 5 aa, Synthetic construct
YVYPW

SEQ ID NO: 62 - protein, 17 aa, Homo sapiens
ETVINPETGEQIQSWYR

SEQ ID NO: 63 - protein, 5 aa, Synthetic construct
GYYIH

SEQ ID NO: 64 - protein, 17 aa, Synthetic construct
EINPSTGGTNYNQKFKG

SEQ ID NO: 65 - protein, 11 aa, Synthetic construct
SYYGIYSWFAY

SEQ ID NO: 66 - protein, 11 aa, Synthetic construct
KASQDIKSYLS

SEQ ID NO: 67 - protein, 7 aa, Synthetic construct
YATSLAD

SEQ ID NO: 68 - protein, 9 aa, Synthetic construct
LQHGESPRT

SEQ ID NO: 69 - protein, 120 aa, Synthetic construct
EVQLQQSGPELVKPGASVKISCKASGYSFTGYYIHWVKQSPENSLEWIGEINPSTGGTNYNQKFKGKATLTVDKSSSTAYMQLKSLTSEESAVYYCTRSYYGIYSWFAYWGQGTLVTVSA

SEQ ID NO: 70 - protein, 107 aa, Synthetic construct
DIKMTQSPSSMYASLGERVTITCKASQDIKSYLSWYQQKPWKSPKTLIYYATSLADGVPSRFSGSGSGQDYSLTISSLESDDTATYYCLQHGESPRTFGGGTKLEIK